SEQUENCE LISTING (205 sequences)

SEQ ID NO: 1 - DNA, 1565 bp, Aedes aegypti
atgtggatcccaacgacagtagccctcagcatggtggtagccttcgcggctgcagccatagaaagtaatccagcgaggccggttctgttcggaagtcagaagacagcgcctcctcgaggaccaccttccgatagtatcgtcgacaacggaaactacacggagtggcgtgtattcgatcaacggcaaagtcactcgaacgcccacagcgtggacatgttcccaatgcgatacgttagcaattcgaagttctatcgaccaggtggtcccattttcctgtttgttggaggaccttgggagttggagcagcattttgtcgaacaggggcattttgtggacttggctgaggagaacaacgcctttgtggtagccaatgagatgcgttattacggagagagcttgcctgtaccgaatgcatctcgcggaaacttgcgtttactccacattgtgcaagcatgtacggatattgccaggcttatcgtgcacataagatacgaagtgttgagagatcctaacgcaagagtgatcgtggctggtgtgggtttctccggctcacttgcacactggacacgactgcgttatcctcatctgattcatggagtttgggcgtccggtgcgatgttgcaagcaaacgaaaattacagagagtttgccgaagaggttggagagtatattcgacgatatggtggcaatgattgctacggtgcactctggagaggtttccgtacggccgagaatttgatcgatgcaggacagtcgcaaaccgttgataaattgttcaaggtatgcactccgatcaacggtaccaacccactggacgttgaagcatttttctacggtattttcaacgaagtcgtttcgaatactttgcgtcccaatctcagacagaacataagaaacatgtgtgatacgttgactcatgaagatcacgatagtagcctaacagggttggccagctggataactggtcagttcccagaagcagagtgccttgcaatggacttggaaagtatcgtacagctgttccaggaaaccgattggcagcacgatgtgcacaaatctggcgaacgacaatggttttaccaacgttgtaccgaactgggatggcctctgacggcagactcgatgaatcaaccgttcggtgtccgaatctcctctaatcttttccaacagctttgtcagcgagttttcgatggctggctcacttcggacgttttccggtcacttgttcgacagacaaatactttgtatggaggaaaccgaccggaaatgaggttcgtgttttacactcatggttcgctcgatccgtggaggttcaccggtgtaactacggtgctctacaacaacaactacgtcaatgtcattcgaggtgctatccatggagaagatttggcctccatttccgatctggactgggctgacttgaggcgcagcaaggaggaggttggtgaaaccattcgtcgctggttggagaggagagtttgaagaggcttacaattgcaattggaggattttcataagccacgtagataaaatttcctccatgtacacttttgttaacacaaaat

SEQ ID NO: 2 - DNA, 1735 bp, Aedes aegypti
ccaatccaaataccagcttcaccggcttccaagatcagctggacaataattagaaccggcttctcgcaacgctgctcgtttctccatagaccgtttacacttcgtccatccaacacacatccgtcatcagtttcgctctcgtgctctttctctccgaacctgcatcacccagtataactaatatgaatcctcccgcctatgcgcagaagacgtccaacgcaaacgacgacttcacgatgtcctacacgcgctcgccacaactcgaattcgagttgtttgttttcggttttttttggcaactcttccactagttctgaatgctggcggtgtggtgcggtactgcagtgagggggctagggttaattgcaaattgccaatgcttaattctgcgcgcgatgttagaaaagatcacggcgagttgtctaccattagacgttagtgtttcggtgactgtgacccagtgtgcatccttttcctgttgtgactgcagaaatggttcttctgaggggagatcctgtgaaattggcgcttccgctgctgatggtgttgagctacggaagttctgtgctgggaaatggactgatacggaacgactgtgtgtgtgatccgctgaatgccttcctcgactcgctgccgcttaatcgtgaacgaacagatcaattgctagagacggatggctatcagggaagagcacacagagttgtcacacaggatggctacgtgctgaagctgtatcagatttggcgcgatcaacagccagtggtgaactcaacacgtgggacgatcctgctgcagcacgggatcatgcattcttcatcggactggttagtgttgggaccgggtcgatcgatcgcctatcagttggtagaccttggttatgacgtgtggctggcaaacagtagatctacgatgaactcgcatcagcacgagaagttctgcacttgttcaaaggagttctgggactacagttggcacgaacaaggcttttacgatctgccggcgatgatcgacacggtcctgaacgaaactcagcagcagcgactccggttgttagtctactcagaaggtggagctatggcaatggtcatgctctcaacccgacccgaatacaacgataaactgatcgcgctggatgccatggctccggcagcgttcgtctccaacacctggtaccgctacctggccataccgttcgccaagatcccgaaagtcttccggtcggcttacgcggtatactcaactaacgaagtcacggtagaggcctgccaaacggagtaccagatatgctcggatctgtactttcaattcctgaacggcgagagcgtcggcatgaatcggtcctgggtggatcgaatctaccaggccatgccggcaggaggatccatcaaagaggtcctgcactacgtccagctgatctggacccgcaagttcgcgcctttcgatcacggtccgtcgaagaacctgcgactctacggacaacgaacgcctccggagtatccgctggatcgagtgtctgtgccggtgaacatccactacggactacgggataagatcgtcgatccggtgggagtgatgcggcttggttctcggctcatcaacagcccaagggttcgcatgaggccgtacgacgagctgcagcactcggactttatctacggcgatgccgcttacaatatggtgtacaaagatgttcttatgtggataacggaataaagtgaatgggttaatggagccg

SEQ ID NO: 3 - DNA, 716 bp, Aedes aegypti
agcgcttgccgtgagtagactgcgccactcgtttgcttcgaacttgttcataattagttgagtaaacaaaaatcgataggaagaagaatgtcgtttgtcatccgcaaaatcatctccactggcaaatgtcctaagcctttggccccttatagccaagcggtggttgccgatcgaaccgtgtactgctcgggggtcatcggtattgagttggacgaaccgaagctagtaaccggtggtgctgctgtgcaaactgccaaagctttggaacatctcacaaatctactcgccgaagctggctccggtatcgataaggtggtgaagacaaccgttctgctagctgacatggaagattacgcagctgttaatgaggagtacaaacgagttttcggaagcaactttccagcgagaacgtgttatgccgtatcgaaacttcctctaggtgctgctgttgagattgaagccgtcgggattgttggagacgtgaaacatgaaatgtgaatgttgaaatgtgtgcaagttttgctcaataaatataaatctcgtttgcaatcttcctccatttatgttcagtgttactatccgagcagtaacataatcaaacaagtatacatgttaagcaatatattactgcccaaaaaccatcttttgtaacacaattttattgcgattcagtgttagaatttaaatatttgaaaaatatataactctatacgctc

SEQ ID NO: 4 - DNA, 709 bp, Aedes aegypti
acaatttcctccttgttcaatagcacgtagctacttgtctagccatcagcaatcgtatcacatatgtgacgatacggtgagccatcctcctacaccctatagatagtggagcatatgcactttgatgtactaatcttcctagtatatcgctatataagcatttgatactaactagagtgaagtacagtccatcgcagcactctcaagtacccagaactgaacaaatgaccgccaccaaagagtttacccgccaggaagtggcgcttcgagacggcaagaacggtagtccaatatggatcatcattcgcgacgtagtgtacgatgtgaccagctacgtggacgaacatccgggagggccagaactggtcaccgatttcgccggccgggacggaacgaaggatttcgatgactttggccattccagtacggcgatggagatgctgaagcagtacaaagtcggcgagttgaattattgtgatcgagctaaatttcaaaagcaaggatcgaattggtcaccggatggagcttgtgttctgccggacaagaaacgcagcaagaagcggaagtttattttctgtggataaattgactaagatatagccgatggaacgttacagattgataattattcgtttaagaaactaagctactttatttattctatttaagtaatttcttgttatgatagctcaaatatacattacgtttt

SEQ ID NO: 5 - DNA, 1503 bp, Aedes aegypti
gtgaaagtgatcacatctcagcgggacgcggtgaaactgtgattgaactgatagaactgtgatcaagatcaactcaacgaagtagaatgttgtcggttctgtcgagagtggctcagcgggggaggagcttatcggctgtatccgcagtgcagcgctgttatgcatccacgggaggaaaggaaccaagcaaaacggctctgtacgaattccatcaggctcaaggtggaaaactggtagactttgccggttactggttgcccgtgcagtacagcgatcagagtattattaaatcgcatcattacacccgcgagtatggatctattttcgacgtgtcgcatatgctgcaaacatatttgagaggcaaggatgtcatcagttgctttgagtcgatttgtactgcagatataaagggactccgaaatggcactggaacgttgaccgtgttcaccaatggaaagggaggaattttggacgatctgattgtgagtcgagtgtcggaggatactttgtatgtggtctccaatgcatcccggaaggatacggatatggcagttatgaccgatgcagtggcatccttcaaatcccaaggcaaagacgtctcggtgcagttcctgtccagcgatgaccaatcactcctggcaatccaaggtccacatgccgtttcagtcctgcaaaagctgtgcaccaaagacctttcccgcttatacttcatgaataccacaacggacactattgccggcgtagaaaactgccgaatcactcgttgtggatacactggcgaagacggagtcgaaatttctatcccatccaacaaagccaccgacattgcttcagcgctcctggagcaaaaagtcggcaaccttaagttggcaggtcttggagctcgcgattccctccgactggaagccggcctatgcctgtacggcaacgacatcgacgagactacgacccccgttgaagctggccttctctggttggtggccaaacagcgccgcgcagaaaacaatttccccggctcggacaagatcaacaatcaaataaaaaacggcgtcactcgacgtcgcgtcggattcaagatgtccgcaggatcggcccccgcaaggcaacacgtggaaattttcgataacgaacatcacaaaatcggagagatcaccagcggctgtccgagcccgtgtctgcagcagaacatcgccatgggatacattcgggaggaatcgaaaaaggtgggcaccgaagtgacgctcaagattcgggacaagttctaccacagccaggtcgccaagatgccattcgtcgcgacacattattatcagcaaccgaaataagaaagtgccgaattgtttgcctctaggttgaataatgtatgctgtgcatgtgtgttttggcgcgtgtagtatttatgtttgcagcttatcagttgaccccggattggacgggatgaatcgtaaaagttaacggcctgacatatgataagcaatcaatagctcttacaacgtatggaaa

SEQ ID NO: 6 - DNA, 787 bp, Aedes aegypti
gttgaggattaaaacaaaaatctcggtataagtagttagtaccttggtagcttttaacatggaattgtgtctcgcggataaaaagattgtagttactggtgcaggccagggtattggcaatgagttgtgcaaaacgctggtcaaattgggcgctaaggtgattgcggtctcccgatcaccaggtccacttgaagcgctgaagacggaatgtccctccgtgcaaattattcaagtggatttgagtgattggggcgccaccagaactgcactggagaagatcgatcgcgttgatgggcttgtgaataatgccggaatcgctattatcaaaccgtatgatgaactgaccgagaaggacttcgatgacacattcaacatcaatatcaaggctgcgttcaacgtgtgtcaaatcctgatcccgaagatgggcccaggtgcaagcattgttaacttgtcctccttggcagggttgaagtccttccaagggcatagcgtatattcgatgacgaaagctgccatcgactccatgacgaaaagcttagctctggaattgggcgagcggcaaattcgagtgaacagcgtcaacccaacggtcatcctaacgcggatggggcgcgacaactggagcgatccagctaaggccggtccactgatagcgaaaataccggctggacgtttcggcgaggtcaacgaagtggtggagcctattatattcctactgagtgacaagtctgcgtacatcaatggccactgcatgccactggagggtggttatttggcaggaaattga

SEQ ID NO: 7 - DNA, 6228 bp, Aedes aegypti
ggaaatataatagttctactgtcttatttcaaatggaaactaaatgttagtaatatgtattatctggaaaaccaatcacatatttattaatcttccattagttatccataataaacagatttacatattcctaaatggtaactaccttggataaacgtgtatcactaccaacactcgattatacatatatccttacttcgatttgcgtctcactcctgtatcgatctcgctattttcctttgtatgatcttgagagcactccacaagctcacgtgtctccgaggaaaatccgcacctggtttaaagcttcccatcttgctccactccaccagccaggtccccattaaagtctcgtaatcaccaccagcagctgcaccatcaaccaaccagcacataaccccgttcgaagaatcaacggttattttcttcgccgcgtgaacaccttcttaagggtaaagacggttgaggtgcagcgcgatcacgctggtggtggaggtggtgctcgtggttcccgtgaagatcgcgctcgctagacagtaacggtgagagagagggatcgcggcgagcagccgccgttgactttataaatacaatccgccaaccgattgaactcatcagtcttcacttgcgctttcgtggtgtcagacctttctctcgcgatcttgaggaaaagcttggagaagtgttgggtgaagaattggggttgagcaaaactgggataaaagcgtgctgaactttcactgtttgcgccgccatcagaagaaaaagaagaaagtgaattccgtttgaactttgcgccattcggccgaatcctaaaatgaaagtgaaactgtattaccgaatcgatcgacggttcgaaagtgacattacctaacaatgctgaacgcagagtgcggacaccgagtgtgttcgaatgattgaaaatgatgtgatataatcttctctgtgattcacatgtgatcccggaaacaaacagcaccaacgtcgagtatgtggccctgggacggcaagttccgccgaagatctcgctcaagcggacgcccctggaggggatgtgtccctcgagggaaccgccgccctgtgttccggccagccgccgctaccggacccacgatggcacgtgcaacaaccggaagaggccacgctggggctcggcccagatgccgtttcatcgcttcctggcgccggagtacactgacggtgtcgaggggatcagacgatccatcgccaacggaccgctcccatcggcacgtttcatcagcctggtagtgcacggatcccgccaagaggaagcccccgtcacgatgatgttggccctgtggggtcaactgctcgatcacgacctgacggccacggcccaaccgcgcagcctcaacgggtccactccgcgctgctgcgggcgcaacgacgaaggcctccatccgtcctgtctgcccatcaaggtcccgctggacgatccgtggctgtcaccgctgggcgtccgctgtctggagttcctccgatcggcaccggcccaaaggcgcgattgcctgctgtcctggcgcgagcaaaccaaccaggttacctcgtacatcgacgcttcgcccatctactcgagcaatccacgcacctcggataacgcacggatcttccgtaacgggttgcttctgttcggacggggaccgcccagcgaagacgtttgcttccgggcggcgcttgccaaccagtgcatccgtccgggagatgcccgaagcggcgaacagccgggattgctgatgctccatatggtctgggtcaacgaacataaccaaatcgccactcagctctccgacatcaatccgcactggagcgatgagaaactgtaccaagaagctcgacggatcgtcggggccatgttccaacacataacctaccgggagttccttccgatcgtactgggcaaagaggtgtgtcatttgttcgaccttgaactggagaccagcggattctacacggggtacgaccccagcgtgaatccaacagtggcgaacgaattctcgtcagctgcgttccgattcgggcactcgttgatacaaggatcgtacatgagagccgacaggcatcaccggttcattccgaacaacgtgacactgcacgaggaaagcgccgaaggagatcttggcgggccgggctcattgcatcgattggtccggggcatggtcaatcaacgtgcactgaagcgcgatgagtttatctccgcggagttaacgaaccacctatttcagacaagaagtttcccgttcggattggacctggcagcgatcaatatacagcggggtcgagatcacggaattcagccgtacatgaactggcggataccttgcgggttaactcccatcaaggattggtcggatctggaccgcgttatgggcccagcatcggctttgcgattacgcaaagcttaccgctcagttgacgatatagatctgtttgttggtggtctagccgaacgtccggttgttggaggtatcgtaggacccaccttttcctgcatcattgcccagcagtttggcaatcttcgtaaaggagatcggttctggtacgagaatcctggattcgagtcctcgttcacgccagcgcagttggagtccatccgccaagtagcgttctcgcaagttctttgccgagcacttggaggtggtaccttgcaaccccacgtatttctaccagctgacgccgtacaaaacgaacgactatcatgcgactcgaaactgatgtcacccattgacctagaaccgtggactgaacgtgacccttttacgagaccaaacgacgagccaaacgccgaacaaagtgacaatgacataagtgcagcacaatccgtattctcaactaccacaaccagaaaaccactgaagaacgccaacataatcaacaaagtagactttattccgaacggcaatactggacaccagataccctccaacggacaaatcgtactgaactctcctagaccttcaacgacaatagtgaacaacaagctagactttaaccttaacactccacgaccgacccctgcacaaccgactcaacctagacctgttccacagaaagacccaattcctacgccatctactgttatagacaacaaactagatctgacaggaacaactgctcctccgaaacggaaacctaccaggaaaactacaccaagacgacgaccgacggctgctacaaacctcatcatcaacaatatagacttcagcttgaaacgtgactctaccgacaacgttaccgacgacgaagaccttgaacattcgttgaaaaaacgtgacttgaacctatcgaagttgtttacaaaagaagacaacgcaacgctactgtctacaaccgacaaaaacgcacttcgaagcggctactacggggcacctgcaccggcaccaccctctgactacgatgacgttaacaactacgactacaacaatgactctgacccgccagtattcgttaaacccggatatggccatgactcaggttacggacacgtgacagaaccaccgccaaacccatactacggaggttactatacgactgaaaaaccaacgccacctcctcacccaccatactgcgcttacaactgctacaatccacctatgacaacctacaatccctacggactggacacccgacgacctaccaatcgacgaacgactcccagacgaacgctttccaacaagatcatcaacgactatgaccacagtcaacagcaggacagacaaacaaccaaacgaccaccacaaacgcaaactcagacaactttccaaagacctggactactagtaataaacaatgttagaccaaatgacaaaccaatctttctatcgtctgctccttctgcgcctttaccttcttcatctagtctcaattctaatgtcttccttcaaaactctcagaaaccttcctcttcatctgcctctcaatctgtcttttctgtcgaccctagacctcagtactcttcttctagacctgcccatcatcaatctgctctaccttcgcctatcttctctcagaaaaagactagtctacgcaaacctcaaaagcatctacagacggacagcaaagaccgactgccttcccgaatcgttcctcctcttcttatgcaagactacgactatgacattccggacgccaaccgacaagatacactgaacatccacgaacacgacggctacctccgacccgaacagaacaagtacgaacctcttaaaaagacctacgcacacgcaacctaccgcaactacagccagtacaacgcacacgaaccgtacatcgacagctatacattacaaccaccgacgaacgcaacgctttggccacgcaagaactttgaacagcaccaacgatacgccgaccggtaccaacaaccaatggaagagacagacgatgaactggaccaatacgttggaccgtacatagaccgggacaaagaaacgaaagaccatgactgcgcagactcagacgtgaccgacaatgttgaccttactgaacaccatactgacgatgaatctaccagagacaatacgaataaacatgatcagactgacgaaacttttacagtacaaaacactgacctggaaaaagctgtgacagacgaagacactgccatgaagtctaagacattcacaacgggcgttaaaacaaaggacattaccaatatcaaaggcaagacaagttcgacgaaacaatacgtgaccacgatagactatcatggacctgatgacccgcaacaaaaacaaactgacaaaaacttatacatcatacgacattatgcagacaaactaacacgtgaacatgacaacgttgacagaaaagtaacagacaatgtagaccttactgatactgatacagaattggcaggaatagacgtgacaaacctcgaaacaattgaccaacgattgaaaagcatgactgataaccaaaaacacgacaaaaccaagttgatagaagcaaatacctcgatcgacgaaactgttgaactagaaactcttaagacactgaacattctggatacggacaatgaaactgatacggaacgccaacatgacgaacaacagtcgactcacctagtacggactgactacaactcaacgtctgaaaacacgacagacgacaacctacaacagactaatgaccaactatcacttactggaatcactcacaaaaatgtactgactaatacgaatagcaagactaaaaaccaagaacataccccaactgagactcagacttcaggagaaggctcatacgaagactacatgaccgttgacaccaacaattcgccagacttatcaattttacaaaccaacttgacgaataaaccaagcttgaccaagacgatcaaaggacaaacgtactatgacgaatatgatgacgatcacgacgccactgccgaccaggatctcctacaaaaacccaacgccgaccaccaaacgaactccgacccaatagaccgactgaccttacaagacatccacgcccgaacgccagaccgagccgacccaatagaatccattctaccactggacgagaccgacaccaccaacgaccctccaaagcccccacaacctgaccaaacaacgcaaagacccacgcgaccggaccgacgcccgacaccccaccaaagaagacgcagaccgacttccaacgcaaaacgaccgacttcagcagcgacgtcgacaactggactcggacacggacaccacgaccgacgacaaccgggaattgcgctggttcccttcgtcctactaactagtatcgaaagaccggacaactgggtgatgttcaacgtggccaagtcgaagaagcgcaatccactgccggatgttccgcagctgaagagcgatgcgttctcgctgagcgagctaccgaaaccgatcagtgcggaagactgataccgtttcggggaatttcttttgtaaacagcatttgattgacctatgggacgacttgctacgggacgacttgctacgggacggaatgactaatactttgactgcacttaaatacggcggacgaagttttacttattactggaaagaccttcaacctttttactgtactaagacctgaccatgactatcacatagcttacaggtattatcaactgaatcgttgacgttctggtgtgcaggttcgattgcatgaatttgttattatttattacactatttatttattgatatttattaaaacaatttatgacaaatgaaa

SEQ ID NO: 8 - DNA, 2058 bp, Aedes aegypti
atggccactggaaatcgtaaattgcttgccttgctgcagcgccccttggagcctacgttctaccctaaggacgatggaaggacggttattgacctgccggaaaactatctcaccgaacgctacaggccaattggtgcgaacattcagaaccgatttggaaacgacgcagacacccgaattccagttcgcaatgttggtagtccgaatattgcctttgcggaggccattcctcgccgtggtggattttccctgtttaaccagagagatcgtagggttgctggagatctaatttcattgttcgtgaatcaaccggatcccgaaacgctgatggcagttgctgcctatacacgagatcgtgtaaatccagtcctgttccagtatgcattggccgttgccattcaacatcgaccggatactagggaactgaatattccctcgttcttggagctgttcccggattcgtttgtagatccctctgtatttcccaagttgcgtgaagaaggttcggttgttcaacaggagaatcggatggtcatcgacattccgttgaacttcactgcttcagaacgcgaagaggaacaacggctggcgttcttccgcgaagatattggagtcaaccttcatcattggcactggcatctggtgtacccaggggatggacccgatgcagttgttcgtaaggatcgtcgtggagagttgttctattacatgcaccaacagctgattgcccgatacaatgtggaacgtttctgtgcacgtcttgcccgagttcgaccgctgaacaaccttcgcgttcccatccctgaaggttacttcccaaagattattagaagtttgaccaatcgtgcattcccaccgcgtcctcaaaatcaaattttgtccgatgtcaatcgagtggatgaccaggtggttttcactataacggatttggaaaactgggaacaacgcatctcagacagtattgatgctggatttgtgatgggaatgaatggtcaacgcatccctctgacagaacagaacggtactgatatactaggtaacattatggaaccttcttcactttcggtcaaccgtcaatactatggaagctatcacggaaatttgcacaatgttattgcatacagccacgatcctgagggtcgtttcttggaaggctatggagttgttggagagttccaaacggccatgagagatcccaccttctatcgtctgcacgctcaagttgataacatgttccatcgccacaaaaacactctgccaccttatacggccagtcaactcaactacaacggcgttcaaatccaatctcttggagttcagttgaatcgtgctaatgctcctgcgaacgtactgctgacctattggcaacgatcgcaagttgatttggccactggattggactttgggccacaaggaaacgtatttgcctcgttcacacatttgcagcacgctccgttcactttccgtctgatggtcaacaattctggcgctagacgtcgtggaacttgccgaatcttcatcggtccaaagactgatgaacgcaacattcctctgacctatcaggaacaacgtattctcatgattgagctcgacaaattcaatgttacattgaaccctggtgccaattctattgtccgtcgttcggagcaatctagcgtgaccatcccttacgagcgtacattccgctcggtagcaatgtccaatcaaccaactggcgctgaacagttccgcttctgtaactgtggatggccatctcacttgctgattcccaagggtactccagagggaatgaccttcgatctgtttgcgatgatttccgacttcaccggcgatacggttaaccaggaattcgatgaaaacgtcgactgcaacgattcgcattcgttctgtgggcttcgggatcgtctgtatccggatcgccgtccaatgggttatccattcgatcgtcctgccactggcgccactcgaaccttgccggatttcattagctcgaacagcaacatggctctcgcgaacgtacaaatccgattcaccaacactgtaattgctagaacctga

SEQ ID NO: 9 - DNA, 1474 bp, Aedes aegypti
gtattgagaaatgcttgatggtctggattagaacagggtccaaaatgaatcaggtttcccaatatattgtttaaaaaaatttatatattttgactacagataaaagcaaaaaataatgatatttttactcatttcctagagaaaatttgttttgagtaacatttttatttctattctgcgattatcgaaattattagcttttcccagtccccgtacatgtttaatcgttaatttacattcattcttatcaaaaccaatccattatcaatagtttaacgttaatatgttaagtataccggctgcgactgtgtaaatgtatgttatcgcaccactgcattcgatcgccagccgatggaaaaattgatcgttccacagttcggtttgatcgttcgatcggtacacttcttcgcagcaggatggcaagcatcgtacgtaaaattgttcacactgccaagtgtccaaaggctttggctccgtataaccaagccgtagtggccgaccgtacagtttactgttccggagttctgggcatggaactaggctcacttaagttggtcgatggaggagctgcagttcaaactgccaaggctttggaacacttgacaacactgctggaagcgtccggatcaagtattgaaaaggtcgtcaaaacaaccattctgctggctgatatgagtgattacggtgccgtgaacgaggagtacaagaaagtgttttccaacaatttcccggctcggacgtgctttgcggtcaacaaacttcccctgggagcatcagtggaaatagaagccattgctcttaccggagaagttattcaagtgtccacatgacttgccacaaatgtctagatcccaagcaagacctgtgctgttaatccaaccaatttgggtaccggtgtgaaatgaactagaaactggaaacgatagacagcatctagcaaagacatttgtgatatgctgctttaagcacatttagtcgagcttggagtgactgtgttatagactggtttattcaataaatatttttagcgatatacattttatttcattttttaataataggcattgcatggccctctatccaagcaacattctgaattttcaaacaatctacaaacatcttcaagagtaagtgaactacggagacacatttgaaaaaaaaaaactttttcaaaggataaaaataataaagttcttcgacaagaagaattagaatccgtggtcagtattacatcatcaggtatttatgttgaaaaaaaaaaaaatcaaaatttttcaaggagaaaaggcgccgtccacaaactatgtaatgcttccaaaaatgggggaggaggtaggctcaagcattacgtctcatacaaaatttaaaaaataaatttcaataaaggcttcaaaattgacagcatggccttctggtgcctttggtggacttgttttgaaacgttatttaatgaaagcgtcatatttgtttaaaa

SEQ ID NO: 10 - DNA, 2317 bp, Aedes aegypti
agatcataagttatgcgagaatttctcttaaaaacctcaccaaatcaaggtgtgccaatttttgccgactttcccctattgttaaattgcggttcccattcccatcccaccccatacggtcaccctcatctatgaccatcagtcgtttcgcatcgattctctcttctacacactgtaggttactatcgtcagtttaggtcgcgccatacacatcgtcagttcgtcgtcgtgccggaatggtgtagcatatatccctcgaaaaagaaagcaaagtatctattatacaaccatctatcggatcgccatcgcacctaccagtgtgtgcatttttcgccggatcgaaagcctttactgtgcggaattgaaaatctagttatcctagttggtccccgtaatccgtaacaacccacttaaggtcaagatgagcatattctccgctgtcgaagttggaccccccgtggaagtgtttgcgctgaaccaggcctgcttgaaggatcccaacccgaataaggtcaacctgggagtcggagcctaccgtacaaacgagggcaaaccctggatccttccggtcgtcaaaaaagccgaagcggccatcgtggccgatggtagcctcaaccacgagtacctcccggtgctgggaatggatagtgtgaccaacgccgccaccacccttctactcggtgacgactccgaagccatcaagagcaaacgtgccttcggtgtccagtgcctgtcgggaacgggagccctgcgggtcggtgccgagttcctgtgtcgcatcctgaagcgaaccacgttctactactcgtcgcccacgtgggaaaatcaccacaaggtgttcgtgtacgccggatttgccgaaccgcgcacctatcgctactggcaccaggaccggcgcggaatcgacttcgagggcatgatcgaagatctgaagggggcccccgaaggtgcggtcatcattttgcacgcttgtgcgcacaatcccaccggtgtggatcccacgcaggaccagtggaagcagattgccgatgtttgcgaggagaagaagctgttcccattcttcgattcggcttatcagggattcgccagcggtgatccaaacaaggatgcctttgccgtgcgctactttgtggagcggggattcgagttgttgtgcgcgcagagcttcgccaagaacttcgggttgtacaatgaacgtattggcaacctgaccatcgtacagaaggacgagtccaccaaggcggccgttgcctcgcagatcacgctgctgatccgtggaatgtactcgaacccaccggcgttcggtagccgcatcgtgaacttggtgctgaacgatgccaccttgagggccgaatggatggaatgcatccagacgatgagttcgcgaattatcaccatgcggaaggcactctacgacgaattggtggccctgaagacccccggaacttgggaacacatcacccagcagatcggaatgttttcgtacactggactcaatgagaaacaggtggacatcctcatcaaggagttcagcatctacctgctgaagaccggccgaatcagcatgtgcggcttgaccgagaacaacgttaaatacgtggcgaaggcgatccatgaggcggttacgcgcgcgtcttccaaaatctagatttaagagcatcgctagcaccctcttatagcaagtctattgttcgtagaacgattgtttgttgtttccttatcaattgtgtgtgattgtgcgcgccatctgcaaaaaagggcagcgcttgttcgattctagatacacgttttcgtcccccaaatccttcaggtctagcttgaggcacgggacaccaaaagcatttatttcgttattattcagtgcaatcggttcgaaaaaatattacattttgcaatcaagtttacaccaatatactcaagtcctccactagaaagcccgcaaccggaagtgaccaaacaaatccataagcaaagcatgtacaaatttttctataaattctggaagattaacaaacaacggtagcattgacattttgataatcaaagtttttctacgcagcgaaaataagaaaaaaaaaatcaaactattacacggcatcgatatgtattccagcatttacgtaatggcactagaatgagcatttagataaagcaaatatatacggtggttattaatcaaaaataaaacagctcaaagagattatctagaccgtggatttgaagaatcatttatcgctagtccaaatactgctgctgtctctgtatcctctcgtttcccgtttttacggctttcgactctaccctcttcttgttcttg

SEQ ID NO: 11 - DNA, 2805 bp, Aedes aegypti
atgcttggagtcgtagttcagaatacgactacacataactttccaccactttcagatcggtttttgcattggttgtggaatatacatttgttcaaccccgatgacccttgcgaacattacctgttgaagtgctgtaaagttccgaaagatctagacgataatgaacaggtcattgcgccgccctcatccagcggtgaactcattattgcaccacctagctcatctggaaatctgatccagcctggagcagaggatcgggatgggtcaggatcagcaggaactgattcttctggcggaccaggatctggcggacatggttcgggaggtgctggttctggtggtctgggatcaggtggacaagagaccggagggctaggatctggcggaaccggatcaggtggacttggaaccggtggagttggaccaggaggccatgggtcaatacctggttcgtggtcagaggggcctggaggtaacggtaattatcatggtggtcatcatcaatacggtggtggtaactacgctggtagtgtttcacaaacaattataattactcacatatgggtcagcacggtccttgtatacctggaacgcatactggaaatgttgggggagcgggaaggcccaatgcaggtggctcatctggtcaaatttcgggtagtggatcatttggaggaaccgcaactggaacatctggtcaaagtcaaactgcaacatcgactggtgtatcttctggttcatcgtctggcactatcggtcatgggtcaactcatggatcaacgggacatggtgcaagctatacaggatcttctactggaggagcacctagctcgttgcaaagtccaaatggtcaaacaagccccacaactggtgtgccttctggatctttacacggatcaactggacacggtacatctaccacgggatctactaccactggaagtgcatctaattatacacaaggttcatataatcaaaatcattcatctggacaagcttcccttcaaactggcacttcgaatgttggagagccagacaggctcaacgctaggtggttcgtctagtcatggagcaagccagtcgggttcatccaccggcagcatgcttgcgccactgaaggattcatctgtgcaaaaccaatcaggcccatctactggaacttctactggatcaagcggacccactacacatcagtccggttcatcagctgcaggaggatctgcacagggttcatatggatcatctgggtctacgcatggatcaaccggccaaagtacaagtcaaggttctttcggacaaagtggttcaagtacagcccaaggatcatctggacaaagtggcttcggatctcataatttgaatggtgcaaacttggttgcaggagatttatacagcggaaatattccaactgggtcagctggtagccatggcagcaataccggaggatccgtacaaacgggaagtgtagtgccgggcgttagtaccgtaacaaaagaccaaattagggatacagaaaccacaagtacgggtttatcatccggtacggctcagcagggacacttgtcatcaagccaacagcagtcttcctcgggaggatcacaacaaacttcaagtggcagccacacaaatgtaggcggtcatgaaggtgagggtatgtcatctggtacggctcagcagggacaattgtcttcaagtcaacagcagtctaccactggaggatcacagcaacagcaaacaagtggcagccatacaaatgtaggtggttatggagtatctggtggtacgcaccactcctctgatggatggaaggatcataccgagaagcatggtcatgggcatggtcatagttatggtcaaggtcacggacatggtggtggttatggcaataattatggtggtggatatagtagctcacatcaaaccgatcatactaccgtgtcaccaatcaaatcgcctcatgacaatgcaggatgtggtttccgcaataaagatggcgtaggtttccgcattacgggcaactcggacggagaggcagaatatggtgaattcccctggatggttgcaattttgagagaggagaaagctttggatcaagtcatcaatgtttatcagtgtggtggatcattgattcatcctctagttgttcttacggctgcccattgtgttcagaataagaagcctcatgaaatcaaagtccgacttggagaatgggacacccaaacaacgaacgaaattcatgatcatcaggatcgtaacgtacttgaaatcgttttccatgaaaaattctacaaaggaggtctattcaatgacgtcggattacttttccttgacaagccagctgagattattgagacagtcaataccatttgcttaccgtcacaggattacaatttcgattacagtcgctgcttcgcttctggctggggaaaggacgtgtttggaaaggaaggcaaatatcaagttatcttgaagaaaattgaacttcctattatgccgtacaacgattgccagaaagctctacgaacaactcgattgggagctagattcagtctcaataagagcttcatatgtgccggaggtgagcccggaaaggacacatgcaagggagacggaggttccccacttgtttgtcccatccctggatcagtcgatcgatactaccaagcaggaatcgttgcttggggtattggatgtggtgaaaagggaatccctggtgtttatgctaatgtggctggatttagaaactggattgacgagcagcttacccagcgtagcattccacataatacctatatcgctgtgtga

SEQ ID NO: 12 - DNA, 1583 bp, Aedes aegypti
gtgtgagcggggtactgaaacatgcgcggatactggtgcactgattgcacatgtacatcaatattatttttaatttaaaaatgtggcttggaaaataagtttatggtataatattctccaaccgaaacacagcgcacaatcattatgttactcgccatacgatttgtgactctgctgcatgtgactttataagttgactaatttaaattaattcacgaaacaatacattgcacattggacattttcaaatgttacgctagtagttgctcccctgttttctacggcgcaacaagtgcaaggaaaacatctaacagaagttcccctttccccatccttacggcatccggggtaatgagctcttcactggatcctttgaagcaggacgagcagagtcccattttgcatcaaacaggcttatatttaaaaggattcaacgatttttcggaatttatccgcagcacttttatggtcgcacacttttcttgtcgttcgcaaattactggtctgttttgatacaagcagccgccaggcgtttgacgtgcggatgttgcatatgattttttttcgctgctagcattctgagtgcgactctgcgttaatttttgacaactgctggcagaacgaccatctttctccttcggtacggaatttagtgcgtgtggtgaaggtagattttgggcacgaaaaaactcgattttcaacaattaaattgcttcaaaatgctttcgagagcagcacttttggccgccgccaagaagcccgcaggcctgatcctggcccgtggatctgcctctgctactgatggcaaccgtccggtgcgagcggaacaccccggcaaagttcgcatgggcttcctgcccgaggaatggttcaccttcttctacaacaaaacgggagtcactggaccgtacgtcttcggcgccggcttgctgacctatctgtgctccaaggagatctacgtcatggagcacgaatactacaacggtctgtctttggccatcatggtcatctacgccgttaagaagtttggaccggctgtggctgcctattgcgacaaggaaatcgatagaattgaaggcgaatggaaggccgaccgcgaaaacaacatccaacagctggcccaggccatggaggacgaaaagaaggaacagtggcgtgccgagggccagaccctgctgatggaggccaagaaggagaacgttgccctccagctggaggctgcctatcgtgaacgcgccatgaccgtttaccgtgaggtcaagaagcgtctggactaccaggttgagcgccagaatgtcgatcgtcgcatctcgcagaagcatatggtcgattggattgtgaaaaatgttgtaaagtccatcaccccggagcaggagaaggaaactctgagccgatgcattgccgatttgggagccattgccgcccgcgccaagtaaatgtgtaatatgtggatcaatgaccgggcgtcggtattcagaccttgtagagcgaagtaagtattcgttttctgaaaagttcgacgggaaagatttgggagtaaacaataaattgaatacttttatcaagttagcgcgatatccaaa

SEQ ID NO: 13 - DNA, 2953 bp, Aedes aegypti
ctcaactgcttcaggggcagcatcctctcgtaggcagccaagatatagactaccctaaatcttatctccaaggactcttagctagtttgtgttaagtttttttttaattcgttctctgtgtactgtgaattttttttaacacaaaaagaaaattagtgatattttttcgataacaattcgtaagcacatgtgaaaattatgtgtcttctagaaacacgtttgtgatacaaacaatcggactacagtgatcgtgtaataatttaatcgtgcgtcgtttgataaccctaaacgaaccagaagaaaagttcacaatgacctccaattgtgagatcacggtcacgagcccacaggaggcccagaccaagagcagtatcatcgtggtgtccaatcggttaccgttcgtgctcaaacgagaccccaagagtggtcagctcagccgcaacgctagcgctggcggtttggtaacagcggtagctccagtcgtcatcaagggcagaggtctttgggtcggatggtctggcatcacgctgtccgataaggacgaaatccccgaatcggatccttcggatcgcacacctaccgcaggacttctttcggatcaggtcgtatcggtcaatgtagaaccggtgctgttcgacagctattacaatggatgctgtaatggaaccttctggccactgttccattcgatgcctggccgggctacattcaatgcagaaaactggagtgcatactaccaggtaaataaggaatttgccaccaaaaccgttgaagcgttagagagatgcttgaagcaaaagaagaacccaggtgtgccaatcgtgtggattcacgattatcatttgatgctggctgcaaattgggttcgcgaggaagctgacgaaaaaaatctaccatgccacatagcgttcttcctgcacattccgttcccaccgtgggatatcttccgcctgttcccatggtcggatgaaattctgcaaggaatgctggcctgtgacatgatcggtttccacattcgagattattgcttgaatttcgtagactgctgccaacgaaacctaggttgccgagtcgaccgtaaaaacctactcgttgagcacggaggcagatccgtacgcgttcgacctctgcctattggaatccccttcgacagattcgtcgaattggccaaaaaggcgaaacgtgtgctcagaaccaatcagaagatcatcctcggtgtagatcgactggactacaccaaaggattggttaatcgccttaaggctttcgaagtacttctggagaaacatccagaacatcgagagaacgtaagcatgctgcaaatttcggttccttcacgtacggatgtcaaggaatatcaagaactaaaagaagagatggatcagcttgttggaaggataaacggtcgattcaccacggccaactggtccccaattcggtatatttatggctgtgttggccaggaagacttggccgctttctaccgggaggcttccgtctgtctcgtgacaccacttcgcgatggaatgaatttggtcgcgaaggagttcgtcgcctgtcagatcaatgaaccaccaggcgttctgatagtatcgccttttgccggagcaggagagaccatgcatgaggctctgctatgcaatccttatgaaatcgaatccgcagcagaggtgatacatcgggccttgaccatgcccgaagacgaacgatcgctacgaatgggtcgcatgagaaggcgagaaatgcaacaggacgtcaacagctggatgaggcagttcctcaaagccatggattcgctggaagaagatgaaatcggtacaacgacgatgcatccggtcacagtggacgatttcgacgattacctgctgaattacgtcggattcaaccacaaactggccctgctgctggattacgatggtaccctggccccaattgcaccccatccggatttggcaaccctaccaccagagaccaagaacgtacttcagcgattgtccaatcactcggacgtgtacgttgccgtcatctccggtcgaaacgtggacaacgtcaaggccatggtcggcatcgaaggtatcacctacgctggaaaccacggtttggagattcaacacccggatggaagcaagtttatccacccgatgccaatggagtacgagggtaaaatgaccaaactgctcaaggccttgcaggaagaggtctgtcatcacggagcatgggttgagaacaaaggcgctttgctgacattccactaccgtgaaacaccgaacgagctacgagctgaactggtctccaaggcacgccaattgatcacggacaatggattcagtgtcaccgaggcgcattgcgccgtagaggccaaacctccggtccagtggaacaagggtcgggcttccatctacatcctccggacggcgttcggtgtggattggagcgaacgaatcaagatcatctacgcgggagatgacgtcactgatgaagatgccatgatggccctcaaaggtatggcagccacgttccgcgtcacgaacgcccagatagtcaagacctcggccgagcggcgtttaccctcgacggattccgtcctgacgatgctcaagtgggtcgagcggcactttatgcgccgcaagccccgatccaacagtctgacctacaagaaccgcaagaaggactgcgtcaagatgcagctggcgttcgaactggttccgagtccaaaccacagtgccgccaacagctccgacgagcgggattaggcgccactggcaactcactcgatgatgttttagcaaaagcaatttagtgtcttaatctagggttatttaaatagtgctaggtttttataaacgcacacaagtgaaatatgattttgctttctgttttactgttttcgaatgtttctcttactgtgagattatttaaataaaaagttgaattttagtctaagatgaaccaa

SEQ ID NO: 14 - DNA, 882 bp, Aedes aegypti
attacattcgttttctgtcattcgagaatttcattgcaaacagtcagcagattttgtgtttcaagttaataaatcaagttatttaccagaaaattgccgtgaaaatgttgggctttgaaaattttcccgaatacgaagttcccggagtgcgccaggttcaattttatccatatgaatccaacggcgggagcgtggttgcgattgctggagatgactttgccgtgattggtgccgatacccgtctcagctcgggttattccatccatacgcgtacgcagaacaaactgttccggatgtcggaccggacggttctggcctctacgggatgttggtgcgacactctggccctgaccagcttggtcaaagtgcgaatgcagatgtacaaggatcagcaccagaagaacatgacgacaccggcggtcgctcagatgttgtccattctgatgtacaatcgccgattcttcccctattacgtgtccaacgttttggccggattggatgcggatggaaagggcgtaatctacagctacgatcccattggacattgtgagaagacaacgtaccgtgccggtgggtcagctggacctttgctgcaaccggttctggacaatcagatcggcttgaagaacatggtcaatgtggctccaactccagtgcagttggacaaggccatctccattatcaaggataccttcatttccgctacggagcgagacatttacaccggagattccgttataatcaacattattactaaggatggcatcaaggaagaaatcgttcagctaagaaaggattaagctaatatggttggtaagggtgggaggtttttcttatgcttttttgatttattaacacccaataaatttgaatgaaggattt

SEQ ID NO: 15 - DNA, 1178 bp, Aedes aegypti
ccccaatggttccgaatttggcagcactgtgccatggtcgcttgagcaatgaccgtgcgcaaagtgtaatcagtccacccgcgaagcgaagataagcgcaactgaggccgataactgattgtgtacaaagctgcgtacacaaacaagcttcggttggagggaaaagagagagggagaaccgcagccgaatcagtttgcgagagtgaaccgagctgcgcgaaggtgttttcgattgcgtgaccaatttcatctgaattaattttattccgaatgatgggcatcaggaatacgggcaaactagccggcaaaacgctcttcatcaccggagcatctcgggggatcggcaaggcgatcgcgctgaaggcggcccgcgacggagccaatgtggtgatcgcggcaaaaactgccgaaccgcacccgaagctgcggggaacgatcttcacggcggcagccgaaattgaagccgccggcggtaaggcgcttgcttgcgtggtggatgtccgcgatgaggtcgccgttcgggccgccatcaagcaggcggtggacaaatttggcgggatcgacatcctggttaataatgccagcgccatttcgttgaccccgacggaacggaccgatatgaaacgctacgatttgatgaataacatcaatacgcggggaacgtttctggtctccaaggaatgcatcccgtacctgaagaaaagcacccacgctcacatcctgaacctgtcaccgccgttgactctgaacccggaatggttcgcccgcaacctagcctacacgatcgccaagtacggcatgtccatgtgcgtcctggggatggccgaggagttccggaaagataacatcgcggccaacgcgctctggccggccaaactggtccacaccgcagccgtggacatgatccacggtgatggagccgtggccgtgtcccggaaaccggagatcatggccgacgccgcctatgccattctctgcaaagatcctcgcacctgcaccggaaacttcttcatcgacgaagacctcctccaggcggaaggcatcaccgatttcggcgagtactcctgtgtgcccggaaacgagcggaacattcgcctctgcggtttcgtcgaccctaatcgatcgaaactgtgaaacattcgaaatcattggaatccattttgaatttcaaaagccag

SEQ ID NO: 16 - DNA, 1194 bp, Aedes aegypti
aatccagtgccagccatcgtcgggaacggttccatagtactgcgcgtggcttcccggtttccatttcgtttttcctttattaagtttagttgtatagtaggcatcggcttttaaggcatatatctaatcaatcagcataccaggctgatttagtgctgctattgtttcataatgaagctatttgttgtgcttagtgtgcttctggtcggggccctcgcccaagatggacccaaagtcactgataaggtaagtgtaacttccgtcaattttagatcgtagcttgttatctttttatgctttcccttcttcgcagttttaaattggttcagtgtattcttctatctgcgatctctgcttgcctagagttatgcatattcctcttacccagacgtctcgtactcttcttttacttgatcaaagtcaatttgcgggggttcactgggcttatgaaccggtttatcccgttgcaaatcattcgatggatctttcaattgccaacaaagaggtcttctttgatatcaccatcggtggaaagccggaaggtcgcattgtgattggtctgttcggtggaaccgttccgaagactgccaagaacttcaaaactctcgccgagaatgcggagggcgaaggctacaagggcagcaagttccaccgcgtcatccaggacttcatgatccagggcggtgacttcacccgtggcgatggaaccggtggccgctcgatctacggtgagcgcttcgccgacgagaacttcaagctgaagcactacggcgctggatggctgtcgatggccaacgccggcaaggacaccaacggatcgcagttcttcatcaccgttaagcagaccagctggctggacggacgccacgttgtgttcggcaagatcctggagggaatggacgtcgttcgcaagatcgagaagaccaaaaccgacggacgtgaccgcccggttaacgatgtggtgatcgccgacagtggttccctgccggtggacgctccgttctccgtggagaaggctgatgcctagattaataagaaaggtggagtacaggggaaagattggttatcttatagaagaatttgcgaacaaaaagtcccccaggagtgcatttattgtgatttaacacaaatgatgatgatggagaagttgataaaggtgaagaataaattgatgaacagtgatttttaatacaa

SEQ ID NO: 17 - DNA, 2139 bp, Aedes aegypti
taaatcgatttaatcctcgatcatgtctcgcagtactccctcgttcgttgctgggatcggtcgtttcccctggtgggctcttcttcaaactagttgcgcgcccttttttccacctacacaatgtcgttcggacaagttttaaacacctgcctaaagggagttatcgatcgaatgggagttctgactgcagccgccacaaggacgaaccgtgaatcggtgattgtacccgttaagaaacaggatatttcgcttaaggcacaaaacgcgcagccgattgtggcgttaaagagtaaagaactctacgagccacgtgtcacactcgacaacataaatccggcgatcgtgaaaatggactatgtagtgcgaggggaggtccttatgcgagccggagctattcagaaggaactggatcagggcgtcgtgaagcctttcaaggaagtgattcgggccagtatcggagattgtcaggctatgggacagcaaccgatcacgtttctgcgacaggtcttgggactgatcacgtatcaaccgcttttcaacgaccctaccatcccagaagatgccaaagaacgagcccgcatcatcctatcggattgcgtgggcaactctgtgggttcctacaccgattcgagcggaatcgaaatcattcggaaacacgttgctcagtacattcagaatcgggacggtggaattccatccgatccgagcaatataatcatgtcggccggggcaaccggtgcaatcaagtcgctaatgtccctatttcggtgtacgatcgatggcaagctgccgggagttatgatcccaattccgcaatatccaatgtattcggcgactatttccgagttggagatggcgcaggtttgctaccacttggacgaagatcgcggatggggcttggacattccaggtttggaacgttcggtgaacgaggcgaaggagacatgtgtacctaggatattggtagttatcaatccgggtaatccgaccgggcaggttctgtcgcgggaaaacatcgaaagtatcattaggtttgcttataatgaagatctggtgatcttcgctgacgaagtctaccagtacaatatccacgaggagaattcggtgttccactcgttcaagaaggtgatgatggagatgggagctccttacaaccgaatggaactgtgcagtttcatgtcctgctcgaagggttacatgggtgaatgcggtattcgaggaggatatgtcgaggttgtgaatatgtgcccgaacgttaaagccatgcttctgaagagcatttcggctttggtatgtccgtccaccatcggacaagcctgcatggatgttatggtgaatccaccgaaaccgggcgaaccttcgtacgagttgttcataaaggagaagaacgccgttctgacttcgttgaaagagcgtgccaaattggtggccgatgcgttcaactcgtttgagggcttctcgtgcaatccggtccagggtgctatgtatgcgtttccacgtattcaactgccggagaaggcaattcaggcggcgaggaaggccggccagctcccggacgttttctacgtgtaccaactgttggaacaaacgggtatctgcgtcgtaccaggatccggattcggtcagaaggatggaacctatcacatccgatcgaccattctgccacagccggaaaaactcaaacatatgttggacgtgttacggttgtaccatgaaaatttcttacggcagtaccagtgatgggctgaattattccttattttatttataagcaccacaaagataagtactgagctaagtgataggcgttgttcaaatttgttgtaaggatataaatatttgaaaataaaaagccatagttagaactggtgttagatcaataaaaacagtccttaagattaagcttttcaacatttgatcgtacttggatacagcatcagcgtagctaggaacccttttttcaatttgggtaataagattgagcacatattgtatgttaataactggaagaacccgttttgctctagattttttttttttaatttttaagtgtattccggattgaaggcagctcaaataagcatttttttgatgtacaatcatctgggaaatacaatggaaatttaaactggtcctggt

SEQ ID NO: 18 - DNA, 3053 bp, Aedes aegypti
atgggtaccttgcaacaattggccgtcatcaagggacagcagcgtggattgatccctgcccttctacatcggaacttcgaggccaacgttgataaattctgtggaaccgatttggcgctgattttcaacgatgacagccgaggcctcaccaagaccaactacaacgttttgaactcgacggccaaccgcgtggcggcggccgccatcgacgatatcgtaaattgtcacaaatcgcaaccgaatcgggacggagattacatcatcgccgtttgtatgcagcctagcgatcggctggtgacgactttgttggcaatttggaaagccggtgccgcctatctcccgctggatccgacctttccgccgaaccgcatcgagcacattctgaacgaggcacaaccggtgctggtcattcacgaggactacgaaaatgtggccgtgttcggcgcaacgccggctgtttcctacaacgatctcaggaaaaaggctagcgacatcagcaatgcaaacattcttccgcagcaaatgctgggcgcgggcatgaacgaactagctttggtactctacacctccggaagtacaggcgttcctaaaggtgtccgattgaatcacgccaccattctcaatcgcttgcaatggcagtggaaccgattcccctactcggaaacggaaaaggtcggtatattcaaaactgcactaaccttcgtcgattccgtcacggaaatctggggtcccctgttgaacgggatggccatcgtggtcgttcccaagaaggtaaccaacaacccggagagactcgttgaactgctcgaggagtacaaaatcgagcgcctggttttggtcccaaccctgctgcggtcattgcttctctatctgccacttcagaacaaacaacttttgtacaacctccgcatctgggtatgctccggagagcctctacagctatcgctggcaaaagaattctttgactacttcgaagagggagttcatcagttgtgtaacttctacggatcaaccgaagttatgggagacgtcacgtacttcgtttgcgaatccaagaagcaactcagcaattttgagaaagttcctattgggtatcctctggataacactgtgatatacatccttaatccagaacttcaaccggtcaaacccggtgatattggagaactgttcgtatcggggtcgaacctggctcagggctacgtcaacggtcgtgatccacatcgctttatcgataacccactggccgtagaccccatctactcgagactctaccgcacgggagatttcgcttcagccacaaagggatgtgtctactacgaaggtcgcactgattcccagatcaagatccgtggtcatcgagtagacttatcagaagtggaaaagaatcttctgtcgctgcctggcgtcgataaaggtattgttctatgctatcacgctggagaaatcgaccaagccttgctcggattcatcacggtcgaaaaggactctaccctacaaactggattacaaattgaaggcgcactggagagtaagcttccacactacatgatcccacaggtcatcattcttgacagcattccactcttggtcaacggtaaaatcgatagacaaagtctgttgaaaatgtatgagaacacaaacaacaacgatgacgccaccatcgagatcgagtatgactacaccggtgttccggagtgcaaaatggctatggcccaggatcttttcaccactgtgggtcaggtcattggacgctctacgcgtagtaaaatttcactgcaaagcaacttctacgaattaggaggaaactcgttgaactcgatcatcactgtaacacaactctgccagaaaggtcacgcgataagtattaccagtttcattggtgccgaaaacctggcagaaattttggataaaatgtacagcaaccagcagcacatttccgaacacgaacaaactgttggaaacggttcttccgttactcaagaatttgaattcgagtacaaattgaagctctctgcgcatccactggccgacgaacacaaagaagacgcgatcaacatcctgacgtacagtttctacgaaaaagctgatctggaacgatggatcaaaccgttgatccgggagcaggactatcgggatattctggagactatttgggatgttctgatcgaaaagcagctcagcttcatgatcaaggatgaaaccggaaaatcggtgggtgtctctctgaatttcgatgctcatgacgaaccggagccagaagtgaacaataacctagtgataatctttgagttccttgaatacgtggaaaaacctattagggaacaaaagttaccacaaggtaagaatcagattctacactcgttcatgatgggtacctgtgccgagctgagcgctcaagaaaacatcgaggctatgcactttatggagagcgaagtgcttaagatcgcgatccggcggaactttgcgggtattctgacgactaacaccagtccgctaacccagcaacttggatcgaacgtttacaagtacgaaaccatgctagattacaaaatcaatcagtacgtctacagtgacggaagccgaccgtttggcaaggcgggagacgatcagcgggtgatcgtgcactggaaggacatccgaaaacagcactgaacgtgttccccaaagatggttgaaagggctattggcataattagtagtgttctaggtaccatatttatcgctgcaataatgatctaatttaatggctacacaagaataggctaagaaaccatggaacagtatgtcgaaatgtaaccaaccgtagatattgatagcaaatgtaaactgtaaggtattgcactgattgtgctgcaagaatctttacgagttagttgtagcactaggtgtgtaagtgatattttaattcttaggttgatcgtacaaacaaggtggtcttagacaagtgagaaaggaaactgtgagaggatgttagttttgtaagttatttgtttctggattttaaggaaactactatttatgacaataaatcgaattatgactcattcgtg

SEQ ID NO: 19 - DNA, 688 bp, Aedes aegypti
ctgtcaaacactcagcctggactggggattcgcgtttgtagaactttccttcaccaaagtgtgtagaagttgtatcgtttccttctgtgaaataatccggattttccgcattcaaaatgtcctacgttgccagaaagggaccgcaagtcgtttcggctcttggtcgatgggcttacaatctgtccggattcaaccagtacggtctgcaccgtgacgattgcctgtacgaggacgacgacgtcaaggaagcgatccgccgtctgccggagaagctgaaggatgagcgcaactatcgcatcacccgggcgttgcacctctcgatgaccaagactatcctgccgaaggaacagtggaccaagtacgaggaggactgcaagtatctggaaccgtacctggaggaagtcaagcgcgagcgggacgagatggccaagtgggaggccaacaagtaaaaggccagacaaagagtgcaccactcattgttatgattgtaaatgcaacctttccgggggggcttccgaaggaatgtccctgggaaacgattcaaaacttaggggaataataaactgaaatctggagcgaagtgctaaagtgtttcattcatttagagtaggtttgtatgctaatgtttcatgaattttacgaatattatttatattcagaaggccttgctttagggagcaggatccta

SEQ ID NO: 20 - DNA, 931 bp, Aedes aegypti
gttcatattttttcaaagttgggttgattcgtggctccgtgtgcactgggtacaacaaaccgatttaatgggctcttcaaagttagcgtgtgaaatgtcattattgacaattattcatttcttgcatttcttacataaaccgagcaaatcaaaaaacataaatttgtgcaaaagattgttgtgtacaatgatgcgttcagcgattgctcctgtcgttctaaatggttggaaatcggtagttcccaaatggacggcagtagacacggtaaatatgaatcaaattcgagagaagggcactctccctgtggtagagaaggataaaccggaaaagttgccatattccccttttggtactaaacaatccaactgggctgattggactgtggctcgtctagatgacgtgttgaactggggccgtaagggctctatttggcctttaactttcggattggcttgttgcgccgttgagatgatgcacattgctgcccctcgttacgacatggatcgctttggagtagtatttcgtgcttcaccaagacaagcggatgtgatcattgtagcaggtactctaaccaataagatggccccagctttgcgaaaggtctacgaccaaatgcctgaacctagatgggtaatttcgatgggcagttgtgccaacggcggagggtactaccattactcatactctgtggttagaggttgtgatcgaatcatccccgtggatatctacgttcctggctgcccacctactgcggaagctttgctgtatggaatacttcaactgcaaaaaaaggtcaagagaatgaagactctacagatgtggtacagaaagtaaagcatataccaattcatagttatttcaagcgtattttgcactgtaaacgcaataaatgtgtgtaaataaaatgcattgcgtaaatatctcgatttg

SEQ ID NO: 21 - DNA, 1568 bp, Aedes aegypti
cgttttcacggtgcagatatcgatcgcaatctagtacttcaccacgcattcctaccgctggataaacattctaccgtttcccagtctctgagctgatctgtcatcgcaaattctcttttcggatcgcgacgaacgacgtccgccatcttcctggacttcattcatcttgcagcaatcgttcgagtgaggaagagtcgcgcgttcgcgagtacgagtttgttcggagtccggtttgtacgattctttttgtggtgcggtgatgaatttgatgtgtgcggtttgattgagcggacgaaagcagaaaaaaaatcctttcctttttacaatttggtggtagcagtttgtgatttcgtcgtcgtgttccattccccggaatcgcattttgttcggttacgtaagaaccctcttctgcagagaaaatagtgaaaaatccgagatccgggacgtcaaccgagaagaagaagtagagtgataagagagaagcaggtggaagaagaagaaattttcacgagtggaattttttcgtcgtcaaggcagcctgaaatatgttgaagaacgcggtagctctggtgactggtggcgcctccggcctgggacgcgcaacggtggagcggttcgccaggactggaagtaaagtagtgctgtgcgatctccccacgtccaagggtggcgacgtcgcgaaggaactcggcgacaatgtcgttttcgtgccggttgatgtcctctcggaaaaggacgtcaccggtgcgctggacatcgccaagaccaaattcggccgtttggatgtgaccgtcaattgtgccggcgtcgccgtagccgtcaagacctacaacttcaacaagaaagccgcccacaaattggaggacttccagcgggtgctgctggtcaacacggccggaacgttcaacgtgattcggctgtccgccggcctgatgggcgaaaacgaacctaaccaggacggccagaggggtgtgatcgttaataccgcctctgtggccgctttcgatggacagatcgggcaggcggcatatgctgcttccaaggccgcggttgttggaatgactctaccgattgcccgtgacctcagcacccaaggtatccgcatctgcacgattgctccgggactgttcaacaccccgatgctgcaagcccttccggaaaaggttcgtgccttcctggccaagaccgttcccttcccgcagcgtctcggcgaaccgtcggaatatgcgctgctggtggaatcgatcgtcgaaaaccctatgctgaacggagaggtgatccgactggatggtgccctgagaatgatgccttaagactaccccccttcaaaggtactaccaccaccatgcttcctcgttcccatacctttcgaacacaataagacggacagatgcatttattagtcctaagtgcgtgttgtagtgtagtagtagttcaccggtgccaatttgcttggtgaatgggattccttgttgggaactgccaagcagctgatgtgtgcatatcgtgcctatttttgaaccaggaataaagttttgctcaggagtgcatttttttgttaacttaca

SEQ ID NO: 22 - DNA, 699 bp, Aedes aegypti
agagtgactaatggacgcgaagccacccctggtcaattcccataccaagtagcgttggttatcagtttcaaccagggcagtggtttgtgtggcggctcaattatctccaataactacgttctgactgcagctcattgcgtccaaggagccagcggtggaaccgctttgtccatcgtgtttgagctcgatggaattaccatccatccggaatatacctcgaataccatcaggaacgatgtagcgaccattcgtttgaccaattcgatcgtgttcaacgatcgcgtccgaccaattcgccttccaagttcgaccgatgtccggaccttctccggccgtatagcaacggtttccgggttcggaagaacatcggacaccagtcccaacacttccgctgtggttttcttcaccaccaatccaatcatcacattcgccaattgtttggatcaatggagcaatgctgtgcacgtgatccaatcgcagaacatctgcctgtcaggtaatggtggacgatcgtcttgcaatggagactccggagggccactgaccgttcaggaagatggcgaaagcctccaggtgggaatcgtatccttcggatcgtcggctggttgtgccatcggaaggccttcggtctacgctcgggtaacgcactttttggactggattttggagaattccgatgttgatgagcaagcagcttga

SEQ ID NO: 23 - DNA, 755 bp, Aedes aegypti
ctccgatccgatctgacaaggatctgaaatttcagatttgcccatctgtttctgtcacactttttccatcgtgagtgtttcgcaagtgctcttggaaaagccagaaagaaaccaaccatgctccgctctgccgctggtcaattgtttggagtgctccggggaaccgtcggtttgaccacttcccgtctgggtgccgctggagctgtccgccattctcacagcaacgagagcgccgaggagttcgatgcccgctatgaggcctacttcggccggtctgacattgaccactgggaggcccgcaagggaatgaatgacctgctcggaatggatctgattccggaaccaaaggttatcgtggccgctttgaaggcttgtcgtcggttgaatgactacgccctggccatccggttcctggagggatgcaaggacaaatgcggcaacaaggtcaacgaaatctatccctatctgctgcaggaaatccgcccaactctgaccgaactcggaatcgaaaccccggaagaactgggctatgatcagccggaattggcgctgaaatcggtgtatgatatgcactagaatgggttgttttggccagtggtgcttttcctccccatttgcagcattagttcaaataagtgaataaagtggacggaactattccgacaagaaaaaaacgttctaagaatggttatgtaatcaatggcgattgttcgtcacagtgaaagaaatgtgaaataaaattggtaaaaatgaaa

SEQ ID NO: 24 - DNA, 1023 bp, Aedes aegypti
gtgattattattttctacttcagatcaatcgattttgcaagtcgccagcaatcatgggcgcgtccgctttaccgatcattattttctcggcgattttcggcgtggttggcatcgtcctgccgatcatcgctccgaagggacccaaccgaggaattgtccagtgcgtcctgatcctgactgcggccacctgctggttgttctggctctgctgttatatggctcagatgaatccgctgattggccctaagctacatcagaacacaatcctgatcatggcacgcgaatggggcaatccccttcccgatatggataactaccatcccgaaccgcacagtgtggccgaacactaagatggggggtttcgtcctggtgcacagggattccacacattccccgtttcattaagcatgaaagtaacatcaatacagttctaggtctgagaatcgacctgcccttttcaaagttcaatcaaggaaagaaccaatcctcccgtacagtggtgacgcaaaagcatttggcgttgcggaaccgaacaccagtccgctcggttcgatgactcattctcatcaaacgtatgctaatgttgaatgcttgagtagtattaatagttaagaaatttcctgtcgcgataaaaaaaaaacaaaatatatctatttgtaactcgattgataatcaaagtaatggaatcacgcatattttgcagggcaaagccatttgggctgggcgattatggcttatcgagaacactcacacaaaaaagtagaaacaatttctcatcacacacatatggttgcgtagaaaattgaagaaaggatcagatgtacttttttccagccaaacattcattattatagtaacacacacacaaaaaaaagttgcaacaaaacagacacacgaacaatattatgctaaacataaattacttatcatgagagtaccgcataaacacatatgttgaatgctcagcagtagtgaagtcaataaaacgtatgccaatagatgaaatcatattttaacagcaca

SEQ ID NO: 25 - DNA, 657 bp, Aedes aegypti
atggctccaattgtgctgtatcatttccccatgtcgccaccatcccggtcggctctgctggtggcccgcaatctcggattggatgtagaggtcaagattttgaacttgatggccggcgagcacatgcaggaggaattcgtcaagattaatccccaacacacggtcccgactgtcgtcgacgacgactacgtcctgtgggaatcgaaagccatcgccacctacctggtggagcagcaccagccggacagcacattgtacccggcggatccgaaacagcgaggcatcatcaaccaacggttgtactttgattcgactgttctgtttgcccgcgcttatgctgctgtcgctccgttaatgcgccaaggtgccacctcgatcccacaggacaaaaaggacgccatcctggaagccctaggaacgttgaacggatacctcgatggacaggattgggtcgccggcgaaaataccaccgtggccgatttgtgccttctggcaaccgtgtcatctctagagaaacttggtgttgacctgagtgatctgccgaacatcactgcctggctggaacgctgcaaatctctgccgggatttgaggaaaacgaagagggtgccagcatgttcggaaatggactgaaatccaagctggaggaacctttctaa

SEQ ID NO: 26 - DNA, 4431 bp, Aedes aegypti
atggatagctcgcggaagccggaaaagaagaatccccgcaagggagcgttcttcctgtcgcagctgctgtacggctggctgttgccgctgttcttcaaaggatgccgccgaggactcagcaaagacgatctcaccaagtgccttaaggaagacaaatcggaagatctgggcgacagattggaagaacaatgggaaaaggaaatcgttcgagcccggaggaaaaaccgcccacctaggcttcgtgacgctctcttccgaacattttataagcagtgcttattcgacggtttcctggtgttttcattcgtattaatcaaatcgattttaccattggttttggcgcaactgctaatccagttccaagcgccgttgaacacaacgcatctgaccacaccaagtaccgatacggtttccacgccgccagttcctgatgtcttccttacaaccacgttgcattttctacaggaccacgactcgaatgacaccaacgtcgatcgcatacgccgtagcatagctgacggaaaacattcaatcggccagaatcatcttaagccgcaagactacggcgatgatgacgagggatacgatgacctcgaagaggaagaaggcccgaccaaggagcgcaagttgacaagcgtagctgactttatcgattacgcgtggaacgatgcccactggttggcgggactattgatactactgacattgctgggatgtttctgtaatcatcactccgatctgagacaacgattggtcggagcgcggatgcgaattgcttgctgctcggcaatctatcggaaaaccttgcgtatgtcgaagaaagccgccggacaaacaccggccggctatctgatcaatctgctatcgaacgacgtcagccggttggactatggatttatatttttacattacgtctgggtgttgccgtttcaggcagttttcacgtgctaccttatctggaggcaagtacaatgggcagcagtggttggagtcgttggtcttctgctgaagaccattccagttcaaacgggacttgggcgactgcagtcggttctacggatgcgtgtagccaagcgaactgatcaacgagtgggtataatgaacgaactgatccaaggtattcaggtgatcaagatgtacgcttgggagaaacctttccacacggttgtttcgttggctcgtaaaaaggaagtccgccagattcggtgggcatcgtacattcgcgggatctatctcagtacgatgatgtttactgaaagatctacgcttttcatagcgatcgcaacttgctattttgagggtcgcccaatcacggcagatattgtgtttcctatggcacagtttttcaatattctccagttaacggcggccattttctatccattggctatttcacttggtgcggaagctttggtgtcgattgatagaattcaagagtttcttggtatggaggaacaagacaaaaagatgattggtttgaagaagaacgagaatgaattggtgaaagtgtgccctactgtagccgtagaattcaacaatgtttccgccagttgggagaataataaggacagaacattgaaggatttaaatctcacagccaaaactggacaactgctggcagttatcgggcccgttggggctggcaaaagctctctacttcagttaattttgggcgagttgcccatactcaacgggacggcgaacatcaacggagaagtctcgtacggctgccaagaaccgtggctgtttacgggaacagttcggagcaatatcctattcggtctaacctatgaccgcaaacgttaccaagaggttgtgaaacactgtgccttattgacagactttgaacagcttccggatggagataaaacagttgtgggagaacgcggaacctcactctcaggtggacagcgcgctagggtgaacttggctcgtgctgtgtacaagaatgctaacatctatctgctggatgatccacttagtgccgttgatacccacgttgggcgccatctgtttgatgaagttatgggccctagaggttatttggcaaaacaacaagcaaccaggattttagtaacccatcaggttcatttcctaaaggaagctgattggattatcataattgatagtggaaaaatactaatgcaaggaacatatcaggagttgtccaacagtgatttagacttcacaaaattgatgggatcttccgaagagaatacagacagcattgaggacgaagaggtagaagaattagcagatgaagaaattcctttcattgatggtgtaaagggcgagagtcataaactgcttaagagctcaacgtccgtaggggcacgaggttcgatgtcatgtgcttctagcgttgctgaagaccttggtcgaactgtcaatgaagatcaagcagaaggtaccctagcgctacgagtgtggactacgtacttcctagcaggcggaaacgtctgtcttcttctgttcactgctttcatgctcattttctcacaagtagtggtcagtggatcggactactttgtaacatactggacacgtcaagaggagcgtcgcattcgtgacctgccggtagattattctaccgaagatttgctcatatcctatggtattatcattattggagtcgtaactttcactatctaccgaggatacctgtttttcaacatatgcatgaaggcatcgcgaactctgcacgatcgtatgtttgcgaagatcctcgcggctccgatgaggtttttcgacaccaatccttcgggacgtatcctgaatcgtttctcgaaagatatgggcgctattgatgaattactaccgaaagcgatcatggatgctgtacaggtgctcttggtaatgatcggtattctggtagtaatcgccatgatgaatcctattctgcttgtggcactgttgggtgcgattgttctgtttgcgatagtgctcaaactatatctaagacctacacaggatctcaaacgactggaaggaatcacgcgaagtcctgtcttttcgcatctttcggcaactttgaccggcctttcaacgattcgagcaaatgcagcccaacagaagatcactcaggagttcgatgcactgcagaacgtacactctgctgtctggcagctaaccatgtccagcaatgccgctttgggattgtggctggattgcatcagcactgcgtttgtcgcctgtgtaacatttagtttcatagtgatgcatggagaaactttcagcgccaatgttggtttggccatttcccaggcaatgattcttaccggtatggttcagtacggtatacgtcaaacagcggaatctatccagcaaatgacttcggtcgaacgcgtgattcagtacacagagatcccatcggaaaatgatcctccgaagattccgccaagcgattggccttggcgtggtcacatagagttccgtgacatgtcattgcggtacgatgccaacagtcccccggtattgaagcacttggatctgacgattgcaccaacctggaaggttggaattgtcggacgaaccggagcagggaagtcatctttgataggagcactgttccgtttggcaccgatagagggaaaaatactgattgatggtattgataccggcattgtatcgttggagtctctgcgatcaaagatctctattatacctcaggatccagtgctgttcagcgctacaattcggtacaatctagatcctttcaatctgtacgatgatgacagtctttggacagcgatcaacgaagtggagctcagaacggctattagtggcttagactacatggttacagaaagcggttcaaacttcagcgttggacaaaggcagttgatctgtctagcgagagcaattctaagaaataacaaaatcttggtgcttgatgaagccacagcgaatgttgatcctcaaaccgatgccctcattcagaagacaataagagaaaagttcaaaaagtgcacggttttgaccgtggcccatcgattacacaccgttatggattcggatcgaatcctagtgatggatgcgggagaggcacgcgaattcgacgtaccgcacatacttctacagcaggagggaggtgtactgagggacatggtggaagcaaccggtccatccgaatctgaatctctcaaacgcattgcagcggaaacgtacgctgcgatggaccccaaccggcatctactgaacggcctgccaaatccttggcgttttagcaaggaaggccaataagcaagagatttcattatttttcatctattttattacctcttaagagtaaaatcaaaccaaattttgataagaaaggcggaaacaagtttacgtagtcgttaagtttttcgataaatatttacattgtttaaatgtaatgaaagctatgaattaagcacgatgaacaataaattgtctgctccgcaaaat

SEQ ID NO: 27 - DNA, 2004 bp, Aedes aegypti
tcgatgaccgtgatagggttgctattcattgcggttgggtccggtggaatcaaaccgtgtgtttcggcatttggaggcgatcaattcaagctaccggagcaagcagttcagctggctaaatttttctccttgttctactttgctatcaatgccggttccctcatatcaaccacattaacaccaattctccgagaagacattcattgtttcaatgaaactgattgtttttcgctcgcctttggagtgccggcagtgctaatgattttctccatactggtgtttgcatgtggcaaatcgatgtactcgatcaaaaaaccagcgggcaatatggttgtactggtattcaaatgcattggaaacgctttgtcaacgaaagtcaaggaacaagatgaaaatccgagatctcattggttggattacgccgagatgaaatatggaagacaaatcgtgcaggatatcaagtcacttatgaagattttggtattgtacattccactgcctgttttttgggcacttttcgatcaacaaggatctcgatggactttccaagctacgaggatgaacggtgaaataggagcattcacaatcaaacccgaccaaatgcaagttatcaatcccctgctaattcttgcatttattccattcttcgaaggtttagtataccccgttttagaaaaggtaggcattcgtactcctttgcaaaagctctctttcggcggcattctggcgggagcagcatttgttctatcagggtttgttgaactcgcactggacgaaactaatgcagttatgcccacaccaatggagtctcagctgagaatatacaacggactgccgtgcgattatcacttcaaatcggaaatacctaacttcaacaacttcaccgtgcaatcaattggcgccttcgaagccatccacgtgagcaccatgcagaacactacattccgattcacagctgaaactacggagagcttttgcgtgaaagcaaatatgtccaacatcacgggaaccttctatctgaaccctggaaatgctgtaagctacttcatcagccagaaagcaaacaaggttaatcttctagagtatgcagactcggcggaaaaggacaaacgtggccttcctcgggtgagattcttggcaaacataaaaaccgccaaaaaagtgctgctcaaaaacagctggaacactgatgctcagtataatgtgtcgttgaacacgtatgatcagtttcctgtagcggatggagaatacgacctgtatagtgacaatggaacgaggaaaattgcttctgtgaagctggaactaggaggagtttacaccgttatactgcacgaggttttggagaacgaatttaaacttcaaagacacaccgtaactccctccaactctctgcacatgctctggctggtaccgcagtacgtggtcatcacagccggagaagtgatgttctcaatcactggtctacagttctcctactcgcaagcgcccgagagtatgaagtccgtcattcaagccttctggttgctgacggttgcaatcggaaacatgctggtcgtgtttattgccgaggccaaattcgtcgaatctcaatcggtcgagttcttcttgtttgcggtgctgatgttcctggatatggcgctcttcatgatccttgcagctcgctatcactactcggaagacgaccacaaggatatcgaatcgggtgatatgaagaaaccggacattgataagatggacgacgttccatcgaagctcgatcaaggtccgttggagaagggattggaagctccaaaggcggcctacacgaacgaagcataccgagaagactgagtgctgtgaaagttagaaactaaattttctctcttatggtattaatttgttttcagtatattttttgaatggttcatttttaacctgttctgttttctgttgtgaaaatattctaaattacaattgtcgatgtaaattgctttgtagtaagatttctttaatttaaagaaaata

SEQ ID NO: 28 - DNA, 1710 bp, Aedes aegypti
atggaggtcaacaccagcacagtcgagaacctattggggattggacgagccgacgatgatcatgaccatgaggaagatgaaggggatattgtggccgccaaggcaactgccatggtagttttgttcgctgtgagcatgatttgcggaattgtgccgttcaaattggcgaaatggttcaaatggacggacagtgcctccgacgcaaaagccagcctcgtggtttccatattactgtcatttggaggaggggcacttctgtgtactactttccagcatctaatacctgagatcaacgaagccattcacgacctcacccagcagggacttatgccagagttgagtttcagtttgggagaatttttaatgtgcgccggtttcttcattatctatctagtcgaagaattagtgcacgtgtatctgcacagacatgagcgcaaaatgcatgaagccaaaatgaacgagctggaagttgccggtggcgctattatgcgaggtacacatgcgagggaaagcattattatgcgaaagaaaaacactgaaaatcgcaacggaagtatttccaccgcagatctaatctcgaatgatatggaaaaccagaagcgggggatttcggtcgcttcaaatgatatgtcagtggcgcattcagggcatccccatgaccaccacgtgcatcatcatatcccaccaacggaaacggacggaaagattgtctcatctattcgtggcctgctgattgtgctggcactttcagtgcatgagctgttcgagggactggcagttggcctggaaggatctgccgcaactgtttggtacatgtttggggcagtggccgcccacaagtatgtgatagctttctgcgtgggagtggaactgattgttgcccgtaccaagttctggttggcagttgcatacatcttcacctattcggtagtcagtccactcgggatggcgatcggaatcctcctcagcaacggttcttcttcggacgatacccaagtcgtttccgttgttctgcagggtttggcctccggaacgttgctgtatgttattttcttcgaagtactctccaaggaccggtccggattgtggcagtatttggccgtgttttgcggattcttcttcatgtttggcatcaagttcctgaccggacatagccacagtcacagccatggactgacgggagatgaccatgaccacgaccacgatcataaccatgatcaccactagcggccgatggagaaccgagaagaagcttatcgtcaccgtttgctacaagtgaacacgtgaatgtaggcagcaggagatcgaagtgacggagggtcgtaacaattgttgccgaacaattattgttaaaaaaaacgagtagaaatatgaacagtatttgcttgaggtgagccggcgtccgaaactctttgcagctgtgatagtgtgaaaacaagttttaaccttacacaaaaggatagaagcttcttatacgatttgatagatgtttaagaaaccatagatagatagcgagttttataatgaatatcggttttagtacaaaccgctggatgcttgaggatgtgattcaacgatacataatgctaaccaacagtaagattttagccgatctttacaattatcaagacttgaattgtatctgtgtaccgtaatagcatttgcttattatttttacgtcagattgtattaatagattagttttaatat

SEQ ID NO: 29 - DNA, 5458 bp, Aedes aegypti
cgatattttgtagttcgacgatttgtcagactcaaccgaaacacgaatcagagccgtagtgaacagctaagtgttttgggaaatgctgacgagtttgcgcactatcacatctcactgtcacccactaccggcatggtccccgtgcgataaagccctttacgtacccaatgattgcaggtgataagagtcgcactgatagggtgattgtggccatttcgataagtttgcgggccgatttgtgggaacaaaagcctacgccttcggtgccgaaatgtgtcagtgctagcgcgaatttcaacgggatcggttcggtaccgggagtacggtgaagatattattggtgttgcgtgggaggaaggaatccgatcaagaggacgaagttcaacgcaattcacctgcaaggtgaaggcgaaggcaacgatacgacgacggcaacgcccgtttcagcgggaaaccccactccaatcaatatgacgtccaacggacaaaagttcctcctgctgctggagaagaattggatcatccagaagcggcactatctgcagacgctgttcgaaatcatcattccggtgctgtgctgttcaatcttgctgttagttcgcggtctcgtagatcctgaatacgtagaaaagagtacaatcttcagcgctttatcaacggattcgttcgccaatcgatttcaaattaatggtgaacagctaaactattcaatagcatattcaccgcagaatgtcatactggatcgaatcatggcagatgctgccgaatcgctggatactcaagtgatcgctttcccggacgctcaaaccatggagtcgttcctcatgagccataacctggtggctggagtggagtttggtgattctctatcaaacgtaatcagccttccggaaaagctcagctacgccatacgcttccccgctgaactccggttcggttttggtctgctctcggactgggaaactcgtctcctcatggtaccgtttacgcctcgtctacgtgatccctactcaaatgacggtggaactcccagttattacgcagaaaatttcctctccgttcaaacagcagtttcaaaggccatcatccgggagcgtaactctgcatcgaaaatgcatcccgtcttcattcagcgtttcccatatccaccgtactacgacgacccgatcctgcaagctatggagcagctgctgtccctggtcatcctgctctccttcttctacacatgcatcgtgatggtcaagcacatcgccgtcgagaaggaacgacagctgaaggaagcgatgaagatcatgggactgcccaactggctgcactgggcggcgtggttcgtcaagaacatcctgctgctagtgattgctatatcgctgataacggtgctactttgtgtctccgtcaacggcagcgccatcctggagtactcggactggaccgctgtatggtttttcctgttcgtgtattgtatcgcgatcatctgcttctccttcatgatgagtgtcttcttcaataaagctaacatcgcctctggtatcgctggattgatgtggttcgtgttcgtgatgccgtacaatgtgaccgcgcagaactacgacggaatgagcactgggagcaaggtcggtttgagtttgttcttcaacagtggaatgtcgtttgcgatgatgagtaccctgcgcatggaagcaaaccaagtgggattgcggtggagcagcttgttcactccggctacagttgatgacggtttcagtgttggtacagcgatcataatgctattggtggacgcgttgatctacctggcgatcgctttgtacgtggaacaggttatgccaggccagtttggagtcgctaaaccgtggaattttctgatcaagaaggagttttggaccaagaaggaatccaaagcggaaaacgttccacgacgactaattgaaagacagaactcgaagtattttgaggcagaacccaatggaacgaatgcaggaattcagacggtcgatttgcgcaaagtgttcaacgggaataaggtggcggtcgaaggattgaatgtgaaaatgtacgaagatcagattacagttttacttggtcacaacggagcaggtaaaaccaccactatgtctatgcttacggggatgttttcgccaacttctggcacagcatatctcaatggacatgacatcagaacggatcttgagggagtccgacaatctatgggattgtgtcctcagcacaatgtactctttgatgagatgaccgtcatcgagcatctgaagttcttcggtaaactgaaaggcgtaccgaaggcagcactcgacggtgagatcgatagatatttgaggatgttggagctgctggacaaggggaatgctcagtcgcaaacactctccggtggtatgaaacgaaagctggcagtggggatcgctctttgcggtggttcaaaagtagtattgctcgacgaaccaacgtcaggaatggatccttcggctcgtagagctctttgggatttactccagaaggaaaagaagaaccgtactatgctgctgtccacgcacttcatggatgaagcggatgttttgggtgatcgtatcgcgatcatggcagatggagtcctgaaaacggttggatctccgttctttctcaagaagacatttggcgttggatatcgactgatttgcgttaagggcccatattgcaacagggatctgctttgtcagatcttaagaaactacattccggatgttcgggtcgaaaccgacattggttcggagttgtcgttcgtgttgaaggaggactatattggagtatttcagaaaatgcttgaagagttggaacggcggatggcggaatgcggcattacaagctttggtatttctctgaccacgatggaggaggtgtttttgaaggctggcagcgacacactttcggaaacagaccattctaatggaacttctattgagacgatcaatcgaaactattcgttgaacaacatgaaccttttgacgggaaatgacctgctgctcagtcaaatcaagggtcaatttttgaagaaaatgttgtcttcccttcgttcgtggggtacgctaatcatccagaacgcgattccaatcttcttcgtcattatgtcctttgtgattgttcaaagcatttccaaagatcaagatttaccaccgttgaagatatctctggatccctacaaagaaactgtgactgtattggaaggtaacgctgtagttgatccacgagtacaagcctatcagaatctattgaacaatgtgggtggatcgcatcgactagagactgtacccaatacggtgtcggagtatatcttggaaaaatcccaaacagccatctccgaagttaacgtccgttacatggtgggcgccacccttaacgatacggcctacaccggatggttcaacaacaaaggctatcacacagccccattggctttgtcgctgattttcaacgccatcatgagcaccgaatgcccaacttgtgagatcacagtcgtcaacaaacctcttccgtttcagctgaaaacaacgctgtccatcgtgaacaccgggctcaacgccggatttcagctggccttcaacaccgggttcgcaatggctttcgtcggtgcactcttcattctgttctacatccgcgaacgaacatcgcgtgcaaaactactccaatatgtcagtggaactaacatcgctctctactggaccgtcgccttcatctgggactacctcatgttcctagtaacctgcttgctgtacattgctacgcttgccatctttcaagaggaaggttggtcgtccttcgaagagcttggacgcgtattccttctcttaatgcttttcggagtagcattcctgccgactacctatctttgctcgttcctgttcacagtaccggccacaggattcgtagtcgtcatgctgatcaacatcgcctccggagcaatattcttcacagccgttacgttgctcaagtttgatggcatcgatctggacgatattggcaatgccctcgagtgggttttcatgttcttcccgaacttcgtactcacacacggactgaacaatatcaaccagatcaccagcaccaattcattttgtagaaagcagtgcgcccttgttgatggctgctctctggacaacacctgcgcatggagcgagcgatgctgcgttccagatgtgttctcctttgaagagcttggtatcaatcgcaacttgctgttccttgcgtttgtgggagtctcgtcgttcttggttattttggcattggagtatcgtttgatacacagaattattgatagtgtattcaaacgaaagcagccttgggcaccggccccagtggatgaagattcggatgtggcagcggagaagaaacgagttcaagctatgacgcatgtcgagcgaaaccagtatagcttggtgatgaaagacttgaccaagtactacaagagtttcctagcggtgaacaatctctcggtggcgatcgaccgttcagagtgctttggattattgggagtaaacggagccggaaaaaccactacattcaagatgatgactggagatgagaacttctcctccggggaagcttgggtcaagggcgttagcttggcgaccaacatgagcaccgtttatcagaagattggttactgtccgcagtttgatgctttgctcgatgatctgacaggacgcgaaacgttgaagatttatgcccttctgagaggagttcgccatgaggacgttggtaatgtatcgctgactctggcggaggacctgaacttcctgaagcacatcgacaagaaaacaaaggagtacagtggcggaaacaagcgaaagctgagcacggctttggccttgatgggtaatccatcggtggtgtatttggacgaaccgacgacaggcatggacccaggggcgaagcgtcagttctgggacatgatctgtaaggtgaggagttcaggcaaatcgatcgtgctgacatcacacagcatggaggaatgcgaagctctgtgcacgcgactagcgatcatggtcaacggagagttcaaatgtttgggttcaactcaacacttgaagaacaagttctccaaggggttcctattgaccatcaaggtgaataggtcagaagacgctcaggaacagcagcgaaggattggtgaggtgaaggcattcgtgatgaatcagtttggcggggcagtgttgaaggaggagtatcaagactcgctgagcttccacgtaccacaaactgatctcaagtggtcggcaatgtttggattgatggagtccaacaaggatcggctgagtattgaggactatgcacttgggcagacatcgttggagcaggtgttcctgttcttcacgaagtaccagcgtatagtctgagcagctgatgaactttgtttatatacacttatttattgtaaagctaatacatacgaatcttgttgtttgaaacaaaa

SEQ ID NO: 30 - DNA, 180 bp, Aedes aegypti
atgaacttcacaaagttatttctcctgatcgccgtggctgttcttctcctgaccgggcagtctgaagcaggtggcctcaagaagctgggaaagaaattgggaggtgcaggaaaacgagttttcaatgctgccgagaaggctcttcctgttgtagctggggctaaagctctaggaaagtag

SEQ ID NO: 31 - DNA, 2638 bp, Aedes aegypti
atcttcaccttaattactgtcagccaatttagcatccaaagaaatacatccaattcacagatgttaacaaacggcaccactgccgtcattcagaacaataatctcgctaatcaccatcactcttgttcaatggcgcgcaaatcgtttcccaatctaataagtaagtagacagcaggttggcatggcgacgcgacgcgggttgctgttaaagtaaacgaattccacatcacatcagctcgcgcaaatctccatcggttcgttgttctctaatctgttctacctacagcagctctctgatgggcccatactgcgatctaagctcactggtcgcccaacagctgagcggagcagagaacaaattgctgcttctagcatcgttatttaatcgctgcagacgatcgcgcgcgcttcaggtgaaatatacaaattttgtcttcagtgcagtctcgttacaatccaaacggaatcatgaagctgttttcatgttagcgaatcgtgtaacgccgcaccatacctgtaacccacttttcgataagataagataaaatgcgctcttatcagcggtggagtaccccaaccgattcaagtataatgcaaccggtgcgattggggctctcatttcgacggtggatcgcgatacgagatattctagaatcgacgtccgcccatcaaagacggaagtgtgtcccaacaggaatcaacgtgcgttgtgggattaggaaatcaagtgcgcaaaagtgactcatcccgtgatccgtaacgttcgaaactcgtgtaggaaaattagttcaattatatccgcaagacagtgactgcagctagtaattaagtgaataccaagacgattgttatgtgcgaatagaagaatttccaatcaacgatgaagattctagtgaagtgttcaatcgtggcgatctgcctgggattgatcttgaccagcacagtaggcgccgccgatgatgacgacgccagcaaaaacagcacaagcgacaaaaaagataactctaccgatagcaatgccaatcaaacatccaacaaggagccaaatcctctatgcaacacgtgctcctgcaacgccaaggagcgaaccttcgactgctcctcgatgaagctgtacaagctgttcaacatgagcgactgggctacgttgaacgacagcggagtcgccaccgacaagatgatcctcagccgtaacggcatcccagaagtgcctcaatttcccaagttcgatgtcaaagtgttggatctgagcttcaacaacatcagcgtcatcgtcaagaaggccttctactacctgaaggatctggaggtgctggatttgtcgcataacatgctgactcgaagagcgctcattccggagatcttcgaaggaagctacaatccggacgagtacctaccgatggaaaagttgcgggtgctacgcttgggcaacaacgaactgcactcgctggatccggatctgttcgagcatttgcccgtcctggaggagctgagcttgaaggagaatgtgttcaagatcatcgatcagtcgacggaggtcgccatcgggaacattatgaccctgagggtgttggatttgagctacatggagctgttggagattccgaagcggatcttccactccccgcaagggctgcagtatttgaacctgacgggaaacttgttgacgagtgttcctgaggcgttggactacgcgatcaatctgaagtggctcagtttggacgagaatccgatagagagtattcagggggaaggcgtattcccggcgatcaagacattggagtacctgagcctttcctacattacgccgttgaaggtgattggacgcggggcgttcagcaagttggaaggtctcaaggagatacacatttgcaacaatccggagctgacgcatctgcacggagaagcgttcagccgggccaacgcggacgatccggggcgaatggagtggccccgagtgaagcagctctacctgcataacaacaatctgagtatgttggactcgcagctgctggcccggtgggacgacatggaggtgattgacgtgcgggtcaacccgtggttgtgcgactgcgacaaccagtggatggtggagaccctgctggcgatcatcgagaagacgacgcccaaaatcatgaacaatattgtatgcgccgcccctgatcagatgaagggcctttcgatggtcgacctggagcacaagcacagccagatgcgttgcctggacaagtacggcaacaggccgcagaacgatggcgctctgttgattggcctgctgatcggcatcttggcgggcattcccctcaccgcggcggtcatcatgatctaccggcggggttgtttcggcctggtccgccggggaccggccgacttttcgcgggctttctacacccgtgccaccaacgacgaaaacttctagttcgtcagcaattgattgtgccaaataatagtaggtctccatagagtgagacgatttctaagtcagggtttgtgatgacattttagttatgcctttccattcaagatagtgttagtaggctatgacttcatcagtgattagtgttaaggtgtgtgtgaagaagcatccaagtgagccataataaaatcgattacagggagtatttctctgacagtgact

SEQ ID NO: 32 - DNA, 489 bp, Aedes aegypti
atgttcaaatacttgcttctggttgcactcgtcccagctgtaatgctgcagaatgccgacaacatcgatcacgaatcgtggcctcaggtttccatccggccatgcgctggagtacgtccaccaccccgtgcagttcggattgaaggatgtgtagagatgccgtgcctgttgccccgtggacgcgatgccaatatggctatggacttcactgctgttcaggacgctaccaacttgcaaacccgtgtgaccgccactgctttgggtatcactgctccctatgagctgccagctgaccgtgccgctgcttgcaactggctggtgcagtcccgttgcccaatctcggccggagaggatctgacttatcatctgagtatgcccgtgactgccatctatccgctggtcagtgtgaccatcgaaatggatttggttgaccagagccagcagtcccacggatgcttcgtcgtggatgcccgtgttgttgcgaactaa

SEQ ID NO: 33 - DNA, 555 bp, Aedes aegypti
atgaaagctttatttcttgccttggtgatcgccatcagtggtgtatcgtgtggtcctatctcagttagtgataacaacattggagacatcatttcggttggagttaacgtagaagccgatatcaagagtgaaattaacgctagcatcgtaaacatactggtcgcgttattggcaaatattgacctcggaccaaacccaccgccactaccaccactaccgccactacccccgttcccaccaactggattgccaccaactggactgccaccaactggattacctccaacaactccggtaacacagccaccaacttccgaagtaactcaacagccaactactccggtaacacagccaccaacttccgaagtaactcagcagccaacgactgtgtcaccctcgccgttctcaagtatccatgttgatgaaatgatcaaaactatccaggatatcaaagagcaggcatccggtgcaaatctgaccaaagagcagagggaacagatcttgcgggaacatgctcaacagttggtgcaaaaattgatggaaaatagacaataa

SEQ ID NO: 34 - DNA, 879 bp, Aedes aegypti
atgagctacatgtatgacgcagaggaggtcgaacacttcaggaaaatcgcctgttcccaaaggaaaattccaaagatcatggcgctgaacgggttcctggagttcttccagcgcgagaagacctttcgccccaagaagaagtttacccaaggaactattcgctattcactacacaagcaagcccatgctagtctgcagtccggaataaatttgagagaagtggttaaactgcctccaggagaaaacatgaacgattggctagccgtgcacgtagtggattttttcaatcgaatcaatctcatatacggcaccatatccgaatactgtaacgaaacgacctgcccaacgatgagcgacccgaccatctctactaaatacgagtatttgtgggccgatggggagatattcaagaagccaacgcaacttcctgctccccggtacgttgaactgctgatggactgggtcgagaatcaaatcaacaatgaagcgctcttccccgtgtcgaccgacgtgccatttcccaaatccttcccgacgctttgcaagaagattctggctagactgtttcgcgttttcgtgcatgtctatatccaccacttcgatcgaatcgtcagtattggagcggaagctcacgtaaacacgtgctacaagcatttttactactttatccaagaattcgatttgatgtcagcgaaggagttggaaccgctagccgtgatgactgctcagatgtgtaaagatgtgtaatttgggttattaaatgagggttttacatgacgcaaaactacattgtataaaacaattcgaacaatagtgagtttttaaatcaggttgttgctagcctttaatgcataataaaagatgatagggaactaaaag

SEQ ID NO: 35 - DNA, 1543 bp, Aedes aegypti
cttttgcgctgatttcataaaagcgacggcaaccaatcagtcaaaaaactcgaactcggcccaaatatcggcaagaaatcgctttttcatccaacatctagcgcagaaaatactccaaaatgctgatcaaagtgaagacccttaccggaaaggaaatcgagatcgacatcgagcccacggataaagtggaccggatcaaggaacgagtcgaggagaaggaaggaattccccctcagcagcagcgtctcattttctcgggcaagcaaatgaatgacgataaaacagcccaggattacaaagtgcagggtggatcggtgctccacttggtcctggccctccgcggaggtcatctgtgaagccgaaaggccgttaaatcgtccggagccggaagcgggaataatttgtgatgcatgtgagaaacagccagcaggacttaaaagtccggaactaagatgaaagctcactttgattctcttttctgataaggttaaattattctgttttatcgtaatttgctgaagaataaaaaatgttcgaaagtgatccgattggaatgtaatgcggtttatcttttaaatttatgtttacttgtaatttagtttagtatttcagagcagtccagggttgaatgtagatctcttcttagagacatgatcactattttaatgtaagcctcaagattttttttttcgtataaaggttctaaattatctattttaacctaaatcaaaattgttccttcagttactttttccttattcttcttgcagtgaatcctagtacattataaaggtcccagagaaatctttatacaactaacccctcagcttcttcaaaaaattctgggagaacattactggaagaaaaacctcagatatgcaactggtgattccactctagatttttttcgtctggaatactttcaaggatttctccaccaattctcccaaaaaatgcttccagcaattttcaagttttgcttgaaggaatttgctccagaaaatccaagaactatctcatcgattcatacagaaaatcctttgagacttctccaagagtacaggattttttcaaagatcctagtagaagttcatcaagggttaccttagaccatattcaagttttaccggttaaccactggttaggattttcttcagaaattcctcttggattgtgtttcaaaattgtctgaaaaacgccattccactgaacacatcctttagggctggtaggtagcagttttcttattagagacgttcataattttaagtctctaacaaaactctattttgatcgaaaattccaattttcagcagcctagcaagaaagtttctaggttttcttctgtaatcaacatattctctagtcacttgtcttctatttatccttactaagaataaaaaaaaaataagagactccctataagccttcaaatactctaacgtgaccattccacaggttcttctcagatttatccaggaaaaaccaagaacattaaaaaaaatcccaaccaattctccaatgaatttgtccagtgaatttt

SEQ ID NO: 36 - DNA, 591 bp, Aedes aegypti
ctatcagatccaatataaatgacccgcatggtgcattctgtcacacacgatttttcactcctatccttaaaatgatgcgcttcgttgttcggatcgccgctgctctagcgatttgggcgtctatcgcctcagctcaggagcttttctgtacctcgccatcaaagttctacgtctccagagttgtgcacaattggatcggtgccgccgaatactgtcatttgcttggtatgcggatggctgtgatcgattccgaagccaaacagaacgaaattgtccggctggtggagcattcgttggtgttcaatgccacccggacggatctgtggatcggagcaagtgatctggccgaagagggcaactttgtgtggcttgaaaccggtatggaggtgagcaggacttacaccaactgggcccgaagtcagccggacaatgccgggactggggagcattgcattcacatgtggtacgaaccgtccaggaatttcacctggcagtggaatgactggcactgcgaacggaaattggtgccggtttgtgagaatataccgtaaagtgcagaaatgcaaagcactttcaaatacttctaataaa

SEQ ID NO: 37 - DNA, 563 bp, Aedes aegypti
ttccgatcagtcacgcgtagtaagtttcgattgaagtgttctgttaaaaaaatgaagtgtctagtactgatttcgctgttggccgttggttctcaggccttcttcaccccggagcaacatgaagtggccaagaggctgacgatggcctgcgcaaccgaaatcggcgaaggactgccggataatgtgggaaaccgattccgggagggtgatcttacgctgaccgatgacaagagcaagtgtttcatgaagtgcgtgttcggtaaggtaggctttatcgacgatgccggaacggtaaacaaggaagttctagtggaaaagctgagcaaaggaaatacccaggctaaggcggaaatgtttgctgagaagtgcaatatgttcgagggagccaacggatgtgaaaaagctcatgggcttttcgagtgttactggaagaataaggagatctttgcgtgagcatcgcaccggaatacgtgaaattgtgaaacgggttgttgttgattgtaaccattggcagtattacgaatgacgaataaagtgatttatgagagtgatatctcgcagtt

SEQ ID NO: 38 - DNA, 828 bp, Aedes aegypti
atcgatcgatcgattgtgtggtccggcagtgacagtgagtgcattcggcaagtgattccaaaaatggctttcctcctgttgggattggttatgagtgtggtgttggccaacgtttccggtgctatctacgaccatcagccgaagtcgaagctttgtgcgaacagttacagtgtgtcttgtttgaagatggagattgtgtcatttttggaacgactttcggaccagaacgagtacagtgttctctccggagtgagtgttgtcagggatgcgaatgtgaatgtcaccaaaacggccgatattatctcggaagtgtcaaggatcttccccaccgatcccaacaagcgattggatgaattcctgatcattaagctgaacgactatctgaagagccactcgctgcggctgaagctaatggataaggaagcggtatcgaaggcgcgagaactgttcgtggggcgtaaaggtggcagcaagctgggcaagaagggcggattggagaccatgattgctgctgcaatgatgatgaaaggaacattaggtgccatcgccatgggagggcttgcgatgctcgccggcaaagcgttgatgacggggctcctggcgatgacactggcaaccgttgttggtctcaaatccctcacttccggcggcggtcagaaatccaccacctacgagatcgtggccaaaccgatgtacacccactcgcactcggaaacggaggaggttagccacggacacggcggaggcggaggctacggaggaagctggggacgttcgcaacggctagatgtgccagcatattccggccgaggcgatctgaagctctga

SEQ ID NO: 39 - DNA, 1947 bp, Aedes aegypti
ggtagtttgtgcctcggaacaggacggttcactaagagaagaaggaagaaccgggtgcgcggtcgagaactcttgattttagcgtgtgtcgtcccgttttggtgaagttacagatagattatacagaacatgtggaaatcgtcggtggtgtgtttggcccttctgggggtcctggggtcggttagcggaacgacgaaactgcaggaacgctacagctggcgtcagttggactttgtgttcccgaaccagcagctgaagcagcaggccttggccagcggagactacgtgcccacgaatggactcccggtcggaatcgaacgctgggagaacaagctctttgtgtcggtcccaagatggaaggatggtatcccgtccaccctgaactacatcgatatgaatcagaccccgtccgggtcgccaccgctgatcccttacccaagctgggccaacaacgtggccggagactgtcagaatggcctgtcgaccgtgtaccgtatcaaggctgacaagtgtggacgcctctgggttctggacaccggtaccgtcggaatcggaaacaccacccagcagctgtgcccgtacgcgttgaacatctttgaccttaagaccaacacccgtctgcgtcggtatgaactgcgtgccgaagacaccaaccagaacaccttcatcgctaacattgccatcgacatgggacgcagctgcgaggacaccttcgcctacatgtccgatgaactgggctacggactgattgcctactcatttgagaagaacaagtcctggcggttcgagcatagcttcttcttcccagatcctctacgcggagacttcaacgttgccggtctgaacttccaatggggtgaggaaggtatcttcggaatgtcgctttcccccctgcaatccgatggcttccgcaccatgtacttctccccgttggccagtcaccgtgaattcatggtctccactcaggtcctgcgcgacgaagaaggtgccgaagagagcttccacaagttcacctacctgaaggaacgaggacccaacagccatactacatcccgagtcatgagcgaaaccggactgcagctattcaacttgattgaccagaacgccgtcggatgctggcattcgtcgctcccttacagccctgaaaaccatggaatcgtcgaccgtgatgatgtggaactggtcttcccagctgacgtcaagatcgatgccgaagagaacgtgtgggtcatctccgaccgtatgccagtgttcctcatcgccgagctggactacagtgatgtcaacttccgcatcttcaccgctcctctgagcactctggtcgctggaaccgtctgcgacgttaggccttcgctccgaccaggtgccatccagtccaaattcggtggatccgatctgtccacctatccaggcaccacccttctcccagttggctacactcaacccatcagctataccccgtcctcgtacgccccaaccgttgcccctgtcaccaagtacacagccgctccagcgtacgatcacccaacctcccacatgtacacgacccaggaatatccgacgaccgccaaggcgtaccacttcaacaagtaccacaacgtagagtaccaatcgcatggaaatggtcaagctgactaccacttcggacacggtggacactaccacgatcacgaccacgcccatgaatacggtcacgatcacggatactacggcggtggtcaacgccgtcattggggagctggcgccaagaagcccgaagcctggaagcagcagctgtactagaatcgagagaatgcaccatattagagatgtaaccgcacaattttcgttttagagaaatttaggtaaattatttgtcagtgtaaaagtactcgtcaagtttcttatgtttaagtgctaagatcgaacgaaccaactcaagtcgaataaagtaagatttttgttacg

SEQ ID NO: 40 - DNA, 730 bp, Aedes aegypti
atatgatcgtatgtttgggcacagaattatcaacaccgtagacggacttcgtccagtgcccggttcggaccgatataaaagcaactgattctaccacatcccggtatcagttccattttcttcttcgcacatcgtagatcgtggcacataccttccgggaatcgtcctagctgagacatcaagatgaaagtgttcattgccgtattcgctctgatcgcagtggctgccgctgagttcaccgtgtccaccacggaggatttgcaacgctaccgtacggagtgcgtctcttcgctcaacattcccgccgattacgttgagaagtttaagaagtgggagttccccgaagatgacaccaccatgtgctacatcaagtgcgtgttcaacaagatgcagctgttcgacgacaccgagggcccactggttgacaacctagttcaccagctggctcatggccgtgacgccgaggaagttcgcaccgaagtgctgaagtgcgtcgacaagaacaccgacaataacgcttgccactgggccttccgtggattcaagtgcttccagaagaacaacctgtcgctgatcaaggctagcatcaagaaggactgaaaagactgagtagttgttgatattgtgtaactaggagtgttaaattgaacagtaagagagatattcgcggaagaggaatttttggaataaattgattttgttggtggcttattgaaagttatatttcatt

SEQ ID NO: 41 - DNA, 491 bp, Aedes aegypti
ctcgcgagtcgtaacttttcaccagccgaaagtgatctccactgctgtggtttagagttttatcgaatattccattcaaatcgatcaagatgctgacgaaccagatccagcaggccgcccgcatgatgggagcccaggcccgccgcaactatggcgtctcggccgtcgtcctgtccaaggcaaccgatccgattcagcagctcttcatcaacaaactccgcgactacaagagcaagagcagcggcggcaagctggtcgacgccacgcccgagatcgaacgcgaactgaagcaggagctggagaagctggccaagcagtacggaggcgccagcggcgtcgacatgaccgccttcccctccttcaaattcgaggaacccaagatgggccccatcaattccagcagcgcctaagaaaaacaaaaaaaaaagctaggccactgctagacgctcttgtaaagttatcgaaataaattatctgaaatcttaatctag

SEQ ID NO: 42 - DNA, 1057 bp, Aedes aegypti
actacacgtaaatcaaaaataatcttgcgtcaaaagaacggcacgcgatttgaaggaatcagtgaagaatatagcttgaaactgaaaagtactttataaaactactcaataattcttttaactagtggtgtataataatcaaaactcaagcgctatgaatatgttagcgaaagcgttccgagtgccatcggtgaagcagttgtcggttgccgtggccccaaccaaaaacattgtcggcaagcgggatttcaccagaactatttggtacatgtgccgttcggaaaacatcaacggatcgccatcggcctttcccaaagaactcctcaaaccgaccatcagttgttcctgcggttgtgccggctcgaagcatatccacaccaaaggcgagcgtgagcttgttgaatttttgaccgaggaaatcgttgccgaaaagaaagctcagaagaagaaaactcttccaacagacatcgatggattcaaagtgaaattgaacggagctgatgtggaactcaccaagcaagctgacaaggaaaaaattgttgtatccttcaacgtaaaccacaccgtcgatgctgaagatgaagcagaaatcgatcccaatgcagataagccagaattcgcagaaatgcgaagcaaaccacaatttgaagtagacatcgtgagaggatcccagaccctgagttttacttgttcgttccttcagggcgaggcacaagaaggagaatacaatgatgtatttggtatcgatgaaataacattgttcgaaggagaatggaacgataaagtttatgcagtagcaggagatgttctggacggatatctgtacgacttgctaatgaacttcctagaagagaaaggtgtgagcaacgagtttgcagaaaaactatccgatttcagtactgcatatgaacataccgcctacataagcttactggagggtatttccaaattcaccatcgaaaagaaatagattcatcgattgtgtagcatttaaatacgttgataaaaactttaattcctaactttagctgtttctcttttatttatgtgaacaaat

SEQ ID NO: 43 - DNA, 1861 bp, Aedes aegypti
atgaaatcggctctggccattgctttgtgcttctgcctattcacggctgctactgctgctccacatccaggacttatcagctctctgaccggtattattggaaccgttgtgaatggcacagcaactgtgatcaacactgccatcactggcacgacgaacatcatcactggcattctgactggcatcactgcaggtatcactggtacgcagacgtcccttaacactgctatcagcaatgtcggaggccttatcgctaacatcacaaccggagttgctaatgccactggaaccgctctgaccaacgccttctccaccttgatcaacgctcatgttgccttgaacaatgttgctgttgccgtcgccggaaacatctctgccgatgcagctaccgccttgaacaaaactgtctcaggcctccaaacccttgctgccaacctgaactccaccattgccaacgccatcagcgccggaatcaccggaacctctaccttcctgaacaacttgaacaacctcacgaattccattaaggctgccattcagaacgttactggaacattgagcacggtccttccgaatattgcttccaacatccagacccagctgaattccacgattgctggcatcgctggacttgtctcaaatattggcgctaaccttggcactgctgttaacaatgctttgacgaatgccaccactgctctaaccaaggctgaatctgtcctcggtacgttgaccagcaacgttctcggaaacttgacaactcaagctcaagcagctgttcaaaccggtgtcaccaacatcaagaacctggccgatgctctcaagacaacgttgaccaatgctgtcagtgctggtactaccactggaactaacctggcctcagccttgaataaccttacgagcgccatcaactcgaccctccaaggtatcactggtattctgtctggatcggttcccactctactcaacaacgtgcagagccagctgaacaacggcatcagtaccctgaccaatcttgctgcatctgttggaaacaccctcggaacagccgctggaactactctcggcaatgtagtctctgctctggctaacactaaggctgccttggccaccgttgccaccaacgtgcttgccaacgctaccgctttggtgtcggccgatgtgaaggctgccgttgcggacatgcagtctatcgctagtaccctgaatgccaccctggccaacgccgttgccaacggagcagccctgaccagtggagttctgtcaacgctgaacactttggcggccaacttgaacgctacagtccaggccgtggccagtgccgtcgttacggctctgaacactaccaccaccacgacgactaccactacgaccaccacggccgctccgacgaccgccgcacccttctgcggagtcctctgccaactgattaaggctcttggataagttattcctgttttagttaacctacatagaaatgcaatttcttcacttgtgaaagttgaagtttcgtaataaatgcgcaaaacaaattataatggaaaatatttattcctcaaattgtgtccatagtaaatcaagaaagtaaacttcctgaaatatggatatacagtttattctcccttgctcgatattcttaatttcgatatcaagttagagaaccatagcacagcgcaacaaaaatacattattgcatgtctcaaggatctgattatgtgtctctagtagatttgggattgctgaatctgatgccgttctcagaaatttcccagcacgtcacaatttttagctacaggtcgccaaagttatataatacattggttttattgatgtttacctaaaatttgaagtacattttatc

SEQ ID NO: 44 - DNA, 555 bp, Aedes aegypti
cgaaaccttgcaatatccaactcatcctcaaaatgaagtccctgttgatcattggcctgagcctgtgctttgtaggcgttatcgtcgcttatcctgaacccttgctgctcggaggcctcaccgatatcctgtcaaccgtgttgaacacagtgaagaccgccctcacgaccgtggctaatttagtcaacctgatcaacaccgccctggccaatgctgtagcctccggaaatgctgccctgcaaacagctctgaccaacgtccaaactgccgctcaatccatcgaatccacactgaaaagcattgtcagctccactctcacttcgggaccgtctactcagcttagcactttgacgacgcagctgtccaacgccctgaacaacttgaactccatcgtcagccaagtcgttgctgctggaagctctgtccctgcgactttggttaaccagctgaacaccgctctggctaccgtatccaatgtggccgcacaagccaagtctcaagttggatccgttccgtctcttctcggtggacttggtggacttctgggatgagttt

SEQ ID NO: 45 - DNA, 17248 bp, Aedes aegypti
ttatttacaaactcgttcgcggtgccgtgcggttgtgtctcgtgtgtgctctccgtgaaaagttgtgttttttcgtgaaaattcgtttcggaccgggtggaaaactagtggccgaccgtgccccgtcacaatggaagtgaactgtgatggaatccggcaaagtttgcgccaattgcgggagattttcgactgccaacaggtggagccatttttaaggttatccaaaatcgcttcgtcggatttggatcgattggtcgtcgttttggggcagctgctggaggatccgaaatacacagaatccatcggggaccgtttcgggagcattttgctgctgatcgtcgccagcgcttttggcgatgtggaatgcgaagatgacaaggaagagaggaagaaggagagcaatacaaaaacagtgaatcgcgaaggtcaccggcgaaagtgcgtcgcgctttcaaagttgatcagccgctgtccggatgtgttgcgattcagcattcagtacttccgtcggaatccagcgccgttcctagcggaagacaccgtagactacacaccgacatcgaagcagaagaaggtcgaactggaagcgataacggatctggaactggtgcagtgttgctatcggtttctagcggcggatccgatctactttcgatcggtgtggaactggtcggattttttccagcagtatggcacccgggtgggatgcagtcggcagcagttctactacaatcggatattggccctggtgagctgtatgacgcagagccaaatcagtcagctggaccgggagttgccgctggaagcggtggttagagagcagttggaaagcgtggaagatgtggggactcagggatcagagacgggggatgagaagctaaagactggagagaaaatcaagtggaattttcgaagcaacgttgtgacgaacgttggagacgtgatgctgccgatattcgatgccggcaactgtgagttcttccgtgggcaggataaggaaccggatgatttggttgtggtggactcgacgcgactgaatgtgagaagtttggcgctgggtgtttcttccgggaaggctatatgcctatccggaccagttggctgcggaaagacttcattggtagagtacttggcgaggaagaccgggaggattgcacccaagacgcaggaaatggaacgtcgcttgaaatctgccctcaaagagaagggcaagaagcgcaaatcggggaagattgaagcggagttggatgtggatatccggaaactgaagaaggaagcgccaaagaatggattccttcggattcagctaggagatcaaaccgatagtaagatgctgctgggtcagtatagatgcaccgatacacccggagagttcgtttggcagccgggcgttctgacgcaggccgttatgaatggctactggttgctgttggaggatttggattctgcaacgcaagacgtgtgcactgtgctcacgaatctgctggagaacaactatctgagcgtgcctggattccgggattgcctccgtatcgaaccgggctttcaactgtttctgacgcttcgaacgcagaaatctggaaccagttcggcgcataacacctacactctgctggaaaagtatctgtacacggtcaatgttctaccgttgtcccgaccggagctgtgctcgatcatttgcactaacttccctaggctgaaaaccgtcgccagtcgaatagtggacatatatctgacgttctccagcggctgtcattcgatcgaaagcgatgagatcagtgagaacgttagcatactggacgaggatcgcaaggtgccattgcaaggcctcgaactggacggagatgacgttctgccgaggttccagtacgacaacatttcgttgagcaacttgccaacctcgggaagactggtatcgactcgcgatttgatcaagctttgccgtagatcggatccaagcttctcggtcaccagttcggagtgcgcctattttgtttttcaaaacgcggtggacatcttctgttcgcatctgccagccggcgaaggaaaacgcaacttgatcgttagcattggtggcaagatcggcatcaataaaagccgttgcgatcacttgtgcgaagactacaaaccggacgttaacgtttctgcagccgaggaggttaagatcggtcgcgttaacttgaaacgcaatttgggaggaagtttcggcgatgaaaacgacgaaaacgatcagagttacacgcacaagaaaatcaagctggataagaacgcttctacgacggtgaaaactcaagatttcaaactccagaataaggcgcctacgttttccttcacaagaccggcgtcctgtctcttggagcgtattgccgtatccgttgcccagaacgaacccatccttttggtcggcgaaaccggcgtgggcaagacaagttccattcaatatctggcctaccagacccaccacaagctggtcgtagtcaacatgaacaatcagtccgacgtctccgatctaattggaggcttcaagccggtggatctgagttacgtcatcaccccgctccggtacgagttcgagttcctattcaacaagaccttcaacgcccagaagaacgaaaagtttctcaccaatgtaagcatctgctacaaccgtggcgagtacaacgtcctcgtcaagctgatgctcaagatcgccgaaaaggtgttcacacggaagcaggacgaacagctccgtctggaaaaggtctacgaaaaatgggccaccctacagcagaagcttcaaaagctcgattcccaactgcgcaactcggttaacgtatcgtttgccttcatccccggctcgttggtcaactgtatccgcaatggcgattgggttctgctggacgaaatcaatctggcctccaccgaaaccctcgaatgtctctcaaccattctggaaccggatggctcgatcatcctcttggagaagggcgattttgtaccagtgaaacgccacccggatttccgaatcttctcctgcatgaatcccaacactgatgttggcaagaaagacctcccggtaggcattcgtaatcgcttcacggaattcttcgtcgacgaactcgtggccgaaaacgatcttctcatcctggtgaatgactacctcagcagcaccggtattcagaaggttcgcattctcaacaccgtaaaactgtaccgccggttgcgatccctgtcccagcttgagctgaacgatggcctcggcaacaagccggtcttctcgctgcgaaccctttgtcgagcactcagcatctgcagcaagaacctttgcggttcaatcgaacgcaatctctacgagagtttctgcctcagtttccttacccaactggatctcaaatcgcacaaaaccgtgctcacctacatccagaaaacactcctcaacgacgtccaccaggcgatctcccagcagattccacgccctccggaaggcgaacagctcaacttcgaaggctactggatcgagaagggacctcgcgatgtccaggaatgcgaaagctacatcctcacggaaagcgtgcggaacaatctgcgcgatctggcacgaatcatctcgatcggaaagcttccggttctgctacagggtccgacctcagccggcaagaccagtctgatcgagtacattgccaaacgcagcggcaacgtctgccttcggatcaacaaccacgagcacaccgatctgcaggagtacatcggaacctatgtggctgacgtgaccggaaagttgaccttcaaggagggagttttggtcgaagcaatgcgcaacggcttctggattattctggatgagctgaacctcgctccgagcgatattctagaagccctgaatcgagtgttggacgacaaccgggaactgttcattccggagacgcaggtcgtagtgaaagcacactccaacttcatgctgtttgccacccaaaaccctcccggattgtacggtggtcgcaaaatgctctcccgggcgttcaagaatcgctttgtggagctgcacttctccgaaatcccccgcgatgaactggaggttattctggagaagcgatgccttatcccgaagtcctacgcccagaagatggtgaaggtcatgtcggaattgcagctgaatcgtcgcagtacggccaagcagaacttcacgctgcgcgatctgttccgatggggcaatcgttacacctatgccgataaagctctcctcgcggacaccagttacgattggaatcaacacttgatcgacgaaggctatctggtgctgtcatcgaaggttcgcaatctgtacgaaaccaagatcatcgaagaggccctttacacccacttccggaagcgggtcgccgaagacaatctgttcagtctgaagaagggcacttctatggtcacgcgaggaattctggaacaactggagaagcaaaccgtctacgagaacattgtctggactttcgacatgcggcggatggctgttttgaccgccaaggccctagaattcaacgaacccgtgcttttggtaggccccaccggatgtggcaaaaccaccatctgtcaaattttggccaccatccgggggcgaaatctaagaatcctgaactgtcacatgcatacggagggtgcagacttccttggaggtttgagacctttccgcgattctgatgattctgcggaaaaagacaccaaacagcagctattcgagtggtcagatggtccgctggtcctctcaatggccgaaggtggcttcttcctggccgatgagatatcgctcgcggaagactccgtcttggagcgattgaattgcgtgctggaaccggaaaggactctgctactagcggagaaaggaggagtgaccgccgaagcatctgaatttgtcatcaccgcagcggaaggtttccaatttttggccaccatgaacccgggaggagactttggcaagaaggaattgtcgccggcgttgaggaaccgactgacggagatctggtgccgagcaaccgattccaaggaagatttgatccggatcgccgaacacatgctgcgcaaaggtatgccaaactgcgaaaccgatcctcgtcaagtggctgaagtgatcatcagcgtcgtgtttgtgcttaagaacaaggtcgacaagctgaacttcagtattcgtgatgtgttagcctgggttggttacttgtgcaagaatcagcaaaacctgtcactcgccgaatcgctaatctacggattagagaccgtcttcttggattcccttgagatgttaccctacgaaacgttcgacgagattctcgctctacgtaaggaaatcctctcggttctgcaccttctgatcaagcagcatctgatgatcaagcagttcgagctcaaacgaggaacggcagttcaatcggacgataccacattcggcatcaccccgtttttcattgatctcaatccagaacaacgtgatcgcaaaactgaatttctcttcagcgccccgactacgcagaagaacctcttccgattactctctgccctcagtctggacaaagcgatcctcctggaaggaccaccgggtgtcggtaagacatccctagttgagaacctcgctcgcgctgcaggatttgccatcgttcgaatcaacctgtgtgagcatacggatctggccgatctgtttggtaccgatctcccagctgacgatcattccctggagatcgctgaagaagaagatcaccatgcgcagaaagcttccaaattgggatcttttgtgtggcgcgatggacctcttctggctgcgctgaaagcgaaaaatacctggattctactggacgaactgaacttggccccgcaaagtgtattggagggactgaacgcgatcctcgatcatcgtggtgaggtgttcatagccgaattgaacaaaactttcaagctgggcaagaagacgcgcatcttcgcggcgcaaaacccgttgcgacagggcggtggccgtaagggtcttcctcaatcgttcttgaatcgcttcaccaaggtatacctgcggaaactcgagcagagtgatctgctgcatgtcgttgacgggaagtacgtacagaagttcggcaagatcggcgaggaaatgatcgatgaccttggaagacgaccaggacctgatgcaagcttttttgacgtcctgaaagcgcccgaggatcatacgatcaattttgaccttgcccagaaaatggtgatcttctctgaaacgctggaaactggaatcgcaaatctggagttcgggtacaagggaggtcccttcgaggcaaatcttcgagatattcttcgatggtgcgagctgttctgcaacaccaaaacgggattcttccgcaagaagttcgccaatcgagaagacacgttccgcaactttttgctggttttgttcgagaagatgaagttggtctactaccaaagaatgcgaaccgacgtcgataaaagttacatcctgtcagcgttttcgcgtgtcttcagttgcgatgccttccaattggaagcgcaagccgaagatatcgggatctactggactgataataagcttttcctgggcgatttggcggtggaaaaggtcaatcgatcggatgaaatgaaccctttggcgtccaaaacacagaaaagtttcccattgctgctagcgagtcaggtggagacgttaaggaacgtggccgagtgtgtccagctggagaaaccggtgatcttgtgcggtccgagcgactgcggcaagaccaaaatcatcaatctactgacgacgctggctaacaagttctgcaacgtggacacgatcgacgactcggttaccggcagcttccaacagttcgatttcaaccgacatctggaggaagtttcgtccaacgtggaagcggtagtgatggacgccgtgaaggacgctgcactgagcgggactgcggaaggccgaaattgtgtagtgcagttgctggcggtttgggagaagtacgctcggttgaacgacggaaaaatggcatttaccgcccaaaatgtccaatccgaggagttggtccttttccgaaaacgtctaaccgctctgcaagatgtcatcgcactgctaacagcccacaaaacccccaatcacaatcacgaccaccttggatctatggcttccgttttgcaagtcttgtccaacatatccaaacacgccgaaacactcaataccggaggtcacttcgagtgggtcgactcgaagatcgtcaaatccctcaaacatggtcagtacatttgcctagagcacgtcaatctgtgttcgtctgccattctggaccggttgaatccgatcttcgagccggatggaagtctgttgatttcggaaaagggcgtcgacggtaataacgagtcggaattgattcgcaaacatgccaacttccgagcgtttttgaccctagaccccaagaacggtgaaatatcccgagctatgcgtaatcgttgcatcgaactggctattcacaaggaagcctatagggatgacgacttaaagcggctggccttcgagagtggagtacgtcaaaattggttgatcaacgtgattttggacattcacaaagccgtcaagaaaatctctgagtttaaccaattcggcatcgctcacatcgtgaagtttgcctacctggttgctcagaatcagagaatgggaatgacggcagaaaaatgcctgatgatcagtgctttggaggtctacgtgcgatcgtccaatacagaccttctcggatacggtatagacttctaccggaacgccttgaaagatgcgatcaaagaagaaatacgaaagtccatcgtaacaccaaacttggtcaattacgacaacatcttgttgaagtccaatgaactcaccagtcttcaattgatcaaactgcaagcggaacccttactctgcgttctgagatgtcacctgaacgggttcgcggatattcctgcagctttggcagacattgctggcgcattccacgatcaagccgaaataaacttgagcaacttgaagtatctgttctatttcctatacgaagcttcgagctatggagatgcaaactgccgaaaaattttcttcgccgaatcgctgagggcactccacaacgttaaaaagatagaacccaagaaagaggtgactcctacggcggaaattactgtagacctagaagttcaagggggcatgggtcaatggagacaaactcctcaaagcctcactgaataccgcgatctgcaacacaccaagcgagaaaaaatggaattcgagatcgcttcatccatcgcgatagatgaagtactacaacagctgattgatttgaacgaaattctgttcactgccgttggcaagtttggcgcgggagaccagcttccttggaatcgatcgcttttgccaaggattcgagaatatctggaagattcctccgtcgaacagtatcaactgagtgcaaccctactgttgaacacaaccctgaaagcgatcgaagtcactagtgctaccaagttgagcaaaattgatgtcctgtcctactcgaaggcagttcatacggagatcatcaacgatacgattggcaacaaactgattgcgcagttgtacgactttttgagcagttttaagccgattatggagaaagctctccgagaagtgaccctggatcaggacacttacgccaaattggtcgggtcgtatttgtggatcaaccggttgatcgatctctcctgccgtagactgttcgtcaataaagagatcgatacgagtcttgtggactatgtcactttgcacttccaatggatccagaaacactgcctctctctgttggatgatcagaactgtagtccgttccgtgctctgaatgactccctgcaagaatctatgcagagttcgacacatccgcttgctaagcataggaagctttatgccaaaacgttctcgtgtagcttaccattctacagcgaagagcaggtagagcactatcagcgcatgatggagttcaacgaaagtcttcggctattcccacgcttaacgcggttgtacgactatgaagaattccttcagaaggtggccctgttgaatcacccgaaaacaatcgacatgaggcgatcgcttctggccaactccaagcagctcttgtcattggatttggatcatcgagaagctgagaccatgttaaatcatgccgaatatcaacaactgatggaattgtgccaaatgttccaggaagaaagcaacgaagattctgcagagcagttgattgcagcaatcgaaaaactgaatgattaccggaaatttgtggctacttatgaagcaccggaggaatacaaccacaaattcccagtcgaaatgatgccactgtatgagtatttcctcctgaagacactaggccgcgacgctgaaatcaactacgactacgttgcaagcgtccgttccttaaatctagactctctccggcttgtgagacttcagggtaattcgcagcatagatcaattctcaaaatgagcaaactacttgatgttttaatcgaatccgatgtagagttgagtgatctcctgcgagccttgccatctggtttctacaagaacataacaacctacctcaatcggttccaccgattcaactatgttttccagatgaactcgttggctatcaatcacatcgtttgctatcagaacgagaccgaagcgagctatgaggcacatctagagcaaatgtccaatctgacgatgcacggtccagtgctgaccaacaattgcctatctatcttactcaacaacagtggagaactgcgatcgactggtttgggtgaacttcctgaatggcgaaagactttgaagacgttacgtgatacgatctggaccaatgctactgtactaagcagagaatacgatcagtattctgtgggattggaaacagctgttggtcaagctaaaaagctgctcgtggagtatgaatgtgttcagaaatggtgccaagacggtcaacgaaacgttgataatgaaaagttcctccaagactttgaatcgctagctgaacagctaaagaaacttgtgaaaaagtctgcggatgtacgcgagaattctagctacgaagatcttcgcacgagttcgaatagaaatctacaatggcagttaagttcagctttaaattcgctacgcggagcgatcgaactgaaccttctcgttttcctaccgctgcttgatccagtcgagaagaacgccctgaaaaaggcgtacaccgacgaagatattcagcactacaaaacgctgatacaagtgtacgactacatggcgatcaccatgaactaccgccgttttggtgaaggcaccaagcagctatttgtcaaccacctcaattcactagaagagaaacgcaacaaactttgccggaaggtagctctgcggccctcagacaatatctacaccgatctggtcaaagacgtcaacaacttcctgatctcctgttgctatccgggaactattagtgaactgttgaagaccgccttacagtgcctggaattcgaagtggataccaaaaacccgatcaagaactgtacccacaccaaagacttccaactgcgtgtcaacgaaacgatcaagcaaatcgaactgtgggtcaacattgccgacaagtttgaacaccatacgctgaagcgctaccaagcgtactaccgggacttcttggagccactttgctgctccattgccaccctaaagtacggattgatcggcatgaagcactgtttgatcatcaagcgggactcgatcgctgtcaagtccaatggagtgttctacgacttgtctgaaaaggatgccttgacgaagattgtggaaaaattcgtgcagttcccagcggtggagacgatcgagttgttcgacgaggaagccagcggacacaaagtgaacgtttattccattttggagaagatagatcatagcgaaagcggatatttccgactgctgaaatcaaaactgcaggaggtgaagaatgctatcacattgcgtggagggatcggtgctgaagaatttgtggaccttgataagatcgttaacgtgtgcaatcaagtttggcagaagcaggaacacctcagacggaagaagcaagccgaagacgacagcttgtaccagacgaagagtcattgtgacgaagaggacgaggaagtgatagcacaacgagaaattgttgatttgttcccgaactatgtagacgaagattttgcggatttcattcaaaacgatacactggaacaggtcatcaagaaagacagcgtcaagaaaaatcaaaagttggcggaaactattactggcgatgattacacgatgatctgcgaaagcttcatctacctgatgaacaaattctcaaagtcgtattactataatccggagacagttgctgatctaccggaggctattgcacccgatttcattagtcccttccagacgaagatcgcgattgtacagcaagtgttgcaaaaataccattcggctttgaacagtaaactggatgaaagcttttatccaggcttgagttttttggtcggactggctcaagacaagtatgaggatttagatttgaaacgatccactgccggcaagtacaatttctacaaagattccaacatcgcagaagtggtgcagtgtgtcgaagtgctcaagaaaatagaggatcgtgttcaaaaactactgcaagaatggcccgatcatgccgtcctcaaggacatcatactgatcttgaaccgtatttatttgctgccttcgacggctccgatcgtccggttcagcacgggtctacagctgttgcggcagaaaatcgacgaatggaacagtgtagctcatcggctgaacaatctacgtgagttagaagcagacatcgtagagttcgtacaccggtggatgcgtatggagctgcaatactggagagaatgtatgagtcaaactcacgacaacgtgcgatccaaagcgtaccgctattggttcttcatgtataatctactgcatgagtatcttcaaagcgattcaaaggccaagctaggcaatctactggactaccgaaaggttgaacgttgtttcggtgagcaagaactcctggaagaagatgaggttgaggaaggaacagtcaagatcgacgatctgattcacgttctgaagcagttcatggaatcgtccaactacgcggaatataccactcgtctacggattttgaaatccttcgagcagtatctcatgtatttaaactacgaagacaaccgtaagaaagaaaccattatcgccgtattgcataacctgcatatgtactacgaccaattctccgacgaaatagaagagtttattagaacgaaacgcacaccaatcgagaagaaactgaaagacttcgtcaagatcgaatccttcaacaaagatctctcgtacttcagtatgcgtaacaacatcgcaagagtccatcggcagctgcacaagttcctcaaagagtttgaaacggagatcagagccaagattctaccggtattcacccccaaagacccagccgcggagatcaacgatgctgaacaacaaaaaggcaaaaatcttcgatcggaagccaagatcacctactacatggtagacgtgaaaaacttcatggcatcactcaaactgaaggaaaagttctcaatcgctagcatatcacaggacaccacaccaaatcaagaactgctggccaagattgatcggttctttgttacctcacgaaccgtagtgaaggaagcgattctgcatgcgccattcccaggtctgatctacaatctggacaccttcatgcatgatcaaatcgaatcggtggagtatctccgaggattggaagttgatcgcagccaaccccgtcccaagcagaagtcacaagccaaacaaattctgaatcaaaaacgtaaagcgttgtccgacttctacaagagtttgacccaattggggttgagtttccgcagcgggttggtggaaaacgcgatcgagtcggatccggtagatgtagcgattaaaccattctcagcggagttactcgcgcacaacagtaagtaccgtaaggtggatcaacatatcgtgttcctgaacgataaacttaacctgtactatgcaaagtgtgtgttcaagatcaaaatgttgacgaaggttctgttgcaaccgaatccagacatcgggaagatcaaccttgaaagaatcaaaggatttgctattgatctcttcttaacggttcaggatcaacgtaaggagctgggcaaatccgttgccagtgtctacgagttgagagatcgtattcaggatctcacgcagcttagtggatgcctggattcaaagaagcgtgaattcgattttgttgcgagcagacgtaagatgaatcatatgcgacactgtacgatcaaggtcagatgtgtattggagcaattcttacagcttctcagttgtgcacctgcagaagtccaagacgagctcaaaatgctaaagaattcacacgaactgactcagaagacgatggagccgattgtgaaaggagctagcgatggcctaaagcaagctgttcgactttgcgaagaattgaagaaatgcgacgaaaactacttgaccggagatctcgttcaatcaatgaacgatcaattcaaatgcatgactaacattcttgaccagttgtgcgaacgccttaagatcggcaattccgatgaacattcagtgtatgctcaaccgattcttagcgttctgaaaatagttaaaaatgtcatcgacgcagaagatcacccggccgctgaagatcgcacaaacgaagacaacctgaacaacgaactagaaaacataatccattcgattttgatcgcaatgcaacacatttacaagaagtactccactgcggcccctacggaatgttcggatgctccgaaggcactcgatgacgctgaggaaaccgaaaatcacgaatcagaatcggaacttcaaacgcagcatctgaaggaaaagatcaacgtggaccttctgcaagatctacaaactcttagcattcgaaaggtggtcggtaaaattgccaatatactctccatgcttgaacaaacaaatcactccgcaccacagttcgtgacaatgatccaaaaaattactcgactggtgccaattctggagcagttcaacttgctagttgagtattacttgatccaacagttgggagcgcacaaaatcagcacaaagatgctatcggtaatgctgacggtgttcattgaactcggtaccaagggcttctgcgtgccgaaggatctcctagaagacgaacaagaagcaggtggggagcaacaggaaggaaacgagcaaggagagaaatttggcttcgaagacggtgaaggtgagaaggatgtttccaacaagattgaaagcgaagatcagcttgacgatgccaagaaacctgggcaggaggacaagaacgacgaagaagaggagaaggacaacaaggaagaaaagggtatcgatatgagtgaagattttgactcgaagctgcaggatgtggaaaaaccggaaggtgaagattcggaagaggaaaaggacgacgaggaagatgcagacaagcagatgggagaaacagaagaaggagcggaaaagctcgatgatcagatttggggtgacgacgaggagaaagaggaaaaagataaggaagaagacatggaagaggaagatggcaaaggttctggtgaagcggatgacaaacataacaatttagattccgagaaggacaagacaccgggagaggaagaggaggggttggatgcggtagataaagcgatggatgacaagaaaaaggagaaacccaaagacattgaagacatggaaaaccaggaaggcgacgaagagcaagagaatccccatcacaatgagcttgaagaaccacctcaaccggaagatatggatattgatgaggagttcaaccttgataacgataaccgtccagataatcagaaggatgaagacgaaaatcaggaaaatccattcgatatcgacaacatgaaagataagggagatgaaaaggatgaagaagaaacagaagctaaggatgatgaacagaaggattccgagaaagatcagcaaaacggcgatgactcctcagatgaagaacaggaacaggagaatccagaaggggcagacaagctggacgataatgaagtcaacccagaaggtgaagaagagcaagaaaagcaggacgatgaggttcaacaaattccggaagaaaacaaagatgaacaaccagctgaaccagaagaaccccaatcagaggaaggtgagaatcagaagaagaagcaggatgaagctcatcatgaatccaaggataaacgatcggaagaggatcgtattgaagcgatgcccgatatagaaaataagggcaccagcgatcaagtggcaaacgaaaaaccagagtccaaacaggaaaacgaaatggacgagcaggataccggagaagataaagatggagttggacaagcggagaacgagcaatcggaaagtggtcatcaaggtattgccgaatcgaaggaaactcgtacaaagcgtgacaagaatgatcagaagcagcaacaagagaagcgacgccaaggcaatacggatgaagacagaacattgggtgatccggataaggccgagaagaagcgattgaagacggtagatgagataaacagggaagatcgagcgaacaaggaagatgaagttgaagaagatgaagacaaggaagatgaatcggaggaatatcagcacgtgaaggacgctaaacaatcggataaaacaacactggacaacgcaactgaggagcagtccaaaaagattcaacatcaagagaacaccaaggaggacgatgaaccagatgaagaacaagagaatgcggatgagctgatggatgtcgatgaacagacggaagaattggaagagaccgataagcaactgctggaaagtgagaaaaacgaaaagaagaaggacaaaccggcaaagaaggatgagaatcaacgggaaaaggtcgaaacgttggaaaacgttgaagtggaaggggagactgttgacacagcaggggtcccaagaggagcagacactacggcgcatactattatggacattttgaacgacgtgacgatcccggaggaacccaccacagcggaaacgttggagatgcgaaagatggtcgagaaggaaaccctagccgtgaatgcggctcaaccagatcacgatgatttcgaccggtggcaacacatttcgaacaagatgcttccaaatgcccgcgagctgtgtgagcagcttcgattgattttggagcccaccaaatgtacgagactgaagggcgactaccggacgggtcgtcggatcaacatgaagaagatcattccctacattgcctcgcaattccgcaaagacaagatctggcttcgtaggacgaaagctgcacagcgtgactacaagatcacaatcgctgtggacgactccaagtcgatggatcacaacaattccaaagatctgacgttgcaagccatttcgctagtttcgcaggcattgactttgttggaaagcggtcgtctgaatgtgatgtcgtttggagagtcaccgaaaattttgctgaagcattccgatcagttcgatggcgccaagttgatcaactcgctgaactttgagcaaaatcaaagccgaatcgccgagttgttggactttgtaaggacgttcaatgcagaggacacatcgtcgtcggataacggcattttcgaaaatttgctactggtgttgagcgatggtcgtaacattttcagcgagggcgagaaaaaggtgaaaaactcggtcaaattagctcgtttgcagaggatattcgtcgtttatgtgatcattgataaccctgagaacaagcactccatcatggatatccgagtaccgttgttcaccgcggatcgaaaccagattgtgatgcaatcctacctggacgtgttcccattcccatattacgtgatcgttcgagatctcggccaattgccattggtgttgagtgatgccatgcggcagtggttcgagctggtcaacagcgagcagtaagggtaacagcttttaaaagctagtaaagttatttgtgttatttcattattattactgataaaaatatttacgattgttccactgtaaatacaatttgaattagatcgcatttcaatatgttatgttgaagtactagaagcagaactttgtagttcgagagcttccactagctggtatattttcgtatacaataaaacatagtttgtactaaaa

SEQ ID NO: 46 - DNA, 1327 bp, Aedes aegypti
aagctcgtttagtcccccttataatcgaaggattcccctcaaatattcacagtggcttatgtagcttagaattttaacaaatcaaattttctcacataaaaacgggccaccttagtgtgcatgccgggaaacagttgaccaccagttggcattagcctgacgaagacccgtaaaagactggcgtgcgaaagagaccttcccccaataatgatccactgccatgcgggcagagaggggcaccacagctcgtctttgtgacttgggggctctcgcttccacgccgaaagaggtgtaggtacgaaacaaaaacaaaaactgcatcagatttagacggttggtatataaagacccatcaatggggatctccgaccgcagtctagatcttactttcttccctgaagcacaattcggttctgaatccaaaccaatttgaaaaagggggtggtgacatttccagtggaaagtgcaaagtgcgtgatctctataaagatgaagttactgattttatcagcgctcctagcttcggcctgcgcttttccgcagcgcaacaatgactacgaccatcaccaacagcatcaacagcaacaccaaccacaacagcaacaacagcaccaacaccagcaaaacatcgaaaagcgcactcaagagaccaccacttggattccgattctgaaatacaacaaggaacagggcgaagatggcagctataaaaccgagtatcaaaccggtaacaacattgtccacgaagaatccggctaccttaaggacttctctgatgcccacccgaacggagtgctcgttcagcagggtgcctactcgtacgaagctcccgacggacagatcatccacgtgcagtacaccgccgacgagaagggattccgtgtgaccggagaccatctgccaaccgaaccaccaatcccggaaggtatccgcaagggtctggaggagatctacgccggaatcaagcgtcgcgagcaggaagccaagtctaacccgaagtacgcggaaacggctgctcagcgagcggaacttgactacaacggccaatactaccatcagtgagcgcggaacgcggcggcgagacttaggatattcgtgtgtgtatttatattaggataaaccgaagcgctgacatcaggcttcctagcttaatgatgatatctttgttgaacctgaacttcttgctatggctcgcggtatcaaatgatggctgcgagaacgacttatctgaaaagaagactgtgatttcctttctctagcgtttagcgtaaacgcaaagaatggcttatccacaagcgatggaataaactttactttattattgaaa

SEQ ID NO: 47 - DNA, 801 bp, Aedes aegypti
ccgactgtgaaagtagatagatcccagcgaaaaacaaagagtgcactcaagaattaacgaaacattcggaaggcctaccgtgaagtgttcagaatgaagtgtttgattgtgttttcgttgttaacattgggctgtgccgtggcgcaggatttcaacgatggacgctattatccggaactgctgaggggcaagtttgatgacggacaatatcgaccggacaatggcggagcttatcgtccaggaagcgctggtggcttcgtgcagagaggatctggtggttccggaaatcgacgaggatccagtggtttcagttctgccaactccgccaatcgctttggctcatccaacggcaacagatttgtaaacagcgctcaagacaaatttgtgccagtatttgctcaggcacccgtaccagtatttgccccggtatcgtctggttttggcgccagcagcaataaccgaggatttggtaactccggaagatcaagtgcatctcgtggcagccaggaaggtgtcaaggaagatacccgtgaattgaacgaagatggttacttctatcggtttttgaccgagaacaatattgaagtcgctcaaaccggacgcatcgagaaccgtggtaccgaaagcgaggttctgcgcgctaaaggattctacgaatttgttggagatgatggtacacgttaccgagtagactacattgccgatgaaaatggattccagccttcgggtgaacacctgccaacaccgcctccaatcccagaggaaatcgttcgttctttgcaacttctgagtcagggataa

SEQ ID NO: 48 - DNA, 1995 bp, Aedes aegypti
atgattggacacttttggatgcggatgcttgtggtagtgtttgtagtggcactgactgtggtcgttgtcgattgtcaaaattatgatcatgagactgttggacgatttccagcattattcgagtacgatgattttgaaagctgtcgtaagacgtatcgtgagcgatacgtttactgtatagttcgagcgaagatagttccggacgatgagtcactattatggcaaaatatgagcatggtctccgaagataacttttattatgatcatcgggtgttggaacggggaatgtgtgttcaaaattgtctcgcaaacgcagacaaagatactgttgccgatctaaatggactgaatcaattgagaacttgcattaatcagcgtctccagaagagtcatgatcttcagattgatccagaccttcatatctaccactgctacaacaaaccaaacgaagagcctccgtatgacgcattggaaatagttttcttcatcacaattgcagtgctaaccctcctcgtctgctactcaacatggtatgacttatcacttcaatcggcgcatcggtataccgataattacttcagcaaatctcacaccactagtcaacaaaagttgctcactgccttctctattccgcggaatctccggcgcataggacacaccgcgaatacggaaattcggcgggatttgaatttccttgagtcgttccgtttcatccaaatgtatcggattatttcgcttcatgtggtgatggcactggtgaaggtacccaagagtaatccggaagaccttgaactgatggtcaatcaacctgcagctattgaatacgttgcagagttccaaaactatgtacaaaccttcttttccataactggaatgttgatgacgatcaactttctggagcacattcgcaaaaaccctcagttcaacatgaaatacttttgggaacgtatccgagcacggatgtaccgaatcgtcccggcttatttgtttatcctcttgctggaaacttccatcactcggcgtttcatggatggacctctggcagagcttatgattgggcaatcgagggctagttgtcgtcaaagttggtggaaaaatcttctatttgtgaacaactacatcgatacagacaaaccatgccttatccaatcttggtatctgtcggctgatttgcaactctttacctttgctcttgggtgcctcatgcttatctggcgatggccattcctgaagaaatacatccttggcattggattaacatggggacttgtcgtcaccacgatagttgcgtacttaggtcgaattccaccagtcatgaccgaagaccttaagctgtacaataactacaatttcggtcatccgtacttccaactgtaccaaccattccacatgaatatcacaatctacttcgccgggatgatcgccggttttgtgtatcatcggtatcgggaatcacgcaaagagtttttcaaatgccagctacagtttaatttgctgcaggtcttcctgttcctgtacttctttaccgtgttcacaggctggtgggtcgttcggaatcaatctgtgatttcgcctattttggtggcaatctacgccacctgtttcaagcacgtctggggcttcctgtgcactatgatccagatacgaacagctctcgccaccagctggtctcgattccgcaacttcttcagccacccgattttcattatacttgggaaactgtgctacagcttctatctcattcacttcaccgtaatagtgcaagtcgtaggttcagctaaacaacccattttctatagtatgagagcgattttcgaatatactttcacaacctgtatatggaccctcatttttggaacattgctctgcattctggttgaattgccttcgaatgtggccctgcaggagttgtttgaatccaggtcggagaaaaagccaaccgatactgatgcaagtacgccaaacagaaaaatacacaaagatcctagccactga

SEQ ID NO: 49 - DNA, 2467 bp, Aedes aegypti
attcctctagaatcagttgtgttttgtgcttcaatcagtctgtttgggaatcataatcgaaaccaacagtgtttgaaaaatcgtcataatggaactctgatattaaccagtgctgtgattcgaaaacatatttctgctgtcttttggaacgggagacgagtaactgtgtaacagaaaagctgcaaggctcatgcgaatctttcgttccggttggagaataatgagcaccagctatccagatgcaccccttgggattcgacttgtaaccgctatatgcgggaagatttgtccccgatggcaaatcaatcgattgctctggtgagttatttgctaacaagtgaaatgattaattactagtataattgacgattatgaggtgcaaaaatttgtgctgataacagataatacgtaggacaatacaaatcaaaacgacaattttaacgttgatcgccttgacattcaatgtttggaatggcaattttgcacaagaaatgttatttaatatacctacctagaatgcaattgtggtgatgaatcatttagatgtggaagagaactgacaagagtgacaatttgaaagctagtgtgattgttgtagacaagctgcatatgcaagcaaatattatatcatgcaggggaagaagatgataacagttgtaccacagttatggaaaaaccacaaatatggattaatgttgtatttaacacagaatttcttttcaattgaataagcacttagatttttttctcactcttttttttaccgatattggcaccaccgagcgctcagcaaacactgttttagacgcgatttcagcaagtatgatgcttttttgctaaggttcaaatgctcggtgctcacgctcacctatctggcgtacatgtgctatcacatgacaaggaaaccgatctccgtggtgaaatccgtactgcatcgtaattgttccggagttcaaattccgccggaattcattcacaacggtggagagcccccgaatgatagcacctggtgtgactatgcgcctttcgatggggcagattctgcggccttgctggggacgctggattcgtcgtttcttttcagctacgcaattgcaatgttcttctcggggttcattgctgaacgggtctctctacgatatttcttgacctttggcatgttgttctctggcattttttgttatctgttcggaattgccaaagtatacgatattcactcgatgatgtacttcgtgctggtacaggcaatggctggtatttttcaaactaccggttggccaggggtggtaaccattgtgggccgctggttcgggaaatcgaagcgtggcttgattttcggtatttggaatagtcatacatcgattggaaacatcatggggacattgatagctggatactacgtggaaaaggattgggcaatgtcatttgtggtgcccggatttgtaatgggcgtgtttggattcattatgttcctgtttctggtggatcaacccgagattgtggattgccacgagaaagtaggcgaccgtagaggaggctataggagaattgaggacggtagaattggagaatccggtggtaactctgatgctgaagaatcggttggaaccagtagtgaacaggatgtaaacgaacgcacaccaattataggcagtattaatcgaacacctgttcaagacgatgcaattggtttcggcggtgcattaaggattccaggagttgtagagttctccatgtgtctgtttttctccaaacttgttagttacacattcctcttctggttaccactgtacattcagtcctctacaacactgggggcagaactaagtgcagatgtatcgactgtgttcgatatcggtggcattgttggcgccatagcggctggcctaatctccgactacactggaatgtctgcatcgacgtgcactggcatgctagcgttggccgcacctgctttgctgatctaccaacaatggggtgctatctctctatcgttcaacattttcttgctcttcattgctggagtgctagtcaatggaccatatgcacttattactacttccgttagtgctgaactaggtcaacatagttctttgaacggcaattccaaagcccttgctactgtaacagcgatcatcgatggaacgggatcaatcggagctgctgttgggccgctgattgctggattgatatcatcgtccggttgggaaaacgtcttctacatgttgattgcatcggatatattggcgctcctgttactactgcggctagtgaccaaagaagtatcgcggcgatcgaggcggcgcaatgtccgaatagagtaacgtaaaacgtagtagatagcaaagcaagctgcgaagcttaaagattttcaaaaatgactacctagctaatatgaactgatttttaagcatctcaaacgaaaaattttaaataatgatcttccttttttgcggaaagcttcaaa

SEQ ID NO: 50 - DNA, 1512 bp, Aedes aegypti
atgggatttgggtacttagtcgggattttgctcattgtcggagtagttcaagtgcgaagtgattcgtgtccagtgtttttgacgttatcctcacttgcgagacatatcgaggatgcgtcgctcgttataaactggggtccagattgtgaaacaccacccgattggataggactctatactcataatccggcattgtcagagtctccgccagttgtgaagatctatgtagaaggccaaaaatccggagaaattgtaacagaacttaagttcggtaaaccacgccttccgggaggatggggctacgacgaagtccttcgtcacatcccgaaacgtcgagctaagccgctttgcttcgacttctacctggttggtcacaatcgggacaacgaacttgagaccttcgactgcctcaaaatccaaccgaattggatgttcacagagcgcagcatcggcaacgtttctctgaaggaccttttcctccctggaacacactgctctgcatgctaccaaaccaaagccaatatcaaccatatattcttgaaacgcgttggctaccgacaagatctggatatctggacacagctcgtttttggcattcgctatctggacttctccgtcggctaccacccatcccacaatggaacgcgcaatttctggatcatgaatgaaagttatcgcgtgggttggatcgtaccgatccttcatgacatccgtcgctttgcgattctctcagaagaaaccttagtgatggacttccgtcgattcccgctgggattccacaaccatccggaacagcacttagagtttctgcaggtcttggagcaggagctgggtgacctgatttaccacaggccgcactttggagatgagcagaatgacgacaccgagtccttccacctgacgatcgaccagatgcgacgggagggaaagtacatcctcgtgacgtacaatcatgcggccagtttgaatgcttccgatctcgtgtgggcgccatggaaaaagcactccagttcgttcctcaagcacaccgagttgggtgagttcatgaagaatctcttctcgcaaaagcacccggatgcaccaaaagacatcggatggagcttgcacggcgtgcagggtttgcagagcagctatgtaatcagttaattaattggttcgaaattccaactaatttagagttcgtttcagacctccgacgagagttacatgatgcccaaggagcgtgccaatctgatcaatccgaagctgatgacgatgctcggtgggccgtggagtttgagggccaacgccgtcctgctggactactttaccaataccaatcttgttgatatggcggttcacaccaatcggtacaagacgttgaagagtttcggagaggaggtggtcgttttggacattcagtgatttcaacaggaagattcatttaggactataagattaaatatgcttcagattaacgatgagattttattgaggaaaagctaatttcaatcatcaagttttaatcatcaa

SEQ ID NO: 51 - DNA, 841 bp, Aedes aegypti
gtctcgcactttcctgctgtcactttctttcaagtcaaaaccgcaaccgaaccaaaacacgtttccgtgttgttcgtgccgaaattcggccttttcaagtgattttcttccggtaaatttgtgcgtagatccccgaccatcatgactgcccgttccaaatcccggcgggacaagaataaccgcatccgccgcgccaaaaacaaggtcaaagagctgaaaaaacttaagaaaaccctcggcctcatcgacgaagatgggatggatattatggaaaaagtgaaggacattaccgaacagcaaaagaagaaggaggaagaggaaaaaatcaagcaggaagcaatggaagaaattatacgcaaggaaaccaacgagctcggcctggagacggaagagtacgttgaagtagaacatcaggaatcaaaagtaaaacacaagtacaacgccaaaacgaaacgcgatcagttcggacagtatccggtgtggtataatgcccgcaaggaacgcaggaagcagctgttgatcgagggtaagatcaagaagaagcgcggccgcccgggaaggaagacgcactttatcgatgccacctgcaattggaggggttcggtttgagtttctcgagcgattcaaacaccactttctttaattgtcttggctaattatttggtttaagtttattcttacgcgctatttctgtaaactggaggaaacacttataaataaacaatgtttcaaaaaatcaaataactagtaatatcaatatccttatgattgaaacacctataactttaactatcgcagcgttgaactcctctccaattattgttttctattttgggaaaca

SEQ ID NO: 52 - DNA, 1183 bp, Aedes aegypti
ttctggtagcttccactcttgcttagcccccgtgtgcagagaaattgatagcagaagtgttgcggtagtgaatcgtttttggttggtgtggattttgatctccagaaagaaatatctgctaaaaaagaggataataaaacataatcaaaaatgtcgctacgacttgtatcatcgccctactttcgccgtttcttactggcaaataatggcaagaccgttccatcacgcgccatgagtcgtgatccttactacgatccccgaggattcactttcaccgatatgaaaaagaacccagcggttatcccactggtcgtcatcattggcgctaccgtaacgggtctgcttgggtactgtacgtacgcttcgcttacccgtccagatgttagccttaataagaaaacctacgaatgggataccatggacgtcatgaatccaaagaagttgaagatgcgcgtcttcaaccaggaatacaaaccgatgccggaactgaacgaagcactttcttaccgggaggggtatgccaagcagcaggagtcttaataagatcaccgcctagatacgaccatatatcgctgttgttgttattaataacaagatcttgaaaacaagtgttgcgctttggtaatacatatagagtcatatagcttcaattaactagcatgagcacatccgtcgttttacaacatcatcctatccgcacgcgcttatcgcttcagcagctgaagtaagaatgcacagcagaatgccgcacgagaactatcgttacttaaggtgaaacatctcggaatccaaagacggccgacttatgacactactcgcgattttgaagtcactaaatataagaaattgtgagtcaacgaagctgaacttcttattttaagtttgtcaatggtgtctgttgcttctttcgcgagatttgtgtgtttagatacataattgacgttggattctataatgtatcaccttaaaacgctccgctcgtgggcttcgatttgctgcagcagcagccagcgttagatgtttcaaaccaataaattattatacctcgtagtgtttagtcgctgttagtaaagttagcaagcagagtgcaaaatataatacagctcagagctcagtagtcggtctcactctcgattcgtgatctaagcgaatcgtgacctggtgagattagagaagcacccgt

SEQ ID NO: 53 - DNA, 657 bp, Aedes aegypti
cattattatcacaccaaagtgtgtcaaatcagtgagaccgttcagcagtttgtagtttcgcgagtagtcgaacatttgctctattcagtgcagttcaaaatgtagcaccatgaagcatcagcagtgcgtaattgatctcgcgatcgtactggttacgattccatctctaattgttgtagcgctgctgttggagtatacgatgaaccataccgactgggctgaccggctggagtcccaaattatcgtgctgggcctggccgtggcattctttgtgctctgcatcatgatggccttgtatctaacgcatcggataggatcatgcctatggaccgggcctcttccagctgatcttcaccgtacgatcttcaccgttgtgcgggatttggagagcggtccacccccggactatgataccatcctgcaggatccaccaccatacgaaaagttggagaagtacttacttctggatgaacctcctccgtcttatgccgcttccatggcgtcattggagggtgctcatatctgataagagggacttgtttgttgatattgtttttgtttggtgtgtaccgaaaacagggatagctacatttcaattagtggtacacagcgggatcttatgagcgaataaactaaacttttgatgtgcactgctag

SEQ ID NO: 54 - DNA, 1563 bp, Aedes aegypti
atgtcagggaaaacggtacttttcctctgtttgcttcaaatccttgagaccataagctctctggggacaaaaacatcgttggctctgaattgtttccaaccgcaaaaagctggaatatgtagtcttcgttttgtcaaagtgttagaaaaagatgttatcgatagtgctacccaagcaacgtcagacaaggtgatacacatcgagcagtcacaactggagtacattccggaagttttgttcaacagatttccacaaatggaaacctttcaagcgattggtgtggggctgaacaagttttctgccgatgctttcgccaaatcgccaaagttgaagcatttggacgtgtcggtcaattcgattagcatcctgccgcaggacgttttcggaacatgtcagcacctggaactgatcaatatggcccagaacagattgcacctcttcaatggaatcgaactcataggatgcaataagcttcgtcaattgaacgtatcatccaaccaacttatatttatcaattgggatccgctgaacgatttgcgtcatttagagcaaattgatttgagtaataacttgatctcctccgttacagttccaagacatgtaaaacgcattgtagcccgcaataatcacatccataaactgaccactgattcaaattccttcatatttttgctggaacatttagatgcatctagaaaccggttgagcaatatagatgttcttgcccgcttcggtaagatgacctacataaatctctcgtacaaccgattactgaatgtggattttgcactgtttaaaaacatgccttccctcgtggagcttaatctggcccataataacatattcgttgtaacgacgtctgatgttaagctcgttgcgctcgagtcggtagatctgtcaaacaacgaactgacacgcttatctgaagccgattgtaaaggattctcacaggcgaggaaactgctcttgaataacaattatttggttaaactggaggttatgaaaggtgcaagcaactttcctagactgcagtcgatttcactgaatggaaatgactgggcttgtccggatatggaggcaatgcttacagaactaggtgcaaagaaagtcactgtcaaacctaccgatgaagcttgtggaacttatcaggtcaagaaaagaggtttatgttgtcgtgatttgagcacttcctttgatgagcttgttttgttggaatcgcaaaaattatccgagatgagacaagtggaccaaagtagagcagtatcagcctcgaccgtaaaaccaacatcggtgacgcgtcagaaacaagttcccgttacagcaaacaaagaatcagtttcccgatactctgaagagatgcacaatctttcgaattcgttgcgtgagactatgagtcgtcttgttatcatcgaagcagagctaaaagcttcgaaaagtgagaaggtcatcttgaaagattccctggaccgagcacagatcgaattgaaagccctgaatgagaagctagctcgttgtaaatctactgttaatcaacggactggacaaactgtgctgattgattga

SEQ ID NO: 55 - DNA, 818 bp, Aedes aegypti
cgtgaaactgttgtgtcataaatcagaaataaataagctttatacgaatgatgttcatctcaagcaatgtgacttcccagatctgtttaagatgccctcgcttagccaaagcaacaacccgatactgctcaagtcaactgtggtccgatgctgctccatcgcgtcggtgggtggttgctgaggggaatacgcaacaatcgccgccggtatctggagctgcaaagggtggcacttggaaaccgattgtttggcctctgttgtgttgttcacagcaacgatcgtacgcggggatctttggaacacgtcgccagttgcatatcagcatcagcagcagtagcagtagtgctagttccatcggatcgactacacaatcagtttcaccggttccagcgtcagccaagaagcgaaaaatggtcgtaaaactaactgaggaccagcgcaaggaacagctgcagcctctgctcgatgccggctggaccatggtcacagggcgtgacgccatctacaaggagtatctgttcaagaacttcaacgaagcgttcggcttcatgactcgcgtggctctgttggccgacaagatggatcaccatccggaatggttcaacgtgtacaacaaggtgcaggtcactctggccacccacgattgcagtggcttgagcgaacgagacgttaaactggccaagttcctggaagagattgccagcaagtactgagaggaatggagagtaatactcaacgcatgtaaattgttgttgtagttgtattttaaccttaagatgttaatcagtcaatatgaataaaatatgtcatatcacact

SEQ ID NO: 56 - DNA, 2542 bp, Aedes aegypti
atgatggtcatttattcattctggcccatcctgagcagtcacttcttcgcgctgtgcaacgacatccagaactcggtgcgccggacaatggccgcctccattagccaaatcgcgctgatcattggccgggaacaggcaacccgcgatctggtggccccgtacaccgagttcctgatggactcggacgagatcaagatcgaggtggtgaacacgttggccgagtttctcaaggtcatcgacggctgcgaacacgagacgctcatggatcagctgagtctgtgcctgcagccgccgctgaacatgatgaactggcgcttccgcgaggccgttgcccgccaaatcatccacctggtgccgatgcacaccaagattcggaaagagaactgcctcctgtacctgaccgggctcgcgatgcgactgatgatcgacaagtacgactttgtgcggaaggccggcgttcatgcgtttgtggcgtgctcccgggagttccaaaagaacgactctgtgttcaaattctttacggaaaactttgctcatagttcaaactggcgacgtcgccagacgtacattctagcggcgggacagatcctcgaaatggaaaccatcgaggtggaaatattccgcaaacacgtgctcaagaacgtcctcatgctggtcaacgacgtggtgccgaacgtgagaatacaagtagccaagtgcctcaaggacgtcattctgctgcatgagagcttctcgccgaccagcccggacgcggatgccatcgagcagacgctgaccaagttacgcgccgatccggatcgtgacgtccggggccataccgatggctacctcaatccagaggaggattccgtagcggccggtgccccagagacgaccgccaatgcctccacgatcgataccgcttcgacatcgtccacttcgtcgacctcctccctgtcgccacccccctcatcaacggcggcgacgacgacgcctatggcccacgacggttccgacaactactcaatagaggaattcgttattccgagtcaatccgatcttgatgccatgggcgtagcgatcaagcacatcttgaccatggaagaaatgaaattgaaagagtacgagttttgagcttggcttccactgggtgtcagtagcggaagtggccgaagcgtcgccggctatccgctgttgcggcacttgctagcaaagggacacaacagctatcatcatccacctaaattcgtttgctattttatcctagttgcaattgtagtgttttcctcttttattttgtaacgatcaaaggcatgatcgttggaccggccgatggccgagatttgatacgtcgagttcgagaacaatatatcaactcgatcgtatctttctacttagagatttttgtatagagccaacaacctggaaaagtttataattgaccattagacgagagacacctttattattagctcgtaaagagggcaaattaagtaacaaatcctattttataattacggtactgtccggcacgaagtttcaaagaaatatagcttactcgttttccctttatgtccataagcaaaagtacaactttgaagattagctaaggcgtaagagttggtggaggaggaggaagagggggtgttcaatagaagcatcaacgaataaagttctataggccaattacactcccattacgaccaaggcttcatattttaatagactgctctatagaaatgactttctttttccactcattacattcaaacattcatacaaaagacacacagcacacatacgtcgtacgtacatgagtatcgccggctggtttgatgaggttgttcatgaatcgctttagtgataaacttctttagctgacgaagtaaagtcaacctctctatcagcagcaacattgaatggggaacataggaatatcttccaaaaatcttcaagttgaccaatgttcaaaatcgactcagattcaaaaagtagaagagtttctgagaacttgtgcaaaaatatcgaaaaacaaaaaacttatagcaatttcaatattttcttgaggcaaaaaaaaatatgttgccgatagaggggcaaatcccatcaagccggcccgttagagtagtaattacacaaataatacaaagacagtatagttaattaatttacaagaaaagttcagttactcgatttcaattatattcaacaaaaacagtatccaattgctatataatataaataggcagtagagcgactatgtaagcgacgcaattgacaagactctaccaccccttaacataactaaaaaaaaactagcgaaaagaacagagaacattgttcaaaaagagatatcctagagttcgacaaattccaggagcttggaatggagcgatgcgagcactcgagtaggaagaggcgctaccgtcgtcggaagtgaaaactttttgtacattttataggcagacgtgggcgcgcgcgcagtgcttcagtaatagaccccgcgccaaacaaagagcgggagagatcgtagaacaataaatgcgagtatagaagaatagat

SEQ ID NO: 57 - DNA, 735 bp, Aedes aegypti
atggaaaacaaacttcaacgcttgctactgtcgtttctggtagctgcggctttggtctccgccgtctgtggcaaagtaggcacccctccagaagacggcgaacctcagctggatccgctggagttctcgtatctcacactgtggaagtataccctggccgagtgcgaaaagcgaggcgtcaatggatcgatcatcacccgatcctggatcggtgtgagaagatgtctgcgcaccaagctggacgtgatcgcgttcaatatggactcgatgcgactggatgtggacaatcagcaggctattttggagaaacactgcccaaacctgagacagacgagtgggtgttttgatccgtttatgaagaacgtgaaaacttgcgtccaggacgataactacgagatcttcgaggctatgcggaattggatcacggacgttctagagcatatctgtgaggacaatggagcacggataaagtacgatcgcgtgaaacacgagaagtgtacggccgaattgggccaatacgtttttgaatgtgctgcccaaaacattattgataagcagtatagtaatcgtaagtcgctttcggaagaagactgcagtatgatcgcgcgtgccaaggattgtcttgtgatcaagctcaaagaatgttcggtattttcggctgccgctcagcttttctacgataacttcatacagatcacctcctgcgctgctgccgccgtcgatgacgatcattga

SEQ ID NO: 58 - DNA, 720 bp, Aedes aegypti
atgaaaagaacaatcctgctacttgcacttttgggctttgcatttgcccaaaggcaacccgcgagggatgttatcaccaaattcaaagaggttgctccacagtacttggacctgttgaaaaccggagaaacagaattgaacaaaatccgatcagaaacaactgacttgattgccaatttccacgtggatgtcattcaaacaaaggagagcttcgttgtggacgccatcagcagagaaactgcaatgcattttcaaatcaataatcagccagaatcggttgacatcgtctgcttgggattcttgaatacgactgtcgatatgagcatgaacctcgctggcgttggattcaccaattgtatcaacaacgtggacgacgccttcaatggactagttggaaactatttcaacgtcctaggagttcaggaaggactgctaaatgaacttcgtctgctggatgttttccgtggagataacgtgttctacactccacaaaacatcatcgacaagctgaacaagaaactgagcgagcagaaaatcaatcccgagacattttcggaagacttggaacaggcggtggagaatctgaaaaatgatttagaaaatattctcaccgcatacggtgggtgcatgacccaggcggatcaattgctgaagaacggcgtggatatgatgatgatgcaactcacatttagctgtctgggcacggtcattgttccgtaa

SEQ ID NO: 59 - DNA, 2588 bp, Aedes aegypti
gtccacggaccactggttgggaaactctggataagaatgaatgctgcaaccggctgtatgactaggtataacgagactctcggcgagaacgcgacgaaaacaaacgcgtgcgcacttacgcgccaacatgttcaattgtcgatgaggtggatgcgaggcaccaaaccgtatatccaccataaataggtctctcacgctagacatgcaatgcagtattattcccaattgtatgggtggtgaaaattactgaggtgttccattgatggaattcaatgactatcgttatttctggaaaattggtacttcaatgacaccgcacttttcggggagatcctgtagtcaccgtgtgtaactgtagtgtatcgtcgaggaggagtacagagtccagcactgaatggctattcttatcgtgcgactttcgctagtgtaggcgaccaccggccgcgtcgattgccaatacatacatttcggttgattagcatggccatcagctcagccaccagcttacagccaagttcggatagatagtctaggtgcaaaagtcgtatcgcgtcttcaaatccaccgggttgtgtcaccgtgtagtcatcgtcattgcatagcacactcgtgaacgtttgctgaattacacgcgtatcgcgtttgccgaggcgttggtttgctcgttggctttgtatggaaatcgttgtatttgttgataagcgcacgaaaggcaagcactgtctatatgtaactgcggaacttggcatttggaagctcattcagtgtcgggccggaaactgtgtcagacggtcgcccggttttatcccttctattcagattattattctttagctttgttgattgagtgcggtgaacttttggtagccgaatcaaaaatgtctgcagaagagttgaaactggccgacgagctattcgacgacgccaagtaccaggatgctgtcgaccttttggacaaacttagcgaaaaggacgtagcggaagtgcagtggcgttttgcgcgggcagtgttcttcgtgtcgaagcaaaccagcaacaacgatgaaaaggcaaagcttgtaaaggaagccttcgggtacgctcaacgagcactcgagttggacgacaagaactttggagccaacaaatggtacggagcaatcctttcggagaaaagcaatctggacggagtgaccgaacggatcaaacagcttgaaaacgtgaagaaacatttcaccatagcccaggaatgcaatccaaccgatcccggaatcttgcacatgttgggccagttcaacttcaagctctcggaagtgaactgggtcacgcgtaagctgatcaattcggttgcagcgaacgcaccaacggcatcgttcgaggaggcgctggaatgtttccagaaggcggaaacactcaaacccggtttctacagccagaaccaactgtatttggggaaaacctatttggcgctgaagcagaaagataaggcccgtgagtggttgcagaaagcttcggaggtcgaagttcgcagcgaagacgatcgcgtgtgtcgtgaggaagctagtgaagcgttaaaaaagctataattcttctgttgtttcgtgcattgccatatgataatttatgagagtaggaacggtgtgatttccatttagcgtgcgccagtgtgctttaaggcagcagttaatcaacgctgcgagtcattgaattctagttatcacacagctgagagtgagagtttttgttttgtcattcttcagaacgctaatgacttaatttctcgttgaattctgagtgtggacagaagcaaaccggatccgtgagaataattattattggcgctgatggtgctttatctttatcggttgttgcgcttgatagatgccatagtgataaatacatgagtgatattccctagaagggcaaatccggcttttgtttcagtaatgaatgcctttgggggataaaaggttgtaggtgcgacctctcacccgacgaaagaatgggtctggtttacaatccctttcaattcacaatctaatctacacaagtggtgtggaatctggggttggaaaagttgtctgatatcttgatttactacgcgtcaattatctatcactttactagtagataaggaaccttaacgtggaaccgctttattatttgtttgtttgttttttttttcagcttgtttatcatcaatctaaatttactatatcaatccatcgcatctctgacacgttgttttggtctaccctttctaatttaaaattcatattgttttacataaattaattccaatgggtataattacagtgcattttcaaaatgttttaacatttttatacgtttgttatatttattttacatttatcaatcgaagttgtttgctggtcattagggagaactgatgttatctacatatgttacgtaagtaacaatagttcttccaatagttcaatactccatttcaagggattgagaattcaatcacacgtaattatggttacataacttatatatccacaaaaaatatgttgcgcataaatgttcttcgagaggtataaacaatcatgaaaaatttcaaaaatattgaaaatcaggttttt

SEQ ID NO: 60 - DNA, 2075 bp, Aedes aegypti
atggccaacattggaagggacaacatgctgaacgaagaacaggaatcgtcccaatcgaacagctccaatggttccggagctcttcaacagcaatcgtccggaggattccagttgaaatcgagcatccttcggcagccagcgactttcggatccggggctggtgctggcggaggattcggtggcatattcgggggtagttctgccgggggatcttccggaccgatcaacagcttcaatctgaagcccagcgttttgaagccttcgcacttcgggggaggattcgtaggtggattcggggacaagaaagacggtgacgaccagaatcaaacgaacagcaatagctttgccgggacgaatccattcctgaagacgcaggtcagcgaagacgacggatcggattcgaagaaggacgaggacaaatcccgggacgggacgggagaaaacggaggagacccgttggccaaattgaaccgagcgacgctgcccaagtcgaatctgtttgccaacgtgaaagcaatcacacagaacacgggcttcgtgttcggacagaatgtccacgagcgggtcaccggtgataatctgcagacggattctggttcggttccgacggccgggctgtcattctccagtgctgtggccaccaccaacaacagtaacgattcgtcctcgtcgaatcagggcggcgtttctgccggtggtcaaacttcagcagcttcctcgggagcaagtggaggaacatcggtggctccctccggtggcaacaaaccagagggaggagacgaaagcttagaggaggctgcccgccgttacgaggaaagccgaggtgccctgaagaggaagtttgacgaagtggaaaccgttactggcgaggaagacgaacggaatgtggtcgaagcgaactgcaagctgtttgcgttcgcgaagagtaactgggaggaacggggccacggaacgctgcggctcaacgataaggacagcaacgagtcgagggtcgtgttccgacaggccggcaatctgcgggtgctgatcaataccaaggtttgggccggaatgacggcggaacagtccagtccgaaaagcttgcgtctgaccgccatggacaacaacggccaggtgaaggtgttccttgtgatgacccgccccgaggtgatcggcaaattgcacaaagagctttccaagcgcatccaggtggccaaggagaacgccccagctgaggtggattccctgttacaggacgacgaagccggtaaagagaacaacaacgacaatgctgccaccaccaccactactagaccgagtgattgtgaagacgattcgacaacagcagataatgcaaccagcaaggaaaccgaaacggaagaaccttgcgcgaaaaagcgactaagttcgtcgtcttaagccaccaaccgtgtcacttttcatcaagctatctattttatctatcgtatctacaaaccaacacaaaaattaaggcaagtcatccgcaggatgatagaaaagaggtcagcactacccaatttgcttagatttccgctcaaaaaaaaacacatacaaaatggttattcaattgcatatactattgaagggtaaaattccttcccccaggatcgagaagataagatttcgggctacgagtttataaatattccactgaaaaaaaaatcattttcttccttatttccaattttaatgtgtcctaaatagatgttgaaattgaactgttcgcatgcagagacggcatgcatctcaaccacagatattgggtttgtggagaactttaaagaatctgctgccctgagcaagtgggagagatataggaggcgaatggcgacatggaactgtaaaaagcaatttcgatctgcgcggttaatcggagtgagaattgagacttcatctagagcgtcatgtcgactgtcgtcagtcaatcaatgctcgtctttgtcgtcgtttaaattattgatactactactatccaacagtagaagtaacattagtaggttcatgtttgtgtaaaaaaatgtaacgatcaaatacagtgaaatacaacaagtaacaatgtggaatgaaaataaact

SEQ ID NO: 61 - DNA, 813 bp, Aedes aegypti
atggctcacaaagtgttcctacttgtctgtgcactgatccagctctcaactgccgcggttgaagtacccggcaagctggttaaaagttcatcggatggaccttttgtagcatttgcagcagctacggaaccaacctgcgatggagtcagcgtttataccacatgtaaggactgtaataacgtgctagtttgcttgggttcaacacaaagcacgaagaactgcactgcggcgactccatccactcctttctgcgtcaacggtgcatgttctgcgacatatgacagcgttgcaggatgtactccttctggagtagcttgtaccggagttggattctacccagacgcaaaaaattgtcagatctaccactactgcgaaggtgttgatgaggaatcttctgtgtacgagtgcccgccaaactatgtgtacaatgcggaaaccactctgtgtaagcagaagatctacgccacggattgcgtaacggtgaaatgtgatgagaacaaaattttcgtcagctatggcactagcaagaaatattacgccttctgtcagttcgaaaacaatgttgccacgagcattcagttgctcaagtgtccggataactgcaactttgatggaaacagctgcgtgttcaaatgtccaggaaccggtaactacgcgcatgcggatccagtcaagtactaccagtgctactactccggagcggtgctaacattcatgacacagagttgccccactggaaagaactacgacaaggacttgcgagtgtgtgtcacaccaaaaacgacagctgctgcaccaacgatcattgaataa

SEQ ID NO: 62 - DNA, 624 bp, Aedes aegypti
attgatctcgttagtcagtctgccgttgactttcagttcaatcgctgtaagcataatgcagagttcgcgtacgattctagttttggtggtggccattttgactacggttggagcggtcccagcaggctttgccgatgtatccgatcagatttcggccacgatcgaggcggagaaaaatgtcggagtgttcaccgttgggaatcaacttccgggaccaagtggcaaagtgggtcaacaactggtctacaccctcggacaacgggtcgtaggtgatcgtctggtgtcgaccaaatcggacagcaaacagtggccaacgctgcaggacgttacgctgagcttggcctatccacagtccggtgttggagctgtcgtgacttacgtccaggtgatcgtcaagcagagcagcaatcttggcaaagggtacgtcgtgtccggaggaatcggtcagcgattcatccatttgaacatcgaagcctacagtaccaattacttcacttacactgttcaaatatttggattgtaaactgtattagtgataagaacttacctcgtaataaaatcccggattatatccggcgccaccgaacaggaagagcaacgcgtggccgctcagtatggtgaaca

SEQ ID NO: 63 - DNA, 504 bp, Aedes aegypti
atgctcaaagccatgacttcaaatcgttgctggatcgcagtggccatcgtttgcctgatgggagtggcagctcaagccggtcccgattttaggaccaagcgggaacagtatgaccacagcaagcaaatgtgcggcaagattcttcggacgccggctgccgacttggagcactacctccgttcggattatccggaaagtcacgacacggcctgcttcatacggtgtgtttccatcctgaatgggggatacgatgatgagacgggagtgaacatgggtgtgctgttcgagacctacggaggaagtttgaccaaggaggaatacgccgatgaagcaaaggaatgcctagcgttgcgggacgaggtcgagtgctactgcatgaaggcgtacaaaccgattctgtgcctgaaggagcagttcaagaagcggaatgtgttgtaaacagaagactgaataaccaattagttgattgttgaataaatgagttcaataggaatattacaaaga

SEQ ID NO: 64 - DNA, 738 bp, Aedes aegypti
aacgtaggtgcaacaagatttttaacagtcggtcaaacggttgcatcaacataaatcggtttgcctaactcgcgagcggacctgattttagtcaaatcgtctgagcgtgtgtgggctgcataaaagaaacggttcacggtctcaaagtcaagagagcaaaacgatttctggcacgtacaaaagcttagctgctcagtcgttaacagcattcgtcagtgagaatacatttttagcgaaagaagtttgtaaacgtatcaaaatcaattatattaataatattagtttccaagtaaattataatcaacataagaaacaatcctcaaaaatttgctgcgtagtttcattgctctcaaggcaacagaacaaagaaaaacaatatgtcggagctacatttcgatgaatacgagaagtacaactttgagttcgataagcacatgttttctggccattcgggaaagcagcgaaccaagaaggaagctagtgaacacaccaaccattatgatcccagtggccactccaggaaaataactactaaactgcgcaatactgagcagaatcggaaagacaaatcgaagaattaatatttgcaagccaacttaaacttgatacattttcacgatgaaataaaatattgtactgtttgggaattaatttgaatacatgttggctttgtttaaaaacaaatccagatttcattctataataaacatgaaatttagattgattaactaccatttt

SEQ ID NO: 65 - DNA, 2040 bp, Aedes aegypti
ctctaattgatataggtagtttcaatagaacgtaggtaaatttgagtatcctggattttaattttttttttattatgttttatttctttttttgattgcaactatgagtagtattactatgaaaaactattcatgtaaaactaaagaatagaaggaccttggacttctacataatctactttgtgtatgtttttattaaagattcttctccggggattcgaaagttcgtaaaagaggctacgttttacataaattgaacgcttcatgacaaattccatgtaaatacatgtttttatttagcgtattgctttcaccgagcactttcataggtaaatgtatttctatagtgactcaaggtgaagatgaaacgaacaacaataacagaaaattgtgaaacccagcaatacactgttcacaagaaagttcctcattatcatattctcaaaattattcaatgccatcctcaccctcagtaatgtgcaatctttcaatcctaatcagtaagtctgcgagtaagtcaggaagtatataaatggcgaacatttcacaatcgctacatttaatcactgtacaccggtcgaactggaaacatcaataacgtctcatcatgagtggcttccagttattcctcgcgtttgcggtagtggcttcgattgctgttacccatgcagcaccgcaatcagccgtggaagctctgctgaaatataaggatgaatgcattgccatgagtggatcagatgtgggttatcgtcaaaccttggtagcgattcccgaagttagagcctgcttattgaaccgtatcgatgtcttcgagatgaaaggggacgcggtgttgttatctgagtcgagcgaaaggagaaaggatttcttcggcaaatattgtccgaagttcaacgaatcggtagactgttttgatgacattttcgaaggaatcgcaaaatgcacgggtgaggaaactgagaagatagtgccagttttcaaggatgtggcctatggcgttgttgatctagtatgtgaaaatgatggacagtttgtgttcgaaacccagaaacccgagttcatggcttgtttaggaacgctcagggaaagcgttacggaatgtaagatttcgaacgtgaccaaatcaatttctttgatacattacggcgaagagcagtgtcgagatgtggaaaattcaagagaatgtgtcaaacaaaaggttgatacttgcagttctcctgcggtttataatattttcgaggttctgtacaatagaataatgaaggcgtcgaattgccatcagtacacaaaaatagttgagctatacgataacaacattgatggtgcttcatcgaatgacgatcctggattcaatggataaatctaaactatactgatgaatggataaaacaataaacttactgaagtttccatccgcttttttttgcattgttgtgtatttgtataaaactcataacagctcaacacagcaccgtctgtcctaaatctactctgttaatataacattttcccacacaaaacgttaaatgaattctaccccattaacccgagtattattacttcgattttcatttccccggaggtaccgttaccccaaatactgaacctgcaatgtttaattgccttgtatcattacctcgggctaatgtgattcgaggaaatggatttcgggattatgtcgttcgagggaataggatagaatcttctacagataccaacggagccaatcttatgagtcatctcaattctgagtcatgagcgactattgtagagggttataaaaatcatgaagcaaagcgtgcttgtacaggtttaagctatcataataattcaagttaacattaatctacggatcggcttactcctcattaccgttaattagtttcacgtaaaaataaattatccacccgatgtcatacaaaatattaacttcttcccagtggttatgttttcggtggaaatggaactatacttacaattatttacaattacaattatttatataatatatgttttagggccttccttagccgagtggtt

SEQ ID NO: 66 - DNA, 306 bp, Aedes aegypti
atgttcaaaatcatcgctttggttgcctgcttggccgtcgttgtctccgcccagtattacggagaagatcacggtcaccacaaggaggaacatcatgcccatcccaagtacaagttcgaatacggtgtcaaggatgcccacaccggagatcacaaatctcattgggaaattcgtgacggagatgttgtgaagggtcagtacacccttacggaggccgatggtagtgaacgcatcgtggactacaaggctgacgaccacaacggtttcgaggctgtggtgaagaacgtaggacatggccaccattag

SEQ ID NO: 67 - DNA, 1666 bp, Aedes aegypti
taaaactaattttatgaaatgttaaatgttatttcataaaaaatcgttattgcataaactgataaagatcaattaagtaattttatatgacaatatttgaatttcaaaggataacataacactaagttgtaaaacagtgatcaattggccccctgattacggtacctaccctagcaacgcgagcggctcatgtttgggagcagtagcagcattagagcatggggcgatgttttttttttttcgttggcgtcttcatcgttgttgttgatgttggttggaagacccacgatgatggtatggcagtccgttggcttagggggagaagatgttgtccagcggtactctgtatggcatagcagcagcgagtacattctccggtcgtctacaagtctacaacggggatgaaccccaaaaccgtacaccccgaagcacccagccagcgtcgccttcttcgatggcttcggcgacgacgtcgccgccagctaggatcatttgaacggtgtataaatatcatttgcaatccaaaaatgagcaacagtcttaattgtgcattgttccaatcgagtacagcagtagtagccagccagtagccaacagcattcttaagaacttaagggtagctgttcttgtccgtggacttaaccctatagccaaccatgaaagtgtttgtagtgttaagtgtcctgttggtggccgcttcggcgcgtccagaacctccggcacccggtggctatccgtcctactcggcgccttccttcagctcgggcggtgatctcggaccctacgcttcgagccaaggtggtggcggcggtggttacaactaccaaccagccacccaggtcgtccagaagcacatctacgtgcacgtgcccccgccagagaaggaagaacccagctatccccgggtagtccagtcggccactccccagaagcactacaagatcatcttcatcaaggcgccgacccctccccagccaaaggccccgatcatcccgccaccggtccagaacgaggaaaagaccctggtctacgtgctgcacaagaagcccgaagagccggaggagatcgtcctgccaacgccaccaccaaccaagcccagcaaaccggaagtctacttcatcaagtacaagacccagaaggagaagaagcccgaatacggcccacccaagcaggaatacggagcaccaaatggcggagccggtggcccatactaagtggacagctggacccggacacggacccagacccggactgcgaccccgacctggacaccaaccgcaacggaagcggccgccctcgacgacgtatgctgatatcgatcctgatgatactgatgttgctgaacaactacaaacgcaaaacatgctgaccaattggtgcgaatgtgcgagcgagagagcgaaccttaattccattttaattcttctgttcctagcctgtaagattatgattaatcctgaactgcgaacgcgttttggtttcagcaaaaccgtgtcgcagttttgcgctttttacgtgtatataggaccgtagaaattcctatgcaaacatctgttcttagcgagccatagtcgaatcaatcattattatgttatttattacgttattgcaaaagcatatctagttgaagtaaaaaaatctaacggtgacaaatttaagaaaaaaa

SEQ ID NO: 68 - DNA, 942 bp, Aedes aegypti
gaaagaacttagaggcgggtgtcgtctgtcatttatggtagtatcagcatagaagacgtatcaaagagcaaaatgcgtaggataataggagtagcgctgttttcggtgatagctttaagcatagttcccacgtcagaagccaaccgctgtgctggaaggcctgacggtttctttattaacgactatactgcatgtgaaggctttttcacgtgtatccgcgagaccccagtgccaggacgttgtccagaaggattctatttcaacgagaatagtcaactgtgtgaccatccatggaatgtcatatgcctgctttgtgttcgcgaggaaacggaaacggaaactgaaccagatacaaataacgtagttacggagtttttcccgattgaaaatgaatgccgcatgtacacgctctgtgtcgacggcgttggtttcctgagagagtgttcgccgggtttgatgttcgatcgggaagctcaacggtgtgatctggaggcgaatgtacagtgcgttgaaagtctttgcccgaacagtgtcaacccagcggtagcatctatggtaccggatccaacggattgctctcagtacttcatttgcttcaacagggttccgaatggaccacattcgtgcaacactggtctcctgttcgacccgatcaccaggcggtgcgacttagaggaaaatgtcgagtgtgaggttgtcacggaaccaccaacgcttaccgattgtccggcctcgggtctgcactacattccagtggaaggcgaatgttcgaacttcttcatttgtctggacggtgacaaaatcggcgaagaagtctgtgccgatggacttatcttcgatgtcaacctgagaaactgccgaccacgaaccgatgaaggctcgcagtgtattaccgatccgccagccgaagtagctgcgaggctctttttggggtacaattga

SEQ ID NO: 69 - DNA, 963 bp, Aedes aegypti
atggtcttcgaagatatcacaaaacgaaactatgaaatgaaatacactcagctggacctagatgaggccaagatagtgtatgccaaactagctcgatggcacgctacctccatgtacctatccgataaggtaccaatcatttcgaagctggacaatagcctcggaaaattgatggacagcgattttagcgagatctggattggacacatcgctaccttggccaagctgtgccgtggatggcctggctacgaatcgtatggcgaacgtttggatcacatgaaaggattcattctatccaaacttaaggacatctatgaagtgaagccatccaacttgttcaatgtgttgaaccacggtgacatgcattccaagaacatgatgttcaaaatcgaagaaggtgtcacgaaggacattctactgctggactaccaaatcagctcctggggaacccctgcaagcgatatcatctactcgttgtataatacgtgctcgatagagacaagagataatcaccgtgacgagctgatcaagttttaccacgatgagctcacagccacattgaacaaaattggttatctgaagaagattcctacgctgctagatgtgcacgttgaaatcgtaaaatgcggacacttggaaacattcctgtgttgcacgttcctccccttcatgatactatcattcgatgagatgatgccagcagcagaaaacccggaagaaggcgtcgaacttgactttgccgacaaggaaaagatgggcgaaatgatgctgaaggtattccagcacccgaagtatgtctctctcatccagcggtatttgccagtattgctcccacaaaggatacctagatcaatgaacaactttatgtagaaaagggagatattatggttgcataatatcagagacatttgttgttgagtatcgtttttgacaaaaataaatcggatcagtattcttaatttt

SEQ ID NO: 70 - DNA, 1209 bp, Aedes aegypti
ttcttatatgcatcattgtggtgccatactcaccgcaccactgagcaaacagctgtaatttctcgcgctcaactaaccagcgtgtgattcgtgcgatgaagttcgtcacttattgagcatttaacaagatttagaagaagcgcctctgtgtttgggtgtgctcacgctctcactataggaacctaccaaccggtggacgttggttggggtttgttggtagctaatgatggggcaaatccgcgtgtgcgagttgccgtgagatgcgcttgttttctctcgcagcgacggcggcggcgtgaaggagaagcaacaacagcaaaaaaaggtggcagaagtttgatttcggcttattctttcggctcttctcagaggcggtttgtttattgtttgcggcgtggtgcgcttgatggcggtatataaacgtttttccgactccgatgggacatcattcgaatagtaggacttgtccttaacggtgtcgaaaagtttgcaacaggattcgctggggttgttctattgagaagatctacgagaagtcaggatgaacaaatttgtggtctttgctagcgtgttcggagtgctggtggcagttagtgcggttttgggggcaccagctccccaggttgatgccggcccaagatcttcggcgcaagatgtgcaaaccgtgcgatactacagtgaaaacaatgggctcgatggttacaagttcacatatgaactgagtgacggtcaaatccgctccgaagtgggcacgtacaaggactcgaaggatgccgatggtaacgacgtcaaggtgctgttcgtccagggatcctactcgttcgttggtcccgatggtcagacctactgggtgaactacaccgccgacgagaacggataccacccgaaggtgggaactggcccaaccggaggtatccaagcgggacaagatgcccccatcaacggtgcgtaatccggaggggagcgagcctaattgtcgacgccacagtctagccttagttgattctaagtcaatgcctacatactccgctgagccacaacaaatgagatatagaatgcgagattttcctattcggataggttagtttcagacaccgtaggatctagtgtaaatataatatgttctagtgtatacgcgcaacgagtgtgttttctctcgtagcaataaataaactgatgatcgagatgaagatggagaaagaagtcgaa

SEQ ID NO: 71 - DNA, 591 bp, Aedes aegypti
gtctctcaaccgttaatcaagccaacgaacgcaggaagcaaccatgaaggttttcatcgttctatcggtggcaattgccctcgtcatcggggcaccagctgacgattccaacaacgcggaaaatggcaaaaaagacaaacgtggactcgtggaaatcgggcacaactttggctacgaagaaccgcttctggttgatcctcttcatacgacacatgtagccaagcacgtgacggttaccaagaatgtgcccgttccgtacccagtagaggtggagaaacacgtcccagttgcagtgaaggttccctatcctgtccactatgagaagcacgttccggtggttgtggagaagaaggtcccagtctacgtcgagaagaaggtccatgtcccagttgatcggccagttccataccccgtcaaggttaaggtccctgtggtccacaaggagtacgtcgaagtggccaagccctatccagtgcatgtcgagaagccctatccggtgtacgtgaaggaaccggtatactacgaacaggctccggtttacgtgaaggaccactactaccagcacaagaagccgtactacggttgaatgag

SEQ ID NO: 72 - DNA, 584 bp, Aedes aegypti
ctttagaaagaacttcccccggaattcataacgcgcatcttcaaactaagtgaaagagttctaggataaatcagatcaaacgttgcaacatccagtcgacatgcagctgttcaagaggaaagcattctacttcgcattgcttctagtcgtactaagcatgtgcttagaagaaaccgaagccagacgtaaaatccttcgaggacggcgtaccatcacccgcactcacaagcgtggaaccgcaatcccggcatgggccatcgttaccttggttggaattgccaacattttactcggaggacttgcctatcttgcgttccggaaacttgtcctgcaaacaccgatcgagaacgtcaattcctacacgccagcgatgatgcaggacgactcgtaactttgttctgagatgcaatccactgcaaaacaataggttaagattcgttttaagaaatacagagcatctcagttgcgtccgtgaaaggttcgatatcttttgagaaacaacatgtaggcatattgtctgtaacttttcagaatccagtcaataaaccctgataacacaatttcatgaaagtagaacgtcgatc

SEQ ID NO: 73 - DNA, 1849 bp, Aedes aegypti
acagcaaggaaaactcttcattttagatttgcaaatttgttgctgaaagtgtgttcgactatgttcatcgtttgttagttctacgaatgttaattcaattaaattcaagagaggtgtgattcatttggtgacacaaatcaagtgtctaacaagtattaaaaatggagaacacatcgtatgggataaatgtggccaatcggtacgaccttttttcaatcgacgatgaaggtgacgatccttttgaaactataactcaaaagaagcaaaaaaatcaaaagaaaaatcaggccaccccgtcaacgaccactgaaaataccgtcgctaaatcggctgcaaaaaaggtaaaaaacgcggaaaaggagaacaaaaccacgcaaaacaaaactattaataatgaaaatcgacatcctgacaaatatgaagatttaaagagttctgccgcaatcaatacttctaatttacataatcagcgtggggaaaagcatggtattaaagaaacccaaaaagataacattcgcacaacacgtgaagatggaaacaaaaatggctacccaggaaaaaatgccgaaagacgccagattaataagagcaactatgcaggtactgaaaaccgcgaagacaaaaataatcgaaagaatcgcgaagctggcaacgatgaaaatggaccccgcaaatcacagagtggaaacaaacgttttgatgttcgcggcaaacgagaatttgatcgtcaatctggttctaataaaactggcgtaaaagcggtcgaaaaacgtgacggaactggctcacataattggggtagtgctaaattagacgcaaaggagttcaacaatttccaagaagagtatatgccacacgaagcggaagaagaaaagggacaagcttcagatcagactaaggaagaacaaactgagcaaagtgatgctaagcctgttgaggaagagcttaaggaaatgacattagatgaatggaaagctcagattgctgctgcaaggtcaaaaccccaatacaatataaggaaagctggagaaggtgaaaatgcagctcaatgggataaaatggtagcattagataagaaaaaagcagaaacagctgaagcagaagaaaatgagactcagaaaactggaaaacagaaacaggtccttgatattgaattccatttcaacgatggtcggcgtggtggattgatgggccgaccgagaggacgaggtggaaaaggtacaccgagaaatccaggaagacgtgagggagagaaaaaagatacgaatgagacacgtgaaaatgaaccagataaacgagaccggcgcactcgtccaccaccaacgaatgatggtaacttcaaagctaaacaaagtacacgtttcaataagaacactgctccaaaagttgacgacgagcatgattttccttcgttaggctaaacattggcttctaagttgcagcatattgatggtgaagaattgtacgatacatttcgtaaacacttgcttactttcagaccagttggtttctttacagaaatattcacaaaataatgaaaatggtgagaataattcatatatcgatctatagtgctgtaacatgtaacaacaaaaatcttcaaagaaagaataaaatactaagtacattgaacatgtgtattactgtatctgaaaaaaaaaaaaaaaaatcaaatagattgtcgttgtttctcccataggcatgcatgaggaaatttgcaattccaaatagaagagagaaccgaagcattttgtattattttttcattgaatagtattgcgcaaaatgtgtaatggcatccgtgtaaaattccacattttttcaatttcataaatttagtcgagtgttg

SEQ ID NO: 74 - DNA, 2947 bp, Aedes aegypti
ttggggatgagatctgacaagcgacacatctggtcacattactttagcaataataatgggttcatattacctgcataaaattccgttgtttattcaaataattaaaaaaagatgacatattatcgttttgaaaacgtgaatcaatctgctatacaaacactatacaataattgtttcatcatttcattcttatttcttgctagagtagaatgctgcaaaatccaaactgggccgtgaatctcttggccaacagattttttctacgaattgagaccgattttgttgttaatgtcaaatatttgcatgtatcgcttaaacgcctgaatataggcaacatcagtatctcaaaaagtaatccttacaatttaaaaacaatatattatatccgacaacgattgatcaaggtttatatcaatgtccgaatttatgttttgtatgatcatttgccaaaatattgaagaaaactttcacgtctgcgggttgcggagtgttctcgctatttttgttccttcacatgcttccaggtataggtattaggatggctgcgtggtgtagtgtattcaccattcaacctttccacattccgtctctgtgatgtgttgtcccatcttctgttcattgtactgaacggtgttattcaaaacatagcacattggtgttggctggtgaaaaatccaagatatattaccggaagctaagcagtgtagttcgaaaactggtgcaaaatatttccaattatggattacgaagaattgacagtggttgaggagcagaaccaaccggagagaaaggaaagcggcaaacgaggacgtaaaccaggccgcaaggcgtctactgagaagatcgatatgaaggcaaagttagaacgcagcagacagagtgcacgggaatgcagagcacgtaagaagctgcgctatcagtatctggaggaactggtcagcgatcgcgagaaggccgtcttcgagttgcgccgagagctggagaaactccactactgggcgttggaagtggacgctggtcgtttccccgagggactgcaggagctgctcgaggagctgggtgccctgaagcaggagtaattgggaggcaactcgaccccgcgatggttcttgatgaagttgggaggctttaatttcttcgtatcagttatccgagattgagtttcctccgttattgagctttagtagtagttatttatttggtttaaacgcgaatcgttaaatttaaattggtttgataccttatgcataggcgtttcgttaaaagaaattaaccttcgttagtcacgtgttaattttaggttagaaattattaggtttatgatttcgtttttatgagttttactataaaaatccgtgaaaagaaaatataaaaaaaaacataaaatttaaaaaaatactacaaaaataagaatgagtaaacgaatatggcttaaatctaaagaagttacctcaaataccgataagttaagtacaatttagtggtacaataactgatgatatattatgaaccgaataaacatttgatgtacataatctatttcgatcaccacaaacacagtgattcatgcaattagtactgcagattctgcgggagcactgatacgaacgtgagtagatccacacttaatagagtttgttattcgattatcctggtagaactccactgattaaagttcaatattaataacctgacgtgtccattgtaaaattgcaaaacatacatatttaaatttgctgaattgttaaattgctgcaaacttccgggcggcacacaagttatataactatttttgtaaaacagattttggatgatatcaataaacaacagcatggaaatgctaccgctaatctatttagtctacagtttttccaaatgacaaatccctgaagcaatacatacaatatttaaataagatcttcgatgtgtttcgatattttaaacatattcctgttctttgcaagtgccttacatgcttcaacgaaattaaatcccttcttaaaagtttttttgtgtgtcgaaagtttgtttttaaatgaagataaatcaaagccaaatttcaaatttccaagaagacaactctggagaaaccaaccagccgttccgctgaaaatttcttcgattggtccccagttgcgagcgaacaatcgatcaagtttttatctagaacggcttcctggttccccaggaaagtgcgcttgaaaatttgaggtgtttcttcgatttatcttcaccttaagtgatactttcgtgcatttgtgcaggatttaccctagagattcagccagatttttgaggaaatttccacagtaattcgtagaataatcttctagatcaatatctatgatccttaaacatatttgtaaaagtctcttgtgaaatgtttgacggattctttgttaaatttatgtgtatcgctgttgatattctttggggaactctgcgtaaggttcacttttctgaagtatctatccagattttctgaagggatcgcttgagcaagcttgtgttcctagtggaaatcctgtgagataaatatcagaaagcagcttgagatatatgataatagctaaggtttccagataaaatcgaggatgtagctagcataggattttgatttacggtgagtgattacaaacaagagctgcagatcccgagaagaaactacactgaaactgcagattttgtacaactgcgattcaaattacttggtaatcaatcattaagtgatttctgaaaccttaatctaaagggaattttatttcattttccttcgggtagatgattaaaaagacccaatgggacgagtcgccattagcgatggaacaaatcttccaaatggcaggaaacgtatcttagcacagtgattttttgccgatttcgtgatcgaagagcgtctaaaccgttgattttagtgaaaaagtttgttcagagaagtttcttgatt

SEQ ID NO: 75 - DNA, 844 bp, Aedes aegypti
atcggactgaatggaaagaatccatctcaacaactcaaaaattggcatcccgcacataacgcctaagttggtaaaatgaactcacttcagggtactttccctgtgcgtgtatcgcttctgacagttgtccataatgtcgacttttagtttgtcattttattttattgaaaacaatgccggtttattcaacgtaatatgatgttgtgcaatgtattttttctattatcgtaaatcattaaatcacgttattcattactaattttaataatttttatcatggggaaagttttatcaatcgctgctcggcaagtgaatcgattcaatgtcgagagccgtgctgaaaaagtgatatcgcagtcgaagccaaaaccagctcctaaatttcaatcaaacctaaaagatcttcagcgagttttggatgaaaatccggatttgctgcaggagctgaacaaaaagcatgcaccgctggatgataacttgaagcgagtgttcgttacgtcaactgatgtgattccagagccgacccacaaagatccgagcaaaccgttgccgttgaatcgaaacgccattgacgaatttgagtacggacatttggaaccacaatcggtgcctaaaggacactgcacattgcggcaagcaattgaatttatttccaagcatcacactgaaccgcaaatatggacgtcaagcaaaatagcgactgaatatgctatgaaggaagcgttggttagcaatattttgcaacattttaaaacgttcgaattacatataccagataaaaccgccgagaagcggaaattattattgaagtcggatacaaataaaactcttgaatagaaatgaa

SEQ ID NO: 76 - DNA, 548 bp, Aedes aegypti
cacaaaagtgcaagtgcagtttggtatcgtttgcttccgatttctcacggacacaccgagttttgtcttggtttccactacattgttggttgtgtaatttcgataaaatgccggccgatatctggggatttgcctacgcagcaacggtcgctgccggtggcattctaggctacgtgaaagcaggttcggtcccttctctggcggccggcgtaacgttcggtgccattctgggttacggtgcattcctgacgtcgcaagatccgccacgtcctctgctgcaggtcggcacttcgctggtgctggccggaatgatgggagccagatgggcccggtccggcaaattcatgcctccgggtattgtttgtgtgctgtcctgtgccgtcttggctcggggcctgctgtttcataatcgttatctgccgatgattggcaagagcgattagaaggggggcgggattatttgttctgcaacgacattattgatgcattatgggtggaggtatttgatagtaagaaataagatgacacaaattctagacttattaaaa

SEQ ID NO: 77 - DNA, 471 bp, Aedes aegypti
atgtatcgctctctagctctggtagcccttctgttagccgtggccttcgcccaagaaggaactcttcatacgcatccttgcaccggtggactcccacatcccaactgggttcggatcgagggatgcacccagatgccctgcaagctgatccgcggccaggacgtcaacatgtggctggagttcactgccctgtccgggttcgacggtctcaccaccaaggtcatgtccacggtcggaaacgagatcaacattccgtatccgcttccgtcggacaagtcgaacgcctgcaactggctggtgaacacccgctgtcccgtcgactacaacgaagacgtcgagtaccatctgaacttcttcgtgagctacatctacccggaggttccagatctgaagctggagatcagcctgatcgataactacaaccacgtgcagtcgtgcttcgtcgtggacacttctgtggtgtcgtcgtag

SEQ ID NO: 78 - DNA, 2050 bp, Aedes aegypti
ccccagtgcttgatcggttaacatctttttccctcagcgcagaggcaaaggtgctgtccggtctacatcagcgtgtttcattctagtgtcgcgcttatactgtgctctgtgctagccgaaaattgcccccagcagaaataggacccatttcaagcgaagaaggtctgacctagcaaccgctcgaaaatcatgggcttcctacgaaatacattcgtattaattacgatttgcgtaatgtcggcacaagtattcgcacagcaaaaagtggatgtcacaaaccaggaaatccgagatgccattctatcgctggtgcactcctacaatcttttggacaacaaattggaacgacacgagcaacgggagcggtcattgggcgaacaagttaaaaagggtctgattacgctgcagaagaaccaacggatattcgatccactcaagggtactatcacaagactggaggaaagagtcagtagtatcgagacggcactccttagccaagatgaaaagaacacgatgcagcagatgaagttgagcgaaactctggagggcgtgctccgctggttgagcgacaatgcgcacgcgctcgagacgagggcccatgtggaatcgcgaccggatgaggaaacggagggcatcggaggaaagattgaggagcttaccgaaagtattagagaattgcgacgggaaatggccgagcttaagtcagatagagattctattgagcaatccaatcgcaacctgttggagcaggcagaaaaattggccaactccaaactgggttcgacggacgacatccttggtaagatcgaggaacgcctttcttcatactacaacaatcccgtagtaatctcccattcgaacgatgccttcgaggagaaggtcatcaagaagttggaagcgatcgatgccaacgtgaaggaaagtcattcatcgctgtcagctccggttagtgcacctgctgcatccattgataaggagttcgttcacgggttgatcaatgaaacgctagaggccatcgaagatatgcgcctggaagtgctgacagcttccgataagagcttctcaaagacggcgactcgtatcaaggagaacaacgaggtactgcagtcctcggtgaatgaaatcctgaagaccctgtcggaagaaatcgtcgacacggaagcattcttctccggtacaaagaaggacattggaacgttgaaggacgaaatcgctgccttaagccgactagagaaacttctactgcaaacgggtgaaaatacgttggatgctaagcgaggcatagaatacggaatccaccagattttgcgagaggtaacggaagttgtgaagactaattcaaaggaactgaactccactgtcaccaagcgtttcgatgaaatcggtgccaccattttggacaatcacaacggagccctgagtaaccttagttcgaagatcgaaaccgaaattagccaggtttggagacagattggtatcatgtatcaagaaatctcgtctagcaaacaagctttggatcggctgcagcagcaaaccgaagcatacgtgaacggtactctggagacaatggatagcatggaaggaaaggtctcgcaaataaccggtcgtatgtccgaggtagactcgaacctaaactacctgttgggacgtctttccctggtaactcaagagttcaacttcattaagaaaggtctcagcaaagcactggatgaaataaagagcagcttcgtagagatacaagaccgcgtgaaagggcccggaccgcacaagatttcctcggatgaggtggatgacttcaaccaagctcctccggcgaaatgaacttacttagactagggaatacatattagagtagaacgaagatttctgtgcttttgtagctgatagaaatgaatggattcatatgattgtaagcagatacagtgtgcttggaaatcaacagattgaactaacttcaaattgattgattttatcgcaatagaattgaattggattttctgtgtattgaaaatgatcaataaagtattttgatttcatttatttcctaagg

SEQ ID NO: 79 - DNA, 2094 bp, Aedes aegypti
caagctttcacagaggtttcatcagatttttcggctggccgcatagaaaaaagaatctgtccgcaggccaaaacctacggtgcgatgaaccaacatttccagaaagcgattcccagtggaaggatcaacaacaaaaagtatcgtccgcggaagtagttttagcggagttttatccgacgaaagaagatgatgaaagtttaatcagtgaagaaacaaagcaattttggagtgaaaggaaatttctggaataaagcaaacggaattctcaaccgagagtcaaaatgagtgcccccatcgtcatcaccgtacccacaaagtacatcccgatcaatctggccccgttggccatcaagaccgagtcgtcgtcgtcccattcgtgttccctttcggatgcggacgagtcgtcgatggatgagttcctggtgcgcggcaagaaacgccgcctggaccatctgacatgggaggagaagctccagaggaagaaattgaagaaccgcgtcgcagcacaaacctctcgcgaccggaagaaagccaaaatggaagaaatggaacaaaccatccaacagcagacagaccaaatcagcgaactgcaactgaaatgcgcctcgctccaagcggaaaaggacacctactacagtaagtgtctcgatctggagaaccgccaagccgagctagagcgacgactcaacgatcttcagcagcacctacagttgaacgccatcaagtcggaaacgacgaccccagcagcctcacacagtgttggctctgcttccacccttctaggatcagcagcatccgcccaaacccctcagcagcagggatctttaatggaagcacagacgacactgagtcaggacaagatgggtctgaagaccgacgaagaaaaggtagcgaccctgtggaagatagttgctctctgcctgctctacaggacatgctcgaagacttcgacgtgtccaagctcgaagagctcgccgaaagccttctggccaatgtcacaagcgaactggaaggaactgatcagggaagcatcccgtctgatgccgaaagcgcaagcaccggctgcggaatgcctggatcaatggtggggacctcaccagaacagctggaatccggcgaaaaagctgatcaaggaagcagcctaattctgaccacccacaactactccaagtcaccctttcatgtggagaaaccgcacagcagcacaagtcagcagtccaaccttgatcaatcgcaatcgacggtcttcggaacctacgacaacgacagtaattgcatcacaatcgtcctcaacgacgacgaccaagaacttccactggaggaggaaatcatttgcgaagaaacgggagaagaattcttcccccacgagcgggacttcaacgtcaagttggtaccggttcgggaactactctcacccatccccagcacctgctccaactattcttcctcggatgaagtgaaacaggcaccggaagatgaggagcagcacttgttcaagtgtcccttgacgcccgtttcgttcgtgtcggatggaggttacgagtcgttgggttcacccaacgggttcacggatttcaacagcagtcccgccagttcgttggacagtttttggaatctcgatctctttcccattttaacagatatgaactaaaatgcaaatgctacccagttgtaaatatttcccgttccttcagtgatgtacaaatttgatactagtgttaagtcgttaggttcctaagcttatctcttttaaggcttcgtgagttgagtagattaaggttagattgctgtacatagtagacacctttaagaaaagattgctatttatcgttatcatttattgttctctctggttagagatggctatttgttctgcaaatgctgtgtagttttttcctagttgatagatttattgttttattttgtgcacaaaataaatgtagaatactatcattgaaacatgatctttagtagtataaggaaaatcgtaaaataggtaatgatgaaaaagtataattgttacatattttgtgagtttcatattactctttgaaaggatgacttgaaaaaaaaataaacaattttaacacacta

SEQ ID NO: 80 - DNA, 781 bp, Aedes aegypti
gtttgacagaggctcgaccaaaacaaaaaaaaatcctgtcaaacttttgttgtgattttgcgaaaattttcggtgttcgctatttcacacaaaaatggacgccttggaggtggaggacttcgattacggcgagttgaagaacgacacggccttcatcaacaagaacgaaaccctgtcccagtgcatcgagcggatccgtcaaaatctggccattgcacgggaggattacaagaactacgaaggtttctcgctggaggaaaaggtcaagtatgatctgcacctgtcgtacagcatcaattcgctgtactggatgtactataaaatcatcggattagatccgaataagcacggaatcaaagacgaactgacgcgcattaaagcggccatgatgcgggagaaggaaatctacgaccaccggttcaatcgccccacgctggaccagggagccgccaagcggttcgtccgggctggattgttcgaccacaagaaccggaataaaccgctggataaggcggacactccgccgaacaagaagatccgcttcgaagactagagggacgcaccgtatagaatgttaaaaacacgcttcctggggggtacaaatattctactttttttgtaaatgtaatttcctgtaatgtctatttataaataggctgttagctgtaatttgtcatgttttgcgccaaaaacggtatcctttccgcgttttacgcgacatttgctaagaattgacttatgaagagatctttatggtaaatatatataatcttaaaacatg

SEQ ID NO: 81 - DNA, 737 bp, Aedes aegypti
cctcgatcaagagtagaacatcgttttcagtgttcagcgtcgttttcagaagaaccatcatgaagtgcctaattgtgctctcgatcatgaccgtgggctgtgcccttgcccagaacgtcaacgatggtcgctactacccagaggtgtatgcctccaagttcgatgacggaaagtggcgcccagataacaacggccaatatcgtggaactccagacaagtttggtgctggttcgggttctggtagccgttttggtggtgcttcaagtgttcctgttgtcaataccttcgttgctccagtagcccctgctcctgctccagttgttgttgctaatcctggtgctggtctcggattcggctctggagcaggattcggatctggtttcggatcggctggcgccaagaagagcagcttcggcagcggttcaagcaatggtcaaaacgttggagtcaaggaagatacccgtgaactgaatgaagatggttcctacttctacaaggtcctgagcgacaacgacatcgaattctctgaaactggacgcgttgagaaccttggagccgatgcctcggccatgcgtgttaagggatactacgaattcgttggcgatgatggtgtccgttatcgcgttgactacatcgccgacgagaatggcttccagcccactggtgatcatctgccaaccccaccaccaatcccagaggaaatctacgaatctctgaaagcccaaggactcatctaa

SEQ ID NO: 82 - DNA, 282 bp, Aedes aegypti
atgttcgctaaactgtttgtccttgccctcgccatcgccgccgtggcttcccgcccaggatattcggccattaccagctatagctctcctgcagtggttagtgctgtcgccccagctgttgttgctccctacgccaccagaacctaccatggagtttcggccccacttacctactccagctacgttgccccaactgcctacgctgctccggtgtccgcttactcctacggtgctgctccagtggcctacagctacggagtgtccccatacaccgtctactag

SEQ ID NO: 83 - DNA, 632 bp, Aedes aegypti
cgataacggtagcacccaacagtcaaaatgttcaagttcctcctgatcgccgctctggtcccagccatcctggctcagaccccagtccgccagtgccccaacggagctggtctgccccagtcggtggacatcaacggatgcacgacctctccctgcccgatccagaatggagcccccatccaggccgtagcccgcggaatcaccagcccagtggccaccaacggaatgtcctcgtacatcgtgatccgtctggccggtctgcagatccccttcccgatgcccgatggactggaggatgcttgcgccgtcggaaccgccccaggaacttgcccagtttcggccggacagaccttcgactacaccctggaccatgagggccaggcgctggctctgaccggagtcaccgtccaggttgaggtcggtctgcgcgccgatgacaacagcgtgatcggatgtctgctgttcgatgctaccattattggttaaataaatgtgattaagttttttatttaattgattgtttgtttatacctttttcttcttcggtcttttgtcacagacttgagaatacttggttttaacttcttcagggagattgaaaacttcgcctatacatttaaaattaaataatt

SEQ ID NO: 84 - DNA, 1511 bp, Aedes aegypti
cgtcactcaaaacgtcaaaatcaccgctgagtagttctgttcgatcggttttctttgctttcaccaggcgatggataatcgcttttagctgttttcgaaaaaataaaatttccaccagcaaataatctatcaagtggtgcaaaatgcgagttctagttaaaattgcaatcctgattgcaatttccctttgcttgttccatcatgtccaaagccaggccaaggggaaaggctcgcagaccctgtcggaaaaggtgcagcaattgctggacatgaacgcgaaacgaccggttatgcgcttcaacgggaatcgtttccgggactttgtcaaatcggccccacggaattactcgatggtcgtaatgttcactgccatggccccggctcggcaatgtgttatctgtcggcatgcccatgatgagtacacgatagtggccaattcctaccggtactcgcagacctactcgaacaagctgttctttgcgatggtcgactttgacgagggttccgacgtgttccaaatgttgcgcctgaacacagccccggttttcattcacttcccggctaaaggcaagccaaaaccggccgataccatggacattcaacgggtgggcgtgtcggccgaggttatcggcaagtggatccaggagcgtaccgacatccaaataagaatcttccggccaccgaattactcggccaccgttgccattttgatgctgacggccttcgttggagggttcctctatctgcgccggaacaacctggactttttgtacaacaaacagatgtggggctttctggcagtcatcttctgcttcgccatggtctccggccaaatgtggaatcacatcagaagcccgccgttcgtccacaagggacagaatggaggaatcgcttacatccacgggtcatcgcagggacagttggtgatcgagacgtacattgtgatgttcttgaatgcaatgattgttgctggcatgattctgctgactgagtccggctggcaaagtgatccccgaaaagggaaaattgccgccatagtcggcttagtactggttgcagttttcttctcgttgatcctgtcgatcttccggtcgaaggctcaaggatatccgtacagcttcttgttcaaataacttggagtcgtttgagtgcaatgaatatcctacgaaaagtgttgacagcgaaacacattgcagtaaaagagtgcgagtgtagaatttgtgagcaccagtttggcacgaatgtgtaggaaaaccctacgtaatcgaatgcgttaagtactaattgtaacaaaccgaaatcaccaaggaaatcacattgatttacacattacctattatgctagtgagaactaatgaattagtgcaaaaaatctgttccgtcaataccatctggaaataattaactttattgtttgtttagtgtttcaaatttactgcggaatttatacttcggtattttacggaataaaacttgacggggtctcacgtataaggccttttaggggttac

SEQ ID NO: 85 - DNA, 612 bp, Aedes aegypti
atgaagtattttcttttgaccaccattatgctgggcatacttagttatgccacggctctctcctgctacaaatgcaccagcgtcgaggacggcacttgcgatggcaaacagctggtagaatgtgatgccgttagtgctgcatccggaatggccttactgctggcgttgaagccttcgattcaggttatctccagcaccaactaccagtgctacaaattggttgccgaacaaaaacagtccgacaatggagtgaccatcaaatcctgcatttatgattcgatagcggtttgcgaaggagctcctaccaatgctgaacaaaaggaatgctacacttgcgcagaagatgagtgcaatggatccgggcgatttggaatcagttttatgatctattacacctacacgtcgtttgtacatgacgttcgtagtggagttgccaacaattacggggcatacggtagttcattgttctacatgagttgtatggccaaccaattctttctgcgaggtttgttctgcgagcagctgacacggagggctcaacggactggtgttctactaaacgagttcatcgaatccgaaattgacgacgatgttaatcaatgcgtgagttaa

SEQ ID NO: 86 - DNA, 1344 bp, Aedes aegypti
aatcagttaaaagtataaattcgttactgctcggaaacaaactcaacagaagtatttcaatatagcaggaatcaagtaaacgtagtgattccgaaattaaattaatcagaatgaaaagttttgcactagctgtttttctccttctgggactcagtgctgtccttggaggacgtccgaaggcggaacaagtgcttgaagaagtagaaaagcttcgaccacgctatcaggagattcaagactacgtaattcataccatcagtgatgcccgattaagcagctcggcaaccctcgccaaattccactccgacattttgaaagtgaaatccacctttgtgatcgatgccatcaagaaggagaacaatatgctataccagatcaataatcaaccagcttcaatagacaaaaattgccttgcttttgttcaaacgttgggcgacagtattatgaacctggctggcaccagtttctctagctgcattgccgattccagtgacagtttggagaagaggattaacgagttttacgccaagctacaacaagatgaaaaggactacattggactcggtttgttgaacgttttcaagggtgagaacattttctacaccccagatgtgctcatcgccaaactgaaatccagatacgaagctttggagagctatccaacctacctgagcgaagatttggctcgtattatcaattcgttgaacgctgatttggagtccttggaagtgagctacgttgtctgtatgaccgaaagtgagcagcgacttcgcgatggtttcatcctggcacacagccacatggaattaacctgcaaagctgtttcggtaccagcaccatcatctccggaaacggtagaacccagcgtgcctgaatcgatgtcatggccaaacaccgaagccgtcagactgcttcgtgaacgttttggactgtaaactgaacgataattgatcgcgtgattatttcaataaagaaactaagttttcatataatactagaatcaaacgttgcttcgccataggatatttggtattgcaaataagaattgctctcatattaacagatattcatactttttagtcgtttccccatttatggtttatgacagaaacgttaaacgaattgccaatacacattgatatatcaaccatgcttttcggctacgcagtttttatttgaacaatggattattatttgcctgacgttttgacacctcatgggtaaatatagttgtcgtttgtagtcctatgttttgtttaactttaactatcaggtctgaatattttaggtacatgacttgatttacatagatagataaactaataaattcgatgctacaattga

SEQ ID NO: 87 - DNA, 438 bp, Aedes aegypti
actgaacgttttataatcaaagtcaactgttcacgcctgacacatcttgtatttgcagcagaaaccattgcgggtgcttttcggaacattttcgtgcaagaaaaccacatttaaaaaatgtcggaagtcacttctaagatcccgaagccagtgctgcgcggattgcacaatgcctccatcaagcgcaatctggtcgtgtctggtgtcctgtgcgttattagtgtagctgctttcaacttcctttacgtcgaaccgaagaagcaagcgtacgctgatttctacaagacctatgacgctaacaagcacttcgaacgtatgaaggatgctggtctgctgcaatccgtgaaggactaaagtagcgccagattggtctataaagatagtttattgccgcttcatgtttacaagtaataaataaaaagatcacttaacataaga

SEQ ID NO: 88 - DNA, 1624 bp, Aedes aegypti
caactgtagggtactgaatggatggaccaagcctccagcccagcacccgattcctatactaactagagagaattagcaccgcgtgcagaagctactacacatagctctggtcgtttccatcccagccagcttcggattctcctaccattgctataaatacgggtggtaacttcatccccgaacacacttctgaacggactttcactgtgtcaacgtcgtcgtgctagaaagattttgccgtagtggcgggttcgatccccaaaaaatttcggagattatcgcgaattgtgcccgtccttgaagggttgtgcaacgtttttagtttcgtttgaatcttatttttttcggtgtagccttctttcctctgtacgtaatatttgaatcaccatgaagccgttcgcattcggagtgctcatcgccgccaccctttgcgtggccaacgttttcgcggccgatgaagcgccaaaggataccgacgaagacacggtcaagatctacaagcggctcatcccggccgatgtgctcagagatttccccggaatgtgcttcgcctctactcgttgtgccaccatcgaacctggaaagtcctgggatctggctccattctgtggacgttcgacctgcgtagtttcggaaactaacccatctcaactgctggaactggttgaagattgcggaccactgccactggccaacgacaagtgcaagctggatacggacaagaccaacaagaccgccccgttcccgtactgctgcccgaagttcacctgcgagccaggcgtgaaactggaatacccagacgtcaagggcccggaagctgctccggaagacgacaagaagaactaagctgggacacccgaaattaaatcctgaaacgaccaacatgagaacaacaaaatagagtattgaacacccaacctcaagagcgtcacacttggttcggtattcttccgttttttttttattatttgtaccgataccggacgtagactattaaggctaatctcaacaaatggcgcaaaaaagtgtattatcgaacaaggaacacaccatatacctattagtcaagtatgtgatattagaaatatgtatttatcagaaaagtactattttaaaacacaatatttcgttaccaaactgttggcgcgaagattgcgcagtttgccgattttcgccgaatatggacaaattgattatactcaaacagtctttttctcgatgttgctaatacttaattttgttttgtcacatgaaaaagtacagattttataaataaagttctcatttttcactaaatctgtttttgaacctatttgatccgaaatacttgtccctgtacatcgtaatttctcttaaattgatataaagatccgattaaccatttagggtcatcccagccaactatgacaaacttctctcaccctttttatatgattattccaaatttttgcatgaagcgcaacacaatgaaaaacatccatcctatcttttattcacaatgggtaaatagagtccagaatgcaacttagatggaacagattttttttttgtattattttaataataatttcaatagaaaacaatgacagttttcctcactatctagccttatttgataaaatag

SEQ ID NO: 89 - DNA, 1011 bp, Aedes aegypti
tgtttaatcaacacgcactgaattaatttcgaatccgagtcctccggccagaggtgcgatcgaaggcgacaacgaagaagaagaactaggtgggggagtaaaagaaaagtgaagagtgaaaataaaaatgaaatcgatcacgtgtctagcggtgttgagtgccgccgccctgttcggagtggtgtcctgtgcgtcgattgcctcgaatctggtggttgatgccacgccaaggatcgccaccagcgagcagcttctgtcctcgattgtgaacgagtgcttcgaggatgattcggtgatgaactgcctcaagggcaaggtgttggtctacctggatggagtgctgggactacgcgaggaacaggctcgtgctttcgaggagcaaaatgtggacaaggtgatcttcgatcgtgtggcacgcatcctggccacccacgagttcaaggttcaactgccggaggataccgtcgtgagctaccgtgccgataagggtgtcaacctggatgttgtgcctgctggacttgccaccgtgctgctgttgctgaagctgaagatgaaggccctcatgccgatcttcgtcgccctgatcggcctcaaggccatgaaggctctgatcctgtccaagctggccatcactctggtgctcggatttatcgtcgcccagctggttaagaagtccggcctgggaatgcccatgatgtcgatgatgccaatgatgcccccagcagccgaatatggtgcccctgctccggccaccaccgagccaccatccagctatggtgccccatcctgggaatcgcaatccggaggaccttactcccgcgtctgggaaccatccacctcctcctcctcccataacctggcctacagctcgtactacccaagcggatccagcagctcctacagctccactgcctacaactctggatcgagcaccagctccagctcctcgccatcctcctcctcttcctcgtcttcctcctcctcaaactcggctcacgcttattaagcgta

SEQ ID NO: 90 - DNA, 1035 bp, Aedes aegypti
atgaagctctcagcatttctgataacaaccctagtcttagcctccgcggccatggccttccctcaacgcccaactggcgatgagtcgcgtctcgagcgtttggatcgcttcctgaacctactgccggaagacctccgcgcagagattgaggactcggttcgcgacgccgtggccaacggaaagcgtccgcaaatttcggacgaagtacgcgatcagatccgggagcacatgtccggcttgaagcaacagcatcagcacaagctgaaccgtttcctgtcgatgctgccggaggaacttcgccgggaggtggaagccgttttggagcagtccgctgccaatggagagttccctaaggcttccgaggagctgcggaacaaaatccgggatcacttcgctgcgatgaaggaagagcgaaagcatcgcttggataagttcatgagtcttttgtcggaagagttgcgagcggaggttgaggggttgctgcgagagcctttggttgatggagagcgtcctcgggcgtcggacgaacttcgcgagaagatccgtgaacatttccagaatctgaagggagctcatcgcgagcgtttggatcgattcctgtccatgcttccggaagatctccgccaggaagttgaagtggtgctgaagcaggctctggtcaacggtgagcgtccgcgggctcctgaagagctgcaggagaaaattcgggagcatttcggagagctgaaggaaactcaccgggaccagctggagctggtgctgaactacctgccgaaggagctgcgcgatcggattgagctggaaatcaagcaaaacagggtgggccacggaatgctgccaaggttttccggagagctgctccaggagattcgcgaatatttcgagcgattgctaccggcgggtctgaaggagcaaattttggccgaagttcagcgggctatcgaggcggatgtgtatccggttttgagtgaagatttacgcgaaaagattcgtcggttcatggagatgcttatgactccccaactgttccattaa

SEQ ID NO: 91 - DNA, 930 bp, Aedes aegypti
ctggagacgatcaagtgatcatcttctttagcgttattctgtgctttgtgatttcaactatcagtgagaccgtgaacttgaaagtaatcatgctaaaatttgtgttaatatctgccctagtggctggagcttcagcaggttatgccagcagtcagtatgcgggagccggttacaacgggggttttgcgtcgcagtacgccggtcccaatggcgtaggagcctctattggatctggagccttcgttcccggatttgcccccttccagccggtgccagcgttccccagcgcgtttgacttcaacagtttctaccagggactgcaatccaactttgccaatctgtattcgcaacagctagcccatcagaatgcactgttcaaccagattcgtcagcaacaggcctttgccgctaatgccggtgcaggagccggttcaggagtcggaggtggatttagttctggtggtacctacggtggtcccggatacgcaggagcttcggcttcctatggaccaggaggaactcaccagacggcccacatctatccgcaaaatcctcaatctccgaacgttgatgtcagcaatagattcggaggtggttcacaacagggaggaggaccaggattcgtcggtgtatcgagcttctcgtcatcttccaacatcaacgggcagtcacatcgtgaagcgtcaacgacggtgaatgacaacggaaaggtcactacttatcacgtgcgcaattaatgttcgccattctggcagaataaacgagaccaggaattgggatgactaacaatggaagagtattaagaatgctgcattttcttcaataaaagacaaaaacgtccaacgtacttacattttaagaatatacttgcccaacgagcaatatgtttcgaacgagactttttgacaataaacaaaattacaacaaaat

SEQ ID NO: 92 - DNA, 822 bp, Aedes aegypti
atcaccgatcgtatgaggagtacatgcaccaccagcagcagcaacaacaacaacagcagaatcaatccagccatcagaatcagtctcaaaacagtgcggccggttctccactaccgggtctacccggacagcactccagcaccggaggatcaatgctggcaccgcagagtcccagcatgctcggctccggcagtaacggcatgatgtcctccccgctcatgcagtcacactctccgctgctgtcgtccaatccctcgttgctgaactcacaatctccgctgctgtccaactcgccgttgatgagccaatacgctccctaccaggatgtgattcccaccatcttgccgcaggacgatcccggaggatggggaggctacgccctcgctccgggaccctatcagagtgagttctgtgcctccaactacggggtgctacaccagcggcagcagtacggaggtccgggcaaaatgggaccccacggggcgttgaaaagcgccaaagaggcacgcatcaggcggcccatgaacgcgttcatggtgtgggccaaggtggagcggaaaaagctcgccgacgaaaatccagatctccacaatgccgatctcagtaaaatgctaggtaagtcgcagtagcattacaaagatgtagtcaacacatataagtgtacagtgttagtacggtaactcaaaactttgttttaattatggttgacctatataaaaaagtgtaggagcaatgtagcattctccatagttttatattcttctatccatatgtgctttcataaaaaatccagaaaaaaaccaacctccaccaaccgaagct

SEQ ID NO: 93 - DNA, 701 bp, Aedes aegypti
atgcttaggctaagtgctgcgtgtgtggtgttggtgctcgtaggagtgtccctggctgaacctccggcatccggtggatatccagatcgtcggaatggtggtggcggtggcggcggcggtggtggctatcagcaggtcaacagtggccagcagacttccgagggtcaaaccgtggatcctcagttgctcgatatggtgaagatgctgttgctgcagcacgaaagtgaatccggaaacggtggaggtggtggtggtggtgctggaggtccatacccctcgtccaatggtggcggaagtggcggtccatatggttcttatggaccaccagctggaagcagctcgcagggtcgtgtgaccggaattgagttggaaaaccccagacagagcatgcaggttgccgaattctggcaaggtggtgatgaacaaagtgctccccggggccaatacggtgccccaggccaaggtgcccccagtggatcttatggcgccccaagtgattcgtatggtgctcccctaggtagaaagtaaattaattaatttatttgtgtgtagtgtgtaggtattgttaggtcagaaaaaaataaatttgtcaccgtgaagccttttgagagtttcgattacagtgcgttttcgccattgcagcggcaaatgggcctaaattgcgatcgggcgcgtgacggtgaatggacgttagacgggca

SEQ ID NO: 94 - DNA, 612 bp, Aedes aegypti
atgggtcttagtactaatcaagttgttatgctgatttgcctgatcgggggcgtcattggcgagatcactgtggattccagagccacgcttgcgttgaagaagagatcagaaatggtgggctactctctgcggtggtgtgacatattctaccaagaagatttcccgaggaacgtagactgtactggaaagaacgatttggtgtgggattacttcccatcctgttgtaacggagcctacaactgcaggaacggtggaatttggaatatctacctatgtgctccaggatacgtttatgatgcgtcaatggaaagctgcgaggagttttcggaagattcgtgtccttacgtcggtggaggaacaggaggagatgacatgactactgaggaggatattcctacgacaactgagtcccaagtagtcatcaactgcagaaccgtagtgaatggccgcattccacaccctactgattgtacgaaattcgtcgaatgcatcgacagagttcccaacgttcgtgattgtctggaaggatacgtttattacgcaccatttgctgtttgtctgcctggcaaaaaagaagcctgtacactgtatacacttgaagatctacagtaa

SEQ ID NO: 95 - DNA, 567 bp, Aedes aegypti
atgttcagattggtggtgttgtctgctgttcttgccgttgccgctgccgccccaggagccgctatcgtagcttcagccccattggcctatcagacgtatgccgcagtcccagctttagtggcccagaaggagatcgcttacgagaagagcatcatcgaggagccaaccgttgcccacgtgggaaacattgaaaagaagatcccaaccggatactcgcaccagagcttcacccagtaccacaacaagcaggtcgccgaacatgtgtatgccccagccgtgaagaagactgtcgtcgaaactcctgttgagaagaccacctaccacgctgcctcggtggccactccagccatcacctatgctgctgccccagttgtggccaagacccactacgttgaagctgccccagctctggcctatgctgctccaattgccaagacccactacgtccaggccgctccagctctggcctacggaaccgcttacgatctcccatacgccaaggctgcctactacagcgaatacccaaccgtgtacagtgctccggctgccgtctacgccaagtactaa

SEQ ID NO: 96 - DNA, 582 bp, Aedes aegypti
atgcagtctttcacatcgctaacctttttagtgatgctcttgattgggctggcttattgcgatgtgtctcacgtgttggcgccagttatagtggaggatgaagtcactactcttcctcccccgccaagaccttatgcatttggatatgtagctggacgtcgtcctggtcatgttgatcgtgctcacagtgaaatttctgatggaagtggcgttgttcaaggatcgttctcctatgtcgaccccaaaaatgaacttcgtacagtggagtacatagctgatgctcacggattttacccacgattgagtcacgaactgaaatctcccgaacaaaccgaagcagtacaacgtgcggcgaataaacattttgcgttgtatgcaaagatcgcggaagctcatgccaatccccacctgcaacctgtcgaaccatcgctccctagagatacagtggccgtatccaaggccaaagatcgacatttcacgctgtacgaaaagatcgcccatgagcatgcccaaattgcagcgcaacgcgaagcagaacgactggctttcgaggcaacttccgaagtcaatggattacactga

SEQ ID NO: 97 - DNA, 1831 bp, Aedes aegypti
cggttatcgcccgctagtccggttggatcatctctccgggataagtgtgcaaaattgcgaaatgaagattctactgaccctactggcagttgcagcctgcacagaggcaacctttagcctaggttcactgctgggcttattacaacctaggccagcgccagctccagtacatcctggatatgcttcatattatcgatatgccgcagctccagttgctgtacaaccgggatatgcttcttactatcgacaagctccagcaccagctcccgctcctgtgcattctggatatgcctcttactaccggcaagctccagcccccaggccggttcaaccggcttatgcctcctactaccgacacgctcctgcaccggctccagtagtccgtccagctcagccagcaccaatcatacgagtagttcagccaatcgtgtacatccctcgtccgtcgcctccggttaccacgtactacgaagagactccagtcataacgagcactttgaccgtgcgagaacccaaaaccgagttcagcatagtgccgtcgaactctttctcgtacacaatcaacgaacctgctaagccagctcgacaaaatgacttcagcattgttccgtctcaaagctattcaatcagctcgccaaaagcccatagcgttcaaagtggctacggcgtagatgtggtgaagtccgaaggtcactcatactcattgcctgcggagcacgtcaccatcgagcagaccttttccccggtttcggatgtttcggtagcccctggaggtaacagtttcaacatcgatgttcgcaaccgaaacggaggaccagctgctattcaatccttggcagatgagaccatccaacaacttcctgtagcagatgaagcgcccgcctttgcatatgaatcagatacactcgctcaaccgatcgttgtcgaacccgcggtagagacacattcggtagctgtggcagactccgaagctgctgcacacgccgaagcagctgcacgcgctgaagctgctgctaacgcacaggctgaagctgctgctaacgcacaggctgcagcaaatgcacaagccgctgcaaaagcacaggctgctgcgaatgcacaggccgctgcaaatgcgcaggctgctgcatatgcgcaggctgctgcaaatgcgcaggctgctgcaaatgcgcaggcctccgcaaaagcggaagcttctacaaaagcacaggctgctcctcagacgatagcagctgtcacgcttgaccacgagcacatcacccagcaaatcctgcaggaggccattccgaacataatccgccaagtggcgccccacgtcattcaacaggctcttcctctggtcgttgatcgagccgttccgcacattgttcaccaagtcggctcaactgtgtcccaccaacagcaagccgaacccgtgcagatagtgacgcccttcaagcagcaccagcaccaacagaatcaggacgagaaggtcgtcgtcatgtacgccacctctgtcccacaaggccagttcgacgaatccgagaacttggtcgtgcccattggcaatcagagacagcaatacactgcaggtcaaagttcagcatcagaacaagcgccaggaaacgcccaagccgaaccgaaggtagtctttgaaaagggccttgagctgggacaccagatccagcaagagcaccgtgatgtcggcaacgtgaaccagcagatcatcaacaacttccagcaggagaatgctgcttatcggaatgaagtcggcggtgctgcaggagcatttccaaacgaccatttccaacgacacaatttccatccgcagcggctgcatcaccatcagtag

SEQ ID NO: 98 - DNA, 1410 bp, Aedes aegypti
ttcctcacgattacaattacagtagttgtgattgtaaacacagctcaaactccgatcacatcgcagtcggcttggataacaacatacctatcagatgtgattataaaaaagtgccaacgtacaaggcctacttggatggcaatttttctcgactgattgttggtggcaaattagagataatttcgttgtaaaccttttgtcctattgaatatcaaacagttgctagtgatatgccactttttgatgatctctcgtttaaagatattttggcatcttctgccactatatcaacagtattgcagtttttgactggatctgtgatatgccacagatatataaggaaaaagtcaaccggagagacgtctgcttttccgtttgtttcgggcttcctgtcatgttctctgtggttgaagtacggattgttgtcggaagaacatacaataatttttgttaacaccatagggtcagccttgttcttcgcatacgtgataatttactttactttttctgtgaacaaacgcaccgtagttcggcaattcttggcagtctgttgctttatactggcgtgtagcgtatacacaaaatacgaaccaaactcggaaacagctctcgaagttattggtcttatttgctgcggcgttggagtgctattctttgcatcgcctctaacagtattagctcaagtgataagaactaagaacacggagagtttaccttttccgattattatttcttcctttttcgtatcccttcaatggtttatatatggaatggtgattgaagatagtttcattcagattccaaatcttttggggtgcattttatcgtcaatccaacttctgctttacgcaatatatccaaatcggaagctatattcggatggtggaccgtcgtaccaacctttacgctcagatgccaatattctttgaaaaggagagacaaagtttactttgtactggtgatatgcagtcaaaatggttgcttgaaaacataataattaagtattcgtttgacatttgtgatcagtatattttgtaaataaaacattatttaattgtattaataaaacgacgattcagcagaacaacttttattaggtaaatcaagagcttccatttggtgagtccgtcaacggaagaattttaaaatacttacataaagctttaaaaaatcctgaaatatctccacaagacgcaattttggtggatatttgtggaggaatcactggaaagtaattctgttgaatacataaaagggttaatattttgagaagaatactccaaaaaattctatgatagaattttcttagaaaattttcagagcaattgtcattgattcagaaacgatctcaacagtcataaactacaacagatttcaaagcttaagcgaagtatgcacttcaacagaaaa

SEQ ID NO: 99 - DNA, 369 bp, Aedes aegypti
atgcacaagatcatcgcagttcttgcctgtctggctgttatggccatggctatgccgtacaaccacccagaatcaaaggaggaacatcatcccgcacagtacaagttcgaatacggcgtgaaggatgagctgaccggcgatcacaagacccagtgggaacagcgggaaggcaagcacgtcaagggacaatacaccctggaagattctgatggtggtcatcgcctggtggagtacgaatccgatgcccatggaggaatcaacttccatgtctccaaggttggaggtggtggtggacacggtggacatgtcgctgctccagctccggctcctgttaagaaacctgccaacttcaagaaatctcactactga

SEQ ID NO: 100 - DNA, 1074 bp, Aedes aegypti
gattaaaaaaaatcacctatgaaccgtttaatcgtaattggggcattgctcctattgtgtggaggaatcagcgcagacgagtttcatcgtccgacaacgcagaggccaggaaattcgactacgcctgcgcctacgccatctcgtaatcgttgcaagggattgccaaacgacactttgtttccttctctaacagattgtgcatattttgtgacgtgccaaaatgggttggaagtagaactcgaatgcagaccggaaggaacgttgttcgattacgtgcgccaagtttgcgatcacccggaattggtcgaatgctacgaaagtaatcggtgctctgcagaggaagacggaaagattatcccatcggagacgtgctccaactttttcatttgtcgaaatgggaagaaaagtgaagaaataacatgcgttccagctggaacattgttcgattacaaacggggtgtttgtgatcatccttccaatgttgtatgctggggctcatccagccccaacttgtgtgtaggcaaaccggacggagctttggtgccttcaatcgaatgttcaaatttcttcgtatgcaagaatgaggaactcgaccaagaaattacttgcgttccggagggaacagttttcgactaccaacgagaagtgtgtgactttcctgagaacgccgtttgctggactccgggtagcggaacaactcccggaccagacgtcaccacagtggcacctacgagaccaccacacccagaagatattccgcaacatttttgccgtggtgttgcgattgaagcttttcctcatcccagagattgcaccaagttcgtcgtttgtatccttgggcagccaacggtaaagcaatgtcctccaaggcacattttctatccacagtttagagtttgtggtttaggaaatacggaaacttgtagaccagaccctcagtgggtcgaagaacacctccgacaacagggtcgtgtggagaagttgaatgatgaatgaagtgacaaatcgtgttttctgttgagatcaggtatcaaatgatatataagcaataaaagtataacagcattctaaaca

SEQ ID NO: 101 - DNA, 507 bp, Aedes aegypti
atggaagaggctgtacaactgtggaaagtcatgcttcctgaacgattgtctttggtttgcttccgagaggggtcggatgcaatcgtgggagtcaacatactaggagtggcttccaaatacgataaggatgaactcaagctcaaaagcgatatcttccgaacaatcatcgctacgattgagtacatctcccaccaggccaacgtgttcgaacgttacggagtggatcagtacctgaacgcgatgggcctgtcggtggacccagtctatcgggggcgtggcattgcaaccgaactgcttcgagcaagagttcccctttgtaagggaatgggtctgaagttgacctcgacatgtttcacggggcccggatcacaggctgctgcccggaaggctgggttcgaagaaaatttcaacatcacctacgcagaactggcaaaagttgatcctcgctttgtgtaccctggacttgatgagaagagctgtaaatatatgagtttgaagattgattaa

SEQ ID NO: 102 - DNA, 2148 bp, Aedes aegypti
ctacattagaagcgcctcatggtgtgttgagaatgaaaatcagcaagagagtgggtgtttaggaaagacatttttgcggatagattggattttcattagcaatgttcggggaggcactttgtttacagtataaatagtatcgagagagccagaacggatcagttctattgtagaatttgggtagttagtaatatatgcaaagcatacgtctaggttctactcgtctgtggggaatagcatgtggtagcagtggcagctaggatcagcaacataggagtacaatcagtctgaatccttccgggaacgtgatcgtgtgcattgaaagaagaactaaagtagtgaggacttatctggagcagctatggcgcggttagccattcttttggcattatgtgcaatgtgcagattaaattgggcacaaaatggctataattatgacagaccagattcaggattgggaatcgccagtaccagtcgtcctggtttccctgggactagtccagcgggatatccaacgccgtcaggattcggtggtgtggcagggtcaactactccatcttatcaatttggaagcaccggatacccttctggtagaccttccggtccaggttttggacaacctggaacagtaggtcccactgggagtgctggatatcctgctgcggggcaacctgattttatcggaactgggtcattccagggaacatcggttggaggtactggtttcggaaatgtaggcactggatctggtttcactggtgacaacgttggattcccttcaattggatcccgacctactagtggaggtgctggtcaacaaaggcctacaggcggtattggttcagtgcaacctagcccatctggaccagctggggattctgttggacggcctggtggttccagtaatgatggtcaatatgagggcggtgactattctgctattcctggcgagcctggtgtagactatcccatcttttctgaaatacccgaaacttcgtttgattgtaagcaacaaccttatccaggatattatgctgatgtcgaagctcaatgtcaagtgttccacatatgcgccttgaaccgtacatttgatttcctttgtccaaatggtacgatattcagtcaggaacacttcgtatgcgtttggtggaatcagttcgactgtgccagtgcaccaggattgtttgagaaaaacgcaaatctgtacgacactccacaaggtagccgtttgccatcagatggttatccaggaggtcaaccaggaaccgccccaggtgttggaagcccaactggtcccactgcaggtggagcaccatatcctggaagaacccagggaggtcgtccttcaggtggtcaaatcggtggactgccaggggcttctcccattggaattcctggtggatctcctgtgggaccaccaactggtgcgtctcttggaccacaaggaactgcgccaggatatccgactggtccggctggtggatatcctagcgctcgcccaacaggtgggttccaacaaggcggtcaaattccatccaatgtgccaacaggctatccggcggccggtgtcccaacaggccctgctttccctagcggaccaagtcaacagggctatccagcgcctggacaaccagaaggtcaattttcgccttcaccgcaactcccaacacgagaataccttcctccaaggcagggctgaagctgaacaatatattgtagaatgaacaggtacgggcaatttatttatattagtataatcattcatacctatgtacggagtagcttgcagaagctgttaacaattgtttatctcaagcattttagcaaatactcatgtgctattcaaaactatcaacttaaactgttaccattcctaagcgatccgatggttctcaatcaagtttctgcattgtttataacgggatgagacctattagtacatattgtgagtagtatgatacgacaggtgcattaggtagcttattgttaatttttaattacatttttacttgaaagccattgtgaatactcacccgcaatcgatattgcaataaaaacaaagcatgacaaaatcgaaacgtgttgcagtgttttattttcaaatattcaacttacacgccgcataggggtttaataaatgcattaaga

SEQ ID NO: 103 - DNA, 873 bp, Aedes aegypti
gtagcactgaaaacaaaaacagaaagttgcttgctttgcttgcgtccatcgttcgcagttaaattttaagtttgtacagaaaattgcagaagtttctaatggagtaattgtctgtatctaccagcccaatctatggaagcgaagctgaacagttatcgttcgcaaaaacgacgccaggcgatcctgaattccttcaaagaccgcctctacaacatggtatccttccagcaggtccgagtggacgaaaagtcgacccacgtcattgtcgaagcggatactgatccgcaaccaaagccaaaaacagcaatcactccggtccaccaaaccaaagtgcccaagagaccgcgaccggtatcggaatcggattcggttgtctcggtaggaagcaccgagtcctctctcgattcggaccaaacggcagctaatcattcccagcaaaccccaaaaccacccagatcgtatctgacctacgtgacctatctggtgtacttcctgttctgggtcacgctctacgcaatcgccatcgagctgcgattcggagtggttttcctgatgatctccgctctggtaggaatctatttcaacacccggacggagaaagaacccggggaaatcagcgcgtacagtgtcttcaacgagaactgcgaatccatcgatggcactttgaaggccgagcagttcgagcgggagattggaataaggcactgatcccggaggacctatttttttaaaccactggtagaataaatgaggtgagtgatttttctacgataaaaattttcacagcacataacgttttaagcagttagtttccttttgagatgttaaacgatctatttatgagaatttcttatagtaaattttttacagtat

SEQ ID NO: 104 - DNA, 2739 bp, Aedes aegypti
atgatatacccaacgctactcgcagtggtagcaatagccttcacaattccggccacttctgcgcagctggagaacttctgccacggagtcattgctggtatttttccacatccggatccgcggctgtgcttcatgtatgtgtcatgtgccttcgaggaagcgttcctgtatcaatgtaatgaaggattcgtcttcgacgaaagcatttcggagtgcgtttccggagaatgggaagattgtggacgggaaataaacccagagttggaactgatctgtgccaacgtttcatatggagtgtttgaatatgaagaggattgcggaaagtttatcttctgtcaacgtggaagggcatcgttgatcgagtgcttggagaatgaaatatggcttcaagagaagggttcttgtgtgttcggagatagagacacttgtcaaccgggagatgtgttttgtatgggaatgccagacggagtgattccccatccagaggggtgtggcaaatttgttgaatgtgttgatgagcgtagcactgtcatgaactgcgacagaggaatgatctttgaagagggtggcgtagactgtgtgatcggaagtttacagacatgtcagtcctcggaaggcatttgcagcgaccaacctaatacactacatccacatccggagttatgtgatctttttatgagatgtgatggtagtgatgcgatactcatgacctgcgggccaaatgaaatattccggccagacattcagttctgtgtgcccggtgatcaagacacttgtgtcccaagtagaccagaagaagcttgtgttggcagacctgatggaatcgtaccgcatccagatcggtgcaatcaatatattgcctgcaacggtggtagctcgtccgtgcacgattgtcctacagggcagattcttcgaccagaagtaccaatttgcgtggcaggtaattcagatacgtgcgagttaatggacggagtttgtactggacgtccagatagatacgtaatagagcacccaaactattgcggatggttcatttggtgtcaaaatggacaagcaagcgtctttcagtgtccggcaaacgaaatacttcgaccagatatgcagttttgcgtgcctggcactgaagaaacgtgtgaagctgctgagatagacgaaatgtgtgatggacgccatggggttatctatccccatcctgatcgatgtgatcagtacatacgttgtgaagaaggtaatctgaacataaactcctgccctccatacatggtcatagaacgaggaaccattcaatgcgttgccggtaatacggaaacctgtgagctttacgttgatctctgtctaggacgaccagatggtattattccgcacccatccagatgtcatttgtatattgaatgtcgatcggggcaggtagatttgaattcgtgccctgaaggactcatcttcgattcatcacattcacaatgcgtccccggtaatacagagacgtgcgatcacttggtagaatattgtatagatcgaccgaatggagtgattccgcatcccaatcgatgtgatttgttcatgatctgtaccaatggaatcacgtcggtacaccagtgtccatggggagaaattcttcgtcccgatatgcaattctgcgttcctggtaacagcgatacgtgccagtttacaccaattgacggaatgtgtaacaaccgcgaaggaactgtaatctacccacatccctacgattgttcacagtttgtgcgatgccaggaaggccaactcagtgttgaaaattgtcgtgaaggaacagtattgcagcccggaacaattcaatgtgtcgctggtaatagggatacttgcgaactgtatctggataggtgtataggagtcgagcaacaagttattccgcatccttccgaatgtcacctcttcatgagatgtgattctggtcaggcttcggtcgactcttgcactcgtggtatggtattcatcgatggagtatgcgtcattggtgatcgcgactcgtgcgaatcttggcaattggtctgtggagctattgcaaaccagataatatcacatccgaatttctgcgatttgtacatcgagtgtcgtgatggactaacgagcatgcgaccttgctcttcaggactaattctgcacccaaatatgcaagtctgcactccaggttttctagacacctgtgagtttattccagaggaggaaatgtgtgatggccgaacccaaggacgtttcccgattcccgatcaaacgcagtgcaatgagtttgtcacttgttcgaatggtgtgggaagcttggattcctgtcaggatggaacggttatgcgaccacgatttattgattgcgttcctggaaacgagctcacctgtgcagcatatccccacatttgcctcttccgaccgaacgagcacattcctcatccagttagatgtgatatgtttgtatcgtgtatctcggaaatgccacatgtcgtgccctgccaacgaggcgaaatatacaacgccgacagagacatgtgcgtccctggaagtgctgcgtcttgcgtatcattcgaacaagtctgtagtgaccaagaagatggattcataccgcatttgaactattgtgatttgttcatcgcctgtacgggaggagttgccaccgtggaagcgtgcccttgtggtgagattttcgtaccggaaatacagcagtgtctaccgggggatgctgaaacttgtgtgctgcagccggtttag

SEQ ID NO: 105 - DNA, 597 bp, Aedes aegypti
cgacttgccctgcccgacccaagaagttgcgctaaccgcgtgcggcacgccacgtaccgagatgcccgtggtgtagcccattcgtacttcttcagttgggagcacaacccaacccgcaacctggaagtggactggcttgatgcccgtaacatctgccgtcgccattgtatggacgccgtctcgatggaaacgccccaggagaacgaattcatcaagcaacgcattgcccgcggaaacgtccgctacatctggacctccggacgcaagtgcaactttgctggatgcgaccgccccgatctgcagccaccaaatgagaacggatggttctggtctggatcgggagtgaagatcggaccaaccacccagcgcaacaccggagactggagctacaccggaggatacggacagcaacaacccgataaccgtgaggcagctcagggtaacgacgaatcttgcctgtcgattctgaacaacttctacaacgacggtctgaagtggcacgatgttgcctgccaccatctgaagccatttgtctgcgaagattccgatgagctgctgaactttgtccgctcccgtaacccaggaatccgtctataa

SEQ ID NO: 106 - DNA, 1463 bp, Aedes aegypti
acagccatgtttggattccgacatgttttaccttaaataaaaatataaactgaacataattgagttgaaatgacaaaacatataacgtaaaagatgtaaaattatcaacatcaaaagtggaaacagacgtggtacaatttttgaggtttgtcagacttttttatacaagtcctgcaacaggatctaaagagcgtgggccgtaatcgagagtggagagtaccgaccatggaccgagttgaacgacgacatatcgtagcgagttgttgctatgccactgcactgtcgtaacaatctctacgtacggcaagatgtcttacccgtttgctgtcttcccggacgaggtcgactatttcgtggaaagtctgaggaaattccaggttaccgatatagggagtgagagctggtttgaacagaatgaaatcgttctgaagttaactcaacaagcgttcatagaggccagtaccaagcaggaagaggttattaaggaaaaacttctagtcgagggaaagcttccgctgctggtccacgaagcctactgtatcctagtttggaggatgaaagttctgccccaactgttggaatcgtctaatttgcaagcttcgtttgtgctctatagcgtcctttactatgaggtcaatgcgattgctctactggaaactctattatttcatcaaacctgttgcgaagctcttggcgacactgccttagatctcattgactattgcgcccaagctctgggacttttagtaggatatgctaacaacaagcacaatgacgtggaagacccgaaagaacttttacaagaacaaaccgtagatgaattcaaacgaatgaagaccgaaatggacttcaagatcggattgaagtgtgttagcattacgagtcatttgatgaataatctaagcgtacttccattgagtgccacgaatcgcaccattaaaacacacgatgtgccttgtttgatcgcggaaatcttgcattccaagccatggcttcgtagaacagccaagggcttcgaaaagttcctagaaggtcactggacgccagtttatggcgatgatatcctaaaagttaccaaggatgaagtccaagcgtggttttgtttatacgcactactgttcaatgaaaatgcaatgcgaaactatgaagcaacggatttcagaagaaaggaaataggaaggtgtgttggcttgttgaatgatcatatattggaccaagttccggttttggcccaactgaagcaatttttgtgcacttttcaaatgggcagtggacaaactggaaagcaaccggataaactattgttggatgaattgccggagataaaggaagccttactaacggcagctaaaaaagttggatggaatgcaataattcacaagcaccggcaaatttttatcgaactatcgccggacgaaataggcgttatggcaaaaaggtaaaataggggaaagtggggcagt

SEQ ID NO: 107 - DNA, 830 bp, Aedes aegypti
aaaagaacagtatattgaacgaaattacctacggacgacgtgtcagtatcgaaccgtttttcacggtgtacgcacaatcaattgaactatttcaaaatggcaaccaaatgggtttggacgaatgcccacgggccctacccgcccaacatggtctccggtggacaggatagtgacggtgcactgctctatgtcggtcgggcaaatcacgccggggatgttcttccggccaaggtgattccccagaaaaatgccgcctacatcgcctacggtggggaggaagtgctagtcgagaacttcgaggtgctctgccagaaggaactgatctgggacagcgcaacgggcggaaacattccaccggatgcggtcgtcggtggtaacaccgccgatggggaaccactgtacatcggtagagcttatcacgagggatctcaaaccattggcaaggtgcaacggtcacacggatgttgctacattccttacgggggagcggaagtatcggtaccgacctacgatgtgctgtgtgagcggtaaaatgaagtgcgtgttcgaatttgagaatgaataaattttaagggacttgttggaggtagattggttgatttataatttcttgtctaacggctggtagcgactgagtgttgcaaaagcgaacgaagtagtgaacgaatgcctgaagcttttcgtcgaggatttcaataaacaatttctctggattctctggaacctctagaattttggaaaaggattcactgtaagcagaaaagggaaaacatggcattagattatagaggccattttggtcaaaacagaaaatatagagaatatccatg

SEQ ID NO: 108 - DNA, 1739 bp, Aedes aegypti
ttccgtgtaggtagtcatcagtgatatcatcagagcttgatgatgacatgtttcaatcagtaataatcattgaatcgacgcgtcatcataacaaaacactgttttcatacttcctagctaaaaatatgtaattttagtagatttcttcaaatttaattttcttttgaatgcaccggtaaataatgatagaaattactgcccggttgatacgtgatcaaagtgcagttttcctgtgcggagaaaccgtggaatgtttgataacgttcaccaatccttcgctacccgagcatagaattagccagagtaacagtgatatcctggaaaatttggcatgggccaccgtacagatccattgttattgcaattctacagtccaggatcgtggaggctccggaacggaagcgtcgagtgccatgagcaagaatgtcatcggttccacatcgctgaatgctagcaaccagctaaaaggggaagtgctcaagtccacggatcccaagattctgttttgcgatctgcggttgtctccgggggaatccagacaatttatgttccgcgaaacattatccatgaatacccccccaacgtatcgtggagttatggtaaagtattactataaaataacggtggccacacaacgtgttgggaccactgtacaagctttgcacatcccgattcgagtgcttcccctgccaccgatcaattgtagcgacgagacgataaccttaaacgatgaatccaatgaggacctggcgccaaataatccttttatcgaaaagaaaaaagctccctcgaagatagaatacgcattgcactatcttcagaacatcaccgcgcggaggagacccaacttctatttgatttcgaataaaagaggtaaggtgggtcggttctgtttgtttaaaccaatctacaaactcggggaagacattgtcggaacgttggactttagctgtggaaccgtgaaatgtgcgcaattatcggttactttgcaatgcgaggaaatcatcaaaacgaaaactacaaaggcaccagagcagtccgccgctccgggtcaagagaaggacaatgcttccctctcggttgggcgcattacgaactatacgaagcaccacgaggtatgcctgggactgctgcaaacgcaaatgatattgcccattccgctgcacgttaccccgactttcgagacggacctggtggatgtgaggtggcgtctgcattttcagttcgttaccagcaccaacgacgagctgaacatggaagtcgacaaggaaactctggagtggcaagcgccaacggatatttccatcgaaactatgatatggaacctgccggttacgctgtatcccacggctccagtgcagattccgcaagccagcggaacatttacgttgaacattaaatagtttctgaagtaaaataatcgaattgtatgttacgttgatcttaataaattaaaaatattaacaatatacggagaaagatgttttgtatgtaatatgtattcttcgagaattttattcagttatgtagaagtctcgcctcggagagaggattagaagcagaatgtcaaaatgccatagccttacagtaaaagttacattggagtttaatttttttttattgtgaggtccaacagtgttaaggttcctagggtatttcaacatggaaattataggagaaacgcaagaaacctcactaaaaataaaaaaaggatgtagcattgatctttctgacggaat

SEQ ID NO: 109 - DNA, 788 bp, Aedes aegypti
cagtgacttcagcagcaacgcaccaaccaccaactccaaaccagcataatggcattcaagtttgtcgtcttccttgcttctctggccgttgccagcgctggatacctggaagccccagcagtgtcgtacgccgctccagtggcccactactcgccagcctcctcggtgagctacagcagcttgtcgcacgccactcctctggcctatgccgctccagttgccaagactgttgcctactcgagcccagccgttggagccacccatgagagcaccatccgtggacatgatgccaccatctcccatcaatccaaggccatcgataccgctttctccagtgtccgcaagtctgacacccgcatcaccaacgaagcccccaagctggcctatgcttcctacgccgcaccagctgtccacacctcctatgctgccccagctgttgccacctatgcccatgccgctccagctgtggccacttatgctgccccagttgccgtcgctaagcaagtgagctatgctgccccagcttaccagacctacgctcatgctgccccagctgtggccacctatgcccatgctgccccagttgttgccgcccacgccaccaagaccctgacctactcgccagctgtggaggtcgcccatgtcagctacgacggaactcatgcccattatggctggtaaattgcactgcctacggaaaccacaaatgatacaatgatcaatcgtcgttgtgtctatatttattaaaaccaaccgttgttgttatgttttgacagcacgctccggac

SEQ ID NO: 110 - DNA, 555 bp, Aedes aegypti
agtcaagcagttaagtcggtcgggttcagtttagttttgcggcattcgctgtgttgttttgttttcccacgaaggcgcgagattcctataatcaacaatggctaacgttctgtggtccatcatctggttgatcctgttgatcatcgtcggtttctgggtggccttcttctgcgccggttggtatgttctgatctacccactgactgtttgcataccggacgttgcggtggtatcggacttcctactgatgggggcccagtttgcgcattactgtgcgaaaaacatgatggagggcaaatcgttattctaagcgtaaatcatcatcgttctagcgtactatcaccgtcgtatagaatcgcgtttatattctattacatcttatatatttagagaagatttttcatatttgtatatcaatcgacaaaaccgcgtaacatcaagtgcaaatggaagtaccatcgattttattaaacaaaaccactacaaccgttcatcgaatatgtaaagctttaacaccatattttttttagaatacaaccactagttgtataaatg

SEQ ID NO: 111 - DNA, 786 bp, Aedes aegypti
gtgtagcacatcgaagagtgatgccttctggatcgccaccgccttgctgtgaatggttgactcctgtgggtcaacaccacatgtgtacataacaccgcaggagtatagaccgaacgaatcaccagttgtaccattcaacgaaatataatagccagatcaagtgcgacgacattagacattcgtacgcgataactcctccagtaagcatctctccaagtgttgtgtgaccgtgcaaagtattagctcagcgagaacttctccagtgtagcactctacagtacaaaaccgagttcagaatggccaaattgggactgttgttcatcgttatcttctgcatcatccagctgactcttgctgcccgcatccgccgtgatgcttttgatgaggctgtagattctgtcaagaagggaatctctgatactttcaccaaggaaaatgttgacaacttcctcggcaaactatcggaactgggagacgtgatcaagtcgaaggctaccgaaatcggatcgactctgcaggagaaggcccaggaagcgatgaagaaggaataattgagtagtgtagttaagtttaacaaccaccagtgtgctgtgcctcccgaataccaaccagggatttaaagaagaaaaaaaacgaaataaagttgacatcgattgatgaaaatgataccaatcaaataaacctattattacaagaaagaaaaaacatatgaattttgatacgtctttcccgaacgggaaatcttaaacataagcgaacaattagatttattagaaattattacta

SEQ ID NO: 112 - DNA, 490 bp, Aedes aegypti
ctctgctgaaatataaggatgaatgcattgccatgagtggatcagatgtgggttatcgtcaaaccttggtagcgattcccgaagttagagcctgcttattgaaccgtatcgatgtcttcgagatgaaaggggacgcggtgttgttatctgagtcgagcgaaaggagaaaggatttcttcggcaaatattgtccgaagttcaacgaatcggtagactgttttgatgacattttcgaaggaatcgcaaaatgcacgggtgaggaaactgagaagatagtgccagttttcaaggatgtggcctatggcgttgttgatctagtatgtgaaaatgatggacagtttgtgttcgaaacccagaaacccgagttcatggcttgtttaggaacgctcagggaaagcgttacggaatgtaagatttcgaacgtgaccaaatcaatttctttgatacattacggcgaagagcagtgtcgagatgtggaaaattcaagaga

SEQ ID NO: 113 - DNA, 1478 bp, Aedes aegypti
ctggaaatttttatcaaaaattagtgtttaaaaatcacctttttaatagaagtttgtaccttgtggtgtagaataatggcctcaaagccagtcctgagcacgaagactccaataacggagctgcaagagctgtgtgtgtccaaaaaagcgccgcatccgctgtacacatttaccggtgaggaaatcgacggaggaaacccgaattccaaagttttcaccacttcggttttggccctcggattcaccagcagcggaattggccggtcaaagaaggattccaaacatgacgctgcctataagttgttgaaattgctgttcgagaaagggctgagcgatattgacgtggacaatgatgagcactttttggcggtcctgtcctcggataaagtgaccgaagtgcgggacatttgcgtgcagcggaacttcgagatgcccgaattcaactgtgtccgcagttcgggaccttcgcatgctccagagtttgaatatgagtgccgcatcggagcgattgtccgccggggaattcacaagacgaagaaaggggccaaacaggcagcgtgcaacgagatgattaagacgctgcaagctatgccagtggaggacagcgaaatgcaggtccagccgctcaatctggccgccgagatcgatttgaacgaggatgagcacatcatacggacctaccgggaactcatcaactcggacatcaagaaaaagctgggcgtaaagattgcggaccgacatcggttcttcgaagaacaggaagaagcgaaaatttccgccgcccgacgaattgctctggacgaaacgctgaacgtggaggacaaatgcaccctgatccccaaggctctgggactgaaattcgaaatgaaacgagacaacagcgatctggtaacgatcgaaggaagaaaactgttcacattcgaattgcagaattcagaatatgattgtttcattttcggaaagggagacaagttttacgagagcgtctacaagtatcttaagaacatgcttaatttcgattacattgattgaatgcgatgtgataattaatgcttcaagcagatcgtttattcggcttactacactgaactggattagaaattacattgattgataacactttaattttatcgtccccttacgccatcggtgtaaggatcgattagttgataaaaatgtgaatacaaacaatatgaacgtcattgagttttattaatggttattactcttgctttttttcttcatccgatttctggtcctctggtttggtctcctctattgcgtcctctgcctttttgttgccgctgtcgatgctttccttcgaatcgctaccaggaaccgttacttcgctgctggtagaatcgtctgatttcttggattcttccgctttcgacttatcgacggaatcctttgccgattccgggtccttatcctttgatttagcatccttgcggcttgagcgatcgcgagagctgcaa

SEQ ID NO: 114 - DNA, 1992 bp, Aedes aegypti
atgcgtaggaatgccgacaaagaatttctggtgaagcagaaattcttcttcgaagtgttccgcaatatccatctgcctctcatgttcgaggagtacatgccatacaccaagacatgggttttcgatgaaactaagtacacgaactacaaggaagtcgtcgagttcttcgactactacaagcttggattcctgccgaagggtgagatgttcaccatctacaacaaggagtacatgaagcagacttatctgttgttcaactttttctacaatgctgccgactgggaaactttctacaagaacgttatttgggctcgcgagaacgtcaacgagggcatgttcatctatgcagtgacaatggcgaccttccaccgtccagacctcaagggcattgtattgccagccatctacgaaatctatccctactatttcttcaacaccgacatgatccacaaggcaatgtacaagacgatgtatgagcccaaattcggatttgctagcaacggtaaatacaacgtagtctactccaacttcaccgctgtgtatccgatggactatttcggcgaagataagctgagctactttgaagactttggtctgaacgcttactactactacttcatgctggactatccattcttcgtcggagacagcaaattcaacctgttcaaggatcgccgtggagagttgtacttgtacatgtaccagcagctcattgcccgttactatctggaacgtcaagccaatttcatgggaccgatcgaagagtttagctgggatcaaccaatcaagaccggatacacaccaaagatcagctactggaatggaatgcctttctatggacgcaacgactattactctctccccaaggatcaatactacaagattgacctgctcaaggactacgaagcaagaatcagacaagttatcgaccagggatacctgtatctagatgatggaaccaagattgacttccgccagcccgaatccattgactaccttggaaatctgattaacgccaacccagacagctacgatacagattactacaagtacattgaattcttcgctcgtctcatgtacagtggctctgactatttctacagcggatcgaaggtctggccaagcgctttgatgcacttcgagacatccatgcgtgatccattcttctatcagctgtactacagaattctgagctactactggcagttcaagagctacttgccatactacaccttcgatcaacttaacttcaagggattcgagatcaagaatgtcgttttcgacaaactgatgacctacttcgagtatttcgatgccgatatcagcaacgccattccatttggcttctcgtcggacaagttcttcgatttctctgtcttcgctcgccagaaacgcatcaaccacaagccattcacctacaccatggatgtcttctctgagttcgccggcaagggtgtcgttcgcatgtacatgggtcctaaattctacgacttcaagcagctgcaatacctgaagaaatacttcgtcgaagttgatcagtacgtgtatgactttgtggttggcaagaacaccgtcgtccgtaactcgcgtgattacttctggagcattcgtgatcgcacctcatacagtgatctgtataagaagatcatgatggcgtacaagggagaagacaagtttgtcctggatatgtcggaagctcactgcggattccccgatcgtctgctgctgccgaagggtcttccgagcggtttcgaaatgactttctacttcatcgtcaccccgtactacgctcccaaggtcgagcagttctccacctatgaatacacctacagctgcggagtcggttcgggctcgaagtatgtggatgacctgccgttcggattccccttcgatcgcgagatcaacttcgcctacttcttcaccaagaacatgtacttcaaggatgtgctggtctatcacatcgacgacatgaaactgaaccaatcgttctaa

SEQ ID NO: 115 - DNA, 1340 bp, Aedes aegypti
ctgggttcgtttcctccgtgggtaagtttgttttcattcaggacgacgaccgattgcccgacgatcggaagaaagcacggcgcgcaatccgtcgcgacattgctttggggtgttgcagcaattgaggaaatgtagagcaattgccgaaaacggccaactgccccaaaaactgcacgggggtggttgatataaagactatcgttccgctccgataggttatcagtagtggcttgtgtttcagcggtgcagactaagtgtccattgggttggcactgttttgaatatgttacttagactgactattttggcttgtctggcaattagcagtgccacagcagagcctcatctagaacatgccgctgcgtatggatacggagcacacctgttcggtgggatcaaccccgaactgtacacatctgccagagcgatggccccggtgcagtatgcctccagtgtgccaatggtgtacgcaaatcagctgatggcaagcccgctgcacagggccatcaaaccggcggtggtcagtgctccgctgcagtacgcagcctccctaccggtaagctacgctgcccctcaggcttccttagcttacagcagcccgtacgcctcccatccccatgctcatctggcttacggagtgcagactccgctaacggctagctacgcggctgctgcagctgcgccctcgacccaatttcacacacctacccaatacgcttcgtacgcagccgctcctgtacaatccggacacgcagcctttctgggatccgcctctgcagccacaccatcatcactcaattacgccgctgctctcgccaacgctcaagcttacggagcaagttctgcctacgcacagcttcagcgacagcatcagcagcagcaacagcaacagcatcatcaacagtcacactatccctttcaacaacaatttcatcaccagcagcagcagcaacagcaatcctatcagcaacagctggacgccgcttttgggcggtctgccgctgagctctacggtcgaggagtaccatttgcatattccggtgagcaagtggcgtccagcagtgcgggctcggagattgctcgccaaatggccgttcaactttctgcgggttaccaccaacaacaggcggcatcggcttacggggacgcacgcgcgcaagctcggacagatagtgagtaggatccgctggctggagaagtacctgcacgtggtgcacttccgtcggaggcactagacctgcgggcgaaccgtgggttcagctgcctgcctggtcagtcttagtcgtatgagtatgtgcgagaatgtgtgataccgtgtaattatttattttgctctaaaa

SEQ ID NO: 116 - DNA, 844 bp, Aedes aegypti
gtgacttcagtgtagaaatacataccccttttccagcttcagcataatggcattcaagttcgtcgtattcctggcttcgttggccgttgccagcgctggatacctagaagccccagtgtcgtatggtgcccccgtagcccactactctccagcttcgtctgtgagctacagcaccatctctcaggctgctccagctgtggccaagactgtggcctatgctgccccagccgtccacgctacctatgctcatgctgctccagttgccacctatgccgctccagttgctaagacctacgcctactccagccctgccgtaggatccacccacgagagcaccatccgcagccatgatgccaccatctcccaccagtccaaggccatcgacactgccttctccagcgtccgcaagtctgacacccgcatcaccaacgaagcccccaagctggcctatgcttcctacgccgccccagctgtccacacttcctatgctgccccagttgccgtcgctaagcaggtgagctatgctgccccagcttaccagacctacgcccatgccgccccagccatccagacctacgcccatgctgccccagcttaccagacctactcccatgctgccccagcttatcagacctacgcccatgccgccccagtcgtcaccaagcaggtcagctatgccgccccagctatccagacctacgcccatgctgccccagctgtggccacctatgcccatgctgctccagttgtttccgcccacgccaccaagaccctgacctactctccagctgtggaggttgcccatgtcagctacgacggaactcatgctcattatggctggtaa

SEQ ID NO: 117 - DNA, 657 bp, Aedes aegypti
atggatctgtattacatgccgatttctccgccatgttggtcgatcctgctactcggacgtcaacttgatcttaccttcaatttgaaggaaatcgattttaaagccgaagagcacaagaagccggaatttttgaagatcaatccagcacatacggttcccacactggcagtaggcgacggctacgctctcagcgaatcgcgtgccatcttggtctatctggtggagtcgttgaaaactgaaggccaggaaaactcgctgtaccctcgggatgccaagacccgtggactcatccacaaccggctggatttcgatctgggaacgctgtaccagcggatcatcgcctactgttcaccccagtggaagagcggctccatgggcacggaggaaaatcgcaccaaggttcaggatgcgttcgaattgttggaagttttcctgagtaagacgaagtacgtggctgccgatcagctgacgattgcggacatttcgctgttcgtcagtgtgagcttgctggatttgtgctactttgatcggtccgggtacgggaaggtcgccgcttggcacgacgttctcaagaaggagctcgttggctacgaagacgttatcgcgaagggattccccgagtggaggaagcacgtcacgaaacccgagacgaattag

SEQ ID NO: 118 - DNA, 1590 bp, Aedes aegypti
atgattttgctcttgctggtggttgcagtaggttatcttatctaccgctggagcgtggcaaccttcgactactttgaaaagttgaatgtaccgtttctcaaaccgtatcccttctttggggcactatggccgtccttaaaaggagaaaaatctccaacggatgccacggcggagggatatcggctgttcccgggaaatcgtttctcaggattcttctcattccgcgagccaggatatctgattcatgatccagagctgatcaagcaaattgccattcgggatttcgatcactttacggatcatgcaaataacgtgcctttggaagtagatccctttttgggaagaggactatttttcaccggtggacaacgatggaaacatggtcgaacggcactcagcccggcattcactggaagtaaaatgcggaatatgtttcaattgctgagcagttataccgatggcgcgatgaaacgcttggtgaaagatgcagctggaggaaaactggagagagaaatgaaggatttgtttcaaagcagattgggaaatgatgtgatgacatcgatttccttaggctttgacactgactccgttcatgatccggacaacgagttctttcagtatggaaagcgtttgtcaaggacctcgggcttacaaggactacggtttttcgtattgacgctcctaccggaaaatatattgaaagtaattgggatacggattattccttcggacattgcgaatttctataacgaagttgttataaaagttataaaagagcgcttggagaagaacattgttcgaccggatttcattcatcttatgttgcaagctcggaaaaatgagttgaaagctgataaaacagacgaatttctgaacgacgcaggcttctcaacagtcaaagaacatttacaatcctcagcgaaaaatcagatcgagtggtctgactacgatattgcagctacttcggcgtcgttcttttttggcggaattgagtctacaaccactttggtgtgtttcgccttatatgaaatagctttgaaccatgatgttcaacagaagctgagagctgaggtcgatgcgactaaactttcacttggggatgctaaactgacttacgaatcgatgcagcaaatgaaatacatggatatggtgatcactgaaacactaaggaaatggcctccatttggagtcaccaaccggcgttgcacgaaggcttatagtttagaaaacgccaacggcaccaaggtaacggtacataaaggccaagtaatattcatcccgatctacgaaatccaacgagatgcgcagtactacccgaacccagaaagatttgatccggagcggttttccgatcaaaatcggggcaacctcaaccaggacacataccttccctttggaattggaccacggaactgtatcgggtcgagattaactctgatgcaggccaagtgttatctgttttacatgttgacgtgctttgaaattcagttgtctacaaagaccgatgtgccgatgcagctggatgcgcgttcctctgcactcaacgccaaaaatgggttttggatgcaattgattccacgaggtgtataa

SEQ ID NO: 119 - DNA, 2372 bp, Aedes aegypti
tccgcaggtggctaccacgacaacatctcccaaacgcagtcgaactatctcgcgatccccccgtcaggtactaccgccattgagccgaccagtctcaataaccttaatgtcgtatccgccattcaatgcgatcaaactgattttaaatatttcattgaagaactgtgcccggtgtgcggtgacaaagtttcaggctaccactacggactgctgacgtgcgaatcgtgcaagggcttcttcaagcgtaccgtccagaacaagaaggtctacacctgcgtcgccgagcggcagtgccacatcgacaagacccagcgcaagcgctgcccgtactgtcgcttccagaagtgtctcgaggtcggcatgaagctagaagctgttcgcgccgatcggatgcgtggcgggaggaacaaattcgggcccatgtacaaacgcgatcgggcgcgaaagcttcaaatcatgcgacaacgacagttggccatccaggccttgcgggggtcgatcggtggcggcgtcggtatcgggcagctcgggtcggacggtgcctcggcgcaactgcagggcatcgactaccaccagccgtactccaacatgcacatcaagcaggaaatccaaataccccaggtgtcttcgttgacgtcctcgcccgatagctcacctagtccgatcgccatcgcgttaggtcaagtgaacccgcagacgacgatatcgctgagcagtggcgtcggaggtaatggcagcagtggaggcggaagcggtggaggtggcagcaacaacagttccggtgcgggcaccggaggttcaaactctacgtctacggctatccagaaccagctatcggagtccaaactatggatgagcgctaactcgacgaccgcctcgccacattcgctaagtcccaaagcgttcagcttcgacagtggaacgaatcccaccagtacggccgactcgggcaacccagcggacaccttaagggtatcgccgatgattcgcgacttcgtacaatcgatcgacgaccgggagtggcagacgtcgctgttctcgctgctgcagagccagtcctacaatcagtgtgaggtggatctcttcgagctgatgtgcaaagtattagaccaaaatctgttctcccaggtggactgggcgcggaacacgatattctttaaggatttgaaggtggacgaccagatgaagctgctgcagaacagctggtccgacatgctggtgctcgatcacatacaccagcggttacacaacgggctgccggacgaaaccaccctgcacaacgggcagaagtttgatctcttaggtttaggactgctgggcgtgccgagcctggccgagcacttcaacgagctgcagaacaagctgcaggagctgaagttcgacgttggcgactacatctgcatgaaattcctgttgctgctgaatcccgtagaagtacgggggatcacgaaccggaaaacggtggtcgaaggctatgagaacgtgcaagcagcgctgctggattacaccctcacctgttatccgtcggtgcctgataagtttagtaagttgctcagtattattccggaaatccacgcgatggccacccgtggcgaggagcacctgtacattaagcactgtgccggcagtgcgccttcccagacgctactgatggagatgctgcacgccaagcggaaatgaagccagcgccgtcatcgcgtcgttagagtgtaaaaaacaaaaatgggttaaaagaaaacaaaatgtgatcgccatgggcgtgggacgggacctgggagggaagagcaactacatattttttgggaagcgaagacaccaacccggaccatccaaaacccccggaagcagcgtagtagtgttagtagcagtgcagcagcggcagtagcaaggatgatgaacgtgttgtggcgaaggcgaccaagcgatccaagtggaaatgtgttcttttcttcgtttcttcagataattgtgtacttttattgtgagttgcatcgaaccgagcgaagcgaagcgattgttctttgcttttgttgttaaactacgtttgtaataatttaacacgttagctaagcgtccattttgagcgaaaggtgtgaatagtccccaaactgaagcgcatagttcaagaaaaaaatccgtaccataattcgtaaacacataacggagcagccagcagtcgtacagccaacgggtctgtatgcgattgaaacactttttaaaaagtgcaccactattaccgcacagttgacacaatgacgttaccatttcatctcagcgattaaaattttaattccttacattacacaactgtgaaacagacgaaccgcatgtaatagtcgcagcaaccgtgtggaagtacagaataaaaaaaa

SEQ ID NO: 120 - DNA, 1566 bp, Aedes aegypti
atggccgcacgctcaacggtagctttggcgctgctgcttttggtagccttctccgaaggagccaagattctcggtgtaatcccaacggcaggaaaatctcacttcaacattggttccggtttcatgaaggccttggccgaggctggacacgatgtcaccgtcatcagcccgtttgcgctcaagaatgctccgaagaactatcgcaccatcgaagtcccggaagtgctggcagagtcgaacttcgaccagaacatgttcaactttaagagctcgggaggcgccattgcacccttatttttgctgttcttgatgtatgaatcgtttgctaaggttatatgcaaatgtatactggataatgcaaatgtagtcaaattgctgaactcagatgaaaagttcgacgtagtgttcgtagagacgttcatcagtgaaccgctgtacggattcgcacagcactttgatgctccgctgattacatattcaacgttcggaaactccatgtggaccaacgatctggtaggaactccggcaccaccatctcacgtagctcacttcatgcttagctacacggacaagatgtcattctggcaacggttccacaatacggctatgaccatcgttgatcgattgtactacgagttgcggtatttacccaatcagaagcgattgtacgacgcagcgttccccaatgctaagatgagcttcgaccaacagatgaagaacgtatcgttggtgtttctgaaccaacacttcagcttgaatagcccgaggccttatccaccaaacatgattgaggccggtggagttcaaattgatgatgcgaaacctttgcctgaggatcttcaaaaatacctggacgaagccaaggatggcgtcatctacttctgcatgggatcaaccattaaatcgacacatttcccagaagagaagcgtaacgctttcttgaagacattctccaaactcaagcaacgcgtcctgtggaagttcgaagacgaaaatatgcccaaccaaccatccaatttgatgatcaaggcctggatgccccagaatgacattctcgctcatccaaacgttaagctgttcatcacacacggaggtctccttggtatgacggaagcactgtaccacggaaagcccatggtcggaattccgatcttcggagaccagccgatgaacatcgaaaaagctgtaagatctgggtttgccgtatttcttgactacgatgacatcaatgaggacaccgtcgataaagcaatcaatgaggttctgaataatccatcgtatgcaaaaaatgttaagcaagtgtccgatcgtttccgagataagcccatgactcctaaggaaacggccgtcttctggaccgagtacgtaattcgacaccgaggagctccccatttacgatcgacggcattggacctttcgttgttccagtatcaactgttggatgtttatgctgtaatgttggttatgttggtagcaatcgttgcaatcgacgtttatatcgcgaaaaaagtcttgaaaaaggtgcttcggaaaatgtgctctgctagtaagaagaagaaacaataa

SEQ ID NO: 121 - DNA, 1172 bp, Aedes aegypti
ccaggatgcagttaccggggatgtctatctttattctgatagccttagtgctagttggctcactaaaggctggacctatcgaaaaaggacttacctacgactcgtaccatccgaggctccaacaacctccattagacacgcttgcaaaaaatgagctggacatcaactactggttgaacaatgcccaaaatactgcacagaagttgttcgaccatgaggaaaacaaacgtgtcgctaagaacgtgatcatgttcctaggtgatggaatgtccatttcaacggtggcgatggcccgtgtttacgcaggaggcgaagagaagcaactagctttttcaaactttctgaacataggaatggcaaagacttactgcgtcaactatcaggttccggattcagcatgtacagcaactgcctacttagcgggagtgaaagccaactacgaaacgataggagtcaatgctcaggttccaagcaatgattgtacggctgaactggataaatcgactcacactcactcgattgccaagtgggcaatagatgccggaaaggacgctggactggtaacgacaacgcgagtaacacatgcgtcgcctgcaggactctatgcccatacttcgaaccggaacagggaaaacgatgctgaagttatgaaagacggatgtgatccagaaactgttgaagatattgcaaagcaactggtgcacggagaagtgggaaaacagctcaaagtaatcctggggggaggtcgaagagagttcttgcataatgacccggatcctgagacgggtgaatctgggaaacggaaggacaaccggaatctgattcaagagtggctcgatttggctgatgaaggtgagaatcgaacttacgtgtggaacaagcaagatctgttgagtgtaaacgtcgatacgaccgatcggttgctggggcttttcgacccaagtcattgtgtatacaatctagaccgggttcataaaactttgactcaagaacccacgttgaccgaaatggtcgacaaagctacggatgttttgagcaaaaatccaaagggcttctttttgttcgtcgaaggaggccgcatagatcatggccatcatgataactgggccaaaatcgtggtggagacattttcggagtggccggtatgggcaacgatcagcttccgtacatga

SEQ ID NO: 122 - DNA, 1616 bp, Aedes aegypti
ttcatcatgaaggcagtccaattgctggttctgagctgcttatggctcggatactctcaagctatcccacttgaaccacaaccccgcagttcggtggactactggaccaacgatgcgcagcaaaccatcgagagcatcctgcgccaacgggagaaccgaaacatcgccaaaaacgtgattctcttcctgggcgatggaatgtcagtttcgacggtggcgatggcccggatgtacgcagaagatgaagccacgaaactgtccttcgagcggtttcccttcattggtatgtccaaaacgtattgcgtgaacaagcaggttccggattcggcctgctcggcgacagcttacctgactggagtgaaaggtaactacgaaacgctcggagtcaacgggaagatccccagttatgactgtacggccgagctagacgagtcgacacacaccttttccatcgctcaatgggccatggatgccgggaaggacgccgggctggtaacgaccacgagagtgacccacgcatcgcctgcagctgtctacgcccatacttccaatcgggatcgggaaaatgataacgaagtgaaaaaggatggttgtgatcctaacgttacaaaggacattgccatgcagttggtgcacgggaagaccggtcagggattgaaggtcattctgggtggcggaaggcgtgagtttatgcacaaacatccggatccggagactggtagcaatggtaagaggaaggacagtaggaatttgattgaggagtggttgaatttggcttcgccgggtgagaacaggacgtacgtgtggaaacgtgatgatttgatgggagtagacccggcgaaaactgacaggttgttcggtatgttcgatcccagccattgcgtgtacaacttggaaagaattcaggagggtttggatcaggaaccaactcttgcggaaatggttgacaaagcgacggatatgttgcgcaagagtgataaggggttctttctgtttgtggaaggaggtcggatcgatcatgcccaccatcgaaactgggctcgtttgtcactggatgagaccgtggagttctccaaagcggttgaactagcaaggaacaagttcagtgaggaggacactctgattctagtcacttctgaccattcgcatgctgttacctattctggttattcggcacgaggcaaggacattctgggtgcccataacgatgcaaacgacgatcttccgtacaaaactgtgagctatgctaacggtttgggattctacgatcacgtcagcccctccgacggaggcagggtcaatgttgcagatttggatgcattgtggcgggagttccgttatccagcaactcttccgctagagtcggaaacacatggcggagaagatgtagctgtatacgcatccggtccttgggcgcacttgttcaagggaacctacgaacaaaacgcgattccacacatgctagcctatgcatcgtgtgtgggtgatggactgaaggcctgctcgtaggatggtgggatagacttgtttttatgtaggagttaagattgacaatctcgtttactattgactattaatttcaattaaatattcattgatataactttttttcatat

SEQ ID NO: 123 - DNA, 1114 bp, Aedes aegypti
gtacgcactagacgaaacggtggaattctcgatggcaattgagtttgctagagtcaagtacagcgaggaggataccttgatcgttgtgtcttcagatcattcccatagtgtcagctacggtggctatgcgaatcgtggtggagacattttcggagtggccggtatgggcaacgatcagcttccgtacatgactgttagctacgcaaatggattgggatactacgaccatgttgatgcggaaaagggtacgagaaggaacatcagtcatctagataccactaaagatgacttccggtttcctgcaactgttccgttaaagtcggagacccatgctggtgaagacgtagcagtctacgcttctggaccttgggcacatctgttcactggcacttacgagcagaacacgataccacacatgatggcatatgctgcttgtatcggcggtggattgaaggcttgtacgtgacgaggagcgtcggtgcaaaatgaactggtactggtgtgtagccgagtgtcccatttagattgataaaaatgaattatccgagtattgaattttcaatttttgtgcttcgatattgcaatacatagtctgcaagtgtatgagagtcatatctttagaacttaagaagctcacaaattctaaatttctgtattttatcaacttatgaacttgtgaatttatgaaactatgaactcctgaagctctcaacttccgaacttctgaacgtctgaacttcataatttctgaatttctaagcttttgagtttctaaactttgttcttctaacttttgaaatactgaacttctgaatttttaaacttttgttgttctgaaattttgttcttctatactatctattcttcttctgaatctctggacttctgaacctctgaatttctgtacttctgagctttcagcttcttaaattttgatcttctgaactttaaaacttctgatctttcgaatttttaaaattgaacgatttagacaatttaaacatttttgaaaattctatttcccaaacattgtgaacttgataattagttttaggttaattctaagtataaattaaaacaattcgaatgaatatatgaacccagatcgacaacc

SEQ ID NO: 124 - DNA, 832 bp, Aedes aegypti
ggaattcggtagaatcggttaattctactttacggctgtgcggtgatcagttaaatctgtttagttttcaaccagcagtaacgctagaagtggtacggatatagctgattgctacatgtgtgacgtaccacgacgaacttaacccggtaatgcaatatattcgagagaggctttccttgaacctttcgacaagtcaacagacggaagtaacagctggcttagcaaatggatctgctaatggaccggacaccacatatactcctccgaagccgccaactcgcaaagtccgagacggtcataaattgtcagaggtgatagtttctccaacgatctcaactatatcagaatccatcagtcagcatggcagcatggaaagtcgttccagaagtgtggacgcttcgggatccacttatgtgacaccttttcaaagccctgtggtatcggaaagatggtcaccgcacagtacagctgctgagaaatcccccacatcaattgataaggttcactttcaaagtcacaatcaatacgtcggctaccccagtagatactcaacgccaatagcaggtggctacgtgaaccactcgttcatcgatgctaccggaagtaatgctacagatttgaatcgtaccagtcatcaagcacagtaagtgaagcgtaccacctgttgggctgttgttgagctataaatctaatcagtctttctgtttctgtcaattagagttgcctttatcggtttatcaatcttcatcgtcaattgtcacatgcattttagcctatcagctgattttttttctcctcgagcgttatcgaagtaaatacagacatcaagtac

SEQ ID NO: 125 - DNA, 955 bp, Aedes aegypti
gttcgaaataataagcactcccaaagaagagtgatgaatctgtatgcaaggaaaatatttttgttcgaaatgagatactgatagtaatcgtaccgtgtatttgccatcgtggaggttttactctgccacgaccagaatagtaaggtaaactgaatcacgctgtagtgttgtcaagtttgaactaatgttacagtgattgttcctcaacaagagtagcgattagtagcatattgccataggaaagttaagtttattttacatcagtcgaaaacagtgcggtaagaaaaatggctgatgaagacaaaggaaaagtcgctggacatccacccgcagtcaaggctggcggtatgcgaattgtgcaacacaaaactcccagtgcagaacggccagctaaggatcccgtagaagtcatcggcttatcgaatccagcgcctaacgtgaacaccggcgaggtggtgcagagctctacgtccaacaagcacaacgagcactcggtcgaagctagtcaggtggctcatgcccagaaacccccggcgcagatcccccagaagccagtgaacaacatccagcagccacggaagttttgaacgagtgattacgaaagcatccacaaacgcaatacgtttgcaatcttttatttttcacatcgtacgtatattgatcttcttgtcggtaatgggaatagtaactgttgcgtttcacatttgctgctttatgaatcagatataaatacctacataaaaatccaacacatatatttaacacctgaagtatgttgttttctaagtttgtataaataaatttatataaataaagaacgttttatgtgagtcatatttagatttatgacgcaaataaaattatgtcattatattgtggtgttatactaaataaaaccaatcgaaagaaagtgaattgaaataaatgctattaatgataacacattttgtagacc

SEQ ID NO: 126 - DNA, 955 bp, Aedes aegypti; identical to SEQ ID NO: 125

SEQ ID NO: 127 - DNA, 955 bp, Aedes aegypti; identical to SEQ ID NO: 125

SEQ ID NO: 128 - DNA, 955 bp, Aedes aegypti; identical to SEQ ID NO: 125

SEQ ID NO: 129 - DNA, 1247 bp, Aedes aegypti
ggtggtattactaatatgtagctagtcatatctcttgtcgtccacgtcgcgtcggtattcggatcagaactcgccactcggactcagtgattcactcccccctttgtttagattgcaacaacaatggcatccaaggtaccacgagtgacgctcaacaatggcaaatcgattccgattctcggtctgggaacgtggggttctcctcccggtgaagtcgcacaggcagtaaaggatgccattgacgttggctaccgtcacatcgatggtgcccatgtgtaccaaaatgaacacgaggttggcgaaggtgttaacgctaagatcgctgaaggcgttgtcaaacgtgaggatctgttcattacgagcaagctgtggaacactttccatcgtccggatctggtcgaaggtgcctgtcgaacgacgctgaagaaccttggactggaatacgtcgacctgtatctgatccactggccgatggcctacaaggaggacggcgagctgtttcccgccgatgccgacggaaagacagcttattccgacgttgactatttggacacctggaaggaaatggaaaagttggtcgaattgggacttgccaagagtattggcatttccaacttcaactcgaaacaggtcgaacgggttttggctattgccaagatcaagccagtgacgaaccaggttgagtgccacccctatctggctcaaactaagctgtcagccttctgcgctagccgagatattgtcattaccgcgtacagtcccctcggttcacccaaccgtccatgggctaagccagaagatccccaactgatggaagacccgaaaattgttgccattgccaagaaatacaacaagacatcagctcaaatcttgattcggtatcagattcagcgaggccacgtagttatccccaagtccgtgaccaaatcacgcatccaatccaactttgaggtgtttgactttgagctaaccgaggacgatatgaagctgatcacgtccttcgactgcaatggacgtttggtgccgattaccagtgctgctggtcacccgtatcatcctttcgagaaggaagaattttagttgcgtccgcacacaaatgtcgctgaaatcatcacaccatactgtccttggaagaacaacccgtttggatggcttcgttgtgaagtatcacttgtactaccgatcgggattcccttttgttaattgtcgctttattataatggaatatactgatttttaacgatttc

SEQ ID NO: 130 - DNA, 1202 bp, Aedes aegypti
acgataccgacaattctcacctcatcccactcgtagagtaagcccaactgacttggaggcgagtgtcgtacactatgcaccgaatgacctacactcgtctgttgagagagtcgcatggagcaggaaaaccggtgttcatcgcggatctatctctgaagcttcaacccaaagcccggttcacgcaacgttccaccaacctaagtctaagtctagagcatccaaaatccccttgctggcatagtgttacttgaccgggtgttgagatagctatgaatatcacaacaacagtcgggtctgctggtgatggtacaaaacaatgttcctgtctgtgaagatttcggccaggatagtagctttgatcgctctagttttgctacttagtacaattgtgatagtccatagtggagcgaacaatcttcacatgtacgatgggattcgtacgcaggatatcatagctggtgacgtgttctttgaaattgtcgatccaccggagctagagtacacctatcgcataaggccagcgaaagactttgggacatcgttcgagtcggcttccttcggagttaaggaaggtaaactagttcctgcgattccatccgatgcctgtgagccagattttgtcaacgagaaggacctgaagggaaatatcgtcctggtggaacgtggagaatgctcattcctgaccaaagcaataaacgtcgagcaggtcggcggtcgtgcggtcatcatcacggaggtcgattccggctcggacgattatgactactatattgagatgatccacgacaaaaccgatcgagacaccaacataccggctgctttcctgctgggcaaaaacggactaatcattcggaaaacgcttgccaagctacatcttcgctacgcgcttatcaatttgccggtgaacttgactttcgttgcgccgcatgagatcaaccagccaccgtggttgcagtggtagtcagcgaaaagttgcagcttgtgttagaataacgaacaaatatttcaccattccgtgtgttagtgagatcaaagtcccagtgtgatactctcttcgaaaaaaactaaacaataaacggtattaatttatggatgttgttaaagagactaattttctagtcatgctgtgagaaacaaggaacgataagcaacaacaaaaacgacaattgcaataaacttactgtagaagctgaacacaaaaa

SEQ ID NO: 131 - DNA, 1032 bp, Aedes aegypti
gaagtgttaggagtaccgtgattgaaatcgggtaaacgagcagcactagcagtgctgcaaatcattcttttacgaaaatcgattgattcgtaaagcaatccgataaaatggccaacgtttgcgagccattaacactggccaaagatgttaagcgttcgattgagcttctggagaagctgcagatgagtggggaggtccctgccaccaaactggccgccctccagaaggtcttgcagagcgacttcctgaatgccgttcgggaagtgtacgaacatgtctacgaaaccgtcgacatacagggatcgctcgatgtgcgagcatctgccactgccaaagccaccatcgcggcgttcgctgccagcgagggacatgcccatccgagggtcgtggagctgcccaaaacagatgagggtctgggattcaacgtcatgggcggaaaggagcaaaactcgcccatctatatttcccgcatcattcccggaggcgttgccgaccgccatggtggattgaaacggggcgatcagctgctctcggtcaacggcgtatccgttgagggcgaaaaccacgagaaggccgtggagctgctgaaacaggccattggctccgtcaagctggtcgttcgctacacaccaaaggtgctggaggagatggagttgcgcttcgataagcaacgggctgccagaaggcgccaacagtactaaaagaaggacgttatcgcgcactctagttgccatacatcaataattatctcttactcgaattagaaagcggtattcagcagaaaccaagttcataccaagattgtcgcgattcgtcgctttcattcggaatttctcatattttcatcaataatggatatcgttttatgttttccattgaagctagatttaatctgacgctcgtttttgtttttttccattcatgtagaaatgatgcattatttacattttacgtttactttaatcgtgttaaactgtcggttcccactaatgattatccataaaataaaaagtatatgtcgcattgatgggt

SEQ ID NO: 132 - DNA, 599 bp, Aedes aegypti
gtttcccagttagcaaaataagaacaagcatagctctgaaccacacaaatattagcaaacatgaaagctatatttcttgccttggtgatcgccatcagcggtgtatcgtgtggtcctatctctattagtgataacaacgttggagacatcattacggttggcgttaacatagaagccgaaatcgagagtgaaattaacgctagcatcgtaaacatattggttgcattattggcaaacattgaccttggacctaaccctccaccgtatccaccaccaccgtatccaccaactggactgccaccaactggattcccaccaactgagttacctccaaccacactgccaccatccactgtcaccccttcaccgttctcaagtatccatattgatgaaatgatcaaaactatccaggatatcaaggaaaaggcttccggtgcaaatttgaccaaagagcagaaggaacagatctggcgggaaaatgcccaacagtttgtgcaaaaattgatggaaaataggcaataaatgtaagagctagcaataaatgtgacactaaataataataatagatatacggtgtgataaacgaaatacccattatt

SEQ ID NO: 133 - DNA, 282 bp, Aedes aegypti
atgaagttcgtctcggttgttctcctggttctgttgagcaccatcctactgtcatccagttcggcacaagattgcatcgtaatttcccgctgccaaccgggtaacgatggatatttgctcccgcattacgaagactgtaaccgatacttccgttgtgagggcggcctagcttgcgtccagaactgccctaccggattgcacttcaatgcgtaccacggggtctgcgaggatccactgacggcctgttgtgacatttatctaccctgtaatcccactcgatag

SEQ ID NO: 134 - DNA, 784 bp, Aedes aegypti
aactccaaataatatgattcgtttaagtcttctctttgtgcttctcggcgccgcagccgtcttcggaggcactctttcgccggagttccttgagtgggaaggccgtattgtcggtggttcaaatgctgcttccggacaattcccgtaccaggtttctctgcgctcatcggccaatgctcacttctgtggtgcttcgatcatcaacaaccgttgggttctgtcggccgctcactgcactgttggacgtaccctggccaatacccgagtcgttgtaggaactcatttgctgaacagtggtggcttctcccacgcctcgtccagaatcgtcaaccacggatcgtacaattccaacaccttggccaacgatgtttcgctggtgcagactgcttcagtcatcggcttcaacactcttgctcagccaattggtctgggatctgtcaacattgccactgcaaccggagccctggcttccggatggggtcgacttggggccaatgccggtatcccgaataacctgcagtggctgagaaccaacatcattaccctggcagattgccgtaatcgtcactcggttgccaacagggccatggtcttcgataacaccatctgtacactgagtccaactggccagggcatgtgcatgggagattccggtggtccattggtgcacagtaacctccagcagggaatcgtctcgtggggtattccatgcggcttgggagctcctgatgtgtttgctcgcgtttcttcgcaccgcaactggattctgaacaacgccaactaa

SEQ ID NO: 135 - DNA, 1241 bp, Aedes aegypti
tgcaacgagtactctcataagcgatcagatattcgagtaagtgagacgtagatttcagtggatccgtcagatacggattgaagaaattgaaaccagaagtgctagaagcgcgaagctctgaccctttctctacctgtatttcagcgctcatctgctgattttgtcgaatcaagaaggttcacaaaatcgtgcgtatccgaggattaccgccaattttccgagttttataactagtgtgtgtaattgtgatcttgcagtaacgagttaacatgaagttcatcctgggaaccgttgccttgctcgccggactctgtatggtccgcgctgacaataatagtaagttacgatttgtttttacaaaaatcaccaaatacatagccaaagtggctgcccttgctattcgagtgcccaggaaaccataattactgcgtcactcagttagggtaaaagtacacccagtgtgatagcttagttgtttactccattccatagattccaccgtaaacttcaccgcccagttttcctctatcgcccacatcggcaatgacctgcagacgttcaccagccagcagctggaaaaatcattcgacttcctgctgcttgcgttcaattttgaccaatacatgattgatcgtcccggatttgagaagctgtaccgcaagatctccgacaaggcctgggaggacacagaaaaattgatcaagtatcagagcaaacgtggcctcaccgtcgagctgaaagatctcaaaggcggagttattggccagctgaacgacggcaaagtcggcggcagtatctcgctgctcgacagcgacgaaatttcctcgctcaaagtcgccctcggctacgagaagatcctggctgaagagagccaccacatccacaagaagatttcccacgcccacgacaacaaggccacgtacgatccggatgtggcccacttcctggatgaggaaatcatcgagtaccagagtggcacgattcgcaagctgaccggctacatttacaacctggacagcatcatcaaggaggacaagaccaaggacttgggcatccacatgtttgacgaatacctggacaaggtggagtagggagcgcgttcgcgctgaagctacgctacgatgcgtagtgagttcagaaacattcttattgcttccttcccatatcaatcattgtgtattagtgcagtacaactatctattgtctgatgaataaaggtctgtagaaaagcggaaagttgc

SEQ ID NO: 136 - DNA, 1241 bp, Aedes aegypti; identical to SEQ ID NO: 135

SEQ ID NO: 137 - DNA, 1241 bp, Aedes aegypti; identical to SEQ ID NO: 135

SEQ ID NO: 138 - DNA, 2175 bp, Aedes aegypti
atgaagcttatcatagtagctgtagccatctccctggccgccttggccagtggatcatacgtccccagcgtcgaaaaggacatcaagtatgctgacaaggacttccttatgaagcaaaagttcttcttcgagatcctccgaaacattcacatgccgcttcagtacgaggaatacatgatgtacaccaagatgtgggtggaggattcgaccaaatacacgaactacgaccaagttgtggaattcttcaacatgtacaaaatggatctgttcctgggcaaaggagagattttcactatcaacaacaaatacctgctgaagcaaacctacatgttgttctcgttcctctacaactctttggactggaacacatactacaagaacgtcatctgggctcgtgagaacatcaacgaaggaatgttcatctatgccatcacgctgaccgtcatgcaccgcaccgacctgcagggattagttctcccagccatctatgaaatctacccgtactacttcttcaataccgacatcatccgtaccgccacctaccgtaagatgtacgaccccaagtttggattctacggcaacggcaagtacaatgttgtgtactccaacttcactatgacctatccatatgaggtgcccttcaaagatatgtacatgaaggacggcatgatgatggattactactacgaagatatcggtttgaactcctactatttctacttcatgatggactatccattcttcctgggtggcgacaaattcggtctactcaaggaacgcagtggtgaaatctatttgtacatgcatcaacagctgctggccagatacaacctggagcgattctcaaacatgatgtacccaaccaagatgctcacctggcagttcccattgaagaccggatacttctcgctgttgagctactggaacggaatcccattcaagtaccgcgacatgaactacatgatcaaggacgaagacttcttcaagctagactggattaaggactgggaaatgagaatgcgcaagatgattgatgatggattcttctacatggacgatggcaccaaagtcgatctgcgtaaatgggagaacattgatttcctcggtaaaatgatgaactgcaacatggataacttcttctgtcagaaattcggattcatcgaaatgttgtccaagatgctgctcagtggaactgatttcatgacaatgaagacttggccatcggttctgatgcactacgaaacgaccctgcgtgacccaatgttctacagtttgtgggaacgtctgatgagcttctattatttgttcaagagctacctgccttactacactttcgacgatctcagcttcaagggtgtcgtcatcaaggacgttgtgatcgacaaactgatgacatactttgagtacttcgattcggatatttccaacgtcattcccatgaccaacgttgacaaatactgggacttcagcgtgctgggccgaacaatacgcctgaaccacaagccattcacctacagtctggatgtgatgtctgagttcaccggcaagggtgtcatccgcgtcttcttgggaccgaagttcgacaaactgatggatctggagtattttagaaagttcttcgttgaaatcgaccactatatggttgacttggtcgtcggcaagaatacctttgttcgcaactcacgtgacttctactggagcgtcaaggaccgcactatgtacaccgagctgtacaagaagatgatgatgtcgattgccggcaaggacaagttcatcctggacatgtcggaggctcactgtggattcccggatcgcctgattctgccaaagggttgggtcagcggaatgaaaatgcagttctacttcatcatcacgccgtacaccatgaccgaaccgaagagcaacatagtcttcgacaacaccttcatgtgcggaaagtacaacttcgtcgacaacctaccgatgggcttccccttcgatcgcgagattgactacacctactggtacacgaagaacatgctgttcaaggatgtgttcatctaccacatggacgagatgaaaatgaacaaggtttattaggattatggagatgcgacccacgaggaatctctgatattttgggactgatttcgctttttagttttgttatgtgaatatatatatatgttgcaagtt

SEQ ID NO: 139 - DNA, 4539 bp, Aedes aegypti
atgaccttcgctagtgaacaggtgatcagttcgtaccgaaagaccaaaggacacacgattcagtggtggtgttgtgtgatgaagctgtggatagctctacttgcccttgtggcagcagtgcattccgtaccggtgaaaaacttcttctacggaaccggtaccgaagagtacgtctatcgttatgtgctagatgtgagatcgggcgttatagaagccaagaagggctcgcaatatgaagtgatggctgatgtgcatattcgaccagatcccactggaaatggtacgtttgtgtatctggttgatcccaaggtttacgtttatacgggaccactcatgatcaaaggtgatcttcctcaagtaaaaacggcagaaaaatcatttgtgccaattgccaaggaagcggaagttatcaaactacctttctacgttacatatgataagtatggaatggtgcacgaagtgttcactgttgctgaggatactctgtggagtatcaatattaaaaagggcattgcagctgcacttcagcttccggtcgacaaagttccagtgaagaaatatgtgaagcctgttgtgttttccaccgtggaagaaagtgtatatggttcggtaaatgtaacctacgacgtggaatcggtacatgatcacattgtggtaaccaaatctcctgattacacaaccttcgataacatgccatacgatgtctacagcaatgtagaggtagaaaaagtctacaagaattatgccatctggaacacaacatatgcagaagcgaaatacatggtggtgagcgacaaagatggtgttaaagtggaggaagtacacaccgatagcatcaaaacagtacaaccatcagtctccgaaggtcaagtgcaatatgttgctactaagcaatattttgtgttcaaatcgactgtggttccatcgactcctttggagttctccaaatttttggtctataatccgctatattttgtaccatccaaaaaatacttcgaatcatatttcccatgcttatcaattgttcctcatgtggtggatatgactgtggttggaccgaaagtgcaagctatgttatatgaagctgttgaatatctcaatgaaaacccgatcacaaacgtggaaaccgagaccaagcacggcctgactattaccaggatagtcgaagtattcaaaccattcaccgtcgaaaattttgaacagttctatactttgattaccaaaaccactactccgaaagacgttgttgcggttgacgtattctacaaaatattacccatcgttggaaccaaaaactcagtggtttttgtgaaaaatctcgtaaagagtcacaaattgaaggattttgtctctagcaccatgcttttctcgctgggactgaacgtacgagtgccgaccgtagagcttctaaaagaaatcgaagaattctaccattttgagaatgaagtgaaaccagaggtcgcccacgctgcagttctgacattcggttcgttggtttataaaacattcaagtacgatacgcactcgccagtgctgcaacagtatgtcaaagtattctacaaaaagttgaaggaagcaaagaccttcgaggagcagttggtatggttacatggtctgaaaaacatagaagtaggaactgtttacgagctacttgctcccattgtaaaaggcgaacaggtcttagatctgccctatgatcgtcatctgcgggttcatgctatatattcattggaaaaaatcctgcactacgactatgatgagctatttgaagtagtctaccccgtatttgtagatgaaactctgcctactgatctacgtgtagctgctttgaaagttttgctgtctacagaagaggttaaatactttgcaaaaattgctacagtcatgggaacagaaaagaatccacaagtatactctttcttcgtaactaccgttgaacacatggccgaatcctcagtacacaaggaaactgatttctggcaatacgtaaagcagacggagaaattcttcaagcacgttgatccttctgtggaaacaaaggcattctactacgattacgttgacaagaagcacttctttggagcatcgatttatggaaacctggtggctgatgacaaacacaataaggttaaccaatttttcatcaacttcagtccatacgtgatggatcgccatgtggatctgtactctgtgttcgtaaagttcgagggtgttgaaaatccattgaccatgctgtggcccaagctattcaatgttgaacccaaaacagtgaaggaacctatcattaagcaccatgaaaaggaaccagtgcatgtagagtttactctgatggccaacggaaaggttgtttacactacatattttgatgagcaaaccatcaaaatgttctacagccacacttgccttactattttaactacgatgaagtaccagtttaccagcgttttgaatcttgctgacgtggaagtgttctctcccacaatggatgggatacaaactaaaactaattttgaactgccattgatatctcagttcaaatacgatgtcattgtacctgtcgccaataatgtgaatgaaatgacgttctctgttcatagctacttccgtatatggatgcacggtttctacggaatttcggtttacaatccgtttgccatgacatggcaaggaacccgacgggtccatgcatttgacttcagtgtaccgttcaatttcgatgtagtcttcaatttccagcaaaactcatttaagtttgtgtggaccaagagcaccaatgatgttcaaaatgtagttggattcaagacccacgttaaaactcaggtatatgccagatctgattctgaagtggattacatgaagaccacctgcccggaatgcaagcatttcttaacgacagtcgtagaacctgtgtataagaaaaaagatgttgtactacacgagacccattcaaagtacactggcctgcacttttatctatccgtctatgacatcgaagaaatgcctcatttcaaatacttcaaaaccctttctgatatgttcgttagtgatgccttctatgaacttccggaaatgccgttcttcaagttcatgatgacctattacacatggttctacactcacttcttcccaccgcagtattcgacttatggagtcgtaggctacatcaacccatgtaaggtctacccattcgataagctcgaagttaccttcaaagtaaacacagaagatgttccgcataagttctggaccactcctaactatcatcaatacgtgaaagtttcagcagttttcaaaaatgaccatgacgttgaacttaaccactgggatatgaatgttcatttcactcacgaatacggggtctatttcaagaaattcaatgtacaagttacgaggaaaactcctggggaaaagaacatgaatgtatgttttgattatgtgaaggatgttgaaacagattcggtaaatggcaagatgacctactatcatggttactcatacgatcacaagtgtgtgaaggacgatatggtcgtttcggtcggatggactggaagcttttcagaaacacagaaaaatttcaccaagagccctaactattcatacaaagaatgcgtgaaacactacgttgagatgtacaaaccacccacacacaacctttcgccaattccaatgacataccattgcgctgaagcccacactactgttcgtaagtacgtgtacaacgtagactatcagcatgttatgccagtgtacttggaagtattcaaaccggtgtggttctgggtacagcaagtgttccctgtttactatgacgtttttgcgcaccatcacgtaccggaaagtagtttcgttgtcgacgtggaatatcctttgtggtgggtagaacacccaacagtggatgttaccatcaccacaccagtagacagtttcgtgttcgagcacgtcccatactacaattggttctggtggttccaacccgatagcgttgttttcagcagatactttgacgttatgaaaaccaccggctacacaaccgaatgcgtagtaaaccctgtgttcaactataccggttatcattacgacgtcagtaaaacgtcggtctacaatgaatggactctatatgccgcagatcatcctacgacctacaattgggctgtctacgttcagaaatcggaaatgagcttagttgctctgaagttcgtgctaaacggtcacgtcgttgtggtgcaacctgtcgttgacaagatgtacgagaagacgggagttatgaagtacacgttcaccgtagatagcgacattatggaagttatgaagcatggagttgtctcgggagtgtatccattcaagtacttcaaggtagacagcaccattgtttttgtgattccaaacgtatcggtttacgttgtctacgatggacaccaagtagtgatagaaactgtcaagagcgataaggtacagtactacggtcattgttatgtttgaaatacattccacttagattttgttgttcaattggtctatcaaatagaagccgaatagtgt

SEQ ID NO: 140 - DNA, 448 bp, Aedes aegypti
tgtttcaagaatggtcaaacaagcctccagtgtgccacagcgattagtataaatattctgtggcgataccaactagctatcagtttagttccagcgttcaatcagaacaacatcaacccagccctaatcaaaaatgttcaagctcattgccctagttgcctgcttggccgtcgtggtgtcggcttacgaagattaccacagccatccgaagtacaagtttgagtatggcgttaaggacccgcatactcacgatcataagagcgcctgggagcaccgagatggagatcacgtacatggacagtacactctagacgaagccgacggaacgcaccgaattgttgattacacctcggatcataaaactggtttccagccccatgtgcaacgcaagggacacgcccaccatccacatggcgagagttacgccaacatccatcagcactactag

SEQ ID NO: 141 - DNA, 1235 bp, Aedes aegypti
cgcgagggagtttgtatcgatgtttgtatactcgtgccagatagccgtttacaagctccaacagacctagatctttccttttctcacatgcacagccgttgcacagcgtggtgatctgtatatcgcagtaaagtgccatgtgtaccgcttacctacttcaaaccgcgtggtcaccgtcgttgttgtcgttccaacaaggaaggggcgtccaaatcaacaaaaagaaagagactcatcaatagcgatcactagtctggtaaaatcttttgcagcgttcagttcatccccaaaatccatcacgggagtaagagagaaagagaggtcccattaagagtacacttggcaatcatgcgtaaatcactcgtcctgattgctctgttggtctgcggagtctatgccgccaacgacgacgattactttgacttcacctgccctcgggaaaacggacagtacgaggatccttatcaatgtgacaaatactacgaatgtcaggatgggcgcccaacggaaaagctttgtcccgatggcttagtgtttgatcctaccagcaaactaccgagcaaatgcgatcaaccattcaacgtggattgccagcagcgtaaagaactgcaggaaccaaaaccgattggagcttgtccgcgacagaatggtttcttcgcccatccagactcatcgatctgcaatgtgttctacaactgcgtcaacggacgggaacttgagatgacatgcgtagccggtcttcacttcaacacagaatccggcacctgtgtctggccagatatggccaatcgtgttggctgtggaagcaatgccaacaaaaaactagcggacggattccagtgcccgaaagactatccgaaagcggataaaaacggacaaagcatcactcatcccaacttcccccacccagaagactgctccaagttttacatctgtctgaacggcgtcgaacccagacagggcacgtgtgatcccggtctggtgtacaacgaggacctccagcgatgtgacgaaccggagaacgttcccgcatgcgaggattggtacgttcaagccgaaaaagcgacggaaaactagatatggtttcttcaattttgcgaagactagtttacataagagataacaccgtagagtcataagaattataagttttataaactaaaacagtcattcgtaccttcgctttgaacagagatttatttacaaatatatgttaggaattttgttaatttaaa

SEQ ID NO: 142 - DNA, 1995 bp, Aedes aegypti
ctgacggcatgttagttatcgataaacgactgctcgcgaacgatggacaatgataataagatgaggccaaccggcgaagtggtggccgctcttcgaagcctgatgaagaatctcccgaacggtctgggatcgatcaacgcctacatcgttccttcggatgacgcccaccagagtgagtatttggccaagcgggatgaacgccgtgccttcatctctgggttcgatggcagcgcaggaacggctgtcgtcaccgacaaggaggcactgctgtggactgacggtcgttactaccagcaggccggtaagcagttggacgaacaatggactctcatgaaggacggtcaaccaaccacccctaccatcgacgcctaccttgccaaggtcctcgagccgggctccaaggtcggcgttgacgcaaatctcatctccacccgagcatggaatcctctgcatacctccctgaaatccgccggatgctcccttcttccaatcacgccaaaccttatcgatctggtctggaaagatcaaccggctgctccacagcaccctaccattccgctctcggtagaatacactggccaaaccgtcgcccagaaactaaccgccgtacgtgaaaagatgaccgacaagcgcgcttccgtcctggtggtcagcgctctggacgaaatcgcctggttcctgaacctccgaggatcggacatcgattacaacccggtgttcttctcctacgtgctggtcaccctagacgaactgtacttctttatcgacgagagcaaactctcgggagccatccacgaacacttccgctcgaacgaagttcaaccgacgattcggccctacggcgacgttcaccaagtcttgaagaccctcgccgagtctgcatcgcaccgcacctggatcagtctgggttccagctatgccctcaccgcgttgatccccgaagagaaacgacttcacgagatcactcccatcaacctaatgaaagcggtcaagaatgacacggaagcggagggcatgcgcgcctgtcatatccgcgacggagtggccttgtgccagtactttgcctggttggaacggtcgctcaaagagggcaaggcgatcgatgagattagcggagccgatcagctggagcagttccgtagtaaacaggacaagtacatgggcctgagcttcacaaccatcagcgcttcgggaccgaatggatcggtgatccactaccatccactgccggagactaatcgtccaatcaccgataaggagatgtacctttgcgattcaggggcgcagtttttggacggtacaacggacgtaacgagaaccatgcatttcggaactcccacggcggaggaagtgacggcatttacccatgttctgaagggtcaaattgcactgggaacggccattttcccgcggaaggtcaagggtcagttcctggacacgatcgcgcgcaaggccttgtgggatgctgggttggactacggtcacggcaccgggcatggaattgggcatttcctgaacgtgcacgagggtcccatgggcatcggcattcggctgatgcccgacgatcctggtctagaggagaatatgttcctctccaatgagcctggatactacaaggtgggcaagttcggcatccgtatcgaggacattgtccaggtggtttcgaccaacatcggggacaactttgacggacgcggagcgttgacgttccacacggtcacaatgtgtccgatccagacgaagctgatcgacgtcaagctgttgacggagaaggaacgcaccagcatcaaccggtaccacaagacggtgtgggaaacgctttcaccgctgctgaagtcggccggagatgccgaaacgttggcctggctggaacgggaaacgcaaccgatttgattggagcgcggagggaccgcaagggcgctcaacaagtgccgatgaaacagatatgtattgaactagatattttgatgaataaatttaaatcgataccgtaga

SEQ ID NO: 143 - DNA, 901 bp, Aedes aegypti
attttccaaccacaacgaacgagacgctgttagcaatcgggaacaatacaaacaattcggaaaccgtattttaccggtgaaaaactagtggaaaatagtcaactggaaaccaaaacgatgtcggcctccactggcaaggttgtgatcccggtggaatccccagcagaacaggaggagcatctgaggaacgcacggaagctgctgaaggacgtacagaacgtgccagccatgaacaaggagaacgttggaaagctgaacaagcttccgatgtatttgtcgcaggctcacaaggaactggccaagaaatcgctgttctcagatgcactggagtcaatcgagctcaaacggaagaagacgacaacggttcagtcccagtccagtcagacatcgccggagaagaaacccgccacggtaacgaagccaacaatcaccgctgaagacctcacatccctggagggacccagtgagcagtactgggaagtgttggccgaaagacgacgactggccctggaagaatctctggcggagaatcaggagctgtacgagaaggtcagctcgctggaagaggagctgaacctgtcgaaggagatgctccaggaggctcgcaatctggtggaggtgctgacggagcttctgcaggagggcgaagcggaaaaggaagaggcggctgctgccggtagtagtaaggagggggtcaagcaggtagacgacagtggcgtcgctccggagcccgaactacccagcgacgacgaagatctgtttccactgtttcgttgccaccggagcggttagtcgccgtgaataatctaacagaagtatatttgtattaattataactgtacgcgaaatgtggaaaagttataaaacaagaaataaatattaattaagcgacctctttttc

SEQ ID NO: 144 - DNA, 453 bp, Aedes aegypti
atggtcaagataatactgcttgtggctgctctctgcgcaacggctctgggcatcgacgtccacagctgtggagacgactatccccacccgacgcaggtcatcgttccgggctgcaatgtgatgccgtgtgaggtgccgaacctgtccgatttcagtttctcggttcggttcgctccgtccttcccgacaggcacgttggccgttgatgtgagcgctacgctgggagatttcaacttgccgtacgaaacgccggatcatctgcgggatggatgcgccaacattgatggaagctgcccattagcagctggccaggaggtgaccctgtcgggaacggctccggtggaagctccactgacgcgtgtgaccgtccgactgcggttcgagatcaccggagatggaggccagaaggcggtttgctttgcggctgacatggtcatggcagtgtatccgtaa

SEQ ID NO: 145 - DNA, 536 bp, Aedes aegypti
atgatcaggctaagcgtagttttcgttttgattcccgcggttgttttggccctgaatccagcgccatgcgttttcaactccggtcccttacctgcgcaggttcgggtggtaggatgtccggcgagcgtcccacagcagccgtgcatcgtcccaatcgggtctatggttgacagcgaaattgacatcgttctggagcgtccgacgactacgctgacggcctccttggatatgttcctcggaaatttccgcatcccgtggagtcttccggagcaccaacaaaacgcctgctctgatctgatgcacggagtgagctgtcctctgcaagcgggccaagctgtcagctacaatctggtgacggaagtgtccgccccctttgccgatgttaccgtcgatatggaactgatgctaaccgatgatcagggagcggccgtattctgctaccgttcgcaggctaccgtcgtgcgagcttagacggacaagttttaccgaactgccattaacctttcatacggaactccggccacggccagcaagca

SEQ ID NO: 146 - DNA, 453 bp, Aedes aegypti
atgttgaaatctgcgattttcgccgttctgttgccagcagctgttttcggcttggctgttcgaccgtgccctaacaatgctccggtcccgctggaggttcgcgtcatcgattgctacgaggaaccgtgtgtgattccgatcggtggaatggtggacatggatctggacttcatcccgaaccgtgacagctcaacggtcgacgccgccctcaatatcttcctcggaaacttccgcgtcccgtacgacttgccggaggatcagcgcaacgcttgcaacttcttcgccggacgtggctgcccggtcagccaaggcgaacagatcaactaccatctgagcaccccggctacggccccgtttgccgatgtcaccgtcgacctggaactacagctgaccggagacgacggtcaaccgctgttctgcttccgttcgtccgcaagaatcacagccgcctag

SEQ ID NO: 147 - DNA, 4164 bp, Aedes aegypti
atggaagtcattttcaccataaatgggaagaccttctcagtcgatcccaagactgtacctgttgatacgtcgctgaacaccttcatccggaatcatgcccatctcagtggcacgaagtttatgtgcttggaaggaggatgtggtgcctgtatcgtcaacgttaacggaatccatccagtcaccaaggagaagtcttcgtgggctgtgaactcgtgcttgttcccggtgttttcctgccatggactggacatcgttaccgtggaaggtattggagacaagcatgatggctaccacgccacccaaaaggtgttggcacacttcaatgggacccaatgcggatattgttcgccgggaatggtcatgaacatgtacagtctgttggaatccaacaagggtcaggtttcgatggccgaagttgaaaacgcctttggaggtaacatgtgccgttgtaccggttatcgacccattctcgatgcattcaaatcactggcctatgatgcggaacctagactgaaagagatctgtatggacatcgaggatctgagcaagatgtgtccaaaaactggatctccttgttcaggaaaatgttctgctgcgggaaaagtgtccgacagaaagggtgtacatatgactttcgctgaagacaaagaatggcacaaagtgtacaacgtaagtgatgtcttcgctatttttgaaaagattggatcaaaaccgtacatgctagtcgctggaaatactgctcatggagtctaccgtcgatgtgacaaacttcaggtattcgtcgatgttagctccatcgaggagctacgatcaaattctcttggtaacaacttgataattggagctaacgtgtcactgacggaactgatgagcatcctgaccaacgcagcgagtaaaaattcgagctttggatactgcaatgagcttgtcaaacacattgatttgattgccaacgttccagtgcgtaataccggaacgattgctggaaatttgagcatcaagaaccagcacaaagagtttccctctgatttgtaccttattctggaagctgttggagctactttaactattatggaatacggcggaaagactagcgttgttacaccttcgcaattcgtcaatctggatatgaagaaaaagctagttttgaacatcattgttcctcagttggatcccaagttctatgttttccgttccttcaagatcatgccacgagcccaaaatgcacatgcttacgttaacgccgctttcttgctgaaattcaatgagaacaaaaccaaagtggaagctgcatccatttgctttggtggtatcaatcccagcttcacccacgccacttcaaccgaaaactaccttgttggaaaaaatctgtttgagaacgcggtcgtccaagaggctctgaaaacgctctcgaacgagcttcaaccagattgggttctaccggatgcatctccagagtatcgcaaaaatctagcaatatctctgttctacaagtttattctgaatatcgccactgatggaaccgaaactcctatcaaaccatcgttcaaatctggtggaacggttcttgaacgcccagtttctactgcttcacaacgtttcgatacgattaaggaaaactatcctctgacgaagaacattcccaagatagaaggccttgcgcaaacatctggcgaagctaaatacgccaacgacttaccggcacttcctaatgagttatacgcggcatatgtgctgggaaccgaaccacatgcccaaattctgaatattgatgcttcggaagctctgaaaattcctggcgtggtggcattcttttcagcgaaagacattcctggcgttaacaacttcatgtatttcgcaggattcatgggcgacgaagtagaaccgattttcagtgctggtaaagtcgaataccatgggcaaccgatcggtatggtcgtcgcggaaacctttgctcttgtgaatcgagctgttaaagccgtcaaagttacgtacagcaagccagaaaagaaggatatctaccctacagttcaggatgtactgaaagcgaaggccaatgagcgcatcaaagaaatgggctacagcactcacggggacaactacgacaaagcttccgaaggggatctgaaagtcaagggacattttgaaatcggcggacagtatcactactatatggaaacacaaacgtgcgtctgtatccccatcgaggacggcatggacgtgtactcttcaacgcaatgggttgacttgacccagatggcaattgctagaatgttgaaagtacctcagaatagtcttaatttgtacgttcgtagacttggtggtggttatggtggtaagggaactcgtgcgaccatggttgcttgcgcttgtgcccttgcggctcatttgaccaaaagaccggttaggttggttatgaccttggaggctaacatggaggccattggtaaacgttatccagttgtctctgactacgaggtagacgttgacaagaatggaaagattgtaaagttgtacaacgaatacgtgcatgattttggatccacattcaacgaatcaatgggccatgcaggagaatttttcagtaattgctatgacaaaagcgtcttcaagacagtcgccaaaggtgttcgtacggactgtgccagcaatacatggtgtcgtgctccaggtacaactgaaggaattgctatgattgaaaccatcatggaacatatcgcttttgctactggaaaggatcccctggaggttcgccttgctaacatgccggaaggtatcaaaatgattgaattgatgccagaattccgtgcggatgttgagtacgataccagaatgaagcaaattgaacagttcaacgaagagaatcgttggcgaaagcgtggaatagccatcgttccaatgagatatcctttgggctactttggatcactgtcagccattgtcaccatttatcatgacgatggaacagttgctgtctcccacggtggtatcgaaatgggacaaggcatgaacactaaagttgcccaagttgttgctcatattcttaacattcctatggacaaagttatcgtcaagcccacaaacaaccttactgcacccaatgcgatttgcactggtggtagtatgactactgaaacggtatcttatgctgctaaacgtgcttgcgaaatcattttggagcgaatgaagccagttcgcgaagaaaataaggacgattcctgggaaacattggttgagaagtgccaccataagagcgttgacctttccgcaacatacatgtacaaagcttcggatttaactccatacattatctggggattgagctgctcggaagtcgaggtggacattcttaccggaaatgtgcagctgcgccgtgtggatattctcgaagatgtcggtgaaagcttgagtccaggaattgacgtgggacaaatcgaaggagcgttcataatgggactggggtactatctaaccgaggcactggtatttgatccagaaaatggagctcttctgacaaaccgtacctggacctacaaacctccgggcgctaaagacattcctgttgatttccgagttcgtttcctgcagaacagtacgaatgctactggagttttgcgttcgaaagcaaccggagagccggcaatgaacatgaccatttcaataatttttgcattgagacgtgctttgttggctgcccgtaaggatgccggattgccatccgagtgggtctcggttggcgcaccatcgacgccagatcaaatctataagttagctggcaactcggttgaacatttcactctaaattaaattggattgagataacatttataattttacaggaatgaacatagtattatgagatgggtaattaaatataaaatattttatatcttgatcatatacccacatttatgtaccaacagtatatcacatatattatgtataaataaataataaactaaaaataactactatttttgagcatttctggtgaaattccttataattattaactatgcataacataatagtaatttatgctattagttgaattattgtacgccaaaattattgtattttgagagactggtgttaaaataactataatcctgcaattgatcaaactgtcctgttggaaataggaacagtaaaacaaaagtcatc

SEQ ID NO: 148 - DNA, 1308 bp, Aedes aegypti
gcacgttgagctcgcgagttgcttttatagagcaacgttaacagatattgaatcgtatgtttagtttatttgctttgactttcgcagtgagaggtcgatctttggtgttcggtttttgaaaatttcaggatcatggctaaggacaatttaaccgccgttctttacggtattgaggacttgagattggagcaacgtcccatcccaacgccgaaggatgatgaggttcttttggatatggactgcgttggaatctgcgggtcagatgtacactatctggtcagaggacgatgcggtgacttcgtggttcagaaaccgatggtgataggccacgaagcatcaggtgtcgtgtcgaaggtgggttcaaatgtgaaacacctaaaggccggtgatcgagtggcgattgagcctggctacggttgccgagtttgcgacttttgtaagggcggtcgctataacctgtgtgcggacatgatcttttgtgcaacgccgccctacgatggtaacctggcccggcactatactcatccggctgatttctgctacaaattaccgccgcatgtgaccatggaggaaggggccctgcttgagcctctgtctgttggagtccatgcgtgtcgcagagctggcgttggattaggatcggaagtgttgattctcggtgccggtccgattggattggtgacgctcatcacggcgaaatctatgggagcaggtaaggtcgtggtgacggatctgctgcagaatcgtttggacgtggccaaagaattgggagctgatggaaccctggtggttgaggtgggagccaacgaaatggaagtggtgaagaaggttcatgatctgttcggaggcgaacctgataaaactatcgactgcagtggagccgaggccacttcgcggctgtcggttttggccacccggtccggaggttgtgccgttttggttggcatgggagcctcggaagtcaaactccctctggcgaacgcactggcacgtgaagtcgacattcgtggagtattccgctactgcaatgactatccagcggcgttgagtctggtggcgagtggaaaaatcgacgttaagcgcctgataacacaccacttcaacatcgaggaaacggcagaagctttcaatacctctcgacatgggcttggtggcgccatcaaggttatgattcacgtgcagcccaaggacaaaaacaatccaaagtgaatcttccgtgttcagtaatgaagtttgttagtgtaggtgtagagtaagatttgattattcataaaacttatcaataaaacttgaaattttgtgtactgt

SEQ ID NO: 149 - DNA, 2216 bp, Aedes aegypti
atgaaggcgatcacattggtagcgttggcgggccttgtagctctggctgcaggttacgtcgtggttccagaagacaagattacctacgcggacaaggatttcctggtgaagcagaaacagctgttggaagttttccagcatgtccaccagcacgaagtccacaccgagctgtgggaggtgtcgaaggagtacaaaatcgaagaaaactacgatcactacactaacgtcgaggcagtgaaggagttcgtcaagttctacaaatatggtatgctgccgttcgatgagatcttcagtgttttccactacacgcatcgcgagcaggccatcgcattgttccacctgttctactatgccaaggattgggaaactttctacaaatccgtcgtctgggctcgtttccacgtgaatgaaggcatgttcgtttatgctgtaacggtcgccatgctgcaccgcaaggatttggccggcctggaacttccagctccctatgaaatctacccctactatttcttcaacaccgaagtggttcagaaggcctcccagtacaagatgcaaggattctatggcatgaagaaggtcgatgatgtctacactgctgtcatcccaaccaactacacgggctggtatgtgcacaccaacgtcgaccagaaggtctcctacttcacagaagatattggcctgaacacgtactactactacttccacgctgactacccattctggatgggcggcaaggaattcggcctgtacaaggaccgtcgtggcgaactgttcctattcaagcaccagcagctgctcgctcgctattacatggaacgcttgtccaacgatctcggaaccatcccggagttctcttggtacaaacctctccccaccggatactatcccaacatgcattactacaatggagtttcgttcccgtcacgcgacaacaaccactacgtctacaccccagaaaactactacgacgtcgacgaactggttgattacgagcaccgtattcgcgaagtcatcgatcaaggatacctggtcttccccaatggaagccacgtcgatctgaccgaccccgaatccatcgaatacctcggaaacgtcatccagggtaacccggatagtgtcaacactcgctactacaaatacgttggacaaatcgctcgcattctgctgggagcttccgtcgaacactttgatgaccataaagtcatcccaagcgtcttggagcactacgaaacctccctgcgtgacccgatgttctaccagctgtacaagcgcatcatccactggtactgggaattcaaggatcacctgccgcactacacctacgacgagctgaacttcgccggagtcaaggttgagtccgttgaggtcgacaaactggtcacctacttcgatcgctccgacgccgacatcactaatgccgttgacgttgaagtcttcgatgaaaccaccatgaagggatcggacatgaagaaattcggcaagatcgctcactaccagggagaggacttcgtcatcaaggctcgccagtggcgtctgaaccacatgccattcaccttcaagctgaacgttgtgtccgagaaggtcaccaagggtgtggtgcgcgtctacttggcacccaagtacgaccaatacggacacgtctatggggtcaacgagaaccgcgagaacttctacctactggacgtgttcccgtacgactttgtcgttggcaagaacgtcatcacccgtgattcgactcagttctcgatgttcgtgcaggaccgcaccccgtactacgagctgtacaagtgggtcatggatgcgtacaacggcgtgaagaagttcccgctggacatgaccgaggctcactgtggtttcccggctcgcctgatgctccccaagggcaaaaagggcggtatgcccttccagctgtacttcatcgtgtcgccgtatcacgctccgagcaccccccaacacgaaggctacgactacacgctgaactgcggtgtgggctcgggagcacgctacgttgactcgctaccgttcggatatccgttcgaccggccaatcgacgagaaggtctggttcaccccgaacatgtactactacgatgcaatggtgttccacaagaacgagaaggagatcaatgccgtccactgaggaggtgggacgcggtcgtgaagggtgtttgaaaatgtaaggaaatggttcactgagtatattggatttatgttgat

SEQ ID NO: 150 - DNA, 1314 bp, Aedes aegypti
cgcacttctggaggtgtgataaggaaattactatataaaattcctggggcgaaaatttggggacagtttatcaaatatttccaagtgatcaacatagaatgaaacagttgcttctagtctttgccgcactagtgtttgccgtggatgcgcagattcatccggatattatagaggacgcccatttggacgcggtatgttcgtgaatgagacgcgctcggactcttacgaaagtacggttatccagccgaagagcacatcgtagaaaccgatgatggctacctcctcggagttcaccgctgcccaggaagcccaatgtcgccaccggcgcccggaaagcccgtagttctgctgcagcacggcatgctgagttcttcagccgactacatcttgatgggaccgcagactagcttggtctacatgctggccgatgccggatacgacgtttggctgggcaatggccgcggcaaccgctactccaatcgtcaccgaacgcggaacaactcgacccagcagttctgggacttttcgtggcacgaagtaggcagcatcgacatcccgaacatgatcgattacatcctcgcgagaacgggacagcaaggtctgcagtacgtgggccattcgcagggaacgaccgcattctgggtcatgatgtcgcagcatccgtactacaaccgtcgagtcaagagcgctcacttgctggctccggcggcttacatgcaccacacccgtagtccctatgtcatcttcctggccactttcctgcacaccaccgagctgatgatgcagatgatgggaacctggtactttgcaccaaccaacgagatggacattcaaggcggactggacaactgccacgatggagcacctttccagcagatgtgtaccatcaatactttcctgattgccggtttcaacactcaggaggtcaactacactatgcttccggtgatccacgcgcactcccctgccggagcgtcggccatgcagatgatccaccacgcgcagacgatccggtcccgaatcttccgccagtacgaccacggacctacgttgaacatggtccggtacggttcgatggttcccccgcgctacaacttcgacaacgtccaggctccgaccctgctctaccacagcaccaacgactggctggcggctccggaagacgtcgaactgctccgtcgggaactccccaatatccacaagcagtacctcgtacgacaaccgcagttcaaccacatggacttcatctgggccatcaacgtgcggccgctactgtacgacgaattgctgtcggatctgagggcatacgcctag

SEQ ID NO: 151 - DNA, 1974 bp, Aedes aegypti
atgaaactcgtttgcatttttgcgcttttattgccgctggtttcggctagctcccctgttagcgacgaatccaaagacgtcgaagcggatgtcaaacgaatacggatagatccggccttgcggaaggcactactacgtgccctgcgacacctcaaggaacgccaggaggagagtgaaagcgagagcgaaagcgaacaacagtacgactccaacaccactgaagaaaacttcgcaaccaccattctggacgagctcctcgaggagagcacaactcgcgatcgcaacatcaagtatcacacttttacggccgatccggacaacagttccaatggtgaaaacgaaatcatcaaaacgatcatcataaccaagcccaagacgactctgtcgccgccgaaggaagatcctctgaccattcccgactctatcaaggacctggacaacgaccttaacacaaaccaagtggttcgcagcgtttcgtccggatcgagtaaccaactccaggaagatgagaaggtctctatcgtgacggccattcccagcagcagtacgacttctcaggcgacgccacctgccacgaccaccacgaccgctgctccgacaacgacagtcaagcttccgactcacaacgccgatggagaaaacatcgaacgagtcaaaagcggagacgtcaagatctaccaggctccactagtggccgccttcaccgtacagcaagaccaacggggcatcccccacaatgtgattcctctgctacagagccttcccaagaacattcccgttgatttccggccatcaaccccagctgccgcgaccaccactacaccaacccaagtccccgtggcacccaaccccacacaaccctccgcaaccgttgctccgatccccctgaacaccaaggcactcgaagaaaaaactcgtcttctggaacaacagctcataaccctccagacccagcagcgcatccaagaacagctcctccgatcaaagatcctccaagagcagcagatcatcctccaacaacagcagcagctccaacatcaacgtgctcgcctcgaggaagaaacgcgccaacgtctgcaaaagttcgaagaggaacaacgcctcttccgtcagcaacagcaacaactccagcagcaacaacaacagctcaagttccaacagcaacaacagcaacttttcaaaccattccccaaccaacaacttcccaacaacctcccacctccacagttccagcaactgctgcaacaacctcagcaacagcagcaaccacagcaacaacagcaatccaaacccgtcattctcccgcaacctcaacagcagcaattcttccaagagctcccacgcaacaccccggccgtccagttcatccccagcgtgtccctacccagcaaacccatcccgatctccgtggaacaacagcttccgttcaaggaagccgtcaacttccaatcgatcctcccccgaaaccccgacatcgaacaaatcaaatccctcagccaacccctccagcagttccccatcatcccgccatcgccgccgcaactctccaaacagcaactgctccccctcaagccgttccagtccttcaacgtgaaccccgcaccagctccgctcaacatccaacctccgctaccgatcgatcagcagctccctctactccagcgcacccgcgtcttccgccatgaatccggaaccggcaacttcggcttcaaccaaccttccaacatcccgttccgaccttcccaaccgatcccgctcgtccagcagaccggcggattccaacccctgcgcaacgataaccaactccagaacctcctggtccagtccggcattacgtcccgctcggccgaagatttcaacatcatcaccaaggtgctggccctgaaccacggaataccccaatcctcctcccagcaaatgttcgccaagctcagtcccgaacagcagcagcagcttctgttccgaaaaaagtag

SEQ ID NO: 152 - DNA, 561 bp, Aedes aegypti
atgcttttaccaatattagtattagtcatgtcactttttggtaccgttttcacgggagatacggaaagccacactacggacactgcccgtcacgacctgcacattcacgaccacttgcacacagccgatctacaccacaaccctctgcatgtagcggatttccaccacgatctgcatcacgatgttcaccacgacctgcaccatgatgtgcaccacgatatccaccaccatgacgtgcacacggcacatgatcttcaccaccacgacttgcaccacccacatcacacggtggccgctgaccacctgcacaccgaagtgcaccaccacgacgtagtccagggccatggggatcacctgcaccactcacaccacgatgtggtagtggccacgccccacgcgtctggcttctggaagaaaagcttcatctggaagccccgttgggtgaagtcctggcacgagaagaaaatctacgtcccggtgtggaagcacgtttggggtccggtgcagatgactgagtgggtgccgatcccgaagcctccaccaggttggcaaaagcattga

SEQ ID NO: 153 - DNA, 778 bp, Aedes aegypti
gtgttcttttgttctgtttgtttcatttcaggatagtcagggtctatcctcactcaagatgggagcctaccgttacgtgcaggaattgtaccgcaagaagcagagcgatgtgatgcgctatctgctgcgtgtccgcgtctggcagtaccgccagctgaccaagttccaccgtgccccgaggccatcgcgcccggacaaggcccgtcgcctcgggtacaaggccaagcagggcttctcgatcttccgcatccgggtgcgccgtggtggccgcaaacgcccagtcgccaagggttgcacttatggcaagcccaagagccacggtgtcaacgagctgaagccataccgctgtctgcagtccgttgctgaggaacgtgtcggccgtcgcgttggaggtctccgtgtgctgaactcctactgggttgctcaggatgccgcctacaagtactacgaggtcatcatggtcgacaccgcccacaacgccatccgtcgggatgccaaggtcactggatcaccgaggccgtgcacagcaccgtgaactgcgtggcctcactccgctggcagagcagcgtggatgagcagggatacgctacgcccagacatcggaggatcccggcgggcatcctggcgccgtcgcaaccgtctgcacatgcgccgttaccgttaagctggtcgttgctaatcctgctgcagctgcagcggcttagtaattacttctactactgcaaatggatactgtttgtagaaattacatcaatatacaaaaagctttaaggtctaa

SEQ ID NO: 154 - DNA, 1170 bp, Aedes aegypti
cgaacctccaacgtatttacagtcgtcgactaagagcgactaagcaggactgaggatagcattagattcagcaacctgtaattaactagagacacttaatttggtatttaggatacacggaaatgttaattttatttcgctgcaatttttacaaaaacaaagaacattatccaattcaattctacatgcatcattatctattcaaacaagacgatttcatgtatatttgagcttgtattacttccagattgatttttgtttattatggaagtgcttgaaattttgccgcaaaattgtcacttctggcccatagtagagtggtaccaaccgataacaacctgtcgccatatactataggttttaaacggccgaacgaaatcgaaagtgcaatgaaccgaacctgaacggcggcgatggcagcggcggcagaacttacgaaatgatttcgctcggttttcgcgatacgaatgtacctgatcagcattttgatcctggcagaggaaaaaaggcccactagttaaggcattataaaaaccgtcccctggtagttggttaggcatcagtcgcttgtagtccaccgttccaacccactgcgtgtaggccaaacttagttcaaattggcaacatcaatcatgaaggctttcgtggtgttttgcgtggctttggccgttacttcctcctcggcagttgacgaatcctcgaagaaggacaaacgtggtgtttgggatctgggctatggtcatcacgattcatatgcttacgatagccatcaccacgatatccatcattacgacagcagccagtggactcccatcaaacactactcggacgatcatcaccacgccgtagagatcgagaagaaggttctggtggagaaacatgtcccggttgtcgtcgagaagaagatcccggtgcacattgaccggcccgtgccatacccagtggaagttaaggtcccagtggtgcacaaggagtacgtcgaagtgccgaagccgtacgccgtccacgtggagaagcacatcccggtgtacatccacaagccggtctacgtcgagaagcctgtgccagtcagcatctacatcaagcaaaagaagaagcattggggatggttctaagacggtggcgttaagtagactgagttaataataaaaattgttgtgttaatgtaaa

SEQ ID NO: 155 - DNA, 2239 bp, Aedes aegypti
ggaacgatgaggtcgattttggcagtgaccgtagtctgtctgtttgctctggcaagcggcagctacatgaccaaggatgttaagtacggtgatgaaattttcctgaagaagcagaaagctattcttgagattttccaacacatccatcagcccgagctgcacacgtcgtcatgggaggaatccaagggcttcgatatcgagcagtatttggatcactacaccaatgccgaagccgtgaaggagttcctgcggttctataagcacggtatgcttcctttggaggagatcttctccattcagaacgagttccaccgcgaagaggtcatcgccttgttccatctgttctactacgccaaagattgggataccttctacaagaccatggtctgggctcgcttccacgtcaacgaaggcatgttcgtgtacgctttgaccgtcgccgttctgcatcgccacgatatggaaggcatcgtcctgccagcgccgtacgaaatctatccgttctacttcttcaacgatgtcgtaatcagcaaggcccaacgctacaagatgcaaggcttttacaagatgaagaagaacaatgacgtctacactgcgtatatccctagcaactacaccggatattacgtccacactggcccagaacagcgcgttggctacttcactgaagatattggcctgaactcgtactactactacttccacgctgactatccattctggatgggtggcaaggaatttggcctgtacaaggaccgccgtggcgaattcttcctcttccaacatcaacaactgctggcccgttactacctggaacgcttgtccaacgacctcggagtcatcccgcccttctcgtggcacaaatccatcaccaccggttactatccataccttcgctactacaatggaattccgttcccggtccgcgacaactattacagtgcctccgtcgacaagagctacgttgtggaacagatcgaagacttcgaacaccgcatcatagaagccatcgactatgggttcatcgttctgcccgatgggaaatatgttaacatcaccactccctctggaattgaatatcttggaaatttgattcagtcgaacggtgacagcgtcaacacccgcttctacggctacttggaaatcctagccaaatacttccttggtggatctttcaagctgcaacatgaataccgagcgattccatcggtcctggagaagttcgaaaccagcatgcgtgatcccatgttctaccaattctacaagcgcatcgtcgagtactactatcgcttccttgatctccttccatcctacaagaaggaagagctggacttccccggagtcaagatcgaaaccgttgaaatggacaaactggtaacctacttcgataagttcgacgcagacattaccaatgccgtggacgtggaggtattcgacgaaaccaccatgaagggaagtgacatgaagaagttcggcaagatcgcccactatcaaggcgaagacttcgtcatctatgctcgcatgcctcgtctcaaccatcttccgttcaccttcaagctgaacgttgtgtccgaaaaggcccaaaaggccaccgtcttcgtctacctgggtcccaaatacgaccagtacggaaacatctacggagtcgacgccaaccgtgagaacttcttcgagctggaccatttcctggttgatctggtcccggggaagaacgccatcacccgcaactcccaggacttcagctggttcgtgaaggaccgcaccacctactttgagctgtaccaacaggttatgaaggccgtcaccggcgaaggcaagttcccgctggatatgtccgaagctcactgtggcttcccagctcgtctgatgctccccaagggcaagaagggtggcatgccattccagttcttcttcatggtcgttccctatcacgcacccaaggttgaacagttcaccggtttcgatcacaccctgtcctgtggagttggatctggtgcccgctacctggacgatctacccttcggctaccccttcgagcgcaagatcgatgaaaatgcctggtttaccccgaacatgaagtactacgacaccctcatctaccacaaaagtgaaactgaagtcaactccgtggtgatacactagttcaaatgcccatccctcatgcgaaacccattcaataaaaattgcattccattatcggctcccgaagcactgcgtaatgccaacaattaaatgaaaa

SEQ ID NO: 156 - DNA, 2139 bp, Aedes aegypti
atgaagacgatcacattggtagcgttggcgggccttgtagctctggctgcaggttacgtcgtggttccggaggacaagattacctacgcggacaaggatttcctggtgaagcagaagcagctgttggaagttttccagcatgtccaccagcacgaagtccacaccgagctgtgggaggtgtcgaaggagtacaaaatcgaagaaaactacgatcactacactaacgtcgaggcagtgaaggagttcgtcaagttctacaaacatggtatgctgccgttcgatgagatcttcagtgttttccactacacgcatcgcgagcaggccatcgcattgttccacctgttctactatgccaaggattgggaaactttctacaaatccgtcgtctgggctcgtttccacgtgaatgaaggcatgttcgtttatgctgtaacggtcgccatgctgcaccgcaaggatttggccggcctggaacttccagctccctatgaaatctacccctactatttcttcaacaccgaagtggttcagaaggcctcccagtacaagatgcaaggattctatggcatgaagaaggtcgatgatgtctacactgctgtcatcccaaccaactacacgggctggtatgtgcacaccaacgtcgaccagaaggtctcctacttcacagaagatattggcctgaacacgtactactactacttccacgctgactacccattctggatgggcggcaaggaattcggcctgtacaaggaccgtcgtggcgaactgttcctattcaagcaccagcagctgctcgctcgctattacatggaacgcttgtccaacgatctcggaaccatcccggagttctcttggtacaaacctctccccaccggatactatcccaacatgcattactacaatggagtgtctttcccatctcgcgacaacaaccactacgtctacaccccagaaaactactacgacgtcgacgaactggttgattacgagcaccgtattcgcgaagtcatcgatcaaggatacctggtcttccccaatggaagccacgtcgatctgaccgaccccgaatccatcgaatacctcggaaacgtcatccagggtaacccggatagtgtcaacactcgctactacaaatacgttggacaaatcgctcgcattctgctgggagcttccgtcgaacacttcgatgaccacaaagtcatcccaagtgtcttggagcactacgaaacctccctgcgtgacccgatgttctaccagctgtacaagcgcatcatccactggtactgggaattcaaggatcacctgccgcactacacctacgacgagctgaacttcgccggagtcaaggttgagtccgttgaggtcgacaaactggtcacctacttcgatcgctccgacgccgacatcaccaatgccgttgacgtcgaagtcttcgacgaaaccacaatgaagggatcggacatgaagaaattcggcaagatcgctcattaccagggagaggacttcgtcatcaaggctcgccagtggcgtctgaaccacatgccattcaccttcaagcttaacgtaatgtccgagaaggtcaccaagggtgtggtgcgcgtctacttggcacccaagtacgatcagtatggacatgtctacggagtcaacgagaaccgcgagaacttctacctactggacgtcttcccatacgacttcgtcgttggcaagaacgtcatcacccgtgattcaactcaattctcgatgttcgtgcaggaccgcaccccatactatgagctgtacaagtgggtcatggatgcgtacaacggcgtgaagaagttcccgctggacatgaccgaggctcactgtggttttccggctcgcctgatgctccccaagggcaagaagggcggtatgcccttccagctgtacttcatcgtgtcgccgtatcacgctccgagcaccccccaacacgaaggctacgactacacgctgaactgcggtgtgggctcgggagcacgctacgttgactcgctaccgttcggatatccgttcgaccggccaatcgacgagaaggtctggttcaccccgaacatgtactactacgatgcaatggttttccacaagaacgagaaggagatcaatgccgtccattga

SEQ ID NO: 157 - DNA, 369 bp, Aedes aegypti
atgttcaaacttctgctgatcgctgctgttttggccccggccgtcctgggtctgaccccggttcgccagtgtcccaacggagctggcctgccccagtcggtggacatcaacggatgcaccggatcgccctgtccgattgtcaacggtcaaccgctgttggcccatgccgtcggaatcaccagcccagtggccaccaacggcctcgaagcttacatcatcatccgtctggccggtctgcagatcccgttcccgatgcccgatggactggaggatgcttgcgcgaacggaaccccggccggaacctgccccgtgtccgctggacagaccttcgactacatctggacttcgagggacaggtcctgcagctga

SEQ ID NO: 158 - DNA, 4144 bp, Aedes aegypti
atgcggaaacgtaccaacaccatggcgaaggtttacgatgttacggcgatcaaggtcaagaacgtcaacaatgggacggtgggaaagccggtcgtcttcttggttgaaacgtcccaagcaggaccaggaaatctggaggtaacggtgaacggtggacgagtgcctacttcggctcaagctcaaggtcagcatacgtacgcgatcagtttcactcctcgggaagcccagaaccacaccgtggagttacgcttcaatgggcaggatgtaccgggaagtcctttcacgtgcaaggtctctccagcagcgcgcatcgtcaccagcgatctgatggataaagtaagcgtggggcatgtgtttgacttccttgtggagtcggatatatctccagttgtggaagttcttggtccagctcgacgtgcagttcgtgccgatgtgattccagtttctggtgcgtatcgggtaaaatttgagccgatcgaggttggtgatcattcggttgaagttcgcttgcctggcagtggtcatgtggaaggaagtccgttcctgttgaaggcctattctgctgaaaaagtggtcgtgaccgatatcagacctggagtcgttgggaagagtgttagtttcgggatcaatgcaagccaagccggagctggaaatttagagatcattgtcgctgtaggtggaaaaaatgtaccgaactttgtccaatctgagggtaacgccaggttcaaagttaacttcaagcctaccgaagctgcaacgcattcattgtcggttagattcaacggatatccagttccagggtcaccgtttgcttgccatgttacacatgctcctgtatcgctctctaaagcaatagctaccggcgaatgtctgagacaagctccggtcaaatgcgagaacatcttcgagctcgaaggattcgatggcgtagatcctcaagtgttgataacttccccgtctggagataccgtttccagtagagtgtcattcagagacgacatccactttgtgtcattcgtacctacttcagttggtagacatttgatcagcgtaactgccaatgatcaacacattaatggatcgccattctcttgcaatgtattcgacgtttcgcgtgttgcaatcagtggactcgatcatcataacactttggcttctttgggagtaccgcttaccttcagtgttgatgctgctggagcaggcgaaggaactctcgaattggttgtatcaactgcaactagcaccgttaaagctgaagtcactgcatgcgcaaggggattgtacgacgtaacatttgtaccgcaaacctgtgaacctcattttgtcaacattacattcaacgatctaccggtagacggaagtcctttccgctgcgaggttcagcaaaatacgcaaaatgtgcaggttggaaacagcacctttatcgagttggttggtgaagatcaaactgtggaaatctacgacccagagaacaagatggtaccgtttactttgtccagaaaggctgccgagttcaaagccaccaaaatcggaagctacatcgttcgttacgttgatcaggaaacccgttcattcatcgccgcaaggacaattaacgtatttgatccttcgatggtaaagatcgtcgaggtcggggaagcttactgccataaaccagcatccctggcggtatcgatcttggatgctggtcaaggaacgcttacttccgtggttcggtgtggtggcttagaggtaccgcattcgattcgaggaccgaccaagaacggtacttgggagattgtgtaccatcctacaagagttgctccacacaagattatgatcctttacaatcaggtaccgatcgctagtaagccgatcgaaataaacgtcttggcaccagctatgagcaaggagatcactgtcaacggcttgggactgtaccaggctcgtgtctccaagaccacctcgtttgccatcgacaccgttggtcatccggcacgggagtttgacgtggtcgtttccggcccgggaggtcaagctctgccggtgcgctgctaccagaccaagggtggccatctgcaggcggaattcaccgtacagaaggttgggcaatgcttgattgacgttctccatcaatcgaaacccttgccaggaagtccgtacacctgcgagtcgtacgatccatctaagatccacctgcagaaagtgccgaagatgaacctttgcacgaactcgcccataacctggaatgtggaatccgaggcggccggcgttgccgaaatcgaagttacggtcctgtcccccaccggacagaatgtgccggtccagttgacccaacaggaagactacgttcatcaggttgaattcttgccactgactgccggtcactacaaagccactgtcatgtacggaggtgaaagtgtaccgggttcaccgattacgttcgccgttcaagccgttggaaacaaaacggatagccatgcgtccggtaatgggttggaagttgcccaccgtggcaaggaaacctcctttgtggttttctgcccgtctatgcccaatgtgcagatcgaacgatgtgacgagcaagcggaaaggatagaacctaagattaagaacttgggtaacaacgaatggaagatattctatactattctgacggttggagagtacgagatcagagcgagttgtaccaaccgaggtcccttgccagggtcgccgtggaccatttcttgcctggatccggctaaggttacaccaatcggaggctggggaagtctgctggataacaacggaaagctactgttaccagcaaagattgccttcgataccagcatggccggtccaggtgaactgacctgttacgtagacagtacagaagttaacgttgaaaagcaaagcaatggacgatgcattctctatttgtcggacgaagaactatctaagggagagcatagctttgatttgacctggtctggactgcccatctctcagagtcccggctacgtattcgtcacaggaaactcatctgccgcggataaagtggtgctcacgggaagaggtcttacatcggctcaagtgggtgaaatatctcatttcactatcgatgcttcggaggcattatctggcaaaccagaagtgcacatggaattcgaggatggagaaaatctaccagtaactctgctacagccaaggccaaacgaactgatctggcttgcgtcatacaatcctcacaaatcaaccggaggacctttgagcctctccgtcgaatggaacggtcgtctaatcaagggatgtcctctgactatttctgttggaccggcagtagatgctgctaaagttctctgttccggcgaaggtcttcgaaatggcgttgttggtagagacatcaaatcctggattgacacgaggcgagctggtccaggtgaactgacagcgcactgtgctggtccacggaaagtggcttactgtgagctatacgatcatggagatgctactttcacattaaacgtgaaacctcaagagccaggtcggcatacgctaacgatcaaatatggaggtcagcacgttcccggatcgccattcacacttcgagtagcaggagctcccgacgccagtaaggtccgcgtctacggtccaggaatcgagcatggcgttctggcgacgttccagtcgagatttattgttgacacgcgaggtgctggtgcaggtcaactgacggttcgcgtacgaggacccaagggcgccttcagagtcgagatgcagcgcgaaagccagaaagatagaactattctatgcaagtatgatccaacggagccaggcgactatcgggtggaagtcaagtgggccggcgaattggtgcctgggtcacctttcccagtgctgatcttcgatacgcaggaggagctcaaacgattcttacacggcaattaacatcaccctttgagagtattgtttataattttaagtgaagctctaatttttaagatgttgactaatttatgctagacaaagacgaaacgttacaaaatcataaagaataacaaacaaagaaggaagcaaattcatatttacacatttcgcggagagcaatttgttccacaacgagcggcttgatttgtgaattattgtatttattacaaaaaaacaaataaagtggcctatatgc

SEQ ID NO: 159 - DNA, 876 bp, Aedes aegypti
atgaaatcgatcacgtgtctagcggtgttgagtgccgccgccctgttcggagtggtgtcctgtgcgtcgattgcctcgaatctggtggttgatgccacgccaaggatcgccaccagcgagcagcttctgtcctcgattgtgaacgagtggttcgaggatgatttggtgatgaactgcctcaagggtaaggtgttggtttatctggatggagtgctgggactacgcgaggaacaggttcgcgccttcgaggagcagaatgtggacaaggtgatcttcgatcgtgtggcacgcatcctggccacccacgagttcaaggttcaactgccggaggataccgtcgtgagctaccgtgccgataagggtgtcaacctggatgtggttcctgctggacttgccaccgaatcgcgcggtcatttactgaagaagaaactcctgcttccagtgctgctgttgctgaagctgaagatgaaggccctcatgccgatcttcgtcgccctgatcggcctcaaggccatgaaggctcttatcctgtccaaactggccatcactctggtgctcggattcatcgtcgcccagctggttaagaagtccggcctgggaatgcccatgatgtcgatgatgccaatgatgcccccagcagccgaatacggtgcccctgctccggccaccaccgagccaccatccagctatggtgctccatcctgggaatcgcaatccggaggaccttactcccgcgtttgggaaccatctacctcctcctcttcccacaatctggcctacagctcgtactacccaagcggatccagcagctcctacagctccactgcctacaactctggatctagcaccagctccagctcctcgccatcctcttcc

SEQ ID NO: 160 - DNA, 1667 bp, Aedes aegypti
atggacccagttacggtgattttgacgattttcgtgggcctaacggggctcgtctacttctttctgaggcgggagcagcagaaatggccacgattaggcgttccgttcgcgaagaatcctcacctactattcgggaacgttcgaggaatattccagaaagagcacagctgcgaaatcctccagcgattgtactgggagttcaagggccgaggattgaagctcggtggaattatgaacttcttccagccggcagtgctggttatcgatccggagatcagcaagagtatcctggtaaaagatttcaacaagtttcatgatcgcggaattttcgtggatccggcgggggatcctctgagtgccaatctgttcagcctggagggagcccagtggaaggcaatgcgtacgaagatgtctccgacgttcacctcggggaagatgaagtacatgttcgagtcggttctgaacgtggccgagcggttgaaggactatttggcggagaattgcctgaaggaggacatcgagctgaagaacattctgcagcggtttacgatggacgtgattggtaatgtggcgttcggggttgagtgtaactcgattaagaacccgagttcggagttccggttgatgggcttaaaggcgaaccggtttgacggggtaaggtttttgaagttcttcatcggaggcgcctacaaaaactttgcgaagaagatcaagctgaaggtcgtggaggacgacgttcacaagttcttcatgagtttggtgcacagtacggtgcattacagggaagggaacaacgtgaaaagaaacgactttttgaatttgctgatggagatcaagaacaagggaaagttcagcgatgaacccaattcgggcggcgagggaatcacgatgaacgagatagccgcgcagtgtttcattttcttcacggccggtttcgagacttcgtccaccacaatcaacttctgtctgtacgagttggctaacaatccggatattcaggatcgattgcggaatgagattgaagatgtagtcgctaaggacggaggagagttgaaatacgatactctgctaggtatgaactatctggatcgagttgtcagtgaaaccctacggaagtactcggcggtggacaacctcttccgcatctccaactcgccgtacactccggacggctgcaacttcaccatccctgcaggaaccctgttccaaattcccatccactcgatgcaccacgatccggagtacttccctgacccaggtcgtttcgatccggatcgattcctaccggaagtggccaaatcccgacacccatactgctacctgcccttcggtgaaggaccccgcgtttgcatcgggatgcgattcgggttgatgcagaccaagatcggattggttacgttgctgcggaacttccgcttcgggccccggtccgagactccggaccgattgcagtttgaagcgaagacgtttgtgctgacgccgcagaccgggatatacctgaagattgaaccgatcgggatatgaaggagtgactgtgtagagtagcttgcagagcgttggatctaaaaaccccaaaaaataaaggtgttaggcttccagcatccatcacagataagctatctatggatttagctctcgttgtctgttttatgattggctgcaataaa

SEQ ID NO: 161 - DNA, 2724 bp, Aedes aegypti
atgtttcggttaggcaagcaaagttttaccaagtttaaagaccagctcagtggtcagcgatcgttggtgggaaggattagcagtgaactgtccaagcgtagtttgtcgtcaggatcggccttaccggaaagtgcggatgtcgtcattatcggaggcggatcagctggatgcaatacgctgtatcagctaacgaagcgaggcatacgtgccgtattgctggagcaggccaagctcacagcgggcaccacatggcatacggcaggcttggtgtggcgcttacgcccaaatgacgtggagatcaagctactggcagcgaccagaaacctgttgatgagcctggaagaagagaccgggtacaattcgggatggatcaacaacggtggcctgttcatctcgcactcggacgaacgcttggatgaatacaagcggttggctacgctcggcaagtgcttcggcattgaaagtgagatcctgacaccgcatgaagcccaaaaggtgttcccactactcgacccagcggccttcacgggagctttgtactcaccgggagatggcgtcgtagatccagctatgatgtgcacagcgcttactaaggcagccttggcaaacggaggaaccgtagtggaggactgcacggccaccaagatcgtaacgggagagaacctgctgggtgtcaaagatgtccgaggcatcgttaccaacaagggaaccatccgtaccaatacggttgtgaacgccacaggcgtttggggaagggacctcatcgaaccgctgggcgtccatctacctctgatcccgatgaagcatgcgtacgtcatctcggaaacgatggaaggcgtccaccaaatgctgcccaacatccgcgatcacgactattcgatatacttccgcattcaaggcaagtccatctgcatgggaggttacgagaacaaccctgtcctgctggatcgagtccccaacgacttccacttcggtttgtacgacctggattactccgtgttcgacactcacatccagggtgcggtgaagctttgccctgaattcggcaccgctggaatcaagagtacaatctgcggaccggaatccttcacgcctgatcacaagccgttgatggggccggatcccctgatcaatgggttgttccacaactgcggcttcaactcggccggtatgatgctgggcgccggatgtgccgaacagctcgctcagtggatcatccacgacagaccggacctgcacatgtttgcgtacgatgtccgacggttttctccgaaacagaagaaagccgtcaactgggccaccgagagaagccacgaggcgtacgccaagaactacagcattgtgttcccgcacgatgaaccgctcgccggtcggaacttcaccgtagatccgttccacaagcaaatgatccaacacggagcggttatggaggaacggcacggttgggaacggccaggatacttcctcccggaagacaccgtggtggtccaatcgtacgattggtacggatactatgactatccgaagaataccaacaccaactacgaggaaactctgaagaaggactacaccttcggattcccggagcatcacgatttgattggagaggaggcactgacttgccgtcaagaagcggctatgtttaaccttagttactttagcaagttgttcctgactggtagccaggccaaggaagccgttgagtggatctttgccggtgatttgaacaaacctatcaacaaaaccgtctatacttgcgcgttgaacaaacgcggtggcgttgagaccgatgtgaccatcagcgttgtggaatcaggccaaggcgaactccacgatccaatcttcaagggacgaggctactacattgtggctggtggtgcttcggcgtaccacaccaagtcgcacatttgggcagcgatccaggagaaggccctgcgtgccgtcgttaccgatcacaccgaagagctgggcgtactctcggtccagggaccgaagagccgcgagatcatccagaagatcaccgatttcgatctgagcgacgaggagaaactgccacccaactccactgccattctgagcatccagatcaatccgttctacaagtgcaacgtacgaatcttgcgggttagcttcgtcggagaactgggctacgagttgcacattccggaggagagttgtaatgacgtgtacaacgcgctgatgaaggccggttacaaggaaggtctgaggaatgccggatatcgtgcactgtactcgttgagcagcgagaagggctaccatctgtggggatacgatttgcgatcggatgatacgccggtggaggcaaatttgggattcacctgtaggaagcgtggcgactaccagggacgggctgccatcgataggcagctggagaatggagtgagcaaacggttggcgttcttcacgctgaacgaacaggttcccatctggggcttggaggcggtgtatcgtaacggagaaatcgtcggacacctccggcgaggcgaatatggctacaccctgcagaagcccatcggacaggcctacattacccggaaggatggcgactacgtcaccaatgagtacataaagagcggacattaccagattgaaatcatgggcaagcttcatgaagctcagtgtcacctcaggagcccattcgatccgaagggcgagcggatattaggaatttataaataaattttaactagttttacaacattt

SEQ ID NO: 162 - DNA, 1240 bp, Aedes aegypti
atgcatttcgtttatttatttcgccatcctcaggtgttgggttcggtgttgctgctggcttctgccgcttccggttcgtacttgggtcatgaagtatcttcgcagtaccaggcccatgatggcatcggtggctactcgtacggttatgctgatccaaattcgcagaaacacgaaactaaggatgctcacggagtgacccacggtggatattcctacgtggacggcgatggccacgtgcaaaccgtcaagtacactgctgatccgattcacggtttccaagttgcggctaccaatctacccaaaggtccagcaccgatccatccgcactacggcagcccatgggtccattcgtacgctcccatcgtgttgggtcacaatggtgcccccttggaaactccagaagttcaagctgccaaagctgctcactttgccgcccatgccgaagccaaggctagactacacaagcgctcgctttactccagctcctggaagttcgtcccagctcacatcccagaggtcaaagccaaccagtacggaggcgtgccagctccccactttgatcgccatcaagaagctcccatccataaatggcaaggacctatccacatcccggtgatccacaacggtgttccagtggaaaccccagaagttcaacatgccagggcattccacgcttcagctcacgctcatggatacgccccagctcatgaacactacccgggaacgccagctgcccatgaggatcaaaccaagtacggtccatggaagggccctgtccacatcccggtcattcatgggggagtcccagtggaaaccccggaggtccaacatgccaaggaagctcacctgaatgctctggccagtgcccacgccgcggcctcacacggtcccgaggacgatggttcctacaaaccggagctgtgggatgaccatcactaccagcaggatcaccgctaccaacagcactagtctcgccaacaccatgccaaaactgccaccgtactagagccaaatcggatttcatttgttataaacgatgattttgagtgccaaacgggaggaatgcttccgccgctacgggccccgttccgcaggttggcggaagtagcttcaacgctctctaactctttccctctctttttttatgtgtaatctcactcttttgcactgttgtataatgttgattgatttgtatgtatgtatgaagagataccgtatgttataatgcaaagcattgatg

SEQ ID NO: 163 - DNA, 2712 bp, Aedes aegypti
atgcaccactgccgacgagctgcgatgctgcgtgacgcaagtctcacatggcgaccggaggacccggacttgaccttctggttccagcgcatcattctacaatgggtgccctgtttcttactgtttctcttcacattctacgaggtgtacggtattgtaaccagcagataccgggacatcccgtggaattggctgaacatctcaaaaattgtcatcaccttcatgctgatgacgctccgtggattgaccatgatcgttaactaccacaaccagcaggatgtgttcgacgtgcagatattggtggccgttttcaacgctctggcttatataacgaccatcgccctatactactttttccggaagtacggtatccgaagcgcgggaacgatcttcatcttctggttcctgaaagccttcctcgacatcatccagatgcgaacggaagctatgaagcacgccaaacgagaaaacccgatcggatccggcaaaacggtcatctttgatgagtaccaatttgtcagttacacactgcaatactcgctgatttgcttgataacgctcttcttcatcaagctgctgtatttgtacttcgatcggttcacgtggaccggtttccgcaagccactgaccgacgacgacatgtacgatctgaacccgcaggacatgtcccgggagctggtgcctccgttcgataagtactggtatgagagtgtcgaaagtggacgtcggaaacagatggctacggataagaaggccggcaaggtgaaccccgtctacaaaccgaatgccgcctcgaataggtcgatccttccggcgatggtcaaggcgtacggtgggccgttctggttcgcggggttgctccagatcggaatctccggactgccatatcttatgcaggagctgatgggtaacatcgccatgaatggacctttctggatcggaatgacgataacgtttgctctgttcttaaactcgctgttgatcgcactgttcaacggtcagtacttccgcaagacgttccttgtcggtttccgcatccgaacgggtttgatcagtgccatctatcggaaagccctccgcatatcgagcttcgctaagaaggacaccaccgtgggagaaatcgtgaaccttatggcagtcgacgcgcagcggttcttcgaattgacgtcctacttgtacgttctgtggtcagctcccatcattatcggagtttgcatcttttttctgtacgagatcctcggcccggcagtgttcgctggactcggagtgatggtctttatgattccgatcaccggagtcatcgctactcggctacgtgatctccaggtgaaacagattaacatcaaggacgagcgtgtgaagaaaatgaacgaaatcttgagcggcatgaaggtgttgaagctttacgcttgggggcccagtttccaggatgacatagtaggcgtccggcacggagaaatcgacattttgcgaaccatggcctactatggggcagcgactttcttagtgtggagtatggcaccgttcctggtgacgctggctttattcgcagtgtacgtcatgatcgatgagaacaacgtactcgatccgcaaacagccttcgtttccctagcactgttcaacatcctacgcttcccgttggctatgttccccatgatgatcacgtttgccatgcaggcgtgggtatcggttcagcgtatcaacaagttcatgaacagtgaagaactggacccgaccaatgtgacacacaaccgcagcgaggatgccctctcgatcaaagacggaacattctcgtggggtgacgagacgccaatcttaaagaatataaatctagcggtgaagaaaggcaagctgtctgcggtggtcggtagtgtcggcacgggtaagagttctcttatatctgcgctgcttggagaaatggagaagatcaagggtacggtcaacaccgatggatcgattgccttcgttccacagcaggcctggattcagaacgccaccttgcgggagaatattctgttcggaaaagcgtataatgcgcagaaatatgatcgtgtcctggaatgctgtgcgctgaaaccggatttggagatgcttccaggtagagataagacggagatcggtgagaagggtattaacttgtccggtggacagaaacagcgagtagccttagctcaggccgtctacgcagatgcagaaatctacctgttcgacgatccgcttagcgccgtagatgctcacgttggcaagcacatcttcgaaaaggttattggacctgagggtatattggtgggacgatcgcgacttctggtaacccatggtatttcgttcctaccaagtgtggaagaaattttcgtggtgaaggacggtgaaatatcggaaagcggatcttaccaacagttacttgatcagaagggcgcttttgccgaattcctcatgcaacacttccaagaacttgacgaagaagacgaggaaattcagattattcaggagattctgaaggatgaagtctctaggaacatcgtaaaacgagcaatgtcaactcgatctcagaggtcttccggtagtggtggaagtgctggtagtgtttgccggaaaacgatgagtcgacaagagtctcgaaacagcaacaagccagtggaagctccagctgcccctgccaagggtacgactttgatcgaaaaggaagaatctgcaacgggtgccgtcagttga

SEQ ID NO: 164 - DNA, 2463 bp, Aedes aegypti
atgtcttcgtacaggaagaacactcttgtagtggacttcagtgttctgccgaagcgtccctcaacggatcaggtggaggcgttcctccgggatttcgtgaaaatcgatatgtccgacgttaggaacctccaactgcatactctaaaaaattgtgtattcatagaaatgcacaatgcaggattacccaaccgtctggcaaaagagcaccatcttcgacactcgtttggacgcaacggtgtgttctactacatccccatctacgtggacggtcctactaccacaataaggatccacgacttatcgccgcagctgtccaacaccaccatctccgatcacttacaacaatacggcaaagtcatctccatcagcaacgaagtttggaaaaattattttgctggtgtaccgaacggggtgcgagttgtacgcatgcgaatggagaagacagtgccttcatacatcacgattgacaatcaatcttctctcgttacctaccccaagggcgagaaaactgcacacgagaataaagctggccaaaaacaacacccagaacaaacatccacctctgttaccaatatcgatcaatcattccacagtaacattccattcggtctgcacgtattagctctggttagaaaggccttatgtgtcgctactacttcgaaggtcaaagcagcagaaaagtcaagcagcccggaaccgaagccgaatcagtcgtttatgatgaacatcttcagcggcaaattgcaaacctcgcaactcttcccatatccagagcctctcaacgcagagcagaaagaatacattcaggcgttcatcgacccagttactcgattttttaatgagaccaacaaccgcatcaagaacgatgaaaccggcaaagtagatcaagagaccatggatatcgtttgggagttgggactcatgggtacttcgataccggaggagttcggcggtttggggttgtcgaacgtacaagctgcacgtttgagtgacatcagtgggggaaacgatcttgctttcacgatccatatcggagcacatcaatcgattggcaccaagggtattctgctgtacggaacggaagcccagaagcagaagtatctaccacagttgagtacgggacgtgtgtttggagcgtttgcgctgaccgaaccatcggtaggatcggatgctgcgtcggtgaaaaccagagctgtgctaagtccctgtggtaaatactacattttgaacggttctaagctgtggtgttctggaggtggaatagccgatatcttcacaacgttcgcccaaacagaagtagtggatccgaaaactggacagaagaaggacaaaatgactgcattcatcgtggaacgcggattcaatggagtgagctctggaccaccagaaaagaaaatgggcctgaaatgttcaataacaacggatgtgtatttcgacgatgtcaaggtaccggtcgagaacgttcttggagaggttggcaatggattcaaggtagcagttaacattctgaactctggtagattcggattgtgcgcaatgttgacaggaacgatgcgagcttgtattgagaaagcagcagagcacgtgtcaagccgagttcaattcaaacgcaagttgatggagtttgaaaatgttcaggagaaactggccaagatggctatgcatcattacgtggctcaatcactggcctacatggtttcaggaaacatggatttaggttcaactgattatcacttggaggcggctattaccaaggtttttggaacggaggcaggatggtatgtttgtgacgaggccatacagctgctgggaggcaacggttacatgcaagctccgggtctagagcagttcttgagagatatgagagttttccgaatcttcgaaggtgctaacgatgttctgagactgttcattgcattgaccggcattcagcacgctggcaatcaactgaaagggttacagaaggccatgaagagtcctcttacgaatctcggaaccgttttgcaggaagggacgaagcgtgtgtcacggaaaatgggggcaggcggtacggacttgagcggatttgtggttgatccgttgaaggattccgcaaagctttgcgcacagagcatcgatctcttcaatcaaaccatcgagtctctgctgataaagcacggtaaaaatatcgtcgaaagacagttccttttggcacgagtggctgatgcggctattgatatctacactatggcttgtacactatcacgagcttcccgatctgtccgcaaaggactcccatctgcggaacatgagattctgatgacccaggcatggtgcactgaagcaaactaccgagtgcaacagaacatccaacgcatccattccggagtattcaacgaaaattacaaacgaatgtcgatgattgcgtccaacatttgcagtcaccaaggtccagcccatacgaatcctcttgaggtggactga

SEQ ID NO: 165 - DNA, 1503 bp, Aedes aegypti
ccgatcctcaatccatagccaggagaaaaacggattcattgataacagagcacaaggtcagccaccaataccaccttaagcaacatggccgatgacaagcagaaacgggtatcctattggtactttggtggtttggccagtgccggagcagcctgttgtacccatccgttggacttgctcaaggtgcaactgcaaacccaacaggagggtaagatctcgctgctccagttgaccggcaaggtcgtccgaactcagggcgtcctggcgctctacaatgggctgtccgcttccctgctgcgacagctcacctactcgaccacccggttcggtatctacgaggtcggcaaacaggccatgggcagcgattccggattcctgggtaaggctttgttggccggtgcagctggtgctgccggaggtttcgtaggaacccctgccgatatggtcaacgtgcgcatgcagaacgatatcaaactgccactggagcagagaagaaattacaaaaacgccgttgatggcctcttccgcgtctaccgggaagagggcttcagacgtctgttctcaggtgcctcaaccgctacctcacgtgctgtgttcatgacaatagggcagctatccttctacgatctggtcaaggatctcctgctgcagtccggctacttcggtgacaatctaacgacgcacttcctgtcctcgctgacggccggggccatcgcaaccactatgacccaaccgctggacgtgctgaagacccgagcaatgaacgccaaaccgggcgagtttgccagccaatgggagctgatccgttacaccgcccggctcggacctctgggcttcttcaagggttacgtgccggcctttgtacggttgggaccgcacaccatcctgacgtttgtgttcctggaacagctgcgcatgaactttggatatctgaagccggagaagaagtgaacctgttcggagttataacttgttgatattagctttttaggatcaaaaactggaaatactgttgcaatagttgctcgatccgggaatcgttgcttggggtttttggttacgggttcagatttaggtgggacgttctaaagtgtattttgttttgttttgcgcaaagtccggacggtagaaggcatccaggtcttctacaagcatttctctgttagcttttatctcactgtttaaagcttgttattttatttataaccgaactgctctttccaatcaacgacaatgctttcacaatctacgaacaaacacacattccaagagacatttaactaattagtagtgcgtttattttatgtatctgttgaaaacagaaaggaacctaggtatcagtagtgctgttcaatcgtttaacgttttgaaagaaaatattttattatactagacatctgcccaattgtgagatcccaatcatttgagaagtaaacttatcaggaaaccgttccgagttggtaaaaagtaggcaataaataaaaatgtaatttatggtaaaac

SEQ ID NO: 166 - DNA, 20 bp, Artificial Sequence
  Single strand DNA oligonucleotide
gcgcgctcgttcagtattta

SEQ ID NO: 167 - DNA, 20 bp, Artificial Sequence
  Single strand DNA oligonucleotide
acacccgttacggcacaata

SEQ ID NO: 168 - DNA, 20 bp, Artificial Sequence
  Single strand DNA oligonucleotide
tcggcattcgtagcttcgtt

SEQ ID NO: 169 - DNA, 20 bp, Artificial Sequence
  Single strand DNA oligonucleotide
gcagctgacagtttgccttc

SEQ ID NO: 170 - DNA, 20 bp, Artificial Sequence
  Single strand DNA oligonucleotide
cagaatcccagacccggaac

SEQ ID NO: 171 - DNA, 20 bp, Artificial Sequence
  Single strand DNA oligonucleotide
ttggcgaaaccgtaccttga

SEQ ID NO: 172 - DNA, 19 bp, Artificial Sequence
  Single strand DNA oligonucleotide
ctggagtcggtgagcgaaa

SEQ ID NO: 173 - DNA, 20 bp, Artificial Sequence
  Single strand DNA oligonucleotide
tacgtatcgtaaacgcgctc

SEQ ID NO: 174 - DNA, 20 bp, Artificial Sequence
  Single strand DNA oligonucleotide
cgccggatgaagatcaactg

SEQ ID NO: 175 - DNA, 25 bp, Artificial Sequence
  Single strand DNA oligonucleotide
tttcatcgttttacacgaaagtagt

SEQ ID NO: 176 - DNA, 19 bp, Artificial Sequence
  Single strand DNA oligonucleotide
tgcaagctgctgatggagc

SEQ ID NO: 177 - DNA, 25 bp, Artificial Sequence
  Single strand DNA oligonucleotide
tcaccaatagtaatgagggtatcca

SEQ ID NO: 178 - DNA, 20 bp, Artificial Sequence
  Single strand DNA oligonucleotide
cctagagttagccaagggcg

SEQ ID NO: 179 - DNA, 20 bp, Artificial Sequence
  Single strand DNA oligonucleotide
catgtccaggaaggccactt

SEQ ID NO: 180 - DNA, 21 bp, Artificial Sequence
  Single strand DNA oligonucleotide
tcactatcgccatccttgcat

SEQ ID NO: 181 - DNA, 20 bp, Artificial Sequence
  Single strand DNA oligonucleotide
gccactacatcatgggcgta

SEQ ID NO: 182 - DNA, 20 bp, Artificial Sequence
  Single strand DNA oligonucleotide
ccctgttctcacctcggaag

SEQ ID NO: 183 - DNA, 19 bp, Artificial Sequence
  Single strand DNA oligonucleotide
cggcactagccgtaaactg

SEQ ID NO: 184 - DNA, 317 bp, Artificial Sequence
  double stranded RNA
tggaaaagttgacattgacgggatcacgacaactgatttccagcttggccgcatacgttcacaaatgggtctcgtatcgcaagaacctgttctgtttgatcgcaccatcgcggaaaatattgcttatggtgacaacactcgcgagataagcatgccggagataatcgaggcatccaaaatggcaaacattcacgaattcatcgtcaatctaccgaaggggtatgacactagcctaggaaccaaaggagctcaattgtctggtggccaaaagcaacgtattgctatagctcgtgctttggttagaaatccacgaatcc

SEQ ID NO: 185 - DNA, 373 bp, Artificial Sequence
  double stranded RNA
ccccgtcgacggccacgaagttacgatcaagatcatttacaagcgcaaacagcgaatgagcgagaacattcaattctacaacattctcttccaacgcattatgaaagtcctgaagatggtcgagatgggtcgtaagaactttgacccctcggccccgaaactgattccgcagcatcgtctggagatctggccgggatacgttagcgctgtggacgaatatgagggcggcttgatgttgaatctcgacgtgtcccatcgtgtgctgctccagacgacggttttggatcacatccgaacgctggcaagggccaatccccaagattacaaaaacatggccaccaaatcgctgctgggagccgttatattgacccga

SEQ ID NO: 186 - DNA, 431 bp, Artificial Sequence
  double stranded RNA
tcttctccaccacagccaatggcgtacaggctggagaacaggcagattggacggcccatttccgtaacaatcccatgttcgctacggtgcggttgacgcgatggtatttgatcgtaccgaaccgggcttcacgtgaggccaacgatttcctgggctgcatgattcaagccgcacgcggtatgcgatttgagattagtaactgtgaggtggtcaccattccggacgacaacccaggtacctatgtgcgaacattggacaatctgctgaataaggacccgcagttggtgatgtgcgtcgtgaccaacaataaggccgatcggtacaccgccatcaagaagaaatgttgcgttgatcgtgccatcccaacgcaagtcatggtacaaaaaacgatcacaccaaagggtggtaatgtacgaacactcatgtccgtg

SEQ ID NO: 187 - DNA, 422 bp, Artificial Sequence
  double stranded RNA
atcctatacgtcgcgtagctatttatattttagtacatccactgttttcattttttataataacaaccattcttaccaattgtatattgatgatcatgcctagcacgccgaccgtcgaatctaccgaggtgatattcaccggcatctacacgttcgaatcagctgtaaaagtgatggcgcgaggtttcatattacaaccgtttacctatcttagagatgcatggaattggttggacttcgtagtaatagcattagcatatgtaactatgggtatagatttgggtaatctcgctgcattgagaacattcagggtactacgagctctcaaaacagtggccatcgttccaggtctcaagaccatcgtcggcgctgtcatagagtccgttaagaatctcagagatgtgataattttaacaatgttt

SEQ ID NO: 188 - DNA, 350 bp, Artificial Sequence
  double stranded RNA
cgccggatgaagatcaactggatattttgtggcagccagtagagaaaatagatcctgatcaggagaaatataacatcagagcgctggacttgaacgaaccatccaaaatggtagaccacccattcgaaaagcgagttaatttttggaaacgattgtttgaaaaatacggtgggaactatttgcttcatcgagcattgcaatgaaatccaacttcttccaaattgtgttcaatcataaaaatgccctttgaatatatataaccctcgatgcttttataaaagaaatactagagaataatataattaactgattgatattaatccaaactactttcgtgtaaaacgatgaaa

SEQ ID NO: 189 - DNA, 206 bp, Artificial Sequence
  double stranded RNA
tgcaagctgctgatggagcgccacaacctggcccagttggtgttgcagccgatgccaacaaggaaagtttagaaaacgctgaaacctttggctatggatactatggctattatcctcgctattactacagcagctactatccacggtattacggatatggctatggatacggtcacggatatggataccctcattactattggtga

SEQ ID NO: 190 - DNA, 274 bp, Artificial Sequence
  double stranded RNA
tgctaagattggtggccctaaagggtatccccttttgggaaactcgatccagtacgggacaaagtccccggtggagtttctacaggaagtgcagaaaactaacgagcagtgtggaaagttctatcgattgtggattgggcccgatttgattttccccataactgacgccaagttggtggaggcgattctcagcagtcagaaattgctggacaaatccgttcagtacgattttatacgaccatggctgggaaatggattgctcaccagtaccgga

SEQ ID NO: 191 - DNA, 271 bp, Artificial Sequence
  double stranded RNA
tactccaacccttggcctctgaagaccagttcggtttggccaacctacggtggactgtacggcggtaaggtcactgtggttccggaagttgcctacgaaaaatacgcttacccgaatgtgtatggaagctggcctgcctacggaaagaactggggatactcttctccggttgttgctgtcggagcaaaggtggttgacaacggcctgtggaattacggaggatatggctacggacatagatacggtctgggatataacaaggagtggctgt

SEQ ID NO: 192 - DNA, 545 bp, Artificial Sequence
  double stranded RNA
ggatgttcttcttcatcaacccggtagctctggcgatggacttggacctgatcaaaagtgttctggtgaaagatttccaatacttccatgatcgttcggtttactataatgagaaagatgatcctctatcggctcatctgttcacgatggagggtgccaagtggaaaaatcttcgagcaaaattgacgccaacgttcacttcagggaagatgaaaatgatgtacccaactatcatcggagtggcggacgaatttcagaagctaatgaaaagtgaagtgtccagcaatgctgaaatcgagatgaaggagattcttgctcggtttacaaccgacgtgattggcacctgtgcttttgggttggaatgcaatagcttacacgatccggatgccaagttcagggccatgggacgcaaaatattttcgtttgccaatgggcggttcctgaaggcagttatcgctcaacagttccgttcacttgcgcgatcactacatatagctttggttgataaagaagtttcagattttttcctgggtgctgtgagggat

SEQ ID NO: 193 - DNA, 2748 bp, Aedes aegypti
actggttttggtcggaagaaaaagaaaaaagaaatacgctacgacgacgaggatgaggatgaaggtccacagccggattccacacttgagcagggagtgccaatccctgttcgaatgcagggcagcttccctccggaattggcctccacgcctctcgaggatatcgacagctattacgcgaatcagaggacattcgtagtagttagtaaaggaaaagatatttttcgtttctccgcaaccaatgcattatatgtactcgatccgttcaatcctatacgtcgcgtagctatttatattttagtacatccactgttttcattttttataataacaaccattcttaccaattgtatattgatgatcatgcctagcacgccgaccgtcgaatctaccgaggtgatattcaccggcatctacacgttcgaatcagctgtaaaagtgatggcgcgaggtttcatattacaaccgtttacttatcttagagatgcatggaattggttggacttcgtagtaatagcattagcatatgtaactatgggtatagatttgggtaatctcgctgcattgagaacattcagggtactacgagctctcaaaacagtggccatcgttccaggtctcaagaccatcgtcggcgctgtcatagagtccgttaagaatctcagagatgtgataattttaacaatgttttcgttatcggtgtttgctttaatggggctgcagatctacatgggcgtgctgacgcagaagtgcatccgggagttcccgatggacggttcgtggggcaacctgtcggacgagaactgggaacggttcaacaataacgactgtgcgtttctttcatggcatacaaagcaattccgtggtccaagccaattggtacttctcggaaactggagacacgcctctttgtgggactcgtcgggtgctggccaatgcgaagaaggatatatttgtttacaaggttatggagataatccaaattacgggtatacaagtttcgatactttcggatgggcattcttatctgcctttcgtctaatgacccaagactattgggagaatctttatcaactggtgttacgatcagctggaccgtggcacatgctcttcttcattgtgattatcttcttgggttcgttctaccttgtaaatttgatcttggccattgtcgccatgtcgtacgacgaactccagaagaaggccgaagaggaagaggccgccgaggaagaagcgcttcgggaagcggaggaagcagctgcagcgaaagcggccaaactcgaggcccaagcagcggcagcggcggccgcagccaacccggagatcgccaagagcccgtcggacttttcctgccacagctacgagctgttcgtgaaccaggagaagggcaacgacgacaacaacaaggagaagatgtcgatccggagcgaaggattggagtcggtgagcgaaatcacaagaacaaccgcaccaacagctactgcagctggcactgcaaaagcccgtaaagtgagcgcggcttcactttcattacctggttcaccatttaatcttcgtagaggatctagaggatcacatcagtttacgatacgtaacggtagaggacgtttcgtgggcgtacctggtagcgatagaaaaccattggtactctcaacatatctcgatgcacaagaacatttgccatacgccgatgactcgaacgcggtcacaccgatgtcggaggaaaatggtgcaatcatcgttccagtatactatgctaatttaggttcgcgacattcatcgtacacatcgcatcaatcgcgcatctcgtacacatcgcacggcgacctgctcggcggcatgacgaaggagagccgactgcggaaccggtcggcgcgcaacaccaatcactcgatcgtgccgccgccgaacatgtccgggccgaacatgtcctacgtggacagcaaccacaaggggcagcgagactttgatatgtcacaagactgtacagacgaagctggcaaaataaaacacaacgacaatcctttcatcgagccctctcaaacacaaaccgtagtagatatgaaagacgtgatggtgttaaacgatatcatcgagcaagctgctggtcggcatagtagagctagtgatcatggagaggacgacgacgaggacggtccaacgttcaaggacaaggccctggagttcacgatgcggatgatcgacgtcttctgcgtgtgggactgctgctgggtgtggctcaagttccaggagtgggttgccttcattgtgttcgacccgttcgtcgagctgttcatcaccctgtgtatcgtggtcaacacgctgttcatggccctggatcaccacgatatggacccggacatggagcgggccctcaagagtggtaactattttttcacggcgaccttcgcgatagaagcaacgatgaagctgattgcgatgagtcccaagtactacttccaagagggctggaacatattcgatttcatcatcgtggcgctgtcgctactcgagctgggtctggaaggtgttcagggattgtcagtattacgttcattccgtttgcttcgagtgttcaagctagcgaaatcgtggccgacgttgaacttactcatttccatcatgggtcgaacgatgggtgcgttaggtaatctgacgtttgtgctctgcattatcatcttcatctttgccgtgatgggaatgcagctgttcggcaagaactacatcgtttctctacatgttggatag

SEQ ID NO: 194 - DNA, 4525 bp, Aedes aegypti
atgacaaacaaagactttcctcaatgcaacggatcgcagccatcggtgaatctcaaaaatggcatcaacaacgacgcgatgtctgtgtccagttacaagggctccaaagacatactgaacgcaaagttcaacaaaccaccggaaaaagctagcaatgcagttaccaacacccaacccgtatcgtacttcaaattgtttcggttcgctacttggggagaaataagtgccacaattctaggagtaatattagcctcgtttgcttctctcggacttccctacggtgttatcctgtacggcgagtacaccactctactggtcgaccgaacaatcgggataggaaaatcgactgacacggccattctgtcgatgtttgggggtggacacgtgttggttaacgccagtgcggaggaaaaccgtctagccatcttgcaggatgccaaagcctttggactgggagtcctctttgtatcggtggtacaattcctcgcagctgcacttagcgtagacatgatcaatcgatccgcaaatcgacaaataagtagaatacggaagttgttcttacgggcagttctacgccaggatatgacctggtacgacctgaacagtgatgataattttgctgtgagaattacagacgatcttgataagttgaaagaagggattggagagaagctttcaattttcacgtatttagtaatgtcgttcactatatctgtgatattttcgttcttctacggatggaaactgacactagtgattttaagttgcgcaccgattataattctagccaccgcaatagttgctaagatgcaaagcacattaactgaaaaagagttgaaggcgtattcttcagctggagcagttgcagaagaagtattgggtagtattcgtactgtagtcgcttttggaggggaacgtaaggagctcgatagatatcgtaaccgtttgagttctgcagagagtaatggtcgtaaaaaaggtctgttttctggtatcggaggaggcataatgtggttcatcatctactgttgctacgcgcttgcattctggtatggaataagcttgattttggaagaccgaggaaaggacgtgaaagattacaccccagcagtactaattattgtattgttcggtgtgttggcaggagctcaaaatctgggtttatcgtctcctcatttggaagccttttccactgcgaaaggttcggcctcatcgattttctcagtcattgataggataccaacgattgattctatgggagacagcggtctaaaacctcattctattgctggaaacattacgttctccggtgtgcactttcgatatccagctagaagtgatgttcaagttctacaaggattgaacttaactattgaagcgggaaaaactgtcgcgttggtcggaccatcaggatgcggaaaatctacatgtcttcaattaattcagaggctgtatgacccattgaatggtaatgttacaatcgatggtacgaagatcaatgacttaaacataacttggcttagatcttttattggagttgtcggccaagaaccagttctgttcgcaactagtatcgcagaaaacattcgctacggaaatccagaagcaacacaaagtgaagtagaaaacgctgccagaatcgcaaattgccacagttttattaccaagcttcctaacggctatcacacactcatcggggagcgtggtgctcaactatcgggaggtcagaaacaacgaattgccatagctcgagcgttagttcgcaatccaaaaatcctcctactagacgaagcgacttccgctctagatccgaattctgaacggcgagttcaggatgcactagaaaaagctagtcgtggccggactacactggttgtgtctcatcgtctatcgactataaccggcgcagataaaatcgtgtatattgagaaaggagtggtcgctgagcagggaacacacgaggaacttatggctaagagaggattgtattacaatttggttcttgccagtggatctcagaaaaaggaagaagatgaagtggaagctatcaaagagataagccaaggaggaccaaaaagtgtatcagcggacgatgatgcgtattcggatgatgaatctgagtcaaacaaatcggccgaagccgtcatggatgacaaagaagacgtatatccggtatctgtgttcagattggtgaaattgaattctcctgaatggccttatattttgtttggttgcggtgcagccatggttgtaggagcatcgtttccgttgtttgctgtgttgtttggagaaatgtatggtattttatctgtagcggatccagagtatgtcaaggaagaatcaaatttttactcgttgctgttcctcgtactcggattagtcactggtctaggaaccttcttccaaacatacctgttcaatatagcaggagtacgattgacatcccgtctgcgacagaaaacctttaaggcaatcatcaatcaggaaatggcatggtttgatgagtctaacaatgctgttggtgcattatgcgctcgtctctcaggcgattgcgctagtgtccagggtgctacaggtactcgtatcggatccttactgcaggctgcgtctaccatatgtatcggtgtaggaatatcgttttactattcatggaatctaacacttgtctcaatagtagccattcctattgtattgggatcgatcatgttagagtcttggtacacggaatccagtggactgaaagagaaacaatctctagaaagtgcaattaaacttgcagttgaagccatttcaaatatccgaaccgttgcaagcttaggtcaagaaccttatgttctggagcgatactataaagaaatagccaaagtggatgaagcttgcaagaagaagagcagattgagaggagtggtgtttgctttaggtcagataatgcctttcatgggatatggcctggcacttttctatggtggtaaacttgtgtccgaagcagaactggaatacaaagatgtcataaaggtatcggaggcattgatttttggagcttggatgcttggacaagccttggcgtatgctccgaacgtaaattcagcaatgctgtctgcaggacgtttaatgaaactactcgatcgtactccaaaaatgcacaacccctcaagttcctacctatcaacttttgagaaccatgaaggtaatatcaaatttacggacgttgaattccggtacccaacgcgaccaacaattccaattctacaaggactgaatcttgatatcaagaaaggaaacacagttgctctagtagggccttcaggttgcggaaaatcgacttgcattcagctgttgctgcgttattatgaccccgacaatggaaaagttgacattgacgggatcacgacaactgatttccagcttggccgcatacgttcacaaatgggtctcgtatcgcaagaacctgttctgtttgatcgcaccatcgcggaaaatattgcttatggtgacaacactcgcgagataagcatgccggagataatcgaggcatccaaaatggcaaacattcacgaattcatcgtcaatctaccgaaggggtatgacactagcctaggaaccaaaggagctcaattgtctggtggccaaaagcaacgtattgctatagctcgtgctttggttagaaatccacgaatcctactgctggacgaagcaacatccgccttggataatcaaagtgaaaaaatcgtccagaatgctttggaccatgctcgcaaaggtcgtacttgcattatcattgcccatcgattaacaactattcaaaatgcggatctaatttgtgtcattcaaagtggagttgttgttgagtgtggaactcatgatgaacttatggcgcaaaacaagatttacgcaaagctttactccatgcaacaagtggcatagaagtgcacttaggctgcgcgctcgttcagtatttattgttactattaaaatagagaagattaatctcgattaaaagatgaaatcagaagacttattgtgccgtaacgggtgtatgttttatgtacatccgatcatgtaaccaatataaacaagagatgagaatatctcagagctagcttcaagcacagatgttatttttatgttagattaagattagttgtgatatttatgaaacgcctctttagtacttagattttacaatatgcgtgtgtaaaaagctttcattttacgacaaaaaatcaaataatgaaacgatacccattttttaaaataatacacaaaatgtttaacgtatgttgaccgcttcttgactaacaaaacacacatcatcatagagcataaattacagagcaatatgtttacaacatacattttaccaaaccatgccaaggaacaaaacgtaagataccagataagaacgcatatatagttactcaatattgatgaatctgcaaagtaaagattttaaagaatttccaccaccttcccactcgctattgctatcactataaatagttatgaaatacatttgtatcaattt

SEQ ID NO: 195 - DNA, 3964 bp, Aedes aegypti
atagcggatctcactaaccaaaaagcttctccgtcgtgtgttgttttgcgtttcgcttcttttttgacatgcttgcaattcactcgcttttgtaactcgaatctgcttactcgtgtcgcgtagtgctcgtcaaattagattaacaaaaaaacgcgttcgaatttatttaccatacattttagattgattcgcgatttgtgcgcacctgttctgatcgttggtgaagagtgcattcgatatctaccggtaaaaggagtttctaaacgtacgacggaaaaatgtcctcgcggttgaatttagttcgcgcgtttctcagtcagtctggttcatccggagatgccgcggaacgcacgaaggccccatcttccggcgaagcccccagcgaaggggtcgacagtggttttcaaacgcgcaccccggaaagcaccccgaccacacaacgtaaaggaattggacgcggccagttgacctcggatgctggcagagccaccggaacatctggagcggattcatcggaatcggatgacaaacagtccagtattggatctgccatgccgtccttgagcggtcgcggaagggctcagtttattcaggcccttatccgtcaaccgttcgaaccggcgccatcggttgtctccgacgattcgtcctccatggtgtctgcaagggtgtcgcaaattgcctgcggcagaggaagattcatccagcagctgctgaacacggctgcggatgccgagagcatcgaaacacaatcgaacggaaaacatgacgaactctccgaagcggtttcacaggtcaccatcgctgaaacggtcgaaacggtggaaaaagcaccggtcatcaagactggatcgagcggaactcctgtccgactgatgaccaactacattcgtttggcttgtgatccggaccgcggaatctacgaatacgaagtacggttccatcccttggtggactcgaaagcaattagagctcgttacatcgcacaacacaaggatgtccttggcaatgctaaaacgttcgacggcgttaagctgttcctgccaaagaagcttccccaagcggaactcaccctccaatcgaagaaccccgtcgacggccacgaagttacgatcaagatcatttacaagcgcaaacagcgaatgagcgagaacattcaattctacaacattctcttccaacgcattatgaaagtcctgaagatggtcgagatgggtcgtaagaactttgacccctcggccccgaaactgattccgcagcatcgtctggagatctggccgggatacgttagcgctgtggacgaatatgagggcggcttgatgttgaatctcgacgtgtcccatcgtgtgctgctccagacgacggttttggatcacatccgaacgctggcaagggccaatccccaagattacaaaaacatggccaccaaatcgctgctgggagccgttatattgacccgatacaacaacaaaacgtaccgtatcgacgacattctgttcgatcagaatccgaccatgactttcgaagccaacgggcagccaatttcgtacctgcagtattacaaacagcagtacaacatcgacatccacgatctgaagcagccgctgctgattaatcgcaaggagcgtcgtgtatctggccaagacaaaccgatggaaatgatcatgtgcttgatcccggagatttgctaccttaccggattgaccgacgagatgcgaagcgatttcaaggtgatgagagacatcgctgccttcacccgggtatcgccgaatcaacgtttaaacagcatgcgtcaattctgtcgcaacgtgaacgaaaacaaggaagcccgggaaattctggaagtctggggcctaacactggacatgaatgctcttatgatggagggacgttgcttcaaagaggaacaggtgaacttcagtggggtgaatgtgggtgttggaaagagtggagatttcaaccgggccgtcaccagtaacaacgttctgcaagcggtcaacattcgcaactggctcttggtgcacacggcgaaggacaccagaattgccaaatcgtttatggattgcgttgaacgcagctgtcgtcccatgggtattcaaattggaccgccagcaattgaggttctgcaggccgacaaaaccgagctgtacgtacagctgctgcgtacgaaaattcgccaggaaacgcaaatcgttgttataatctgcccaacgtcaagagatgaccgatacgctgctatcaaacgtatttgctgttcggaaatcccagtcccatcgcaggtcatcaacgctcgcactctgagtaacgaggccaaaaaccgagccattgtgcagaagatcatcctccagatgaactgcaaactgggtggaacgctgtggagcattcgtatcccgttcgacaacgtgatgatcgccggcatcgatacctatcacgatccgaagcagaagagcaactccgtttcggcattcgtagcttcgttgaatggtgactacaccagatggtattcacgtgcttgcattcagagcaagaaggaggagttcatcaacggattgtgtgcatcgatggagaagtctttgaaggcttaccagaaggcaaactgtcagctgccgaagaagattatcattttcagagatggcgttggagacggtcagctgcgaatgtgctccgagtacgaaattccacaactgcaggagtcgtgtaagctagtggaaccggattacaatccggagataacattcattgtcgtccagaagcgtatcaacacccgtatgttccgtatcgacggtaacaacttggaaaacccgaaccctggcaccgttctggatcacaccatcacccgacgcaaccacttcgattacttcctggtgccgcagtcggtccggcagggcagcgtctcgcccactcactacattgtggtccacaaccagtcgaaccactcgccggacatcctgcaacggttgagctacaaactgtgctatctgtactacaactggcccggaagtgttcgagttcctgcttgctgtcagtacgcccacaaaatggcctatctcattggacagtcggtgaaacggaatccggacgaggcgctgaacgacaaactgttctacctgtgaatggcgtcatcggtttaccgccagctgggagttttgttttatccaaagggactgagttgttttcgatttatgttttcttctcgaaatgagatgtgttttatcaagtttctatagagagagagatacctaaaatgagtgccttgttaatcagcgacatgattttcccgctgagataacctataaaatgtttttgttacaacatattcggtaactaaagactgaattccatctatttttgagttacctcatcaatgacgcgttttgagtgaactagaagagaaaaatctaacgtctttgagctcctcgctcgccctggttttcaatttcaggcactcgatacattttaaaacatttcaacacatctgaacacgccaaatacgtcgttggctgtttgtaccagagtcgtacaaattacattctccacaatatgttaacttctaacgttggactgagcaatgataacgttctactaacctacatttattagaatgaaataacagttagtgcataatcaaggaagtttttcgttatttttttataaactccaatcctatcggaaggtacctacaggatttaagtgcccatccgatggatcgttagggagaaatctcatattttatctaataaaataaacataaattcataaatatgaacctgtattcattaattatccgaaactcgaaaacagttaaactattgtaaatcctcgcaaactatctcaaacatcttattggtagcgctatattacgttttaggtgcaccaaggtggtgagaccgtcacaaacaattttcatcttgctccgcctacccctaacacttatcgacatatgtttaaccttcgggctgtcgcg

SEQ ID NO: 196 - DNA, 2583 bp, Aedes aegypti
atgtctgaccgttactctcaagggcgctaccgtgcgcgtggttacacagaagtggggtcgccccacgaaggccgtggaagtccttcttctcgcccagcctacagttcgcgcgatgaaggtcgatcgagggaaccgagagaacaccaccgtgaatcatcggcaggtggccgtgaacggtccggaaacggtaatggaggagatcgtcctggtggtgaacgcggcgaacgacgccaacgcagtcgccgtggcgttggagatacgctccgtacccgtgctctggatgctccatcgaagcatggtaccacgggtcaggctctccagctgaacagtaactatttcaaactgttgaaacacatcgagtggactctgtaccagtatcggttggacttctccccgcaatgcgccagcatgcgtctcatgcagggattggtcaacgaacacaagaaaatcttcggaggattcctgtttgacggaactcaactgttcatggtcaacaaattgagaagcgatcaactgacattgcaatccagacacgaacgtactggagatgtctaccagctgcgcattgtccatacgggctccgtggatatgaccaacgaagccggtatccaagtgctgaatttgattttgcgtcgcgctatggcgggtttgaatcttcagctggtcggtcgtaacttgtacgacgcggctgccaagatcgccattcgtgagtatcaaatcgaactctggccaggttacattaccagtattcgtcagcatgaacaggacattctggtgtgttgcgaaattgcacataaaactatgcgtatgcagacatgctacgacatcttgcgcgattgtcagaagcacgatcgtaactacaaggattcgttcaaacgtgccgtacttggtgtcgttgtactgaccggttacaacaataaaacctataccattcacgacgtcacgtttgaaaccactccggagagtacgttcgataccaaggccggtaaaacgtccttcattgagtattacaaacagaagtacaacattcgcattcgtgatccacatcagcctatgttgctgtcgcgagccaagaaacgcgatctgcgcgctggaggcagcgaactcatggccctcgttccagaactgtgccagatgacgggtttgaccgatcaaatgagatctgatttcagaatgatgcgtgccatggccgatcacactcgtctcaacccggatcgacgtattgaacgattggagacgttcaaccgtcggttgcaaacgtcgccagagagtatggaagtcttcaagacctggcagatggagcttgaccgacgtcttatcgatcttcctggacgattgttaccacaggaaatgatcttcttctccaccacagccaatggcgtacaggctggagaacaggcagattggacggcccatttccgtaacaatcccatgttcgctacggtgcggttgacgcgatggtatttgatcgtaccgaaccgggcttcacgtgaggccaacgatttcctgggctgcatgattcaagccgcacgcggtatgcgatttgagattagtaactgtgaggtggtcaccattccggacgacaacccaggtacctatgtgcgaacattggacaatctgctgaataaggacccgcagttggtgatgtgcgtcgtgaccaacaataaggccgatcggtacaccgccatcaagaagaaatgttgcgttgatcgtgccatcccaacgcaagtcatggtacaaaaaacgatcacaccaaagggtggtaatgtacgaacactcatgtccgtggctacaaaggttgtcatccaaatgaactgcaaactgggcggtgttccatggaaggtcaagatcccactcaacggtttgatgaccattgggttcgacgtttgccatgatggaaaggacaagtccaaatccttcggagccatggttgccacgttagatcacgataacaagggtactccgaaattcttctcgactgtgagtcagcacacgcacggtgaagaaatttccaattatctgcccatcaatacggtcaaggcattgaacgaatatcgtaaggaattcggagagctgcccaaacgtatcatcttctaccgagacggtgtcggcgagggtcaacttcactacgtgtacgagcatgaagttaaatccatcgttgagaagctgaaccagatctacaaatccgctggaatcgatcaggatgtgttgctcaccttcttcattgtgaacaaacgaatcaacacacgcttcttcgaccatcgtcagaatcccagacccggaacagtggtcgatgatgtcgtcaccctaccagaaagaactgatttctaccttgtgtcgcaatcggtccgtcaaggtacggtttcgccaacggcgtacaacgtgatctacgatacatcaggcctgaagatcgatcacctgcagatgctgtcgtacaagcagtgtcacttgtactacaactggtccggaacgactcgtgtccccgcggtatgccagtatgcacacaagctgtccttcctggtggcgcagttccttcaccaggcaccgagcaatctgcttgaaaagaagctgtacttcttgtaa

SEQ ID NO: 197 - DNA, 2135 bp, Aedes aegypti
atgtccactttggaacgtgttataactcaactcagtgccggtaaagttcaaggttgcaaatctgcactaccgaacgggaatagctattgcttatacagaggaatcccatatgccaaacctcctgtcggtgagcttcgttttcggccacctgttgctttggaaagattcgaggaagaagtgctggattgcagttatgagcggagttcctgttatgcgtatatgcatttcccacccacggtggccgttagcgaggattgtctgtacgcaaatgtctacaccccgatagatcctaccgaaatcgaacggacaattccacttcctgtaatggtttggatccatggtggaggttttatagcaggaagcggcgatgcagctatttatggtcctagatatatagtgcaggagggggtcattgtggtgacgttcaactatcgattgggtcccttgggattcctatatttcccagagaaaggaatttacggtaatatgggattgaaggatcaacggttccttctgaaatgggtacaagaaaacatttcaaaattcggaggagatcccgataatgtcacgatttttggtgaaagtgcaggtggtgcatcatgtcatttgcaatatctgtgcgaatcctctcggaagtatttccacaaggcaatatgtcagtcgggagtagctgtcactgtgtggaccgagcagatggatgcagtttccaaaacacgcaacttggccaagttcgtaggctgtgtagcagagagcgatgatgaaatttacgaaaccctcatgagtgcacccgctgacgatctcatagctggcagtgaacaatgtgtttcagatctagatcgttcgatttacagaatctttgcattttctccagtagtagaaccaaaggaatcagaagatcctttcataaccgaaaactacgtggatattctaagaaacccgaatatgacgaatattccactaatactgggcttaaccagtaatgaagccatttgcttcattcaaaacctctcggttgaactttatgcaaatgatgcaaaactgttcgtgcctcctcaattggctgttccagaagaccgattgcttcaagttggagaagaagtcaaacggttttattttgaaaatcgaactgtttcatctgaaaacttgcagtttcttttggattttgtttctgactgtatgtttgtcataccggtttgtgtggcaagcgaattgcattcgagatatcagcacagtgctccacaatatttctactgcttttcatttgaaggtaaatcgaatcacataaaagcaatgttccaaaacgactaccaactagaaggtgcagctcatgcggatgaattgacatacatgtttgaggcaagctttttcaccaccgaggagatggataccgatagccaggaatacaagatacgcaaagctatgtgccgtatgtggacaaattttgcaaaatgtggcaatccaacgccggatgaagatcaactggatattttgtggcagccagtagagaaaatagatcctgatcaggagaaatataacatcagagcgctggacttgaacgaaccatccaaaatggtagaccacccattcgaaaagcgagttaatttttggaaacgattgtttgaaaaatacggtgggaactatttgcttcatcgagcattgcaatgaaatccaacttcttccaaattgtgttcaatcataaaaatgccctttgaatatatataaccctcgatgcttttataaaagaaatactagagaataatataattaactgattgatattaatccaaactactttcgtgtaaaacgatgaaaataaatttattctcaatgttttgaatacatttttgaatgcataaccttagaacatgttcatgtgggtgatgtggatgctgtgcactaccttaataaggatgtgcttgaagttgttgaagacttcttcggggatacgactgcgatgattatgttctaggttttggctttcagtctattattaaattatgtacaaatatacacagaaaaaaatatttaattttcaatgtgatgtaaactgaagtctactgtaaaaataaatcaaattcgtgtgttgttacagcatcatgtaaattcaaatgaatatacattcaattttcaactag

SEQ ID NO: 198 - DNA, 1284 bp, Aedes aegypti
atggacggacatcacagccgatcgggatatacatgcaaggcatgccaccgcccagactcgtcaagtgctcatatgatcatctgcgatcaatgccgcctgtgggagcacttcagctgtaccggcgagagcgagtccgtgccgagtagaccgtttatctgcaggcagtgccggggagagaatgcagcaggatcgactatctcttctcgactgcgatcacaagcgaaacgagcgccgtcaggtaacgtgtcagggggcaaacctgcttcaaatattggcagcaagatttcgtcggtagcatcgagtagccggtcgtcgatcgtgaaggcgcggctggagctaatcgaggaagaagagaggatgaaacagaaagagcttgaagaagaggaagcgattaagaaactggaacatgaagaggcccagagacaactggaagaaaagaagaagcgaatggaagagcaaaggaagttggctgaggaagaatcgcttttaaggcagagcaaacttgtggcagagaaagcacgtctgctcaaacagcagtcgatacgccgtgaatcactggagaagaaaaatgaagtcattctgcagatttccgaacgtggcagtgtggtggaatcggcaacaagttcccgagaaaaggtcgcgagttggttgacggctaatatgccgaacgggaagtccgacaggaatacattgggaaatcatggcaacctccaatatgattcggttcctgcggccccgcatagtgtagcgtcgttgatcatcaatgtaccttcaagagtaacggagataccgtcagaattaccgcctgcaccccaagtggcagaaactcataattcttctccacgtcagcgtttccctgggcccactggcattcctcatgtgagtggtaggatcgggtatcacgttcgatctactacaatccgtgaaccgttccagcagttcgtaactagttcaaggcctttgcaaacaaatcctttgccagttcacccatatccaccgtcgattcattcatctcgtggacacagtgatgggggtgaacacactccacagatatttcgccgacaagagccttcttggaccacacctggtctgtgcgacgcaattcacgatccacctctaacacgtcgatccgtagagccaaatcttttctctcacgaatctcgtccggagcaatttcgtcacttgggtggaccaacgtgttcacgggaggggggatgtcacgtcacccccctgctacaaatctactgtggtcgattcactgtggccgttcaagtggtcaactaa

SEQ ID NO: 199 - DNA, 1512 bp, Aedes aegypti
atgcttatcttattggcttcggttgttgtgctctcgatcggtttagccgtgtacttttatcagcagtttgcgaatcgtttgcactatgctgctaagattggtggccctaaagggtatccccttttgggaaactcgatccagtacgggacaaagtccccggtggagtttctacaggaagtgcagaaaactaacgagcagtgtggaaagttctatcgattgtggattgggcccgatttgattttccccataactgacgccaagttggcgattctcagcagtcagaaattgctggacaaatccgttcagtacgattttatacgaccatggctgggaaatggattgctcaccagtaccggaaggaaatggcactccaggcgaaagatcatcactccgacattccatttcaagattttggaacagtttgttgagatttttgatcaacagagcaatatcttcgttggtcagttgaaatcgaaagcgcagagtggggaggactttgatgttttcccagtggtaacattgtgcgctctggatgtgatctgtgaatccgccatgggaacaaaggtcaacgctcagctcaattcggattcgaaatacgtgagagccgttaaggacatggctactgttgcgatggctcgctcattcaaagcatttgcacggttcaacttcacgttctacttcaccccgtaccgaagaatgcaagacaaggcgctgaaagttctccacgactacacagatagcgtcattcgttcacggcgcctagagttagccaagggcgccttcacaaagtcagacgaaaacgagaatgatgttggaatccgtaagaaagtggccttcctggacatgcttctgcaggccacagtagacgggagacccctggatgacctcgaagttcgcgaggaagtcgatacgttcatgtttgaaggtcatgacacgacaacttcggccatctcgttcctcatcggcatcttggccaagcacccggatgtgcagcagaaagtctacgacgaagtacgaaacgtgattggcgatgatctgaatgtgtcagtcacgttgtcaatgctgaaccaattgaactacctcgacttggtaatcaaagagaccctaagactgtacccatctgtccccatttatggtaggatgttgttggaaaaccaagaaatcaatggcactgtgttcccggcaggatccaacttagcaatattcccatacttcatggggcgtgatccggaatactttgaaaaccctctggaattcagaccggagcgattcgcggtggaaacgtcagccgaaaaagctaatccctaccgttacgttccgttcagcgccggaccaagaaactgcatcgggcaaaagtttgccgtggcggaaatcaagagtttgatcagcaaactggtacgacactacgaggtgttgcctccaaagcagcccaattcggagcggatgattgccgagctggtgctaaggccggaaggaggcgttccagtgaggatcagaagcagggtgcggtag

SEQ ID NO: 200 - DNA, 360 bp, Aedes aegypti
atgaagttcactatcgccatccttgcattgattctggccatcagcgctgaagtatccccagttccatacgcccatgatgtagtggcctactccaacccttggcctctgaagaccagttcggtttggccaacctacggtggactgtacggcggtaaggtcactgtggttccggaagttgcctacgaaaaatacgcttacccgaatgtgtatggaagctggcctgcctacggaaagaactggggatactcttctccggttgttgctgtcggagcaaaggtggttgacaacggcctgtggaattacggaggatatggctacggacatagatacggtctgggatataacaaggagtggctgtaa

SEQ ID NO: 201 - DNA, 1482 bp, Aedes aegypti
atgttgatttatctcaccgtgttggcgctgaccttggcggtcttgtggattcgcaagcgctactcatactggatggatcgtggaataccgtatgtggagccaagcttcccagccgggaacttacgaggaatgggacggaaggaacatctatcgtcccaaatgcaacgatgttataaggagctcaaaggcaaaggtccagttggggggatgttcttcttcatcaacccggtagctctggcgatggacttggacctgatcaaaagtgttctggtgaaagatttccaatacttccatgatcgttcggtttactataatgagaaagatgatcctctatcggctcatctgttcacgatggagggtgccaagtggaaaaatcttcgagcaaaattgacgccaacgttcacttcagggaagatgaaaatgatgtacccaactatcatcggagtggcggacgaatttcagaagctaatgaaaagtgaagtgtccagcaatgctgaaatcgagatgaaggagattcttgctcggtttacaaccgacgtgattggcacctgtgcttttgggttggaatgcaatagcttacacgatccggatgccaagttcagggccatgggacgcaaaatattttcgtttgccaatgggcggttcctgaaggcagttatcgctcaacagttccgttcacttgcgcgatcactacatatagctttggttgataaagaagtttcagattttttcctgggtgctgtgagggatacaatcaagtatcgtgaagagaataagatcgagcgaaatgatttcatgagtttattgatgaagctgaaggatgatggaaataccggcaacacagagacattgactgttgaggaaattgctgcacaagcattcgtgttcttcttagctggtttcgaaacttcatcaacagcaatgtcgtactgtttgtacgaactagcgcaaaattctgaccttcaaaataaagcacgcaaaagtgtgatggattcgatcaaaaagcacggttctttaacatatgaggcgatgcaagacatgcagtacatcgatcaatgtatcaacgagtccctaaggaagtatcctccggcatcaaccttgaccagatcggtttccaaggactacaaactccccaacagcaacgttgtccttcagcagggatccactttgatcgtgccggtgtacgccctccaccacgacgcagaatactatcccgatccagaaaagtacaacccggatcgctttacaccggaagaagtggccaagcggaatccttattgtttcctgccgttcggtgaaggaccccgcatttgcatcggaatgcgcttcggtatgatgcaagcccgcgtcggattggcctatctgttgagggatttcagtttcacactgtccagcaagactccggttccgttgaaaatctccccgcgcagccctgttctcacctcggaaggtggactgtggttaaaggtccagaagctgtag

SEQ ID NO: 202 - DNA, 1605 bp, ;Aedes aegypti
atggaagtggaactcctacatgtgggagtactggtggcgatcgtcgcctttctgtaccgctggataacgcgcaacaatgactacttccacgataaaccgatcccgtcgatggccgttacgccgttcctgggtgcttcgggacctctcctgctgcgaaaggtcaccttcaacgactttgtccaatcaatttacaacaaatatcctggagtgaaagttttcgggatgttcgagacgatcacgccgttcttcgtgattcgcgatccggagttgatcaagcagatcggcatcaaggatttcgatcacttcgttgaccaccgtccgacgttcggactggacgatgaaacggcggagcatccgaaggcgctgtttcgcaagacgctgttctcgatgaccggtcaacggtggaaggagatgcgcgctacgctgagtccggccttcaccggcagtaagatgcgacaaatgttctcgctgatgagcgaatgctgcgacgaaatgatgaagcactacctggataaggcgaagggttcgggaagggtggaagtcgaaatgaaggacttgctgagtcgaatcagtatcaacgtgattgcgtcgtgtgcttttggaatcaaggtggactgcttcaaagagcaggaacatgagttcttgtatcacggcaggaagatgatgggcttcggccggccgattgtgattgcgaggatgttggccatgcgtgtcttcccgaagttcgctgcgaagttcggaatcgacttgctcgatcgagagcaagcgaactacttcacacatgttttccaagagactattagggctcgggaaagccatggttacattcggcatgatatgatcgacctgttgctgcaggctcgcaagggtacgctgaagtatcaggaagagaaggatgaccaggaagggttcgccaccgtacaggagtcagatgtaggcaaagcagacgtgtcgaagagtatgaccgaagcagagatgatcgcacagtgcttgatattcttccttggtggattcgacactgtgtctacctgtgccatgttcactgcctacgagttggtccgcaatccggaagttcaacacaagctgtacgaagagattaagcagacagaaaaggagttggaaggaaagccgctgagctacgatgcacttcagaagatgaagtacatggatatggtagtgtcggaaacactgagaatgtggcccttggctccggctacggatcgtctctgcacccaagactacaccattgatgacggccagggcgtgagattcacgatcgacaagggaacgtgcgtttggttcccggcagctggactgcaccacgatccacagtatttccccaatccggagaagttcgatccggagcggttcaatgacgaaaacaagcggaacatcaacctgggagcgtatctgccgtttggtatcggcccaaggaactgcatcggatcgcggtttgcgctgatggaggtcaaggccgtcatgtatttcattctgctcaagttttcgtttgtgcgaggcgcgaagacccagataccgatgcagctacggaaggggttcactaatttgggcccggagaatggaatgcacgtggagctgaagctgcgg

SEQ ID NO: 203 - DNA, 451 bp, Artificial Sequence
  double stranded RNA
cttcaacgacttcgtccgatcaatttacaacaaatatcctggagtgaaggttttcggaatgttcgagacgatcacgccgttcttcgtgattcgcgatccggagttgatcaagcagatcggcatcaaggatttcgatcacttcgttgaccaccgtccgacgttcggactggacgatgaaacggcggagcatccgaaggcgctgttccgcaagacgctgttctcgatgaccggtcaacggtggaaggagatgcgcgctacgctgagtccggcttttaccggcagtaagatgcgacagatgttctcgctgatgagcgaatgctgcgacgaaatgatgaagcactacctggacaaggcgaagggcacgggaagggtggaagtcgaaatgaaggacttgctgagtcgaatcactatcaatgtgattgcgtcgtgtgcttttggaatcaaggtggac

SEQ ID NO: 204 - DNA, 20 bp, Artificial Sequence
  Single strand DNA oligonucleotide
ccgtttggtatcggcccaag

SEQ ID NO: 205 - DNA, 18 bp, Artificial Sequence
  Single strand DNA oligonucleotide
gtctttgcgcctcggacg

(19) United States
(12) Patent Application Publication
(10) Pub. No.: US 2017/0051285 A1
(43) Pub. Date: Feb. 23, 2017

(54) COMPOSITIONS AND METHODS OF USING SAME FOR REDUCING RESISTANCE TO MOSQUITO LARVICIDES

(71) Applicant: Forrest Innovations Ltd., Caesarea (IL)

(72) Inventors: Nitzan PALDI, Moshav Bar Giora (IL); Humberto Freire BONCRISTIANI JUNIOR, Odenton, MD (US); Eyal MAORI, Rishon-LeZion (IL); Avital WEISS, Karkur (IL); Emerson Soares BERNARDES, Sao Paulo (BR)

(21) Appl. No.: 15/306,772

(22) PCT Filed: May 4, 2015
(86) PCT No.: PCT/IL2015/050467

§ 371 Date: Oct. 26, 2016

Related U.S. Application Data
(60) Provisional application No. 61/988,234, filed on May 4, 2014.
(60) Provisional application No. 61/988,235, filed on May 4, 2014.
(60) Provisional application No. 61/988,246, filed on May 4, 2014.
(60) Provisional application No. 61/988,236, filed on May 4, 2014.
(60) Provisional application No. 61/988,237, filed on May 4, 2014.

(51) Int. Cl. C12N 15/113; A01N 65/03

(52) U.S. Cl. CPC C12N 15/113; A01N 65/03; C12N 2310/14; C12N 2320/32

(57) ABSTRACT

A method of enhancing larvicide susceptibility in a mosquito larva is provided. The method comprising introducing into the mosquito larva an isolated nucleic acid agent comprising a nucleic acid sequence which specifically reduces the expression of at least one larvicide resistance gene product of the larva, thereby enhancing larvicide susceptibility in said mosquito larva.

### FIELD AND BACKGROUND OF THE INVENTION

The present invention, in some embodiments thereof, relates to compositions and methods of using same for reducing resistance to mosquito larvicides.

Mosquitoes are the major vectors for a large number of human and animal diseases, including malaria, yellow fever and dengue fever. Over 1 million people die from mosquito-borne diseases every year, and hundreds of millions more experience pain and suffering from illnesses transmitted by mosquitoes. Mosquitoes of the genus Anopheles, Aedes, Mansonia and Culex are the greatest health concern.

Malaria, the most widespread mosquito-borne disease, affects 350-500 million people each year. The most recent figures by the WHO indicate that malaria causes an estimated 660 000 deaths each year. Dengue Fever infects between 50-100 million persons, causing an estimated 25,000 deaths and an enormous economic cost in affected countries, which rivals that of Malaria. Other serious illnesses transmitted by mosquitoes are on the rise. Chikungunya, Yellow Fever and Eastern Equine Encephalitis are painful and debilitating diseases, which can in some cases prove fatal; all are increasing in prevalence. Although Malaria is transmitted by several different types of mosquito, Dengue Fever is primarily passed on by just two related species, called Aedes aegypti (the Yellow Fever Mosquito) and Aedes albopictus (the Asian Tiger Mosquito). Both of these mosquitoes also transmit Chikungunya, Yellow Fever as well as several other viruses, making these tiny insects some of the most dangerous creatures on the planet. Another medically important mosquito genus is Culex. Culex and Mansonia species are vectors of lymphatic filariasis (elephantiasis), Japanese Encephalitis, Rift Valley fever and arboviruses, such as the West Nile Virus.

There is neither specific medication nor vaccine for Dengue fever. The only way currently to control the disease is to control the mosquito, Aedes aegypti, which spreads the disease. There is no cure for yellow fever but there is a vaccine; however it is expensive and not available in many parts of the world. No currently available drug regimen guarantees 100% protection against Malaria, and prevention of infection involves taking symptomatically effective antimalarial medication in addition to prevention of mosquito bites.

In order to prevent human disease caused by the viruses and parasites mentioned above, integrated mosquito management programs (IMM) have been developed, to optimize the control of mosquitoes in an economical and environmentally friendly way, exploiting the known vulnerabilities of mosquitoes in order to reduce their numbers to tolerable levels while maintaining environmental quality. Prudent mosquito management practices for the control of immature mosquitoes (larvae and pupae) include such methods as the use of biological controls (native, noninvasive predators), source reduction (water or vegetation management or other compatible land management uses), water sanitation practices as well as the use of registered larvicides. However, larvicide efficacy is now threatened by the rise of resistance in target populations, occurring worldwide in all major disease vector mosquito species and spreading at a rapid rate.

### Larviciding is an Ecologically Safe Approach at Controlling Mosquitoes

Larviciding is a general term for killing immature mosquitoes by applying agents, collectively called larvicides, to control mosquito larvae and/or pupae. Most mosquito species spend much of their life cycle in the larval stage, highly susceptible to both predation and control efforts, as they are concentrated within defined water boundaries, immobile with little ability to disperse, and accessible.

Larvicides may be grouped into two broad categories: biorational pesticides (biopesticides) and conventional, broad-spectrum chemical pesticides. Conventional pesticides are generally synthetic materials that directly kill or inactivate the pest. The chemical compounds mostly used as larvicides are: (1) Organophosphates and; (2) Surface oils and films. Biopesticides are certain types of pesticides derived from such natural materials as animals, plants, bacteria, and certain minerals. Biopesticides fall into three major classes: (1) Microbial pesticides consist of a microorganism (e.g., a bacterium, fungus, virus or protozoan) as the active ingredient. The most widely used microbial pesticides are subspecies and strains of Bacillus thuringiensis, or Bt. Each strain of this bacterium produces a different mix of proteins, and specifically kills one or a few related species of insect larvae. (2) Plant-Incorporated-Protectants (PIPs) are pesticidal substances that plants produce from genetic material that has been added to the plant. (3) Biochemical pesticides are naturally occurring substances that control pests by non-toxic mechanisms. Biochemical pesticides include substances, such as insect sex pheromones, that interfere with mating, as well as various scented plant extracts that attract insect pests to traps.

Larvicides Currently in Use with the Goal of Controlling Mosquito-Borne Diseases

From the earliest days, two types of larval control were employed: Larviciding as a temporary control method and ditching as a permanent control method. Historical larvicides include waste oil or diesel oil products, Paris green dust, an arsenical insecticide, use along with undiluted diesel oil, and dichloro-diphenyl-trichloroethane (DDT), used as both an adulticide and a larvicide. Mosquitoes became resistant to DDT, and its use was discontinued in the late 1950s. As resistance to DDT increased, the use of malathion, an organophosphate (OP) compound, increased, but resistance was soon observed.

The term organophosphate (OP) refers to all pesticides containing phosphorus, acting through inhibition of the activity of cholinesterase enzymes at the neuromuscular junction. Temephos is currently the only OP registered for use as a larvicide in the US.

Surface oils and films used as larvicides include oils and ethoxylated isostearyl alcohols. Larviciding oils are non-selective, and mosquito control efficacy is limited to those species, which breathe air at the water surface. They have a low toxicity, however, both their odor and appearance may be objectionable, precluding widespread use in some areas.

Biolarvicides are comprised of two major categories: (1) Microbial agents (e.g., bacteria) and (2) Biochemical agents (e.g., pheromones, hormones, growth regulators, and enzymes). Biolarvicides are generally highly target specific, and inherently less toxic than conventional pesticides, effective in very small quantities and often decompose quickly, thereby resulting in lower exposures and largely avoiding the pollution problems caused by conventional pesticides. Regarding microbial agents, controlled-release formulations of at least one biological pesticidal ingredient are disclosed in U.S. Pat. No. 4,865,842; control of mosquito larvae with a spore-forming Bacillus ONR-60A is disclosed in U.S. Pat. No. 4,166,112; novel Bacillus thuringiensis isolates with activity against dipteran insect pests are disclosed in U.S. Pat. Nos. 5,275,815 and 5,847,079; a biologically pure culture of a Bacillus thuringiensis strain with activity against insect pests of the order Diptera is disclosed in U.S. Pat. No. 5,912,162; a recombinantly derived biopesticide active against Diptera including cyanobacteria transformed with a plasmid containing a B. thuringiensis subsp. israelensis dipteracidal protein translationally fused to a strong, highly active native cyanobacteria's regulatory gene sequence is disclosed in U.S. Pat. No. 5,518,897 and a formulation of Bacillus thuringiensis subspecies Israelensis and Bacillus sphaericus to manage mosquito larvicide resistance U.S. Pat. No. 7,989,180 B2.

Biochemical agents such as Insect Growth Regulators (IGRS) which control flies by interrupting their life cycle, rather than through direct toxicity, are also considered to be a biochemical pesticide. The IGRS mimics naturally occurring insect biochemical that are responsible for insect development (e.g. Methoprene, a juvenile hormone (JH) analog), preventing the mosquito larvae from developing into adult flies.

### Continuous, Long Term Use of Larvicides/Adulticides Create Selection Pressure for Mosquitoes to Develop Resistance

Resistance has been defined as ‘the developed ability in a strain of insects to tolerate doses of toxicants that would prove lethal to the majority of individuals in a normal population of the same species’. Although individuals with resistant genes to a given insecticide are rare in normal populations, widespread use of a toxicant favors the prevalence of the resistant individuals. These individuals multiply fast in the absence of intraspecific competition and, over a number of generations, quickly become the dominant proportion of the population, rendering the insecticide no longer effective. Historically, the patterns of insecticide use for controlling mosquitoes has led to the evolution of insecticide resistance to the chemical compounds DDT, BHC/cyclodienes, organophosphates, carbamates, and pyrethroids. Not all species or even all populations of any species are resistant, but the pattern of adoption of new insecticides, followed by the eventual loss of use of that insecticide due to insecticide resistance, has occurred frequently enough and widely enough to serve as a grim reminder that insects, especially mosquitoes, have the genetic capacity to evolve and adapt to adverse conditions.

Resistance of insects to insecticides can be the consequence of various physiological changes, such as mutations of the proteins targeted by the insecticide (target-site insensitivity), reduced penetration or sequestration, increased biodegradation of the insecticide due to enhanced detoxification activities (metabolic resistance) or behavioral resistance resulting from behavioral alteration (e.g. changes in feeding patterns). Mutations in the target site proteins are probably the best understood pyrethroid resistance mechanism found in insects, and involve non-synonymous mutations of the gene encoding the paratype voltage-gated sodium channel (VGSC) expressed in the insect central nervous system targeted by pyrethroids. By contrast to target site resistance, metabolic resistance involves potent regulation of the mosquito detoxification system in order to counteract the chemical aggression caused by insecticides. Metabolic resistance consists of elevated levels or enhanced activities of insecticide-detoxifying enzymes in resistant insects, resulting in a sufficient proportion of insecticide molecules being metabolized before reaching their target in mosquito nervous system. Detoxification enzymes typically linked to insecticide resistance include 3 major gene families, the cytochrome P450 monooxygenases (P450s or CYPs), the carboxyl/choline esterases (CCEs) and the glutathione-S-transferases (GSTs), but other enzyme families may also be involved such as UDP glucosyl-transferases (UGTs). Cuticular resistance is characterized by a modification of the insect cuticle leading to a slower penetration of the insecticide reducing the amount of insecticide molecules within the insect. In Aedes aegypti, selecting mosquito larvae in the laboratory for several generations with the neonicotinoid insecticide imidacloprid led to the constitutive over-transcription of multiple genes encoding cuticle proteins. Of these three types of mechanisms, metabolism and insensitivity at the site of action are the most important. However, a reduction in the rate of penetration aids the other types of mechanism in a synergistic way.

In addition to pesticides and insecticides, chemicals commonly used in agriculture also include fertilizers, herbicides, fungicides and various adjuvants that increase their efficiency. Although these compounds are usually non-toxic to insects, their presence in breeding sites has been shown to affect tolerance to insecticides via the modulation of their detoxification systems, such as enhanced GST activity, induction of P450s and the induction of CYP genes.

Resistance has also been described against biolarvicides. Specifically, the development of resistance in Culex quinquefasciatus to the Biopesticide Bacillus sphaericus (B.s.) has been noted by Rodcharoen et al., Journal of Economic Entomology, Vol. 87, No. 5, 1994, pp. 1133-1140. In addition, resistance to methoprene was soon demonstrated in several species. As a result, it is essential that new insecticides, be able to overcome larvicide/insecticide resistance by blocking detoxification enzymes and increasing the penetrance of conventional pesticides into the larvae/adult mosquitoes.

Externally Delivered dsRNA can be Effective in Gene Regulation and Provide Phenotypic Effects in Adult and Larvae in Mosquitoes

In studies involving insects, direct injections of in vitro-synthesized dsRNA into virtually any developmental stage can produce loss-of-function mutants.

Studies on feeding dsRNA revealed effective gene knockdown effects in many insects, including insects of the orders Hemiptera, Coleoptera, and Lepidoptera. US Patent Application Nos. 20120309813, 20120041053 and 20130237586 to Whyard et al. disclose compositions comprising dsRNA and different transfection for delivery of dsRNA to arthropods, including by feeding of arthropod larvae. Feeding dsRNA to E. postvittana larvae has been shown to inhibit the expression of the carboxylesterase gene EposCXE1 in the larval midgut and also inhibit the expression of the pheromone-binding protein EposPBP1 in adult antennae. The feeding of dsRNA also inhibited the expression of the nitrophorin 2 (NP2) gene in the salivary gland of R. prolixus, leading to a shortened coagulation time of plasma.

Direct spray of dsRNA on newly hatched Ostrinia furnalalis larvae has been reported. The studies have shown that after spraying dsRNAs (50 ng/ml) of the DS10 and DS28 genes (i.e. chymotrypsin-like serine protease C3 (DS10) and an unknown protein (DS28), respectively) on the newly hatched larvae placed on the filter paper, the larval mortalities were around 40-50%, whereas, after dsRNAs of ten genes were sprayed on the larvae along with artificial diet, the mortalities were significantly higher to the extent of 73-100%. It was proposed through these results that in a lepidopteron insect, dsRNAs are able to penetrate the integument and could retread larval developmental ultimately leading to death.

After feeding dsRNA to bees, siRNAs levels increased for a period of two weeks, suggesting that the silencing effect can last for at least that period of time.

In mosquitoes, RNAi method using chitosan/dsRNA self-assembled nanoparticles to mediate gene silencing through larval feeding in the African malaria mosquito (Anopheles gambiae) was shown. Oral-delivery of dsRNAs to larvae of the yellow fever mosquito, A. aegypti was also shown to be insecticidal. It was found that a relatively brief soaking in dsRNA, without the use of transfection reagents or dsRNA carriers, was sufficient to induce RNAi, and can either stunt growth or kill mosquito larvae. Furthermore, dsRNA targeting RNAi pathway genes were described to increased Dengue virus (DENV) replication in the Ae. Aegypti mosquito and to decrease the extrinsic incubation period required for virus transmission. The authors describe targeting the sequence of the gene AAEL011753 (Seq ID NO: 113) (r2d2) bp 76-575, which is one of the proteins of the silencing complex.

dsRNA can also be delivered to insects via ingestion of feed. U.S. Patent Application No. 20030022359 to Sayre teaches the use of transgenic algae and microalgae as a delivery system for recombinant peptides or proteins to host animals by ingestion, particularly animals feeding on algae. U.S. Patent Application No. 20130315883 to Sayre teaches the expression of exogenous dsRNA in transgenic algae, for downregulating expression of specific genes in host organisms feeding on the algae, including arthropods such as mosquitoes and mosquito larvae. The methods described, however, require the release of genetically modified microalgae into the environment.

U.S. Patent Application Nos. 20030154508 and 20030140371 provide pesticidal compositions that contain one or more compounds that interact with organic solute transporter/ligand-gated ion channel multifunction polypeptides (e.g. CAATCH protein) in the pest (e.g. mosquito), and/or alter amino acid metabolic pathways, and/or alter ionic homeostasis in the pest (e.g. mosquito). Upon exposure to a target pest, these compositions either compromise pest growth and/or cause the death of the pest. The compositions of U.S. 20030154508 and 20030140371 may contain one or more amino acids and/or amino acid analogs, or alternatively may comprise antibodies, antisense polynucleotides or RNAi.

U.S. Patent Application No. 20090285784 provides dsRNA as insect control agents. Specifically, U.S. 20090285784 provides methods for controlling insect infestation via RNAi-mediated gene silencing, whereby the intact insect cell(s) are contacted with a double-stranded RNA from outside the insect cell(s) and whereby the double-stranded RNA is taken up by the intact insect cell(s). U.S. Patent Application No. 20090010888 provides the use of cytochrome P450 reductase (CPR) as an insecticidal target. Specifically, U.S. 20090010888 provides methods of pest treatment (e.g. mosquitoes) comprising administering an agent (e.g. dsRNA) which is effective in reducing an activity and/or expression of the pest's CPR.

One method of introducing dsRNA to the larvae is by dehydration. Specifically, larvae are dehydrated in a NaCl solution and then rehydrated in water containing double-stranded RNA. This process is suggested to induce gene silencing in mosquito larvae.

### SUMMARY OF THE INVENTION

According to an aspect of some embodiments of the present invention there is provided a method of enhancing larvicide susceptibility in a mosquito larva, the method comprising introducing into the mosquito larva an isolated nucleic acid agent comprising a nucleic acid sequence which specifically reduces the expression of at least one larvicide resistance gene product of the larva, thereby enhancing larvicide susceptibility in the mosquito larva.

According to an aspect of some embodiments of the present invention there is provided a method of enhancing larvicide and/or adulticide susceptibility in a mosquito, the method comprising introducing into a mosquito larva an isolated nucleic acid agent comprising a nucleic acid sequence which specifically reduces the expression of at least one larvicide and/or adulticide resistance gene product of the mosquito, thereby enhancing larvicide and/or adulticide susceptibility in the mosquito when a pupa or an adult mosquito.

According to some embodiments of the present invention the larvicide resistance gene is selected from the group consisting of SEQ ID NOs: 1-111, 114-165, 193-202.

According to some embodiments of the present invention the larvicide resistance gene is selected from the genes in Tables 1A-B.

According to some embodiments of the present invention the enhanced larvicide susceptibility is enhanced as compared to identical mosquito larva not receiving the isolated nucleic acid agent.

According to some embodiments of the present invention the enhanced susceptibility is expressed as reduced LD so for the larvicide.

According to some embodiments of the present invention the mosquito is a mosquito capable of transmitting a disease to a mammalian organism.

According to some embodiments of the present invention the mosquito larvae are of the genus Culex.

According to some embodiments of the present invention the mosquito larvae are of the species Culex quinquefasciatus or Culex pipiens.

According to some embodiments of the present invention the mosquito larvae are of the genus Aedes.

According to some embodiments of the present invention the mosquito larvae are of the species Aedes aegypti or Aedes albopictus.

According to some embodiments of the present invention the mosquito larvae are of the genus Anopheles.

According to some embodiments of the present invention the mosquito larvae are selected from the group consisting of Anopheles gambiae, Anopheles stephensi, Anopheles albimanus.

According to some embodiments of the present invention the introducing comprises feeding, spraying, soaking or injecting.

According to some embodiments of the present invention the introducing comprises soaking the larva with the isolated nucleic acid agent for about 12-48 hours.

According to some embodiments of the present invention the larva is selected from the group consisting of first instar larva, second instar larva and third instar larva.

According to some embodiments of the present invention the introducing comprises feeding the larva with the isolated nucleic acid agent for about 48-96 hours.

According to some embodiments of the present invention the mosquito larva carries an infection selected from the group consisting of a viral infection, a nematode infection, a protozoa infection and a bacterial infection.

According to an aspect of some embodiments of the present invention there is provided an isolated nucleic acid agent comprising a nucleic acid sequence which specifically reduces the expression of at least one mosquito larvicide resistance gene product of a mosquito larva.

According to some embodiments of the present invention the larvicide resistance gene is selected from the group consisting of the genes in Tables 1A-B.

According to some embodiments of the present invention the larvicide resistance gene is selected from the group consisting of AAEL013279 (Seq ID NO: 16); AAEL001626 (Seq ID NO: 28); AAEL005772 (Seq ID NO: 40); AAEL012357 (Seq ID NO: 112), AAEL014445 (Seq ID NO: 102), AAEL008297 (Seq ID NO: 193), AAEL010379 (Seq ID NO: 194), AAEL007823 (Seq ID NO: 195), AAEL007698 (Seq ID NO: 196), AAEL005112 (Seq ID NO: 197), AAEL003446 (Seq ID NO: 198), AAEL007815 (Seq ID NO: 199), AAEL002202 (Seq ID NO: 200), AAEL009124 (Seq ID NO: 201) and Cytochrome p450 (CYP9J26) (Seq ID NO: 202).

According to some embodiments of the present invention the larvicide resistance gene is selected from the group consisting of AAEL008297 (Seq ID NO: 193), AAEL010379 (Seq ID NO: 194), AAEL007823 (Seq ID NO: 195), AAEL007698 (Seq ID NO: 196), AAEL005112 (Seq ID NO: 197), AAEL003446 (Seq ID NO: 198), AAEL007815 (Seq ID NO: 199), AAEL002202 (Seq ID NO: 200), AAEL009124 (Seq ID NO: 201) and Cytochrome p450 (CYP9J26) (Seq ID NO: 202).

According to some embodiments of the present invention the nucleic acid sequence reduces the expression of two mosquito larvicide resistance genes.

According to some embodiments of the present invention the two mosquito larvicide resistance genes comprise a sodium channel gene and a P-glycoprotein gene.

According to an aspect of some embodiments of the present invention there is provided a composition comprising at least one nucleic acid agent which specifically reduces the expression of two mosquito larvicide resistance genes.

According to some embodiments of the present invention the larvicide resistance gene comprise a sodium channel gene as set forth in SEQ ID NO: 193 and a P-glycoprotein gene as set forth in SEQ ID NO: 194.

According to some embodiments of the present invention the nucleic acid agent is a dsRNA comprising SEQ ID NO: 187 and a dsRNA SEQ ID NO: 184.

According to some embodiments of the present invention the isolated nucleic acid agent is a dsRNA.

According to some embodiments of the present invention the dsRNA is selected from the group consisting of SEQ ID NOs: 184-193, 203.

According to some embodiments of the present invention the dsRNA is effected at a dose of 0.001-1 μg/μL for soaking or at a dose of 1 pg to 10 μg/larvae for feeding.

According to some embodiments of the present invention the dsRNA is naked dsRNA.

According to some embodiments of the present invention the dsRNA comprises a carrier.

According to some embodiments of the present invention the carrier comprises a Polyethylenimine (PEI).

According to some embodiments of the present invention the dsRNA is selected from the group consisting of siRNA, shRNA and miRNA.

According to some embodiments of the present invention the nucleic acid sequence is greater than 15 base pairs in length.

According to some embodiments of the present invention the nucleic acid sequence is 19 to 25 base pairs in length.

According to some embodiments of the present invention the nucleic acid sequence is 30-100 base pairs in length.

According to some embodiments of the present invention the nucleic acid sequence is 100-800 base pairs in length.

According to an aspect of some embodiments of the present invention there is provided a nucleic acid construct comprising a nucleic acid sequence encoding the isolated nucleic acid agent of some embodiments of the invention.

According to some embodiments of the present invention the nucleic acid construct of some embodiments of the invention further comprising a regulatory element active in plant cells.

According to an aspect of some embodiments of the present invention there is provided a cell of a mosquito larva ingestible organism comprising the isolated nucleic acid agent of some embodiments of the invention.

According to some embodiments of the present invention the mosquito-larva-ingestible organism is an algae.

According to some embodiments of the present invention the algae is a microalgae.

According to an aspect of some embodiments of the present invention there is provided a composition comprising the isolated nucleic acid agent or the cell of some embodiments of the invention and an insecticidally acceptable carrier.

According to some embodiments of the present invention the composition is formulated in a form selected from the group consisting of a solid (e.g. particles), a semi-solid, a liquid, an emulsion, a powder, a paste and granules.

According to some embodiments of the present invention the composition is formulated in a semi-solid form.

According to some embodiments of the present invention the semi-solid form comprises agarose.

According to some embodiments of the present invention the composition of some embodiments of the invention further comprises a mosquito larvicide and/or adulticide.

According to some embodiments of the present invention the larvicide is selected from the group consisting of Temephos, Diflubenzuron, methoprene, Bacillus sphaericus, and Bacillus thuringiensis israelensis.

According to some embodiments of the present invention the adulticide is selected from the group consisting of deltamethrin, malathion, naled, chlorpyrifos, permethrin, resmethrin and sumithrin.

According to some embodiments of the present invention the composition further comprises mosquito larva feed.

According to some embodiments of the present invention the isolated nucleic acid agent further comprises a cell penetrating agent.

According to an aspect of some embodiments of the present invention there is provided a solution comprising the isolated nucleic acid agent, cells or the composition of some embodiments of the invention for soaking mosquito larvae.

According to an aspect of some embodiments of the present invention there is provided a mosquito or mosquito larva comprising at least one exogenous isolated nucleic acid agent comprising a nucleic acid sequence which specifically reduces the expression of at least mosquito larvicide resistance gene product.

According to some embodiments of the present invention the at least one exogenous isolated nucleic acid agent comprises the isolated nucleic acid agent of some embodiments of the invention.

According to some embodiments of the present invention the mosquito is selected from the group consisting of Anopheles genus, Aedes genus, Mansoni genus and Culex genus.

According to some embodiments of the present invention the mosquito or mosquito larva is at risk of infection with Dengue virus.

According to an aspect of some embodiments of the present invention there is provided a cell of the mosquito or mosquito larva of some embodiments of the invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart depicting introduction of dsRNA into mosquito larvae L1 via soaking and treatment of the larvae with temephos. In short, first (L1) instar larvae were treated (in groups of 100 larvae) in a final volume of 3 mL of autoclaved water with dsRNA (0.5 μg/μL). The control group was kept in 3 ml sterile water only. Larvae were soaked in the dsRNA solutions for 24 hours at 27° C., and were then transferred into new containers (200 larvae/1000 mL of chlorine-free tap water), also maintained at 27° C., and were supplemented with lab dog/cat diet (Purina Mills) suspended in water as a source of food on a daily basis. After soaking procedure, the larvae were reared until third instar and divided in 6 replicas with 10-20 larvae in each cup (final volume of 100 mL). Six replicas were treated with the lethal concentration 50 (0.00573 ppm) of temephos and 2 replicas with ethanol only (control group). Mortality was recorded after 24 and 48 hours.

FIG. 2 is a flowchart depicting introduction of dsRNA into mosquito larvae L3 via soaking and treatment of the larvae with temephos. In short, third (L3) instar larvae were treated (in groups of 100 larvae) in a final volume of 3 mL of autoclaved water with dsRNA (0.5 μg/μL). The control group was kept in 3 ml sterile water only. Larvae were soaked in the dsRNA solutions for 24 hours at 27° C. and were then divided. Six replicas were treated with the lethal concentration 50 (0.00573 ppm) of temephos and 2 replicas with ethanol only (control group). Mortality was recorded after 24 and 48 hours.

FIG. 3 is a flowchart depicting introduction of dsRNA into mosquito larvae via feeding and treatment of the larvae with diflubenzuron. In short, third instar larvae (in groups of 10 larvae) were exposed to one concentration (2.5 μg/L) of diflubenzuron pestanal (Sigma) in a final volume of 100 mL of chlorine-free tap water. From the beginning of diflubenzuron treatment, larvae were fed with agarose cubes containing 20 μg of dsRNA once a day for a total of 4 days. The plastic cups were covered with a nylon mesh in order to avoid adult escape. The evaluations were performed every other day by recording the mortality of the larvae and the number of emerged adults per replication as previously described. The test was terminated when all the larvae became pupae in the control group.

FIG. 4 is a flowchart illustrating schematically dsRNA production.

FIG. 5 is a table illustrating the susceptibility of Rockefeller and Rio de Janeiro (RJ) A. aegypti strains to temephos.

FIGS. 6A-E are graphs illustrating the gene expression profile of Ae. aegypti resistant (RJ) and susceptible (Rock) mosquitoes strains exposed to temephos. 3^{rd}-instar larvae from resistant (Rio de Janeiro—RJ) or susceptible (Rockfeller-Rock) strains were exposed to temephos (LC50). After 24 hours, larvae mortality was evaluated. Total mRNA was isolated from non-exposed larvae (before treatment) and temephos surviving larvae, and the mRNA expression levels of (FIG. 6A) AAEL009124, (FIG. 6B) AAEL005112, (FIG. 6C) AAEL003446, (FIG. 6D) AAEL007815 and (FIG. 6E) AAEL002202 were determined by qPCR. The relative level of mRNA expression were normalized to the level of S7 or tubulin mRNA expression *p<0.01; **p<0.001; ***p<0.0001 (paired student's t-test, bar=S.D, n=4).

FIG. 7 is a graph illustrating that larvae feeding of P-glycoprotein dsRNA induced gene silencing. Larvae from A. aegypti Rock strain (2^{nd} instar) were soaked for 24 hours in 0.5 μg/mL P-glycoprotein dsRNA or only water. 3^{rd}-instar larvae (previously soaked with dsRNAs at 2^{nd} instar) or only water were collected immediately before and after (only live larvae) treatment with temephos and analyzed for P-glycoprotein mRNA expression by qPCR. ***p<0.0001 (ANOVA, bar=S.D, n=4).

FIGS. 8A-B are graphs illustrating that larvae feeding of Sodium channel or Ago-3 dsRNAs induced gene silencing. Larvae from A. aegypti RJ strain (3^{rd} instar) were soaked for 24 hours in 0.5 μg/mL of P-glycoprotein, Sodium channel, AuB or Ago-3 dsRNAs. Larvae soaked only in water were used as control. Larval bioassay was conducted on sets of 25 early 3^{rd}-instar larvae placed in cups with the predetermined LC50 dose of insecticide. 3^{rd}-instar larvae previously soaked with the indicated dsRNAs or only water were collected immediately before and after (live larvae) treatment with temephos and analyzed for Sodium channel (FIG. 8A) and Ago-3 (FIG. 8B) mRNA expression by qPCR. *p<0.01; **p<0.001 (ANOVA, bar=S.D, n=4).

FIGS. 9A-B are graphs illustrating that feeding of A. aegypti larvae with Sodium channel dsRNA or PgP dsRNA increases its susceptibility to diflubenzuron insecticide. FIG. 9A, 3^{rd} instar larvae of Rockefeller strain were exposed to diflubenzuron (DBZ) pestanal larvicide (2.5 μg/L) in plastic cups containing 100 mL of dechlorinated tap water and fed simultaneously with dsRNA-containing food. Larval mortality was determined each 3 days. FIG. 9B, larvae were soaked with Sodium channel dsRNAs and exposed to diflubenzuron as described in FIG. 9A. Effects of diflubenzuron on adult emergence is shown as the percentage of emergence inhibition (100-100(T/C), where T is emergence (%) in DBZ-treated groups and C is emergence (%) in control groups. *p<0.01 (ANOVA, bar=S.D, n=4).

FIGS. 10A-B are graphs illustrating that concomitant feeding of A. aegypti larvae with Sodium channel or PgP dsRNA reduced significantly the viability of mosquito larvae 4 days after treatment with DBZ. 3^{rd} instar larvae of Rockefeller strain were exposed to diflubenzuron (DBZ) pestanal larvicide (2.5 μg/L) in plastic cups containing 100 mL of dechlorinated tap water and fed simultaneously with dsRNA-containing food targeting PgP (FIG. 10A) or dsRNA-containing food targeting Sodium channel (FIG. 10B). Larval mortality was determined each 3 days. Effects of diflubenzuron on adult emergence is shown as the percentage of emergence inhibition (100-100(T/C), where T is emergence (%) in DBZ-treated groups and C is emergence (%) in control groups. *p<0.01 (ANOVA, bar=S.D, n=4).

FIG. 11 is a flowchart illustration depicting introduction of dsRNA into mosquito larvae via soaking with “naked” dsRNA. In short, third instar larvae were treated (in groups of 100 larvae) in a final volume of 3 mL of dsRNA solution in autoclaved water with 0.5 μg/μL dsRNA. The control group was kept in 3 ml sterile water only. Larvae were soaked in the dsRNA solutions for 24 hr at 27° C., and then transferred into new containers (300 larvae/1500 mL of chlorine-free tap water), which were also maintained at 27° C., and were provided with lab dog/cat diet (Purina Mills) suspended in water as a source of food on a daily basis. As pupae developed, they were transferred to individual vials to await eclosion and sex sorting. For bioassays purpose only females up to five days old were used. Then, mosquitoes were subjected to pyrethroid adulticide assay.

FIG. 12 is a flowchart illustration depicting introduction of dsRNA into mosquito larvae via soaking with “naked” dsRNA plus additional larvae feeding with food-containing dsRNA. After soaking in the dsRNA solutions for 24 hr at 27° C. (as indicated in FIG. 1 above), the larvae were transferred into new containers (300 larvae/1500 mL of chlorine-free tap water), and were provided agarose cubes containing 300 μg of dsRNA once a day for a total of four days. The larvae were reared until adult stage. For bioassays purpose only females up to five days old are used. Then, mosquitoes were subjected to pyrethroid adulticide assay.

FIG. 13 is a flowchart illustration depicting introduction of dsRNA into mosquito larvae via feeding with food-containing dsRNA only. Third instar larvae were fed (in groups of 300 larvae) in a final volume of 1500 mL of chlorine-free tap water with agarose cubes containing 300 μg of dsRNA once a day for a total of four days. The larvae were reared until adult stage. For bioassays purpose only females up to five days old are used. Then, mosquitoes were subjected to pyrethroid adulticide assay.

FIGS. 14A-C are graphs illustrating the dose-response curves for 3- to 5-day-old Aedes aegypti female mosquitoes on insecticide-susceptible Rockefeller strain (FIG. 14A) and on insecticide-resistant Rio de Janeiro strain (FIG. 14B). Mosquitoes were exposed to different concentrations of deltamethrin in 250-mL glass bottles for up to 24 hours and the percentage of mortality for each time point is shown. FIG. 14C, comparison of the mortality rates of female mosquitoes from Rockefeller (Rock) and Rio de Janeiro (RJ) strains exposed to 2 μg/mL of deltamethrin for different time-points. Data represent mean values of three replicates with standard deviation.

FIGS. 15A-B are photographs illustrating allele specific PCR for genotyping kdr mutations in the Aedes aegypti Rio de Janeiro strain. FIGS. 15A-B represent reactions for the 1016 and 1534 mutation sites, respectively. Amplicons were resolved in a 10% polyacrylamide gel electrophoresis and stained with Gel Red. FIG. 15A, amplicons of approximately 80 and 100 bp correspond to alleles 1016 Val⁺ and 1016 Ile^{kdr}, respectively. FIG. 15B, amplicons of 90 and 110 bp correspond to alleles 1534 Phe⁺ and 1534 Cys^{kdr}, respectively. Rockefeller Ae. aegypti mosquito strain was used as positive homozygous dominant control for both mutation sites. C−: negative control.

FIGS. 16A-C are graphs illustrating that sodium channel gene silencing on Ae. aegypti mosquitoes (RJ strain) results in increased susceptibility to Pyrethroid adulticide. FIG. 16A, larvae from Ae. aegypti RJ strain (3^{rd} instar) were soaked for 24 hours in 0.5 μg/μL of sodium channel dsRNA or only in water, and then reared until adult stage. Adult females were exposed to deltamethrin (0.5 μg/bottle) for different time-points, as indicated, and mortality rates for each time point is shown. Data show the mean±standard deviation of four replicates, and is representative of 3 independent experiments. FIG. 16B, adult mosquitoes (males and females) previously soaked with sodium channel dsRNA or only water were collected before the treatment with deltamethrin and analyzed for sodium channel mRNA expression using qPCR method. FIG. 16C, live and immediately dead female mosquitoes were collected after exposure to deltamethrin and the mRNA expression of sodium channel was determined by qPCR analysis. ***p<0.0001; ****p<0.00001.

FIG. 17 is a graph illustrating that sodium channel gene silencing on A. aegypti mosquitoes (RJ strain) results in increased susceptibility to Pyrethroid adulticide. Larvae from Ae. aegypti RJ strain (3^{rd} instar) were soaked for 24 hours in 0.5 μg/μL of sodium channel dsRNA or only in water, and then were fed 4 times with food plus agarose 2% containing dsRNA until they reach pupa stage. After emergence, adult females were exposed to deltamethrin (0.5 μg/bottle) for different time-points, as indicated, and mortality rates for each time point is shown. Data show the mean±standard deviation of four replicates, and is representative of 3 independent experiments. *p<0.01; ***p<0.0001.

FIG. 18 is a graph illustrating that feeding CYP9J29 dsRNA to larvae affects the susceptibility of adult Ae. aegypti mosquitoes to Pyrethroid adulticide. Larvae from A. aegypti RJ strain (3^{rd} instar) were soaked for 24 hours in 0.1 μg/μL of target #3 (CYP9J26) dsRNA or only in water; and then were fed 4 times with food plus agarose 2% containing dsRNA until they reach pupa stage. Adult females were exposed to deltamethrin (0.5 μg/bottle) for different time-points, as indicated, and then percentage of mortality for each time point is shown. Data represent the mean±standard deviation of four replicates. **p<0.001.

FIGS. 19A-C are graphs illustrating gene silencing in A. aegypti larvae. 3^{rd} instar larvae from Ae. aegypti were soaked for 24 hours in 0.5 μg/mL of (FIG. 19A) P-glycoprotein (PgP); (FIG. 19B) Ago-3 or (FIG. 19C) sodium channel dsRNA. Larvae soaked only in water were used as control. At 6, 24 and 48 hours after the end of dsRNA treatment, larvae were collected and analysed for PgP, Ago-3 and Sodium channel mRNA expression by qPCR. Data represent the mean±standard deviation of four replicates. *p<0.01 **p<0.001; ***p<0.0001; ****p<0.00001.

FIGS. 20A-B are graphs illustrating P-glycoprotein and Ago-3 expression in Ae. aegypti adult mosquitoes soaked with dsRNA. Third instar larvae from Ae. aegypti were soaked for 24 hours in 0.5 μg/mL of (FIG. 20A) P-glycoprotein (PgP) and (FIG. 20B) Ago-3, and then reared until adult stage. Adult mosquitoes (males and females) previously soaked with the indicated dsRNA or only water were collected and analyzed for PgP and Ago-3 mRNA expression using qPCR method. Data represent the mean±standard deviation of five replicates. **p<0.001.

### DESCRIPTION OF SPECIFIC EMBODIMENTS OF THE INVENTION

The present invention, in some embodiments thereof, relates to isolated nucleic acid agents, and, more particularly, but not exclusively, to the use of same for enhancing susceptibility to larvicidal compounds in mosquito larvae and adults.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 184 is expressed in a DNA sequence format (e.g., reciting T for thymine), but it can refer to either a DNA sequence that corresponds to an endo 1,4 beta gluconase nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

Mosquitoes pose an important threat to human and animal health. Mosquitoes are vectors for numerous pathogens, including viruses, bacteria, protozoa and nematodes. One method of controlling mosquito populations is directed at eliminating mosquito larvae, which are more vulnerable to eradication efforts than adult mosquitoes due to larvae being an aquatic, surface-dwelling stage of the mosquito life cycle, feeding predominately on algae.

However, due to the vast numbers and rapid turnover of arthropod populations, even effective insecticides, including larvicides and adulticides, create selection pressure for insecticide-resistant mosquito and mosquito larval phenotypes. Introduction of novel insecticides, larvicides and adulticides both, initiates the cycle of selection and development of resistance, with the inevitable impairment or even complete loss of lethality of the insecticide.

While reducing the present invention to practice, the present inventors have uncovered that feeding dsRNA to mosquito larvae, wherein the dsRNA specifically downregulates an expression of a mosquito gene, wherein a product of the mosquito gene participates in resistance of the larva, or adult mosquito to an larvicide insecticide, results in mosquito larvae and mosquitoes more susceptible to the lethal effects of the larvicide insecticide and eradication therewith, reducing the effective dosage and increasing the “effective life expectancy” of the larvicides, and reducing the need for frequent introduction of novel insecticides/larvicides.

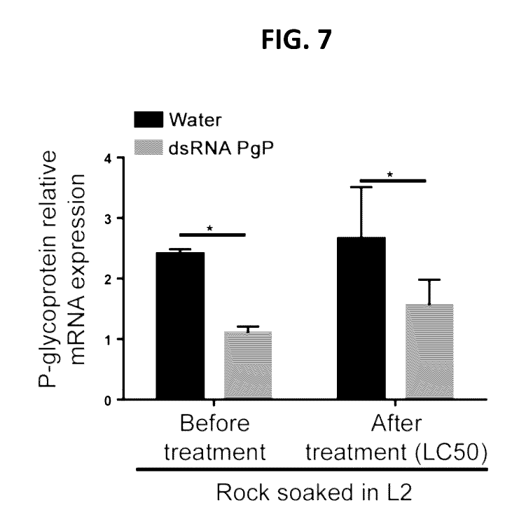

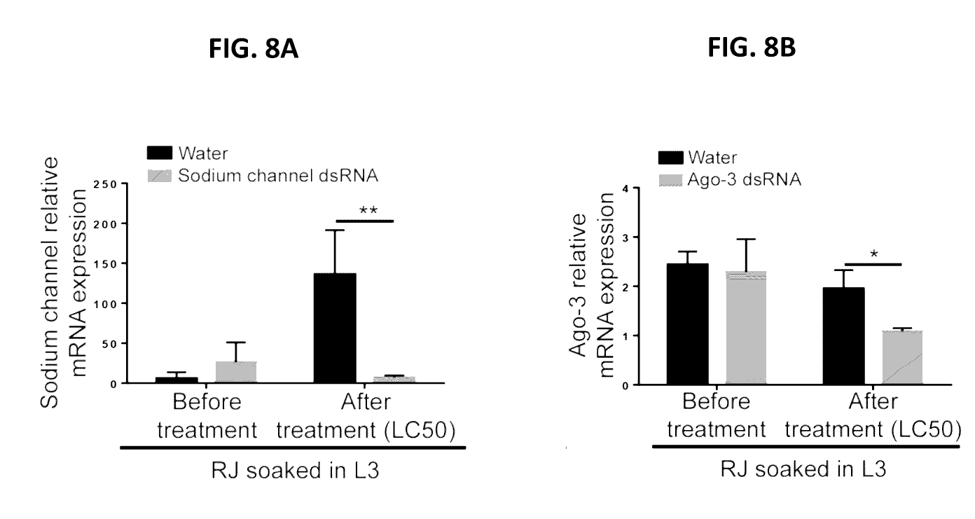

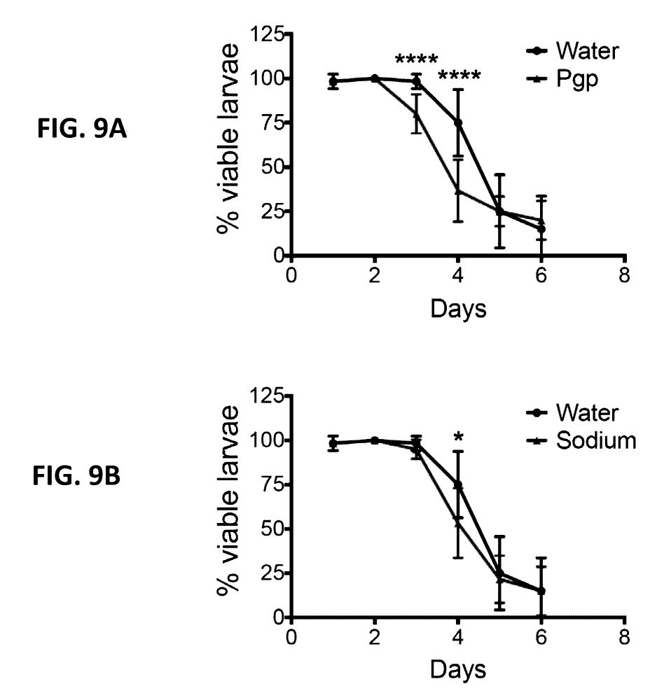

Specifically, the present inventors have shown that soaking mosquito L1 or L3 larvae in dsRNA targeting specific genes (e.g. P-glycoprotein, sodium channel, Ago-3) or feeding the larvae with agarose cubes containing dsRNA efficiently decreases gene expression (FIGS. 7, 8A-B) and results in higher susceptibility to larvicides as indicated by reduced adult emergence and reduced numbers of viable larvae (FIGS. 9A-B, 10A-B). Importantly, feeding mosquito larvae with a combination of Sodium channel dsRNA and P-glycoprotein (PgP) dsRNA increases its susceptibility to diflubenzuron insecticide (FIGS. 9A-B).

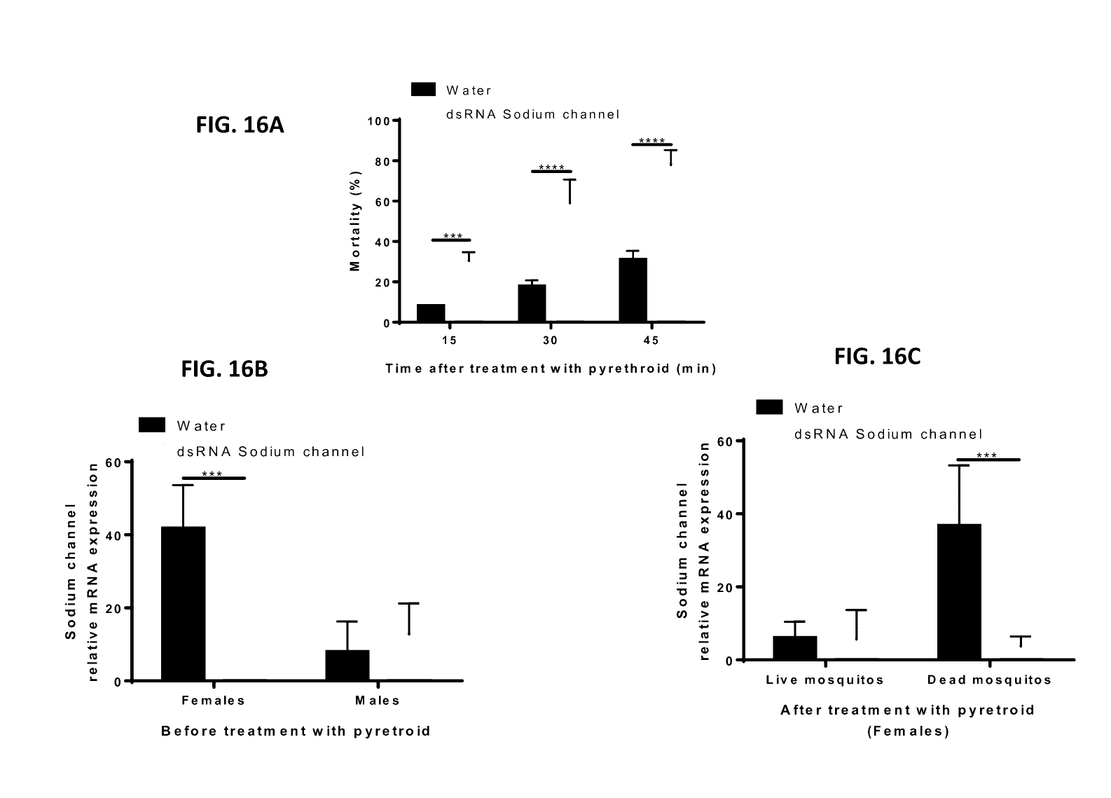

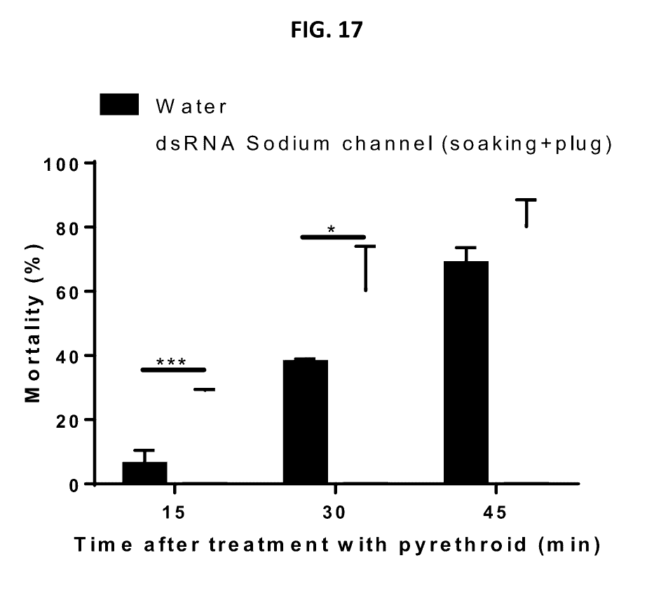

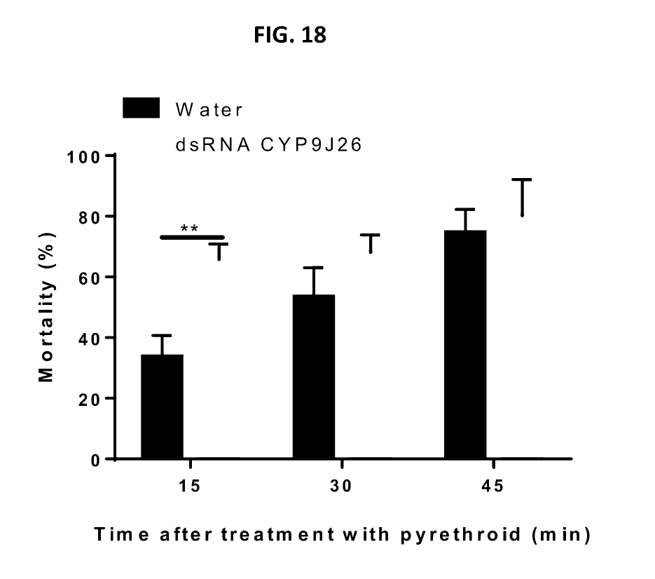

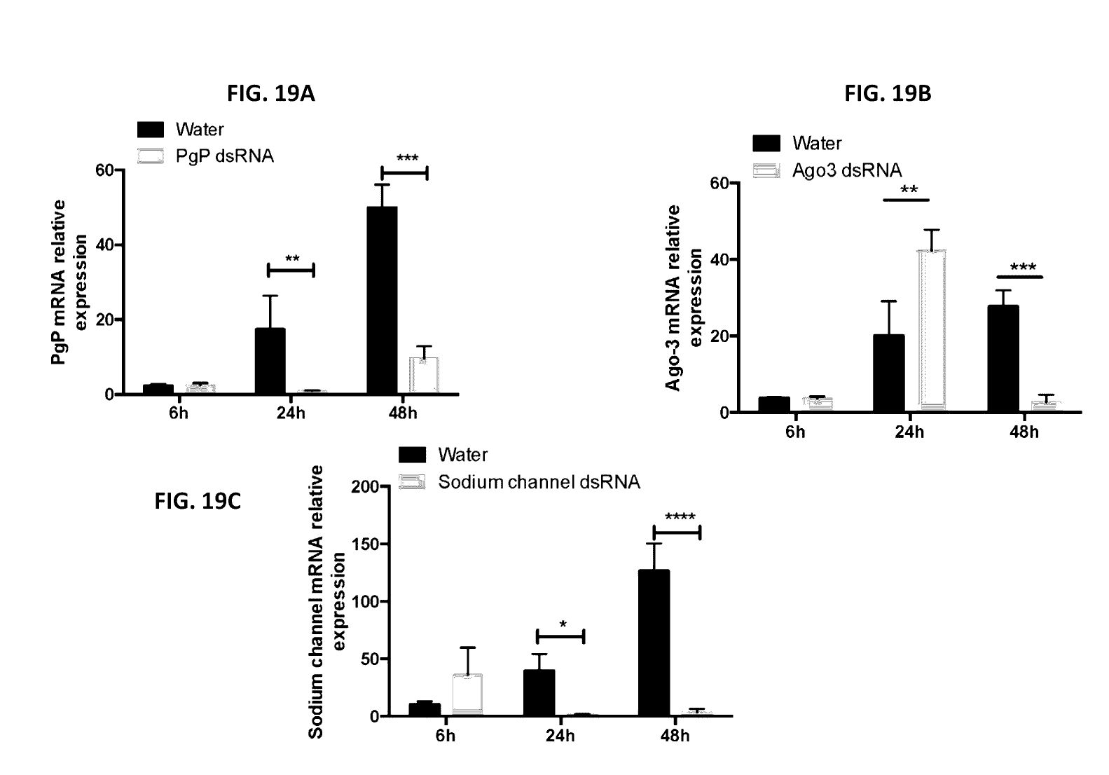

Moreover, the present inventors have illustrated that feeding mosquito larvae with dsRNA targeting specific genes for two to four days (via agarose cubes, until they reach pupa stage) with or without previous soaking with dsRNA for 24 hours (e.g. sodium channel, PgP, ago-3 and Cytochrome p450) efficiently decreases gene expression (FIGS. 19A-C) and results in higher susceptibility to the adulticide deltamethrin (FIGS. 17, 18) in adult mosquitoes. Importantly, female mosquitoes showed a decreased expression in the mRNA level for sodium channel before deltamethrin treatment (FIG. 16B) and dead female mosquitoes previously treated with dsRNA showed a striking decrease in mRNA expression level for sodium channel (FIG. 16C).

As is shown hereinbelow and in the Examples section which follows, the present inventors have uncovered that downregulating genes which are involved in larvicide resistance in a mosquito, e.g. genes responsible for enhanced esterase activity; enhanced glutatione-S-transferase activity; enhanced p450 monoxygenase activity, genes responsible for modification of acetylcholinesterase; modification of the GABA receptors; and modification of the sodium channels, can be effective in enhancing the mosquito larval and adult susceptibility to insecticides (larvicide/adulticides), and consequently reducing transmission of mosquito-borne pathogens to humans and animals.

Thus, according to one aspect of the present invention there is provided a method of enhancing larvicide susceptibility in a mosquito larva, the method comprising introducing into the mosquito larva an isolated nucleic acid agent comprising a nucleic acid sequence which specifically reduces the expression of at least one larvicide resistance gene product of the larva, thereby enhancing larvicide susceptibility in the mosquito larva.

As used herein the term “enhancing susceptibility of a mosquito larva” refers to increasing the sensitivity or reduction in tolerance of a mosquito larva to exposure to or any of the negative effects of a larvicide. Enhancing susceptibility can include, but is not limited to, reducing metabolic resistance mechanisms, reducing target site resistance mechanisms, enhancing penetration of the larvicide and reducing behavioural larvicide resistance mechanisms of the mosquito/larva. Accordingly, enhancing susceptibility of mosquitoes/larvae to larvicides reduces their damage to human health, economies, and well-being.

In some embodiments, enhanced susceptibility of the mosquito/mosquito larva to the larvicide/adulticide is a function of a reduced dosage requirement for toxicity or lethality of the larvicide, expressed as a reduction in the mean toxic dose (TD₅₀) or mean lethal dose (LD₅₀), that dose of the larvicide at which effective toxicity (TD) or death (LD) of 50% of a treated sample occurs. In some other embodiments, the enhanced susceptibility is a function of reduction in the exposure time to the larvicide required for toxic and/or lethal effects on the larva/mosquito. Additional and other mechanisms are conceived.

The term “mosquito” or “mosquitoes” as used herein refers to an insect of the family Culicidae. The mosquito of the invention may include an adult mosquito, a mosquito larva, a pupa or an egg thereof.

An adult mosquito is defined as any of slender, long-legged insect that has long proboscis and scales on most parts of the body. The adult females of many species of mosquitoes are blood-eating pests. In feeding on blood, adult female mosquitoes transmit harmful diseases to humans and other mammals.

A mosquito larvae is defined as any of an aquatic insect which does not comprise legs, comprises a distinct head bearing mouth brushes and antennae, a bulbous thorax that is wider than the head and abdomen, a posterior anal papillae and either a pair of respiratory openings (in the subfamily Anophelinae) or an elongate siphon (in the subfamily Culicinae) borne near the end of the abdomen.

Typically, a mosquito's life cycle includes four separate and distinct stages: egg, larva, pupa, and adult. Thus, a mosquito's life cycle begins when eggs are laid on a water surface (e.g. Culex, Culiseta, and Anopheles species) or on damp soil that is flooded by water (e.g. Aedes species). Most eggs hatch into larvae within 48 hours. The larvae live in the water feeding on microorganisms and organic matter and come to the surface to breathe. They shed their skin four times growing larger after each molting and on the fourth molt the larva changes into a pupa. The pupal stage is a resting, non-feeding stage of about two days. At this time the mosquito turns into an adult. When development is complete, the pupal skin splits and the mosquito emerges as an adult.

As used herein, the term “larvicide” or “larvicidal activity” refers to the ability of interfering with a mosquito life cycle resulting in an overall reduction in the mosquito population. The larvicidal composition acts (down-regulates gene expression) at the larval stage. The activity of the larvicidal composition may be manifested immediately (e.g., by affecting larval survival) or only at later stages, as described below. For example, the term larvicidal includes inhibition of a mosquito from progressing from one form to a more mature form, e.g., transition between various larval instars or transition from larva to pupa or pupa to adult. Further, the term “larvicidal” is intended to encompass, for example, anti-mosquito activity during all phases of a mosquito life cycle; thus, for example, the term includes larvicidal, ovicidal, and adulticidal activity, all of which stem from the activity at the larval stage.

It will be appreciated that a “larvicide” or “larvicidal composition” may also be effective in non-larval stages of the mosquito, and therefore may also be, for example, an “adulticide” or “adulticidal composition”. Thus, as used herein, the term larvicide encompasses both “larva-specific” larvicides, and non-larva-specific larvicides.

In addition the term may refer to rendering a mosquito at any stage, including adulthood, more susceptible to a pesticide as compared to the susceptibility of a mosquito of the same species and developmental stage which hasn't been treated with the larvicide.

According to one embodiment, the larvicide is selected from the group consisting of Temephos, Diflubenzuron, Methoprene, or a microbial larvicide such as Bacillus sphaericus or Bacillus thuringiensis israelensis.

According to one embodiment, the larvicide comprises an adulticide.

Exemplary adulticides include, but are not limited to, deltamethrin, malathion, naled, chlorpyrifos, permethrin, resmethrin or sumithrin.

As used herein, the term “larvicidally effective” is used to indicate an amount or concentration of a larvicide which is sufficient to reduce the number of mosquitoes in a geographic locus as compared to a corresponding geographic locus in the absence of the amount or concentration of the composition.

Thus the methods and compositions of some embodiments of the invention down-regulates a target larvicide resistance gene selected from the group consisting of genes:

(i) affecting larval survival when contacted with a larvicide;

(ii) interfering with metamorphosis of larval stage to adulthood contacted with a larvicide;

(iii) affecting susceptibility of an adult mosquito to an adulticide/insecticide.

As used herein the term “affecting” or “interfering” refers to a gene which plays a role in the above mentioned biological activity. According to a specific embodiment, the target gene is a non-redundant gene, that is, its activity is not compensated by another gene in a pathway. When needed, down-regulation of a plurality of genes (e.g., in a pathway) participating in at least one of the above-mentioned activities is contemplated (as further described hereinbelow). Alternatively, according to a specific embodiment, the plurality of target genes are from groups (i) and (ii), (i) and (iii), (ii) and (iii) or (i), (ii) and (iii).

According to one embodiment, the mosquitoes are of the sub-families Anophelinae and Culicinae. According to one embodiment, the mosquitoes are of the genus Culex, Culiseta, Anopheles and Aedes. Exemplary mosquitoes include, but are not limited to, Aedes species e.g. Aedes aegypti, Aedes albopictus, Aedes polynesiensis, Aedes australis, Aedes cantator, Aedes cinereus, Aedes rusticus, Aedes vexans; Anopheles species e.g. Anopheles gambiae, Anopheles freeborni, Anopheles arabiensis, Anopheles funestus, Anopheles gambiae Anopheles moucheti, Anopheles balabacensis, Anopheles baimaii, Anopheles culicifacies, Anopheles dirus, Anopheles latens, Anopheles leucosphyrus, Anopheles maculatus, Anopheles minimus, Anopheles fluviatilis s.l., Anopheles sundaicus Anopheles superpictus, Anopheles farauti, Anopheles punctulatus, Anopheles sergentii, Anopheles stephensi, Anopheles sinensis, Anopheles atroparvus, Anopheles pseudopunctipennis, Anopheles bellator and Anopheles cruzii; Culex species e.g. C. annulirostris, C. antennatus, C. jenseni, C. pipiens, C. pusillus, C. quinquefasciatus, C. rajah, C. restuans, C. salinarius, C. tarsalis, C. territans, C. theileri and C. tritaeniorhynchus; and Culiseta species e.g. Culiseta incidens, Culiseta impatiens, Culiseta inornata and Culiseta particeps.

According to one embodiment, the mosquitoes are capable of transmitting disease-causing pathogens. The pathogens transmitted by mosquitoes include viruses, protozoa, worms and bacteria.

Non-limiting examples of viral pathogens which may be transmitted by mosquitoes include the arbovirus pathogens such as Alphaviruses pathogens (e.g. Eastern Equine encephalitis virus, Western Equine encephalitis virus, Venezuelan Equine encephalitis virus, Ross River virus, Sindbis Virus and Chikungunya virus), Flavivirus pathogens (e.g. Japanese Encephalitis virus, Murray Valley Encephalitis virus, West Nile Fever virus, Yellow Fever virus, Dengue Fever virus, St. Louis encephalitis virus, and Tick-borne encephalitis virus), Bunyavirus pathogens (e.g. La Crosse Encephalitis virus, Rift Valley Fever virus, and Colorado Tick Fever virus) and Orbivirus (e.g. Bluetongue disease virus).

Non-limiting examples of worm pathogens which may be transmitted by mosquitoes include nematodes e.g. filarial nematodes such as Wuchereria bancrofti, Brugia malayi, Brugia pahangi, Brugia timori and heartworm (Dirofilaria immitis).

Non-limiting examples of bacterial pathogens which may be transmitted by mosquitoes include gram negative and gram positive bacteria including Yersinia pestis, Borellia spp, Rickettsia spp, and Erwinia carotovora.

Non-limiting examples of protozoa pathogens which may be transmitted by mosquitoes include the Malaria parasite of the genus Plasmodium e.g. Plasmodium falciparum, Plasmodium vivax, Plasmodium ovale, Plasmodium malariae, Plasmodium berghei, Plasmodium gallinaceum, and Plasmodium knowlesi.

A “host” may be any animal upon which the mosquito feeds and/or to which a mosquito is capable of transmitting a disease-causing pathogen. Non-limiting examples of hosts are mammals such as humans, domesticated pets (e.g. dogs and cats), wild animals (e.g. monkeys, rodents and wild cats), livestock animals (e.g. sheep, pigs, cattle, and horses), avians such as poultry (e.g. chickens, turkeys and ducks) and other animals such as crustaceans (e.g. prawns and lobsters), snakes and turtles.

According to one embodiment, the mosquito comprises a female mosquito being capable of transmitting a disease to a mammalian organism (e.g. an animal or human). According to another embodiment the female mosquito is pathogenically infected.

Non-limiting examples of mosquitoes and the pathogens which they transmit include species of the genus Anopheles (e.g. Anopheles gambiae) which transmit malaria parasites as well as microfilariae, arboviruses (including encephalitis viruses) and some species also transmit Wuchereria bancrofti; species of the genus Culex (e.g. C. pipiens) which transmit West Nile virus, filariasis, Japanese encephalitis, St. Louis encephalitis and avian malaria; species of the genus Aedes (e.g. Aedes aegypti, Aedes albopictus and Aedes polynesiensis) which transmit nematode worm pathogens (e.g. heartworm (Dirofilaria immitis)), arbovirus pathogens such as Alphaviruses pathogens that cause diseases such as Eastern Equine encephalitis, Western Equine encephalitis, Venezuelan equine encephalitis and Chikungunya disease; Flavivirus pathogens that cause diseases such as Japanese encephalitis, Murray Valley Encephalitis, West Nile fever, Yellow fever, Dengue fever, and Bunyavirus pathogens that cause diseases such as LaCrosse encephalitis, Rift Valley Fever, and Colorado tick fever.

According to one embodiment, pathogens that may be transmitted by Aedes aegypti are Dengue virus, Yellow fever virus, Chikungunya virus and heartworm (Dirofilaria immitis).

According to one embodiment, pathogens that may be transmitted by Aedes albopictus include West Nile Virus, Yellow Fever virus, St. Louis Encephalitis virus, Dengue virus, and Chikungunya fever virus.

According to one embodiment, pathogens that may be transmitted by Anopheles gambiae include malaria parasites of the genus Plasmodium such as, but not limited to, Plasmodium falciparum, Plasmodium vivax, Plasmodium ovale, Plasmodium malariae, Plasmodium berghei, Plasmodium gallinaceum, and Plasmodium knowlesi.

Enhancing susceptibility of a mosquito to a larvicide is achieved by downregulating an expression of at least one mosquito larvicide resistance gene.

As used herein, the term “mosquito gene” refers to any gene whose product is involved in larvicide tolerance and/or sensitivity. According to one embodiment, the mosquito gene is essential for an effect of the larvicide.

As used herein, the phrase “gene product” refers to an RNA molecule or a protein.

According to one embodiment, the mosquito gene product is one which is essential for the larvicide's effect upon encounter with the mosquito/larva. Downregulation of such a gene product would typically result in enhanced susceptibility, reduced tolerance and/or enhanced toxicity/lethality within the mosquito/larva.

Typically, larvicide, adulticide or insecticide resistance in the larva/mosquito results from changes in larvicide metabolism, changes in larvicide target site, barriers to penetration of the larvicide and alteration of behavior, often leading to reduced exposure to the larvicide. Best known are metabolic and target site resistance.

Thus, according to some embodiments, there is provided a method of enhancing larvicide susceptibility in a mosquito larva, the method comprising introducing into the mosquito larva an isolated nucleic acid agent comprising a nucleic acid sequence which specifically reduces the expression of at least one larvicide resistance gene product of the larva, thereby enhancing larvicide susceptibility in the mosquito larva.

It will be appreciated that introducing the agent into the mosquito larva can have an effect on later stages of mosquito life cycle—such as pupa/adult, etc. Thus, introducing the nucleic acid agent of the present invention into a larva can effectively enhance susceptibility of, for example, a pupa or adult mosquito to larvicide and/or adulticide. Thus, according to some embodiments, there is provided a method of enhancing larvicide and/or adulticide susceptibility in a mosquito, the method comprising introducing into a mosquito larva an isolated nucleic acid agent comprising a nucleic acid sequence which specifically reduces the expression of at least one larvicide or adulticide resistance gene product of the mosquito, thereby enhancing larvicide or adulticide susceptibility in the adult when a pupa or an adult mosquito.

Metabolic resistance involves the sequestration, metabolism, and/or detoxification of the insecticide, largely through the overproduction of specific enzymes such as carboxylesterases (efficient against organophosphate and carbamate insecticides), glutathione-S-transferases or GSTs (efficient against organophosphates, organochlorine, and pyrethroid insecticides) and cytochrome P450-dependent monoxygenases (efficient against most insecticide types, frequently in conjunction with other enzymes). The overproduction of these enzymes may be achieved via gene amplification and/or gene expression via modifications in the promoter region or mutations in trans-acting regulatory genes. In addition, in some mosquito species, carboxylesterase resistance to the insecticide malathion has been associated with a qualitative change in the enzyme.

In contrast, target site resistance is achieved by point mutations that render the actual targets of an insecticide less sensitive to the active ingredient. Most insecticides developed to date are neurotoxic and are directed to either acetylcholinesterase, c-aminobutyric acid (GABA) receptors, or sodium channels. Acetylcholinesterase is the target of organophosphorous and carbamate insecticides, the GABA receptors are the main targets of cyclodiene (organochlorine) insecticides, and the sodium channels are the targets of pyrethroid and organochlorine insecticides. Mutations in all three of these can confer resistance.

Thus, according to one embodiment, the larvicide resistance gene product is associated with metabolic larvicide resistance, such as, but not limited to a carboxylesterase gene, a glutathione-S-transferase (or GST) gene and a cytochrome P450-dependent monoxygenase gene.

In another embodiment, the larvicide resistance gene product is associated with penetration larvicide resistance, such as, but not limited to a mosquito larva or mosquito cuticle-associated gene, such as, but not limited to, a chitin gene or a chitin metabolism gene.

In yet another embodiment, mosquito larvicide resistance gene products that may be downregulated according to another aspect of the present invention are target-site-related genes, including, but are not limited to enhancing the sensitivity of acetylcholinesterase, c-aminobutyric acid (GABA) receptors, or sodium channels to organophosphor and carbamate larvicides, cyclodiene (organochlorine) larvicides, and pyrethroid and organochlorine larvicides, respectively.

Tables 1A-B, below, provides a partial list of mosquito genes associated with larvicide resistance, which can be potential targets for reduction in expression by introducing the nucleic acid agent of the invention.

TABLE 1A

| | Seq ID | Gene Symbol | Annotation |
|---|---|---|---|
| Enzymes | 1 | AAEL012664 | prolylcarboxypeptidase, putative |
| | 2 | AAEL002909 | lysosomal acid lipase, putative |
| | 3 | AAEL005127 | ribonuclease UK114, putative |
| | 4 | AAEL012636 | cytochrome b5, putative |
| | 5 | AAEL010276 | aminomethyltransferase |
| | 6 | AAEL013640 | lung carbonyl reductase |
| | 7 | AAEL005416 | oxidase/peroxidase |
| | 8 | AAEL013499 | prophenoloxidase |
| | 9 | AAEL003716 | ribonuclease UK114, putative |
| | 10 | AAEL012579 | aspartate aminotransferase |
| | 11 | AAEL002600 | serine protease |
| | 12 | AAEL005610 | mitochondrial ATP synthase b chain |
| | 13 | AAEL006446 | trehalose-6-phosphate synthase |
| | 14 | AAEL008770 | proteasome subunit beta type |
| | 15 | AAEL001427 | short-chain dehydrogenase |
| | 16 | AAEL013279 | peptidyl-prolyl cis-trans isomerase (cyclophilin) |
| | 17 | AAEL009875 | alanine aminotransferase |
| | 18 | AAEL005793 | AMP dependent ligase |
| | 19 | AAEL007868 | ubiquinol-cytochrome c reductase complex 14 kd protein |
| | 20 | AAEL008072 | NADH-plastoquinone oxidoreductase |
| | 21 | AAEL009324 | hydroxyacyl dehydrogenase |
| | 22 | AAEL008217 | serine-type enodpeptidase, |
| | 23 | AAEL014944 | cytochrome c oxidase polypeptide |
| | 24 | AAEL010819 | vacuolar ATP synthase subunit H |
| | 25 | AAEL010500 | glutathione-s-transferase theta, gst |
| Transport | 26 | AAEL005929 | ATP-binding cassette transporter |
| | 27 | AAEL008381 | oligopeptide transporter |
| | 28 | AAEL001626 | zinc/iron transporter |
| | 29 | AAEL012702 | ATP-binding cassette sub-family A member 3, putative |
| others | 30 | AAEL015515 | antibacterial peptide, putative |
| | 31 | AAEL002295 | leucine-rich transmembrane protein |
| | 32 | AAEL009556 | Niemann-Pick Type C-2, putative |
| | 33 | AAEL005159 | latent nuclear antigen, putative |
| | 34 | AAEL007325 | Mob3B protein, putative |
| | 35 | AAEL000679 | NEDD8, putative |
| | 36 | AAEL009209 | galactose-specific C-type lectin, putative |
| | 37 | AAEL001826 | odorant-binding protein 56a, putative |
| | 38 | AAEL002961 | Osiris, putative |
| | 39 | AAEL006830 | yellow protein precursor |
| | 40 | AAEL005772 | odorant-binding protein 99c, putative |
| | 41 | AAEL002813 | coupling factor, putative |
| | 42 | AAEL011090 | complement component |
| | 43 | AAEL012230 | flagellar protein, putative |
| Hypothetical proteins | 44 | AAEL011252 | conserved hypothetical protein |
| | 45 | AAEL014506 | conserved hypothetical protein |
| | 46 | AAEL003216 | conserved hypothetical protein |
| | 47 | AAEL003241 | conserved hypothetical protein |
| | 48 | AAEL007507 | conserved hypothetical protein |
| | 49 | AAEL003064 | conserved hypothetical protein |
| | 50 | AAEL010678 | conserved hypothetical protein |
| | 51 | AAEL000269 | conserved hypothetical protein |
| | 52 | AAEL006053 | conserved hypothetical protein |
| | 53 | AAEL008750 | conserved hypothetical protein |
| | 54 | AAEL010128 | conserved hypothetical protein |
| | 55 | AAEL002898 | conserved hypothetical protein |
| | 56 | AAEL007631 | conserved hypothetical protein |
| | 57 | AAEL003479 | conserved hypothetical protein |
| | 58 | AAEL013777 | conserved hypothetical protein |
| | 59 | AAEL003428 | conserved hypothetical protein |
| | 60 | AAEL014529 | conserved hypothetical protein |
| | 61 | AAEL012645 | conserved hypothetical protein |
| | 62 | AAEL004809 | conserved hypothetical protein |
| | 63 | AAEL004343 | conserved hypothetical protein |
| | 64 | AAEL003160 | conserved hypothetical protein |
| | 65 | AAEL012357 | conserved hypothetical protein |
| | 66 | AAEL009009 | conserved hypothetical protein |
| | 67 | AAEL013793 | conserved hypothetical protein |
| | 68 | AAEL002623 | conserved hypothetical protein |
| | 69 | AAEL010163 | conserved hypothetical protein |
| | 70 | AAEL002449 | conserved hypothetical protein |
| | 71 | AAEL002302 | conserved hypothetical protein |
| | 72 | AAEL008039 | conserved hypothetical protein |
| | 73 | AAEL008073 | conserved hypothetical protein |
| | 74 | AAEL007444 | conserved hypothetical protein |
| | 75 | AAEL005171 | conserved hypothetical protein |
| | 76 | AAEL006771 | conserved hypothetical protein |
| | 77 | AAEL015140 | conserved hypothetical protein |
| | 78 | AAEL001851 | conserved hypothetical protein |
| | 79 | AAEL005558 | conserved hypothetical protein |
| | 80 | AAEL002933 | conserved hypothetical protein |
| | 81 | AAEL003225 | conserved hypothetical protein |
| | 82 | AAEL001692 | conserved hypothetical protein |
| | 83 | AAEL007592 | conserved hypothetical protein |
| | 84 | AAEL005457 | conserved hypothetical protein |
| | 85 | AAEL006494 | conserved hypothetical protein |
| | 86 | AAEL013780 | conserved hypothetical protein |
| | 87 | AAEL009257 | conserved hypothetical protein |
| | 88 | AAEL000445 | conserved hypothetical protein |
| | 89 | AAEL002955 | conserved hypothetical protein |
| | 90 | AAEL002875 | conserved hypothetical protein |
| | 91 | AAEL000304 | conserved hypothetical protein |
| | 92 | AAEL000792 | conserved hypothetical protein |
| | 93 | AAEL003936 | conserved hypothetical protein |
| | 94 | AAEL006686 | conserved hypothetical protein |
| | 95 | AAEL001677 | conserved hypothetical protein |
| | 96 | AAEL000419 | conserved hypothetical protein |
| | 97 | AAEL007648 | conserved hypothetical protein |
| | 98 | AAEL006270 | conserved hypothetical protein |
| | 99 | AAEL013377 | conserved hypothetical protein |
| | 100 | AAEL002619 | conserved hypothetical protein |
| | 101 | AAEL012866 | conserved hypothetical protein |
| | 102 | AAEL014445 | conserved hypothetical protein |
| | 103 | AAEL001065 | conserved hypothetical protein |
| | 104 | AAEL011333 | conserved hypothetical protein |
| | 105 | AAEL011078 | conserved hypothetical protein |
| | 106 | AAEL010315 | conserved hypothetical protein |
| | 107 | AAEL005270 | conserved hypothetical protein |
| | 108 | AAEL004449 | conserved hypothetical protein |
| | 109 | AAEL000896 | conserved hypothetical protein |
| | 110 | AAEL010724 | conserved hypothetical protein |
| | 111 | AAEL008802 | conserved hypothetical protein |

TABLE 1B

| SEQ | | |
|---|---|---|
| ID | | |
| NO | Gene symbol | Gene Name |
| 114 | AAEL000765 | hexamerin 2 beta |
| 115 | AAEL000887 | conserved hypothetical protein |
| 116 | AAEL000890 | conserved hypothetical protein |
| 117 | AAEL001054 | glutathione transferase |
| 118 | AAEL001807 | cytochrome P450 |
| 119 | AAEL002062 | Betha Ftz transcription factor isoform A (nuclear |
| | | receptor) |
| 120 | AAEL003102 | glucosyl/glucuronosyl transferases |
| 121 | AAEL003286 | alkaline phosphatase |
| 122 | AAEL003297 | alkaline phosphatase |
| 123 | AAEL003317 | alkaline phosphatase |
| 124 | AAEL003667 | hypothetical protein |
| 125 | AAEL003986 | conserved hypothetical protein |
| 126 | AAEL003986 | conserved hypothetical protein |
| 127 | AAEL003986 | conserved hypothetical protein |
| 128 | AAEL003986 | conserved hypothetical protein |
| 129 | AAEL004088 | aldo-keto reductase |
| 130 | AAEL004406 | conserved hypothetical protein |
| 131 | AAEL004846 | conserved hypothetical protein |
| 132 | AAEL005156 | hypothetical protein |
| 133 | AAEL005546 | conserved hypothetical protein |
| 134 | AAEL006627 | serine-type enodpeptidase, |
| 135 | AAEL007383 | secreted ferritin G subunit precursor, putative |
| 136 | AAEL007383 | secreted ferritin G subunit precursor, putative |
| 137 | AAEL007383 | secreted ferritin G subunit precursor, putative |
| 138 | AAEL008045 | hexamerin 2 beta |
| 139 | AAEL008598 | conserved hypothetical protein |
| 140 | AAEL008968 | conserved hypothetical protein |
| 141 | AAEL009580 | conserved hypothetical protein |
| 142 | AAEL009764 | xaa-pro aminopeptidase |
| 143 | AAEL009773 | geminin, putative |
| 144 | AAEL009953 | Niemann-Pick Type C-2, putative |
| 145 | AAEL009954 | Niemann-Pick Type C-2, putative |
| 146 | AAEL009956 | conserved hypothetical protein |
| 147 | AAEL010382 | aldehyde oxidase |
| 148 | AAEL011112 | alcohol dehydrogenase |
| 149 | AAEL011169 | hexamerin 2 beta |
| 150 | AAEL012344 | lipase 1 precursor |
| 151 | AAEL012434 | conserved hypothetical protein |
| 152 | AAEL012526 | hypothetical protein |
| 153 | AAEL012736 | ribosomal protein L15 |
| 154 | AAEL012828 | conserved hypothetical protein |
| 155 | AAEL013757 | hexamerin 2 beta |
| 156 | AAEL013759 | hexamerin 2 beta |
| 157 | AAEL013837 | conserved hypothetical protein |
| 158 | AAEL014591 | filamin, putative |
| 159 | AAEL014727 | conserved hypothetical protein |
| 160 | AAEL014893 | cytochrome P450 |
| 161 | AAEL014936 | sarcosine dehydrogenase |
| 162 | AAEL015119 | cuticle protein, putative |
| 163 | AAEL017209 | hypothetical protein |
| 164 | AAEL017453 | hypothetical protein |
| 165 | AAEL017508 | hypothetical protein |
| 193 | AAEL008297 | |
| 194 | AAEL010379 | |
| 195 | AAEL007823 | |
| 196 | AAEL007698 | |
| 197 | AAEL005112 | |
| 198 | AAEL003446 | |
| 199 | AAEL007815 | |
| 200 | AAEL002202 | |
| 201 | AAEL009124 | |
| 202 | | Cytochrome p450 |
| | | (CYP9J26) |
| | | JF924909.1 |
| | | XM_001649047.2 |

The present teachings contemplate the targeting of homologs and orthologs according to the selected mosquito species.

The term “species homolog” or “homolog” as used herein refers to one that has an amino acid or nucleotide homology with a given gene in a given species (e.g. at least 60% homology, at least 70% homology, at least 80%, at least 85%, at least 90%, or at least 95% homology). A method for obtaining such a species homolog is well known to one of skill in the art.

The term “ortholog” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Further, comparing human Cystatin A (a cysteine protease inhibitor) and rice Oryzacystatin, only three short amino acid motives are conserved which are believed to be critical for interaction with a protease of target, and the other portions have very low amino acid similarity. However, both belong to the superfamily of cystatin genes, and have genes of common origin, and thus, not only the cases where there are high amino acid homology, but also in cases where there are only a few common amino acids in a particular region of these protein structure, these may be called orthologs to each other. As such, orthologs usually play a similar role to that in the original species in another species.

In some embodiments, the larvicide resistance gene products include, but are not limited to sequences of AAEL013279 (Seq ID NO: 16); AAEL001626 (Seq ID NO: 28); AAEL005772 (Seq ID NO: 40); AAEL012357 (Seq ID NO: 112) and AAEL014445 (Seq ID NO: 102). According to one embodiment, the larvicide resistance gene product that is downregulated comprises any one of the nucleic acid sequences as set forth in SEQ ID NO: 1-111 (or orthologs thereof, dependent on the target of interest).

According to one embodiment, the larvicide resistance gene is selected from the group consisting of AAEL008297 (Seq ID NO: 193), AAEL010379 (Seq ID NO: 194), AAEL007823 (Seq ID NO: 195), AAEL007698 (Seq ID NO: 196), AAEL005112 (Seq ID NO: 197), AAEL003446 (Seq ID NO: 198), AAEL007815 (Seq ID NO: 199), AAEL002202 (Seq ID NO: 200), AAEL009124 (Seq ID NO: 201) and Cytochrome p450 (CYP9J26) (Seq ID NO: 202).

It will be appreciated that more than one gene may be targeted in order to optimize or maximize the larvicide susceptibility of the mosquitoes/larvae.

Thus, a combination of two or more silencing agents e.g., dsRNAs, for a single target gene or distinct genes is contemplated according to the present teachings.

Thus, for example, a combination of dsRNA targeting the genes AAEL002202 and AAEL003446 is contemplated herein. Alternatively, a combination of dsRNA targeting the genes AAEL005112 and AAEL007815 is contemplated. Alternatively, a combination of dsRNA targeting the genes AAEL008297 and AAEL010379 is contemplated. When referring to targeting together it is understood that the larvae may be administered two silencing agents, e.g., dsRNAs, concomitantly or subsequently to one another (e.g. hours or days apart).

As used herein, the term “downregulates an expression” or “downregulating expression” refers to causing, directly or indirectly, reduction in the transcription of a desired gene, reduction in the amount, stability or translatability of transcription products (e.g. RNA) of the gene, and/or reduction in translation of the polypeptide(s) encoded by the desired gene.

Downregulating expression of a larvicide or adulticide resistance gene product of a mosquito can be monitored, for example, by direct detection of gene transcripts (for example, by PCR), by detection of polypeptide(s) encoded by the gene (for example, by Western blot or immunoprecipitation), by detection of biological activity of polypeptides encode by the gene (for example, catalytic activity, ligand binding, and the like), or by monitoring changes in the mosquitoes (for example, reduced LD₅₀ or TD₅₀ of a larvicide on the larva/mosquito etc). Additionally or alternatively downregulating expression of a pathogen resistance gene product may be monitored by measuring larvicide levels (e.g. molecules or larvicide activity etc.) in the mosquitoes as compared to wild type (i.e. control) mosquitoes/larvae not treated by the agents of the invention.

Thus, according to some aspects of the invention there is provided an isolated nucleic acid agent comprising a nucleic acid sequence which specifically downregulates the expression of at least one mosquito larvicide resistance gene product.

According to one embodiment, the agent is a polynucleotide agent, such as an RNA silencing agent.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

In some embodiments of the invention, the nucleic acid agent is a double stranded RNA (dsRNA). As used herein the term “dsRNA” relates to two strands of anti-parallel polyribonucleic acids held together by base pairing. The two strands can be of identical length or of different lengths provided there is enough sequence homology between the two strands that a double stranded structure is formed with at least 80%, 90%, 95% or 100% complementarity over the entire length. According to an embodiment of the invention, there are no overhangs for the dsRNA molecule. According to another embodiment of the invention, the dsRNA molecule comprises overhangs. According to other embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed.

It will be noted that the dsRNA can be defined in terms of the nucleic acid sequence of the DNA encoding the target gene transcript, and it is understood that a dsRNA sequence corresponding to the coding sequence of a gene comprises an RNA complement of the gene's coding sequence, or other sequence of the gene which is transcribed into RNA.

The inhibitory RNA sequence can be greater than 90% identical, or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-16 hours; followed by washing). The length of the double-stranded nucleotide sequences complementary to the target gene transcript may be at least about 18, 19, 21, 25, 50, 100, 200, 300, 400, 491, 500, 550, 600, 650, 700, 750, 800, 900, 1000 or more bases. In some embodiments of the invention, the length of the double-stranded nucleotide sequence is approximately from about 18 to about 1000, about 18 to about 750, about 18 to about 510, about 18 to about 400, about 18 to about 250 nucleotides in length.

The term “corresponds to” as used herein means a polynucleotide sequence homologous to all or a portion of a reference polynucleotide sequence. In contradistinction, the term “complementary to” is used herein to mean that the complementary sequence is homologous to all or a portion of a reference polynucleotide sequence. For example, the nucleotide sequence “TATAC” corresponds to a reference sequence “TATAC” and is complementary to a reference sequence “GTATA”.

The present teachings relate to various lengths of dsRNA, whereby the shorter version i.e., x is shorter or equals 50 bp (e.g., 17-50), is referred to as siRNA or miRNA. Longer dsRNA molecules of 51-600 are referred to herein as dsRNA, which can be further processed for siRNA molecules. According to some embodiments, the nucleic acid sequence of the dsRNA is greater than 15 base pairs in length. According to yet other embodiments, the nucleic acid sequence of the dsRNA is 19-25 base pairs in length, 30-100 base pairs in length, 100-250 base pairs in length or 100-500 base pairs in length. According to still other embodiments, the dsRNA is 500-800 base pairs in length, 700-800 base pairs in length, 300-600 base pairs in length, 350-500 base pairs in length or 400-450 base pairs in length. In some embodiments, the dsRNA is 400 base pairs in length. In some embodiments, the dsRNA is 750 base pairs in length.

The term “siRNA” refers to small inhibitory RNA duplexes (generally between 17-30 basepairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550, SEQ ID NO: 1) and 5′-UUUGUGUAG-3′ (Castanotto, D. et al. (2002) RNA 8:1454, SEQ ID NO: 2). It will be recognized by one of skill in the art that the resulting single chain oligonucleotide forms a stem-loop or hairpin structure comprising a double-stranded region capable of interacting with the RNAi machinery.

As used herein, the phrase “microRNA (also referred to herein interchangeably as “miRNA” or “miR”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, present in any plant cell and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising an imperfect double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nucleotides in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the dsRNA molecules may be naturally occurring or synthetic.

In some embodiments, the dsRNA is provided dsRNA, without additional agents (for example, transfection agents).

According to a specific embodiment, the nucleic acid agent is provided to the mosquito in a configuration devoid of a heterologous promoter for driving recombinant expression of the dsRNA (exogenous), rendering the nucleic acid molecule of the instant invention a naked molecule. The nucleic acid agent may still comprise modifications that may affect its stability and bioavailability (e.g., PNA).

The term “recombinant expression” refers to an expression from a nucleic acid construct.

As used herein “devoid of a heterologous promoter for driving expression of the dsRNA” means that the molecule doesn't include a cis-acting regulatory sequence (e.g., heterologous) transcribing the dsRNA. As used herein the term “heterologous” refers to exogenous, not-naturally occurring within a native cell of the mosquito or in a cell in which the dsRNA is fed to the larvae or mosquito (such as by position of integration, or being non-naturally found within the cell).

The nucleic acid agent can be further comprised within a nucleic acid construct comprising additional regulatory elements. Thus, according to some embodiments of aspects of the invention there is provided a nucleic acid construct comprising isolated nucleic acid agent comprising a nucleic acid sequence which specifically reduces the expression of at least one plant pathogen resistance gene product.

The dsRNA can be a mixture of long and short dsRNA molecules such as, dsRNA, siRNA, siRNA+dsRNA, siRNA+miRNA, or a combination of same.

The nucleic acid agent is designed for specifically targeting a target gene of interest (e.g. a mosquito pathogen resistance gene). It will be appreciated that the nucleic acid agent can be used to downregulate one or more target genes (e.g. as described in detail above). If a number of target genes are targeted, a heterogenic composition which comprises a plurality of nucleic acid agents for targeting a number of target genes is used. Alternatively the plurality of nucleic acid agents is separately formulated. According to a specific embodiment, a number of distinct nucleic acid agent molecules for a single target are used, which may be used separately or simultaneously (i.e., co-formulation) applied.

For example, in order to silence the expression of an mRNA of interest, synthesis of the dsRNA suitable for use with some embodiments of the invention can be selected as follows. First, the mRNA sequence is scanned including the 3′ UTR and the 5′ UTR. Second, the mRNA sequence is compared to an appropriate genomic database using any sequence alignment software, such as the BLAST software available from the NCBI server (wwwdotncbidotnlmdotnihdotgov/BLAST/). Putative regions in the mRNA sequence which exhibit significant homology to other coding sequences are filtered out.

Qualifying target sequences are selected as template for dsRNA synthesis. Preferred sequences are those that have as little homology to other genes in the genome to reduce an “off-target” effect.

It will be appreciated that the RNA silencing agent of some embodiments of the invention need not be limited to those molecules containing only RNA, but further encompasses chemically-modified nucleotides and non-nucleotides.

According to one embodiment, the dsRNA is selected from the group consisting of SEQ ID NOs: 184-192, 203.

The dsRNA may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

Although the instant teachings mainly concentrate on the use of dsRNA which is not comprised in or transcribed from an expression vector (naked), the present teachings also contemplate an embodiment wherein the nucleic acid agent is ligated into a nucleic acid construct comprising additional regulatory elements. Thus, according to some embodiments of the invention there is provided a nucleic acid construct comprising an isolated nucleic acid agent comprising a nucleic acid sequence.

For transcription from an expression cassette, a regulatory region (e.g., promoter, enhancer, silencer, leader, intron and polyadenylation) may be used to modulate the transcription of the RNA strand (or strands). Therefore, in one embodiment, there is provided a nucleic acid construct comprising the nucleic acid agent. The nucleic acid construct can have polynucleotide sequences constructed to facilitate transcription of the RNA molecules of the present invention operably linked to one or more promoter sequences functional in a mosquito cell. The polynucleotide sequences may be placed under the control of an endogenous promoter normally present in the mosquito genome. The polynucleotide sequences of the present invention, under the control of an operably linked promoter sequence, may further be flanked by additional sequences that advantageously affect its transcription and/or the stability of a resulting transcript. Such sequences are generally located upstream of the promoter and/or downstream of the 3′ end of the expression construct. The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream, within, or downstream of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, pausing sequences, polyadenylation recognition sequences, and the like.

It will be appreciated that the nucleic acid agents can be delivered to the mosquito larva in a variety of ways.

According to one embodiment, the composition of some embodiments comprises cells, which comprise the nucleic acid agent.

As used herein the term “cell” or “cells” refers to a mosquito larva ingestible cell, for example, a cell of a mosquito larva ingestible organism. Mosquito larva ingestible organism can be a unicellular mosquito larva ingestible organism, or a multi-cellular mosquito larva ingestible organism.

Examples of such cells include, but are not limited to, cells of phytoplankton (e.g., algae), fungi (e.g., Legendium giganteum), bacteria, and zooplankton such as rotifers.

Specific examples include, bacteria (e.g., cocci and rods), filamentous algae and detritus.

The choice of the cell or organism may depend on the target larvae.

Analyzing the gut content of mosquitoes and larvae may be used to elucidate their preferred diet. The skilled artisan knows how to characterize the gut content. Typically the gut content is stained such as by using a fluorochromatic stain, 4′,6-diamidino-2-phenylindole or DAPI.

Cells of particular interest are the prokaryotes and the lower eukaryotes, such as fungi. Illustrative prokaryotes, both Gram-negative and Gram-positive, include Enterobacteriaceae; Bacillaceae; Rhizobiceae; Spirillaceae; Lactobacillaceae; and phylloplane organisms such as members of the Pseudomonadaceae.

An exemplary list includes Bacillus spp., including B. megaterium, B. subtilis; B. cereus, Bacillus thuringiensis, Escherichia spp., including E. coli, and/or Pseudomonas spp., including P. cepacia, P. aeruginosa, and P. fluorescens.

Among eukaryotes are fungi, such as Phycomycetes and Ascomycetes, which includes yeast, such as Schizosaccharomyces; and Basidiomycetes, Rhodotorula, Aureobasidium, Sporobolomyces, Saccharomyces spp., and Sporobolomyces spp.

According to a specific embodiment, the cell is an algal cell.

Various algal species can be used in accordance with the teachings of the invention since they are a significant part of the diet for many kinds of mosquito larvae that feed opportunistically on microorganisms as well as on small aquatic animals such as rotifers.

Examples of algae that can be used in accordance with the present teachings include, but are not limited to, blue-green algae as well as green algae.

According to a specific embodiment, the algal cell is a cyanobacterium cell which is in itself toxic to mosquitoes as taught by Marten 2007 Biorational Control of Mosquitoes. American mosquito control association Bulletin No. 7.

Specific examples of algal cells which can be used in accordance with the present teachings are provided in Marten, G. G. (1986) Mosquito control by plankton management: the potential of indigestible green algae. Journal of Tropical Medicine and Hygiene, 89: 213-222, and further listed infra.

### Green Algae

Actinastrum hantzschii, Ankistrodesmus falcatus, Ankistrodesmus spiralis, Aphanochaete elegans, Chlamydomonas sp., Chlorella ellipsoidea, Chlorella pyrenoidosa, Chlorella variegate, Chlorococcum hypnosporum, Chodatella brevispina, Closterium acerosum, Closteriopsis acicularis, Coccochloris peniocystis, Crucigenia lauterbornii, Crucigenia tetrapedia, Coronastrum ellipsoideum, Cosmarium botrytis, Desmidium swartzii, Eudorina elegans, Gloeocystis gigas, Golenkinia minutissima, Gonium multicoccum, Nannochloris oculata, Oocystis marssonii, Oocystis minuta, Oocystis pusilla, Palmella texensis, Pandorina morum, Paulschulzia pseudovolvox, Pediastrum clathratum, Pediastrum duplex, Pediastrum simplex, Planktosphaeria gelatinosa, Polyedriopsis spinulosa, Pseudococcomyxa adhaerans, Quadrigula closterioides, Radiococcus nimbatus, Scenedesmus basiliensis, Spirogyra pratensis, Staurastrum gladiosum, Tetraedron bitridens, Trochiscia hystrix.

### Blue-Green Algae

Anabaena catenula, Anabaena spiroides, Chroococcus turgidus, Cylindrospermum licheniforme, Bucapsis sp. (U. Texas No. 1519), Lyngbya spiralis, Microcystis aeruginosa, Nodularia spumigena, Nostoc linckia, Oscillatoria lutea, Phormidium faveolarum, Spinilina platensis.

### Other

Compsopogon coeruleus, Cryptomonas ovata, Navicula pelliculisa.

The nucleic acid agent is introduced into the cells. To this end cells are typically selected exhibiting natural competence or are rendered competent, also referred to as artificial competence.

Competence is the ability of a cell to take up nucleic acid molecules e.g., the nucleic acid agent, from its environment.

A number of methods are known in the art to induce artificial competence.

Thus, artificial competence can be induced in laboratory procedures that involve making the cell passively permeable to the nucleic acid agent by exposing it to conditions that do not normally occur in nature. Typically the cells are incubated in a solution containing divalent cations (e.g., calcium chloride) under cold conditions, before being exposed to a heat pulse (heat shock).

Electroporation is another method of promoting competence. In this method the cells are briefly shocked with an electric field (e.g., 10-20 kV/cm) which is thought to create holes in the cell membrane through which the nucleic acid agent may enter. After the electric shock the holes are rapidly closed by the cell's membrane-repair mechanisms.

Yet alternatively or additionally, cells may be treated with enzymes to degrade their cell walls, yielding. These cells are very fragile but take up foreign nucleic acids at a high rate.

Exposing intact cells to alkali cations such as those of cesium or lithium allows the cells to take up nucleic acids. Improved protocols use this transformation method, while employing lithium acetate, polyethylene glycol, and single-stranded nucleic acids. In these protocols, the single-stranded molecule preferentially binds to the cell wall in yeast cells, preventing double stranded molecule from doing so and leaving it available for transformation.

Enzymatic digestion or agitation with glass beads may also be used to transform cells.

Particle bombardment, microprojectile bombardment, or biolistics is yet another method for artificial competence. Particles of gold or tungsten are coated with the nucleic acid agent and then shot into cells.

Astier C R Acad Sci Hebd Seances Acad Sci D. 1976 Feb. 23; 282(8):795-7, which is hereby incorporated by reference in its entirety, teaches transformation of a unicellular, facultative chemoheterotroph blue-green Algae, Aphanocapsa 6714. The recipient strain becomes competent when the growth reaches its second, slower, exponential phase.

Vazquez-Acevedo M¹Mitochondrion. 2014 Feb. 21. pii: S1567-7249(14)00019-1. doi: 10.1016/j.mito.2014.02.005, which is hereby incorporated by reference in its entirety, teaches transformation of algal cells e.g., Chlamydomonas reinhardtii, Polytomella sp. and Volvox carteri by generating import-competent mitochondria.

According to a specific embodiment the composition of the invention comprises an RNA binding protein.

According to a specific embodiment, the dsRNA binding protein (DRBP) comprises any of the family of eukaryotic, prokaryotic, and viral-encoded products that share a common evolutionarily conserved motif specifically facilitating interaction with dsRNA. Polypeptides which comprise dsRNA binding domains (DRBDs) may interact with at least 11 bp of dsRNA, an event that is independent of nucleotide sequence arrangement. More than 20 DRBPs have been identified and reportedly function in a diverse range of critically important roles in the cell. Examples include the dsRNA-dependent protein kinase PKR that functions in dsRNA signaling and host defense against virus infection and DICER.

Alternatively or additionally, an siRNA binding protein may be used as taught in U.S. Pat. Application No. 20140045914, which is herein incorporated by reference in its entirety.

According to a specific embodiment the RNA binding protein is the p19 RNA binding protein. The protein may increase in vivo stability of an siRNA molecule by coupling it at a binding site where the homodimer of the p19 RNA binding proteins is formed and thus protecting the siRNA from external attacks and accordingly, it can be utilized as an effective siRNA delivery vehicle.

According to a specific embodiment, the target-oriented peptide is located on the surface of the siRNA binding protein.

According to specific embodiments of the invention, whole cell preparations, cell extracts, cell suspensions, cell homogenates, cell lysates, cell supernatants, cell filtrates, or cell pellets of cell cultures of cells comprising the nucleic acid agent can be used.

The composition of some embodiments of the invention may further comprise at least one of a surface-active agent, an inert carrier vehicle, a preservative, a humectant, a feeding stimulant, an attractant, an encapsulating agent, a binder, an emulsifier, a dye, an ultra-violet protector, a buffer, a flow agent or fertilizer, micronutrient donors.

According to a specific embodiment, the cells are formulated by any means known in the art. The methods for preparing such formulations include, e.g., desiccation, lyophilization, homogenization, extraction, filtration, encapsulation centrifugation, sedimentation, or concentration of one or more cell types.

Additionally, the composition may be supplemented with larval food (food bait) or with excrements of farm animals, on which the mosquito larvae feed.

In one embodiment, the composition comprises an oil flowable suspension. For example, in some embodiments, oil flowable or aqueous solutions may be formulated to contain lysed or unlysed cells, spores, or crystals.

In a further embodiment, the composition may be formulated as a water dispersible granule or powder.

In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained-release, or other time-dependent manner.

Alternatively or additionally, the composition may comprise an aqueous solution. Such aqueous solutions or suspensions may be provided as a concentrated stock solution which is diluted prior to application, or alternatively, as a diluted solution ready-to-apply. Such compositions may be formulated in a variety of ways. They may be employed as wettable powders, granules or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like).

The formulations may include spreader-sticker adjuvants, stabilizing agents, other pesticidal additives, or surfactants. Liquid formulations may be employed as foams, suspensions, emulsifiable concentrates, or the like. The ingredients may include Theological agents, surfactants, emulsifiers, dispersants, or polymers.

As mentioned, the dsRNA of the invention may be administered as a naked dsRNA. Alternatively, the dsRNA of the invention may be conjugated to a carrier known to one of skill in the art, such as a transfection agent e.g. PEI or chitosan or a protein/lipid carrier.

The compositions may be formulated prior to administration in an appropriate means such as lyophilized, freeze-dried, microencapsulated, desiccated, or in an aqueous carrier, medium or suitable diluent, such as saline or other buffer. Suitable agricultural carriers can be solid, semi-solid or liquid and are well known in the art. The term “agriculturally-acceptable carrier” covers all adjuvants, e.g., inert components, dispersants, surfactants, tackifiers, binders, etc. that are ordinarily used in pesticide formulation technology.

According to one embodiment, the composition is formulated as a semi-solid such as in agarose (e.g. agarose cubes).

As mentioned, the nucleic acid agents can be delivered to the mosquito larva in various ways. Thus, administration of the composition to the mosquito larva may be carried out using any suitable or desired manual or mechanical technique for application of a composition comprising a nucleic acid agent, including but not limited to spraying, soaking, brushing, dressing, dripping, dipping, coating, spreading, applying as small droplets, a mist or an aerosol.

According to one embodiment, the composition is administered to the larvae by soaking or by spraying.

According to one embodiment, the composition is administered to the larvae by feeding.

Feeding the larva with the composition can be effected for about 2 hours to 120 hours, about 2 hours to 108 hours, about 2 hours to 96 hours, about 2 hours to 84 hours, about 2 hours to 72 hours, for about 2 hours to 60 hours, about 2 hours to 48 hours, about 2 hours to 36 hours, about 2 hours to 24 hours, about 2 hours to 12 hours, 12 hours to 24 hours, about 24 hours to 36 hours, about 24 hours to 48 hours, about 36 hours to 48 hours, for about 48 hours to 60 hours, about 60 hours to 72 hours, about 72 hours to 84 hours, about 84 hours to 96 hours, about 96 hours to 108 hours, or about 108 hours to 120 hours.

According to a specific embodiment, the composition is administered to the larvae by feeding for 48-96 hours.

According to one embodiment, feeding the larva with the composition is affected until the larva reaches pupa stage.

According to one embodiment, prior to feeding the larva with dsRNA, the larvae are first soaked with dsRNA.

Soaking the larva with the composition can be effected for about 2 hours to 96 hours, about 2 hours to 84 hours, about 2 hours to 72 hours, for about 2 hours to 60 hours, about 2 hours to 48 hours, about 2 hours to 36 hours, about 2 hours to 24 hours, about 2 hours to 12 hours, 12 hours to 96 hours, about 12 hours to 84 hours, about 12 hours to 72 hours, for about 12 hours to 60 hours, about 12 hours to 48 hours, about 12 hours to 36 hours, about 12 hours to 24 hours, or about 24 hours to 48 hours.

According to a specific embodiment, the composition is administered to the larvae by soaking for 12-24 hours.

Thus, for example, larvae (e.g. first, second, third or four instar larva, e.g. third instar larvae) are first treated (in groups of about 100 larvae) with dsRNA at a dose of about 0.001-5 μg/μL (e.g. 0.2 μg/μL), in a final volume of about 3 mL of dsRNA solution in autoclaved water. After soaking in the dsRNA solutions for about 12-48 hours (e.g. for 24 hrs) at 25-29° C. (e.g. 27° C.), the larvae are transferred into containers so as not to exceed concentration of about 200-500 larvae/1500 mL (e.g. 300 larvae/1500 mL) of chlorine-free tap water, and provided with food containing dsRNA (e.g. agarose cubes containing 300 μg of dsRNA, e.g. 1 μg of dsRNA/larvae). The larva are fed once a day until they reach pupa stage (e.g. for 2-5 days, e.g. four days). Larvae are also fed with additional food requirements, e.g. 2-10 mg/100 mL (e.g. 6 mg/100 mL) lab dog/cat diet suspended in water.

Feeding the larva can be effected using any method known in the art. Thus, for example, the larva may be fed with agarose cubes, chitosan nanoparticles, oral delivery or diet containing dsRNA.

Chitosan nanoparticles: A group of 15-20 3rd-instar mosquito larvae are transferred into a container (e.g. 500 ml glass beaker) containing 50-1000 ml, e.g. 100 ml, of deionized water. One sixth of the gel slices that are prepared from dsRNA (e.g. 32 μg of dsRNA) are added into each beaker. Approximately an equal amount of the gel slices are used to feed the larvae once a day for a total of 2-5 days. e.g. four days (see Insect Mol Biol. 2010 19(5):683-93).

Oral delivery of dsRNA: First instar larvae (less than 24 hrs old) are treated in groups of 10-100, e.g. 50, in a final volume of 25-100 μl of dsRNA, e.g. 75 μl of dsRNA, at various concentrations (ranging from 0.01 to 5 μg/μl, e.g. 0.02 to 0.5 μg/μl-dsRNAs) in tubes e.g. 2 mL microfuge tube (see J Insect Sci. 2013; 13:69).

Diet containing dsRNA: larvae are fed a single concentration of 1-2000 ng dsRNA/mL, e.g. 1000 ng dsRNA/mL, diet in a diet overlay bioassay for a period of 1-10 days, e.g. 5 days (see PLoS One. 2012; 7(10): e47534).

Diet containing dsRNA: Newly emerged larvae are starved for 1-12 hours, e.g. 2 hours, and are then fed with a single drop of 0.5-10 μl, e.g. 1 μl, containing 1-20 μg, e.g. 4 μg, dsRNA (1-20 μg of dsRNA/larva, e.g. 4 μg of dsRNA/larva) (see Appl Environ Microbiol. 2013 August; 79(15):4543-50). Thus, according to a specific embodiment, the composition may be applied to standing water. The mosquito larva may be soaked in the water for several hours (1, 2, 3, 4, 5, 6 hours or more) to several days (1, 2, 3, 4 days or more) with or without the use of transfection reagents or dsRNA carriers.

Alternatively, the mosquito larva may be sprayed with an effective amount of the composition (e.g. via an aqueous solution).

If needed, the composition may be dissolved, suspended and/or diluted in a suitable solution (as described in detail above) before use.

The nucleic acid compositions of the invention may be employed in the method of the invention singly or in combination with other compounds, including, but not limited to, pesticides.

Compositions of the invention can be used to control (e.g. exterminate) mosquitoes. Such an application comprises administering to larvae of the mosquitoes an effective amount of the composition which renders an adult stage of the mosquitoes lethally susceptible to a pathogen, thereby controlling (e.g. exterminating) the mosquitoes.

Thus, regardless of the method of application, the amount of the active component(s) are applied at a effective amount for a larval stage of the mosquito to be lethally susceptible to a larvicide, which will vary depending on factors such as, for example, the specific mosquito to be controlled, the type of larvicide, the water source to be treated, the environmental conditions, and the method, rate, and quantity of application of the composition.

The concentration of the composition that is used for environmental, systemic, or foliar application will vary widely depending upon the nature of the particular formulation, means of application, environmental conditions, and degree of biocidal activity.

Exemplary concentrations of dsRNA in the composition (e.g. for soaking) include, but are not limited to, about 1 pg-10 μg of dsRNA/μl, about 1 pg-1 μg of dsRNA/μl, about 1 pg-0.1 μg of dsRNA/μl, about 1 pg-0.01 μg of dsRNA/μl, about 1 pg-0.001 μg of dsRNA/μl, about 0.001 μg-10 μg of dsRNA/μl, about 0.001 μg-5 μg of dsRNA/μl, about 0.001 μg-1 μg of dsRNA/μl, about 0.001 μg-0.1 μg of dsRNA/μl, about 0.001 μg-0.01 μg of dsRNA/μl, about 0.01 μg-10 μg of dsRNA/μl, about 0.01 μg-5 μg of dsRNA/μl, about 0.01 μg-1 μg of dsRNA/μl, about 0.01 μg-0.1 μg of dsRNA/μl, about 0.1 μg-10 μg of dsRNA/μl, about 0.1 μg-5 μg of dsRNA/μl, about 0.5 μg-5 μg of dsRNA/μl, about 0.5 μg-10 μg of dsRNA/μl, about 1 μg-5 μg of dsRNA/μl, or about 1 μg-10 μg of dsRNA/μl.

When formulated as a feed, the dsRNA may be effected at a dose of 1 pg/larvae-1000 μg/larvae, 1 pg/larvae-500 μg/larvae, 1 pg/larvae-100 μg/larvae, 1 pg/larvae-10 μg/larvae, 1 pg/larvae-1 μg/larvae, 1 pg/larvae-0.1 μg/larvae, 1 pg/larvae-0.01 μg/larvae, 1 pg/larvae-0.001 μg/larvae, 0.001-1000 μg/larvae, 0.001-500 μg/larvae, 0.001-100 μg/larvae, 0.001-50 μg/larvae, 0.001-10 μg/larvae, 0.001-1 μg/larvae, 0.001-0.1 μg/larvae, 0.001-0.01 μg/larvae, 0.01-1000 μg/larvae, 0.01-500 μg/larvae, 0.01-100 μg/larvae, 0.01-50 μg/larvae, 0.01-10 μg/larvae, 0.01-1 μg/larvae, 0.01-0.1 μg/larvae, 0.1-1000 μg/larvae, 0.1-500 μg/larvae, 0.1-100 μg/larvae, 0.1-50 μg/larvae, 0.1-10 μg/larvae, 0.1-1 μg/larvae, 1-1000 μg/larvae, 1-500 μg/larvae, 1-100 μg/larvae, 1-50 μg/larvae, 1-10 μg/larvae, 10-1000 μg/larvae, 10-500 μg/larvae, 10-100 μg/larvae, 10-50 μg/larvae, 50-1000 μg/larvae, 50-500 μg/larvae, 50-400 μg/larvae, 50-300 μg/larvae, 100-500 μg/larvae, 100-300 μg/larvae, 200-500 μg/larvae, 200-300 μg/larvae, or 300-500 μg/larvae.

The mosquito larva food containing dsRNA may be prepared by any method known to one of skill in the art. Thus, for example, cubes of dsRNA-containing mosquito food may be prepared by first mixing 10-500 μg, e.g. 300 μg of dsRNA with 3 to 300 μg, e.g. 10 μg of a transfection agent e.g. Polyethylenimine 25 kDa linear (Polysciences) in 10-500 μL, e.g. 200 μL of sterile water. Alternatively, 2 different dsRNA (10-500 μg, e.g. 150 μg of each) plus 3 to 300 μg, e.g. 30 μg of Polyethylenimine may be mixed in 10-500 μL, e.g. 200 μL of sterile water. Alternatively, cubes of dsRNA-containing mosquito food may be prepared without the addition of transfection reagents. Then, a suspension of ground mosquito larval food (1-20 grams/100 mL e.g. 6 grams/100 mL) may be prepared with 2% agarose (Fisher Scientific). The food/agarose mixture can then be heated to 53-57° C., e.g. 55° C., and 10-500 μL, e.g. 200 μL of the mixture can then be transferred to the tubes containing 10-500 μL, e.g. 200 μL of dsRNA+PEI or dsRNA only. The mixture is then allowed to solidify into a gel. The solidified gel containing both the food and dsRNA can be cut into small pieces (approximately 1-10 mm, e.g. 1 mm, thick) using a razor blade, and can be used to feed mosquito larvae in water.

According to some embodiments, the nucleic acid agent is provided in amounts effective to reduce or suppress expression of at least one mosquito gene product. As used herein “a suppressive amount” or “an effective amount” refers to an amount of dsRNA which is sufficient to downregulate (reduce expression of) the target gene by at least 20%, 30%, 40%, 50%, or more, say 60%, 70%, 80%, 90% or more even 100%.

Testing the efficacy of gene silencing can be effected using any method known in the art. For example, using quantitative RT-PCR measuring gene knockdown. Thus, for example, ten to twenty larvae from each treatment group can be collected and pooled together. RNA can be extracted therefrom and cDNA syntheses can be performed. The cDNA can then be used to assess the extent of RNAi by measuring levels of gene expression using qRT-PCR.

Reagents of the present invention can be packed in a kit including the nucleic acid agent (e.g. dsRNA), instructions for administration of the nucleic acid agent, construct or composition to mosquito larva.

Compositions of some embodiments of the invention may, if desired, be presented in a pack or dispenser device, which may contain one or more dosage forms containing the active ingredient. The pack may, for example, comprise metal or plastic foil, such as a blister pack. The pack or dispenser device may be accompanied by instructions for administration to the mosquito larva.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Experimental Procedures

Mosquito Maintenance

Mosquitoes were taken from an Ae. aegypti colony of the Rockefeller strain or from a mosquito field population of Ae. aegypti isolated from urban area of Rio de Janeiro, Brazil. Both lineages were reared continuously in the laboratory at 28° C. and 70-80% relative humidity. Adult insects were maintained in a 10% sucrose solution, and the adult females were fed with sheep blood for egg laying. The larvae were reared on dog/cat food unless stated otherwise.

Introducing dsRNA into a Mosquito Larvae

Two different approaches were evaluated for treatment with dsRNA according to the larvicides Temephos or Diflubenzuron:

1. Soaking with “Naked” dsRNA and Bioassay with Temephos

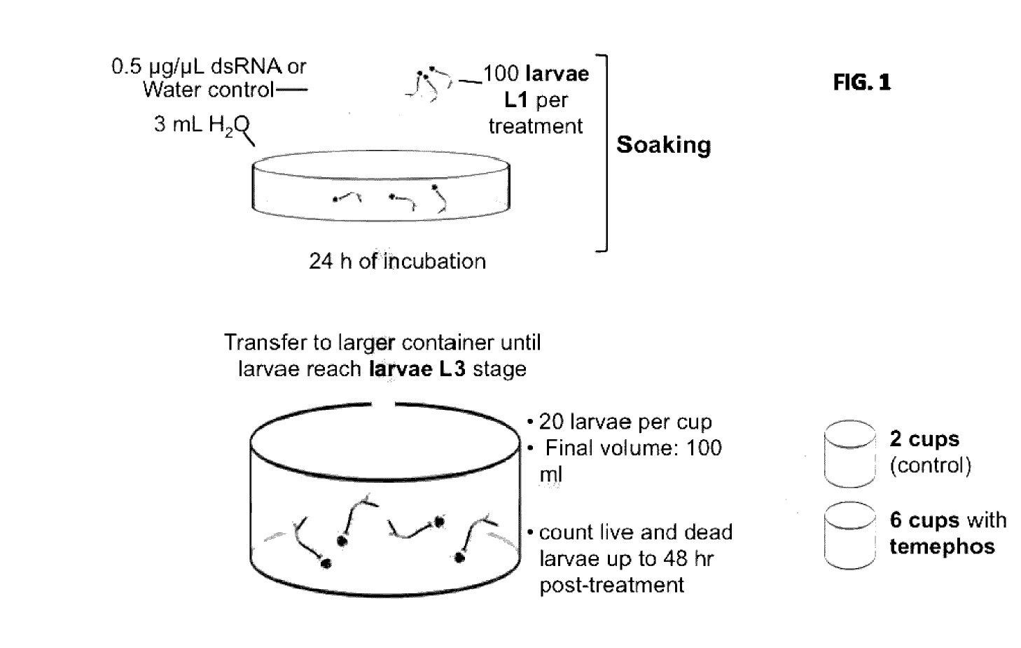

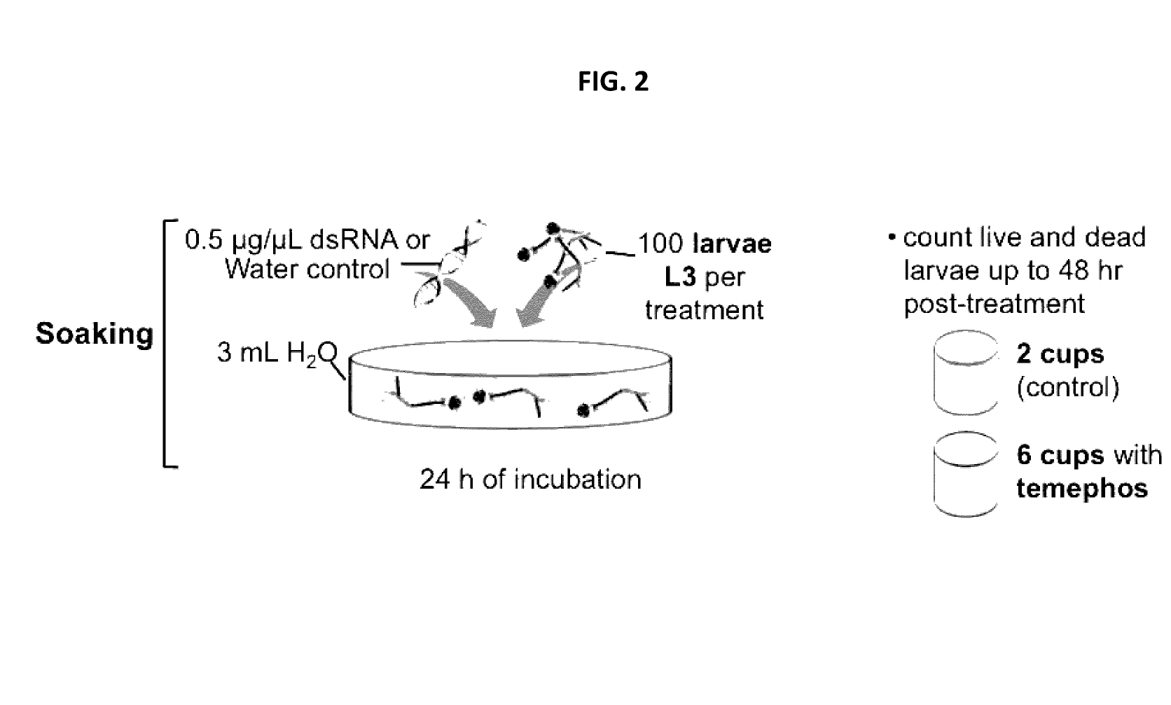

First (L1), Second (L2) or Third (L3) instar larvae were treated (in groups of 100 larvae) in a final volume of 3 mL of autoclaved water with sodium channel (AAEL008297), PgP (AAEL010379), Ago3 (AAEL007823), and Aub (AAEL007698) dsRNA (0.5 μg/μL), or AAEL005112, AAEL003446, AAEL007815 and AAEL002202 dsRNA (0.1 μg/μL). The control group was kept in 3 ml sterile water only. Larvae were soaked in the dsRNA solutions for 24 hours at 27° C., and were then transferred into new containers (200 larvae/1000 mL of chlorine-free tap water), which were also maintained at 27° C., and provided 6 mg/mL lab dog/cat diet (Purina Mills) suspended in water as a source of food on a daily basis. After soaking procedure, the larvae were reared until third instar and divided in 6 replicas with 10-20 larvae in each cup (final volume of 100 mL) or were divided immediately after soaking. Six replicas were treated with the lethal concentration 50 (0.00573 ppm) of temephos and 2 replicas with ethanol only (control group). Mortality was recorded after 24 and 48 hours. See Flowcharts 1 and 2 (FIGS. 1 and 2, respectively).

2. Larvae Feeding with Food-Containing dsRNA and Bioassay with Diflubenzuron

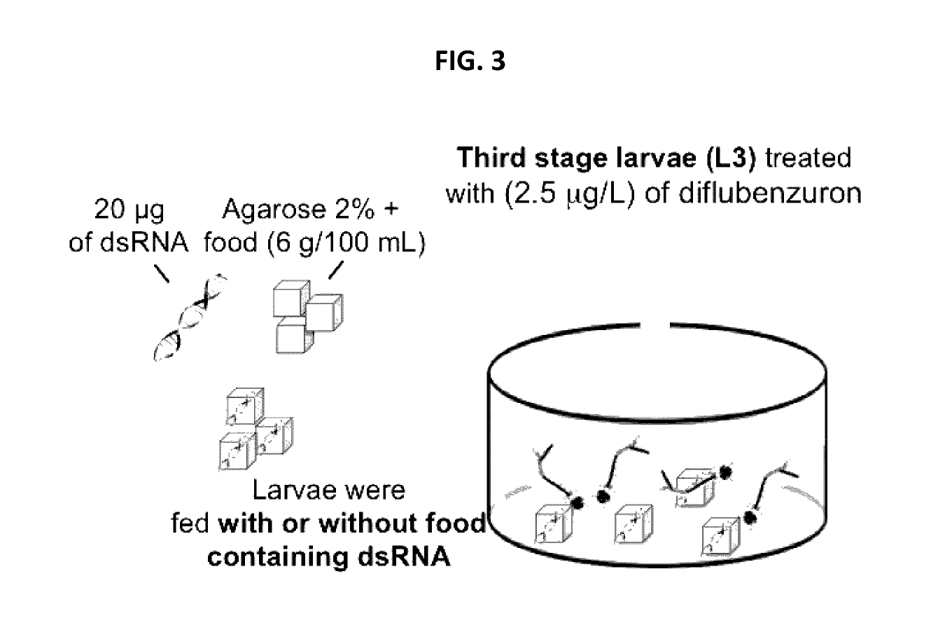

Third instar larvae (in groups of 10 larvae) were exposed to one concentration (2.5 μg/L) of diflubenzuron pestanal (Sigma) in a final volume of 100 mL of chlorine-free tap water. From the beginning of diflubenzuron treatment, larvae were fed with agarose cubes containing 20 μg of dsRNA once a day for a total of 4 days. The plastic cups were covered with a nylon mesh in order to avoid adult escape. The evaluations were performed every other day by recording the mortality of the larvae and the number of emerged adults per replication as previously described [Mulla et al. (2003) J. Vect. Ecol. 28, 2:241-54]. The test was terminated when all the larvae became pupae in the control group. The inhibition of emergence (%) was calculated according to the following formula: 100-100(T/C) where T is percent emergence in treated groups and C is percent emergence in control groups [Mulla et al. (1974) Proc. Papers Calif. Mosq. Contr. Assoc. 42: 175-176]. Mortality was recorded every other day during 2 weeks. See Flowchart 3 (FIG. 3).

Preparation of Mosquito Larval Food Containing dsRNA

Cubes of dsRNA-containing mosquito food were prepared as follows: First, 20 μg of dsRNA were mixed with 10 μg of Polyethylenimine 25 kDa linear (Polysciences) in 50 μL of sterile water. Alternatively, 2 different dsRNA (20 μg of each) plus 20 μg of Polyethylenimine were mixed in 50 μL of sterile water. Then, a suspension of ground mosquito larval food (6 grams/100 mL) was prepared with 2% agarose (Fisher Scientific). The food/agarose mixture was heated to 55° C. and 50 μL of the mixture was then transferred to the tubes containing 50 μL of dsRNA+PEI or water only (control). The mixture was then allowed to solidify into a gel. The solidified gel containing both the food and dsRNA was cut into small pieces (approximately 1 mm thick) using a razor blade, which were then used to feed mosquito larvae in water.

RNA Isolation and dsRNA Production

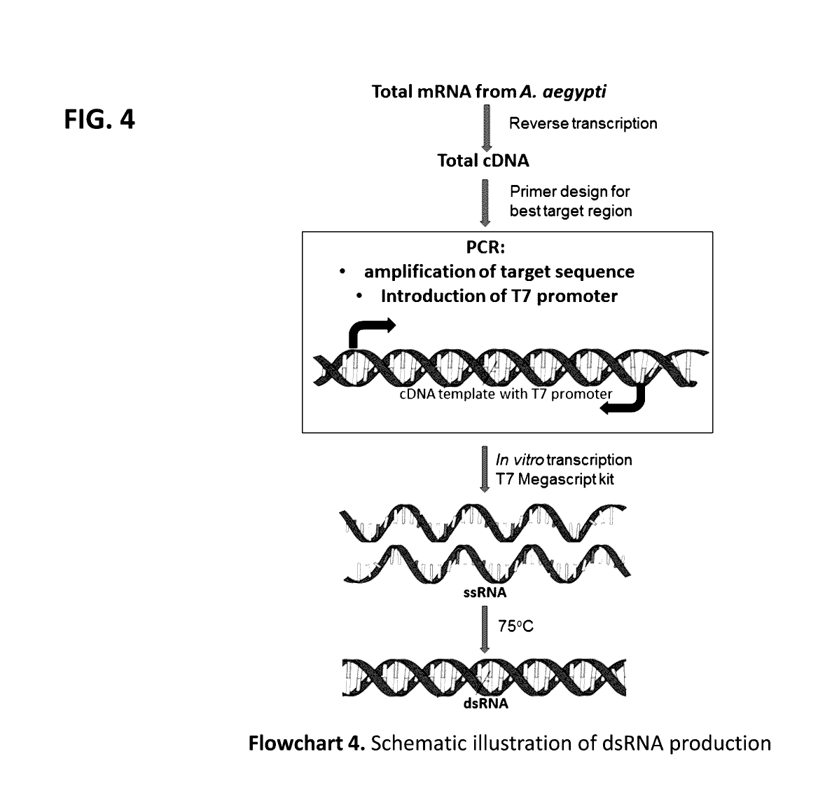

Total RNA was extracted from groups of five Ae. aegypti fourth instar larvae and early adult male/female Ae. aegypti, using TRIzol (Invitrogen, Carlsbad, Calif., USA) according to the manufacturer's instructions. RNA was treated with amplification grade DNase I (Invitrogen) and 1 μg was used to synthesize cDNA using a First Strand cDNA Synthesis kit (Invitrogen). The cDNA served as template DNA for PCR amplification of gene fragments using the primers listed in Table 2 (below). PCR products were purified using a QIAquick PCR purification kit (Qiagen). The MEGAscript RNAi kit (Ambion) was then used for in vitro transcription and purification of dsRNAs (see Flowchart 4, FIG. 4) and dsRNA sequences (Table 3, below).

TABLE 2
qPCR primers for larvicide targets

| Target gene | Accession number | qPCR primers (5′-3′) |
|---|---|---|
| P-glycoprotein | XM_001654442.1 | F: GCGCGCTCGTTCAGTATTTA |
| AAEL010379 | | (SEQ ID NO: 166) |
| | | R: ACACCCGTTACGGCACAATA |
| | | (SEQ ID NO: 167) |
| Argonaute-3 | XM_001652895.1 | F: TCGGCATTCGTAGCTTCGTT |
| AAEL007823 | | (SEQ ID NO: 168) |
| | | R: GCAGCTGACAGTTTGCCTTC |
| | | (SEQ ID NO: 169) |
| AuB | | F: CAGAATCCCAGACCCGGAAC |
| AAEL007698 | | (SEQ ID NO: 170) |
| | | R: TTGGCGAAACCGTACCTTGA |
| | | (SEQ ID NO: 171) |
| Sodium channel | XM_001653136.1 | F: CTGGAGTCGGTGAGCGAAA |
| AAEL008297 | | (SEQ ID NO: 172) |
| | | R: TACGTATCGTAAACGCGCTC |
| | | (SEQ ID NO: 173) |
| Alpha esterase | XM_001650322.1 | F: CGCCGGATGAAGATCAACTG |
| AAEL005112 | | (SEQ ID NO: 174) |
| | | R: TTTCATCGTTTTACACGAAAGTAGT |
| | | (SEQ ID NO: 175) |
| AAEL003446 | XM_001663587.1 | F: TGCAAGCTGCTGATGGAGC |
| | | (SEQ ID NO: 176) |
| | | R: TCACCAATAGTAATGAGGGTATCCA |
| | | (SEQ ID NO: 177) |
| Cytochrome p450 | XM_001652879.1 | F: CCTAGAGTTAGCCAAGGGCG |
| AAEL007815 | | (SEQ ID NO: 178) |
| | | R: CATGTCCAGGAAGGCCACTT |
| | | (SEQ ID NO: 179) |
| AAEL002202 | XM_001654908.1 | F: TCACTATCGCCATCCTTGCAT |
| | | (SEQ ID NO: 180) |
| | | R: GCCACTACATCATGGGCGTA |
| | | (SEQ ID NO: 181) |
| AAEL009124 | XM_001653624.1 | F: CCCTGTTCTCACCTCGGAAG |
| | | (SEQ ID NO: 182) |
| | | R: CGGCACTAGCCGTAAACTG |
| | | (SEQ ID NO: 183) |

TABLE 3
dsRNA sequences

| Target gene | Accession number | dsRNA sequence |
|---|---|---|
| P-glycoprotein | XM_001654442.1 | SEQ ID NO: 184 |
| AAEL010379 | | |
| Argonaute-3 | XM_001652895.1 | SEQ ID NO: 185 |
| AAEL007823 | | |
| AuB | | SEQ ID NO: 186 |
| AAEL007698 | | |
| Sodium channel | KC107440.1 | SEQ ID NO: 187 |
| AAEL008297 | XM_001653136.1 | |
| Alpha esterase | | SEQ ID NO: 188 |
| AAEL005112 | | |
| AAEL003446 | | SEQ ID NO: 189 |
| AAEL007815 | | SEQ ID NO: 190 |
| AAEL002202 | | SEQ ID NO: 191 |
| AAEL009124 | | SEQ ID NO: 192 |

qPCR Analysis

Approximately 1000 ng first-strand cDNA obtained as described previously was used as template. The qPCR reactions were performed using SYBR® Green PCR Master Mix (Applied Biosystems) following the manufacturer's instructions. Briefly, approximately 50 ng/μl cDNA and gene-specific primers (600 nM) were used for each reaction mixture. qPCR conditions used were 10 min at 95° C. followed by 35 cycles of 15 s at 94° C., 15 s at 54° C. and 60 s at 72° C. The ribosomal protein S7 and tubulin were used as the reference gene to normalize expression levels amongst the samples. Raw quantification cycle (Cq) values normalized against those of the tubulin and S7 standards were then used to calculate the relative expression levels in samples using the 2^{-ΔΔCt} method previously described [Livak & Schmittgen (2001) Methods. 25(4):402-8]. Results (mean±SD) are representative of at least two independent experiments performed in triplicate.

### Results

### Characterization of Temephos Resistance in a Field Population (Rio De Janeiro) of Aedes aegypti Mosquitoes

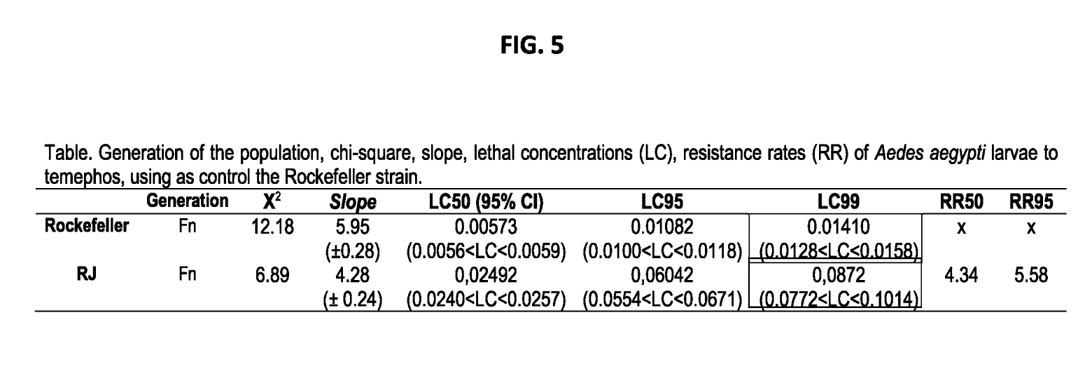

Susceptibility tests were carried out according to the methodology proposed by the World Health Organization (WHO). In order to characterize the status of Temephos resistance in Ae. aegypti (Rio de Janeiro) strain, the present inventors first determined the lethal concentration LC99 (concentration able to kill 99% of the larvae) using the susceptible strain (Rockfeller) (see FIG. 5: 0.01401 ppm). Then, bioassays with the Rio de Janeiro strain were performed using a diagnostic concentration of 0.0282 ppm of Temephos, which corresponded to twice the lethal concentration, 99% (LC99), determined for this susceptible strain. According to WHO, when the field population is treated with the diagnostic dose and the mortality rates are equal to or higher than 98%, the population is considered susceptible, between 80% and 98%, with alterations in susceptibility, and below 80%, resistant [WHO (1981) WHO/VBC/81.807]. The Rio de Janeiro field population displayed 60% mortality and was considered resistant to Temephos. The resistance ratio of the populations in Rio de Janeiro and Rockefeller was 5.58, which means that RJ is about 6 times more resistant to Temephos.

### Comparison of Gene Expression Profile Between Temephos-Resistant and Susceptible Ae. aegypti Strains

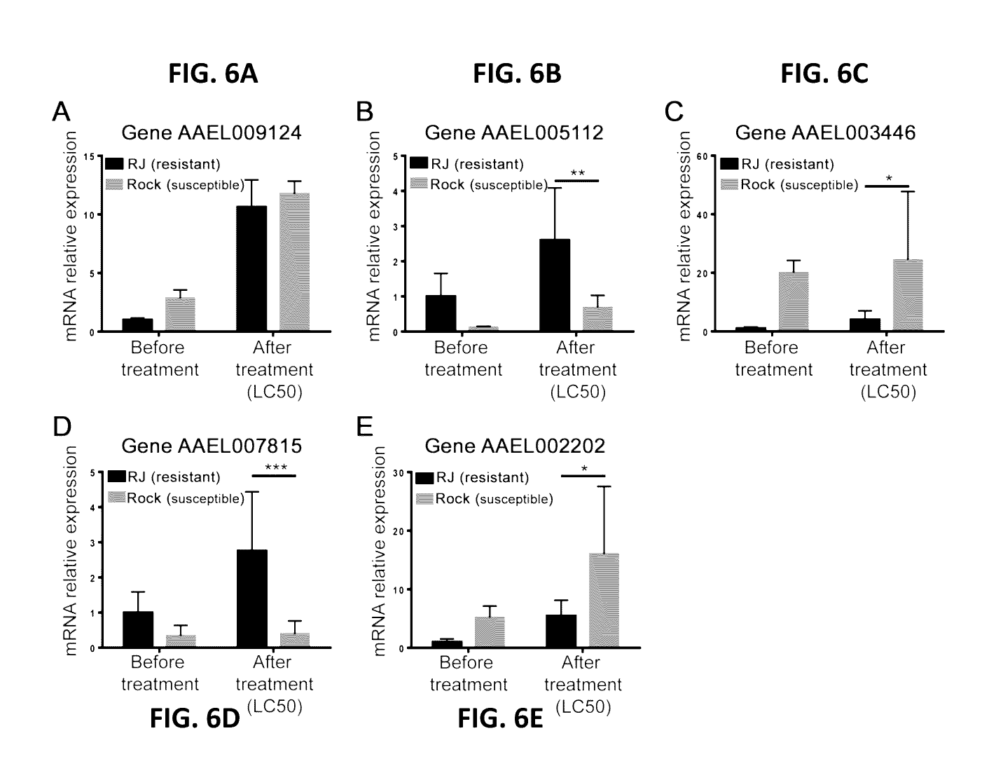

In order to compare the expression pattern of genes related to insecticide resistance, 3^{rd}-instar larvae from resistant (Rio de Janeiro—RJ) or susceptible (Rockfeller-Rock) strains were first treated with LC50 of Temephos during 6 hours. Then larvae were collected and RNA extracted for qPCR analysis. As seen in FIGS. 6A-E, five genes were differentially expressed before and after treatment with Temephos in both resistant and susceptible strains.

### Effect of dsRNA Treatment on Susceptibility of Ae. aegypti Larvae to Temephos

In the first approach, First instar larvae (Rio de Janeiro strain) were soaked with dsRNA against P-glycoprotein, Sodium channel, AAEL007815 and AAEL005112 for 24 hours. Temephos bioassay was then carried out when larvae reached Third instar stage (2-3 days after soaking). A decrease in mRNA expression level for P-glycoprotein was detected (FIG. 7).

The present inventors also tested soaking using third instar larvae for 24 hours. After soaking, larvae were immediately treated with Temephos and mortality was recorded. A reduction in the mRNA levels was detected for Sodium channel and Ago-3 after treatment with Temephos (FIGS. 8A and 8B, respectively).

Soakings tests using targets AAEL005112, AAEL003446, AAEL007815 and AAEL002202 in L1 and L3 larvae, followed by Temephos treatment as described above, were also examined (data not show).

### Effect of dsRNA Treatment on Susceptibility of Ae. aegypti Larvae to Diflubenzuron

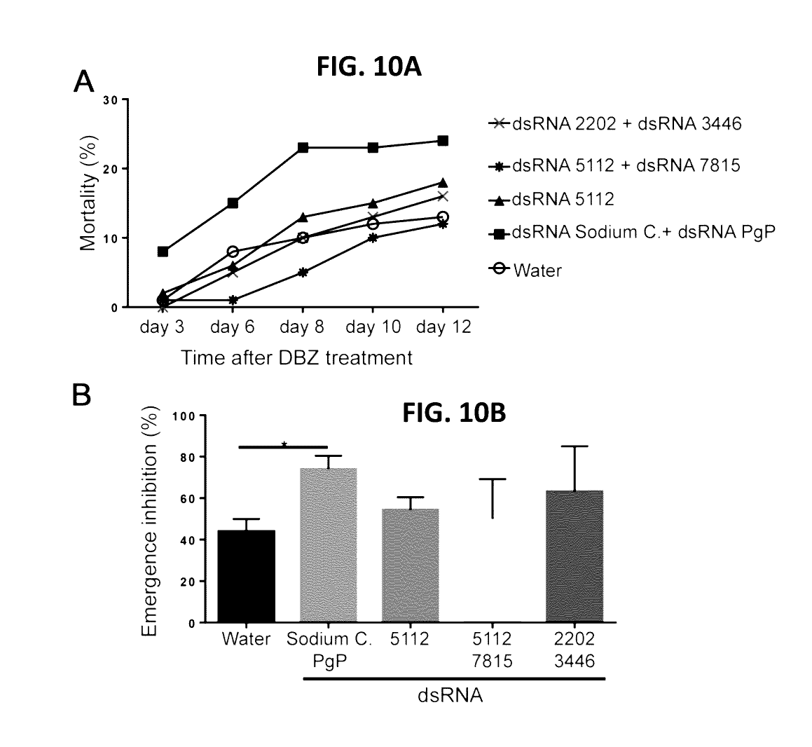

Diflubenzuron affects larval development and, therefore, induces larvae mortality by a different mechanism in comparison to Temephos. Using a different approach to delivery of dsRNA, mosquito third instar larvae were fed with dsRNA (or a mix of two dsRNAs) during treatment with Diflubenzuron. As can be seen in FIGS. 10A-B, feeding of A. aegypti larvae with Sodium channel or PgP dsRNA reduced significantly the viability of mosquito larvae 4 days after treatment with DBZ. In addition, when a combination of Sodium channel and PgP dsRNA was tested, the effect on larvae mortality and adult emergence was even more pronounced, leading to an increased susceptibility of larvae to Diflubenzuron (FIGS. 9A-B). Other dsRNA combinations were also tested but no significant results were obtained (FIGS. 9A-B).

### Example 2

### Materials and Experimental Procedures

Mosquito Maintenance

Mosquitoes were taken from an Ae. aegypti colony of the Rockefeller strain or from a mosquito field population of Ae. aegypti isolated from urban area of Rio de Janeiro, Brazil. Both lineages were reared continuously in the laboratory at 28° C. and 70-80% relative humidity. Adult mosquitoes were maintained in a 10% sucrose solution, and the adult females were fed with sheep blood for egg laying. The larvae were reared on dog/cat food unless stated otherwise.

Introducing dsRNA into a Mosquito Larvae

Three different approaches were evaluated for treatment with dsRNA:

A) Soaking with “Naked” dsRNA

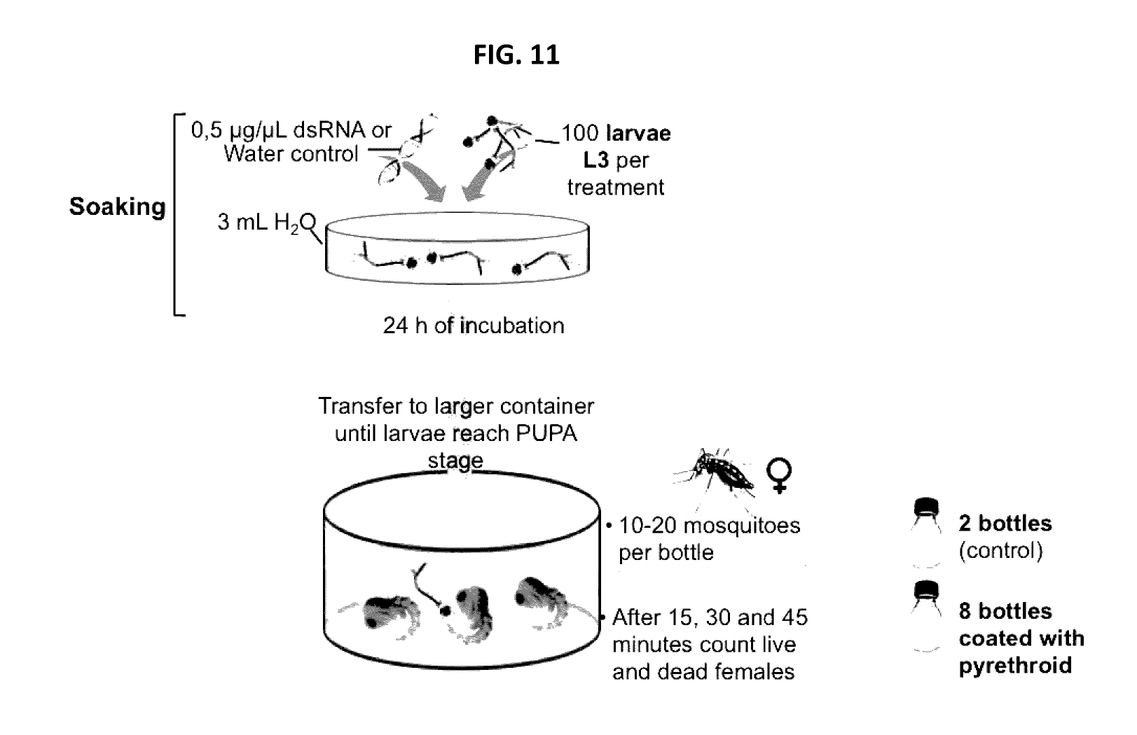

Third instar larvae were treated (in groups of 100 larvae) in a final volume of 3 mL of dsRNA solution in autoclaved water (0.5 μg/μL for sodium channel (AAEL008297), PgP (AAEL010379) and Ago3 (AAEL007823) dsRNA, or 0.1 μg/μL for CYP9J26 (JF924909.1). The control group was kept in 3 ml sterile water only. Larvae were soaked in the dsRNA solutions for 24 hr at 27° C., and then transferred into new containers (300 larvae/1500 mL of chlorine-free tap water), which were also maintained at 27° C., and were provided 6 mg/100 mL lab dog/cat diet (Purina Mills) suspended in water as a source of food on a daily basis. As pupae developed, they were transferred to individual vials to await eclosion and sex sorting. For bioassays purpose only females up to five days old were used. See FIG. 11 for detailed explanation of this experiment.

B) Soaking with “Naked” dsRNA Plus Additional Larvae Feeding with Food-Containing dsRNA

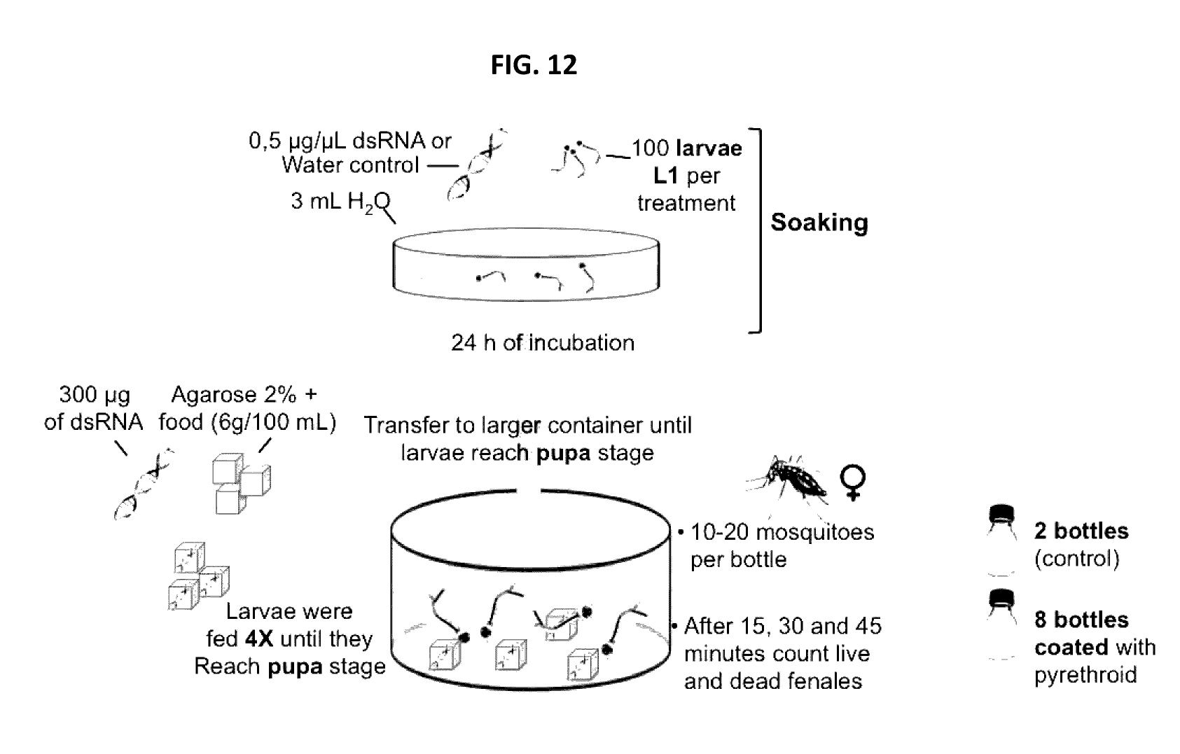

After soaking in the dsRNA solutions for 24 hr at 27° C., the larvae were transferred into new containers (300 larvae/1500 mL of chlorine-free tap water), and were provided agarose cubes containing 300 μg of dsRNA once a day for a total of four days. The larvae were reared until adult stage. For bioassays purpose only females up to five days old are used. See FIG. 12 for detailed explanation of this experiment.

C) Larvae Feeding with Food-Containing dsRNA Only

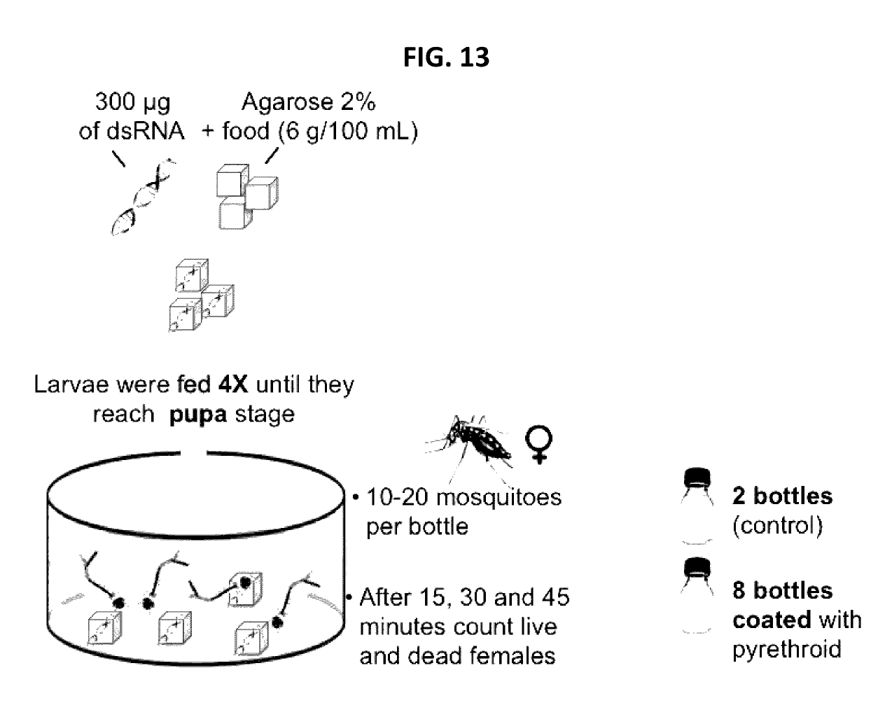

Third instar larvae were fed (in groups of 300 larvae) in a final volume of 1500 mL of chlorine-free tap water with agarose cubes containing 300 μg of dsRNA once a day for a total of four days. The larvae were reared until adult stage. For bioassays purpose only females up to five days old are used. See FIG. 13 for detailed explanation of this experiment.

Bioassay with Pyrethroid

CDC Bottle Bioassays—

Bottles were prepared following the Brogdon and McAllister (1998) protocol [Brogdon and McAllister (1998) Emerg Infect Dis 4:605-613]. Fifteen-twenty non-blood-fed females from each site were introduced in 250 mL glass bottles impregnated with different concentrations of deltamethrin (Sigma-Aldrich) in 1 ml acetone. Each test consisted of four impregnated bottles and one control bottle. The control bottle contained acetone with no insecticide. At least three tests were conducted for each insecticide and population. Immediately prior to use, all insecticide solutions were prepared fresh from stock solutions. At 15, 30 and 45 min intervals, the number of live and dead mosquitoes in each bottle was recorded. The mortality criteria included mosquitoes with difficulties flying or standing on the bottle's surface. Mosquitoes that survived the appropriate dose for insecticide were considered to be resistant [Brogdon and McAllister (1998), supra].

Preparation of Mosquito Larval Food Containing dsRNA

Cubes of dsRNA-containing mosquito food were prepared as follows: First, 300 μg of dsRNA were mixed with 30 μg of Polyethylenimine 25 kD linear (Polysciences) in 200 μL of sterile water. Then, a suspension of ground mosquito larval food (6 grams/100 mL) was prepared with 2% agarose (Fisher Scientific). The food/agarose mixture was heated to 55° C. and 200 μL of the mixture was then transferred to the tubes containing 200 μL of dsRNA+PEI or water only (control). The mixture was then allowed to solidify into a gel. The solidified gel containing both the food and dsRNA was cut into small pieces (approximately 1 mm thick) using a razor blade, which were then used to feed mosquito larvae in water.

RNA Isolation and dsRNA Production

Total RNA was extracted from groups of five Ac. aegypti fourth instar larvae and early adult male/female Ac. aegypti, using TRIzol (Invitrogen, Carlsbad, Calif., USA) according to the manufacturer's instructions. RNA was treated with amplification grade DNase I (Invitrogen) and 1 μg was used to synthesize cDNA using a First Strand cDNA Synthesis kit (Invitrogen). The cDNA served as template DNA for PCR amplification of gene fragments using the primers listed in Table 4, below. PCR products were purified using a QIAquick PCR purification kit (Qiagen). The MEGAscript RNAi kit (Ambion) was then used for in vitro transcription and purification of dsRNAs. See Flowchart 4, FIG. 4 for detailed explanation production off dsRNA.

TABLE 4
qPCR primers and dsRNA sequences for adulticide targets

| | Accession | dsRNA | |
|---|---|---|---|
| Target gene | number | sequence | qPCR primers (5′-3′) |
| P-glycoprotein | XM_001654442.1 | SEQ ID NO: | F: GCGCGCTCGTTCAGTATTTA |
| (AAEL010379) | | 184 | (SEQ ID NO: 166) |
| | | | R: ACACCCGTTACGGCACAATA |
| | | | (SEQ ID NO: 167) |
| Argonaute-3 | XM_001652895.1 | SEQ ID NO: | F: TCGGCATTCGTAGCTTCGTT |
| (AAEL007823) | | 185 | (SEQ ID NO: 168) |
| | | | R: GCAGCTGACAGTTTGCCTTC |
| | | | (SEQ ID NO: 169) |
| Cytochrome p450 | JF924909.1 | SEQ ID NO: | F: CCGTTTGGTATCGGCCCAAG |
| (CYP9J26) | XM_001649047.2 | 203 | (SEQ ID NO: 204) |
| JF924909.1 | | | R: GTCTTTGCGCCTCGGACG |
| | | | (SEQ ID NO: 205) |
| Sodium channel | KC107440.1 | SEQ ID NO: | F: CTGGAGTCGGTGAGCGAAA |
| (AAEL008297) | XM_001653136.1 | 187 | (SEQ ID NO: 172) |
| | | | R: TACGTATCGTAAACGCGCTC |
| | | | (SEQ ID NO: 173) |

qPCR Analysis

Approximately 1000 ng first-strand cDNA obtained as described previously was used as template. The qPCR reactions were performed using SYBR® Green PCR Master Mix (Applied Biosystems) following the manufacturer's instructions. Briefly, approximately 50 cDNA and gene-specific primers (600 nM) were used for each reaction mixture. qPCR conditions used were 10 min at 95° C. followed by 35 cycles of 15 s at 94° C., 15 s at 54° C. and 60 s at 72° C. The ribosomal protein S7 and tubulin were used as the reference gene to normalize expression levels amongst the samples. Raw quantification cycle (Cq) values normalized against those of the tubulin and S7 standards were then used to calculate the relative expression levels in samples using the 2^{-ΔΔCt} method [Livak & Schmittgen, (2001) Methods. 25(4):402-8.). Results (mean±SD) are representative of at least two independent experiments performed in triplicate.

### Results

### Characterization of Insecticide Resistance Using Two Different Strains of Aedes aegypti Mosquitoes

Vector control strategies employed for Aedes control are mainly anti-larval measures, source reduction and use of adulticides (pyrethroids). Pyrethroids are a major class of insecticides, which show low mammalian toxicity and fast knockdown activity. Unfortunately, the intensive use of pyrethroids, including their indirect use in agriculture, has led to reports of reduced efficacy. One of the mechanisms of resistance in insects against pyrethroids is knockdown resistance (kdr) which is conferred by mutation(s) in the target site, the voltage gated sodium channel (VGSC). Several kdr mutations have been reported in many insects of agricultural and medical importance including Ae. aegypti. In Ae. Aegypti, eleven non-synonymous mutations at nine different loci have been reported [Med Vet Ent 17: 87-94; Insect Mol Biol 16: 785-798; Insect Biochem Mol Biol 39: 272-278], amongst which mutations at three loci, i.e., Iso1011 (IRM/V) and Val1016 (VRG/I) in domain II and F1534 (FRC) in domain III are most commonly reported as contributing to pyrethroid resistance.

Using a population of mosquitoes that shows increased pyrethroid resistance, the present inventors target (during larval stage) several genes associated with resistance to pyrethroid in order to break resistance to insecticide at the adult stage.

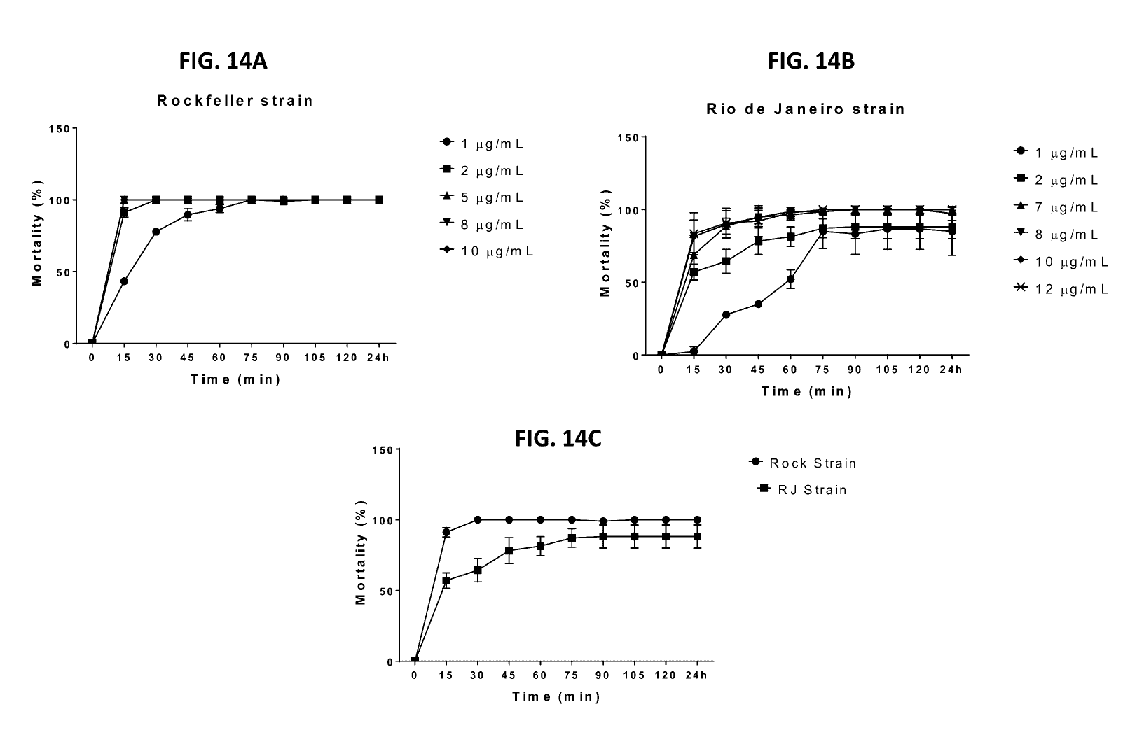

A diagnostic dosage (DD) was established for the insecticide using the Rockfeller reference susceptible Ae. aegypti strain and a resistance threshold (RT), time in which 98-100% mortality was observed in the Rockfeller strain, was then calculated. Using the DD (2 μg/mL of deltamethrin) (FIGS. 14A-C) is was possible to demonstrate that this dose killed only 63.95% of the Rio de Janeiro strain whereas 100% of the mosquitoes from the Rockfeller strain were dead. Therefore, it was concluded that 36.05% of the mosquitoes in this population (RJ) are resistant to deltamethrin.

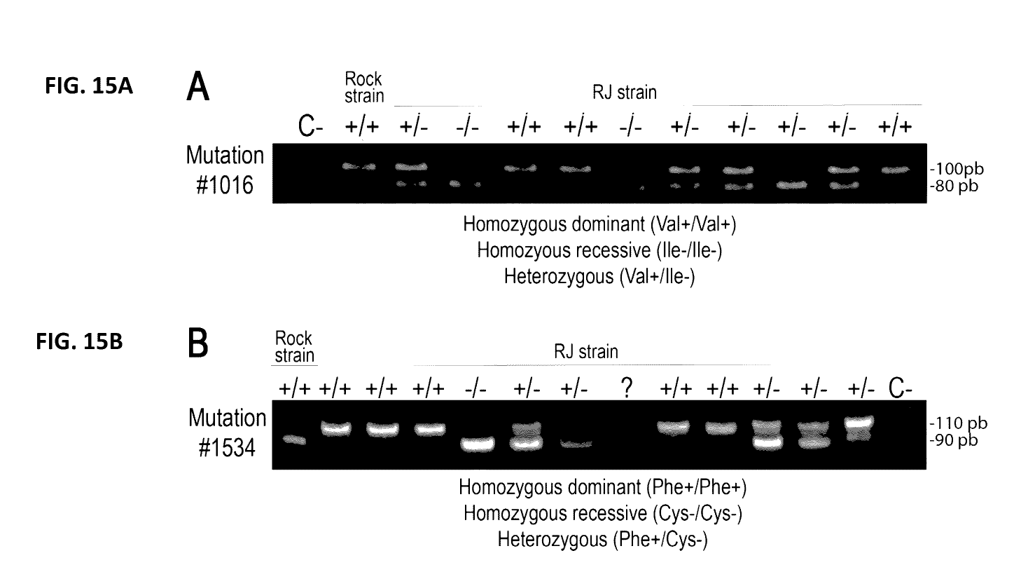

To further confirm the resistance status of the Rio de Janeiro strain, the kdr mutations reported as contributing to pyrethroid resistance were assessed. In FIGS. 15A-B, the present inventors show that V1016G and F1534C were both detected in the RJ strain. Indeed, the V1016G and F1534C mutation were detected in 49% and 60% of the mosquitoes from Rio de Janeiro strain, respectively.

### Silencing of Sodium Channel During Larval Development Increases the Susceptibility of Adult Mosquitoes to Pyrethroid

Using the first approach (soaking with “naked” dsRNA), mosquito larvae (RJ strain) were treated with three different dsRNA: Ago3, P-glycoprotein and Sodium channel. Treatment with dsRNA against sodium channel increased substantially the susceptibility of mosquitoes to the insecticide (FIG. 16A). Interestingly, female mosquitoes showed a decreased expression in the mRNA level for sodium channel before deltamethrin treatment (FIG. 16B). When compared to water-treated mosquitoes only, dead female mosquitoes previously treated with dsRNA showed a striking decrease in mRNA expression level for sodium channel (FIG. 16C).

In order to test the second approach (soaking with “naked” dsRNA plus additional larvae feeding with food-containing dsRNA), mosquito larvae (L3) were first soaked with dsRNA (sodium channel, 0.5 μg/μL) for 24 hours. Then, larvae were treated 4 times with food-containing dsRNA and reared until adult stage. Although there was no obvious advantage in using this approach when compared to soaking with naked dsRNA alone, treatment with dsRNA against sodium channel increased the susceptibility of mosquitoes to deltamethrin (FIG. 17).

This approach was also tested using dsRNA to target Cytochrome p450 (CYP9J26). As can be seen in the FIG. 18, dsRNA-treated mosquitoes were more sensitive to deltamethrin during the first 15 minutes of contact with deltamethrin.

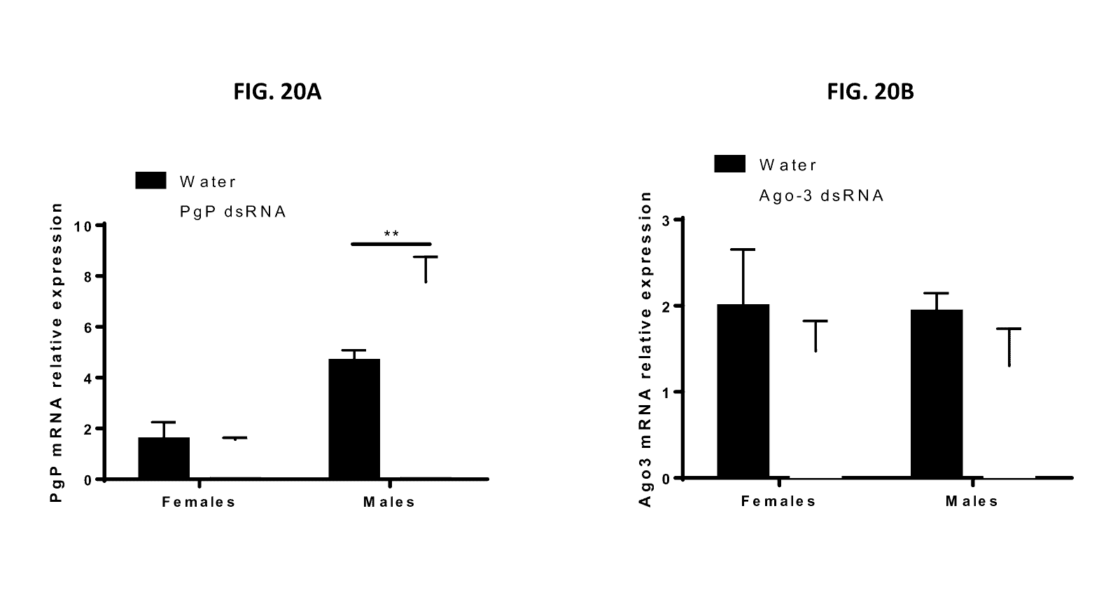

It is important to note that that 24 and 48 hours after the end of dsRNA treatment, decreased mRNA levels were detected in mosquito adults that were treated with PgP, Ago3 or sodium channel dsRNA as larvae (FIGS. 19A-C). However, PgP and Ago3 mRNA expression reached normal levels when mosquitoes became adults (FIG. 20A-B, respectively).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of enhancing larvicide susceptibility in a mosquito larva, the method comprising introducing into the mosquito larva an isolated nucleic acid agent comprising a nucleic acid sequence which specifically reduces the expression of at least one larvicide resistance gene product of the larva, thereby enhancing larvicide susceptibility in said mosquito larva.

2. A method of enhancing larvicide and/or adulticide susceptibility in a mosquito, the method comprising introducing into a mosquito larva an isolated nucleic acid agent comprising a nucleic acid sequence which specifically reduces the expression of at least one larvicide and/or adulticide resistance gene product of the mosquito, thereby enhancing larvicide and/or adulticide susceptibility in the mosquito when a pupa or an adult mosquito.

3. The method of claim 1, wherein said larvicide resistance gene is selected from the group consisting of SEQ ID NOs: 1-111, 114-165, 193-202.

4. The method of claim 1, wherein said larvicide resistance gene is selected from the genes in Tables 1A-B.

5. The method of claim 1, wherein said enhanced larvicide susceptibility is enhanced as compared to identical mosquito larva not receiving said isolated nucleic acid agent and optionally wherein said enhanced susceptibility is expressed as reduced LD₅₀ for said larvicide.

6. (canceled)

7. The method of claim 1, wherein said mosquito is a mosquito capable of transmitting a disease to a mammalian organism.

8-9. (canceled)

10. The method of claim 1, wherein the mosquito larvae are of the genus Aedes.

11-13. (canceled)

14. The method of claim 1, wherein said introducing comprises feeding, spraying, soaking or injecting.

15. The method of claim 1, wherein said introducing comprises soaking said larva with said isolated nucleic acid agent for about 12-48 hours.

16. (canceled)

17. The method of claim 1, wherein said introducing comprises feeding said larva with said isolated nucleic acid agent for about 48-96 hours.

18. The method of claim 1, wherein said mosquito larva carries an infection selected from the group consisting of a viral infection, a nematode infection, a protozoa infection and a bacterial infection.

19. An isolated nucleic acid agent comprising a nucleic acid sequence which specifically reduces the expression of at least one mosquito larvicide resistance gene product of a mosquito larva.

20. The isolated nucleic acid agent of claim 19, wherein said larvicide resistance gene is selected from the group consisting of the genes in Tables 1A-B.

21. The isolated nucleic acid agent of claim 19, wherein said larvicide resistance gene is selected from the group consisting of AAEL013279 (Seq ID NO: 16); AAEL001626 (Seq ID NO: 28); AAEL005772 (Seq ID NO: 40); AAEL012357 (Seq ID NO: 112), AAEL014445 (Seq ID NO: 102), AAEL008297 (Seq ID NO: 193), AAEL010379 (Seq ID NO: 194), AAEL007823 (Seq ID NO: 195), AAEL007698 (Seq ID NO: 196), AAEL005112 (Seq ID NO: 197), AAEL003446 (Seq ID NO: 198), AAEL007815 (Seq ID NO: 199), AAEL002202 (Seq ID NO: 200), AAEL009124 (Seq ID NO: 201) and Cytochrome p450 (CYP9J26) (Seq ID NO: 202).

22. The isolated nucleic acid agent of claim 19, wherein said larvicide resistance gene is selected from the group consisting of AAEL008297 (Seq ID NO: 193), AAEL010379 (Seq ID NO: 194), AAEL007823 (Seq ID NO: 195), AAEL007698 (Seq ID NO: 196), AAEL005112 (Seq ID NO: 197), AAEL003446 (Seq ID NO: 198), AAEL007815 (Seq ID NO: 199), AAEL002202 (Seq ID NO: 200), AAEL009124 (Seq ID NO: 201) and Cytochrome p450 (CYP9J26) (Seq ID NO: 202).

23. The isolated nucleic acid agent of claim 19, wherein said nucleic acid sequence reduces the expression of two mosquito larvicide resistance genes.

24. The isolated nucleic acid agent of claim 23, wherein said two mosquito larvicide resistance genes comprise a sodium channel gene and a P-glycoprotein gene.

25. A composition comprising at least one nucleic acid agent which specifically reduces the expression of two mosquito larvicide resistance genes.

26. The isolated nucleic acid agent of claim 24, wherein said larvicide resistance gene comprise a sodium channel gene as set forth in SEQ ID NO: 193 and a P-glycoprotein gene as set forth in SEQ ID NO: 194.

27. The isolated nucleic acid agent of claim 26, wherein said nucleic acid agent is a dsRNA comprising SEQ ID NO: 187 and a dsRNA SEQ ID NO: 184.

28. The isolated nucleic acid agent of claim 19, wherein said isolated nucleic acid agent is a dsRNA.

29. (canceled)

30. The isolated nucleic acid agent of claim 28, wherein said dsRNA is effected at a dose of 0.001-1 μg/μL for soaking or at a dose of 1 pg to 10 μg/larvae for feeding.

31. The isolated nucleic acid agent of claim 28, wherein said dsRNA is naked dsRNA.

32. The isolated nucleic acid agent of claim 28, wherein said dsRNA comprises a carrier.

33. The isolated nucleic acid agent of claim 32, wherein said carrier comprises a Polyethylenimine (PEI).

34-38. (canceled)

39. A nucleic acid construct comprising a nucleic acid sequence encoding the isolated nucleic acid agent of claim 19.

40. The nucleic acid construct of claim 39, further comprising a regulatory element active in plant cells.

41. A cell of a mosquito larva ingestible organism comprising the isolated nucleic acid agent of claim 19.

42. The cell of claim 41, wherein said mosquito-larva-ingestible organism is an algae.

43. (canceled)

44. A composition comprising the cell of claim 41 and an insecticidally acceptable carrier.

45. (canceled)

46. The composition of claim 44, wherein said composition is formulated in a semi-solid form.

47. The composition of claim 46, wherein said semi-solid form comprises agarose.

48. The composition of claim 44, further comprising a mosquito larvicide and/or adulticide.

49-50. (canceled)

51. The composition of claim 44, further comprising mosquito larva feed.

52. The method of claim 1, wherein said isolated nucleic acid agent further comprises a cell penetrating agent.

53. A solution comprising the cell of claim 41 for soaking mosquito larvae.

54. A mosquito or mosquito larva comprising at least one exogenous isolated nucleic acid agent comprising a nucleic acid sequence which specifically reduces the expression of at least mosquito larvicide resistance gene product.

55-58. (canceled)

59. The method of claim 1, wherein said isolated nucleic acid agent is comprised in a cell.

60. The method of claim 2, wherein said isolated nucleic acid agent is comprised in a cell.
